(19) United States
(12) Patent Application Publication
(10) Pub. No.: US 2025/0319056 A1
(43) Pub. Date: Oct. 16, 2025

(54) METHODS FOR TREATING CANCER

(71) Applicant: THE TRUSTEES OF INDIANA UNIVERSITY, Bloomington, IN (US)

(72) Inventor: Steven Michael JOHNSON, Indianapolis, IN (US)

(21) Appl. No.: 19/175,233

(22) Filed: Apr. 10, 2025

Related U.S. Application Data
(60) Provisional application No. 63/632,385, filed on Apr. 10, 2024.
(60) Provisional application No. 63/636,733, filed on Apr. 20, 2024.

(51) Int. Cl. A61K 31/341; A61K 31/345; A61K 31/423; A61K 31/428; A61K 31/47; A61P 35/00

(52) U.S. Cl. CPC A61K 31/341; A61K 31/345; A61K 31/423; A61K 31/428; A61K 31/47; A61P 35/00

(57) ABSTRACT

The present disclosure relates to HSP60 inhibitor compounds, pharmaceutical compositions containing the compounds, and methods of using such compounds to treat cancer.

### RELATED APPLICATIONS

This application claims the benefit of U.S. Provisional Application Nos. 63/636,733, filed Apr. 20, 2024, and 63/632,385, filed Apr. 10, 2024, the entire disclosure of each of which is incorporated herein by reference.

### BACKGROUND

According to Center for Disease Control and Prevention (CDC) data for leading causes of premature death in the United States in 2021, cancer is the second highest cause of premature death (605,213 individuals), the highest being cardiovascular diseases (695,547 individuals) and the third highest being COVID-19 (416,893 individuals), with many other health conditions and diseases coming in just under those. The incidence of cancer throughout the world is rapidly growing, with mortality steadily increasing over the last 20 years. The number of new cancer cases in the United States in 2022 is estimated to be ˜1.9 million, with the highest being breast (290,560 cases), second highest being lung and bronchus (236,740 cases), and third highest being colorectal cancer (151,030 cases). The 5-year survival rate for an individual that may have any of these cancers depends on the cancer stage at which the individual is diagnosed. For breast cancer, if an individual is diagnosed at stage I, which is when the cancer is localized, the 5-year survival rate is 90%; however, if an individual is diagnosed at stage IV, which is when the cancer has metastasized, the 5-year survival rate is just 15%. For lung and bronchus cancer, the 5-year survival rate if the patient is diagnosed at stage I is 60%; however, it is not common to be diagnosed with lung cancer at an early, localized stage. The 5-year survival rate if the patient is diagnosed at stage IV, is 9%. And for colorectal cancer, the 5-year survival rate if diagnosed at stage I is 92%, whereas if the cancer is diagnosed at stage IV, the 5-year survival rate is 14%. Given that the overall 5-year survival rates are so poor for later stages of all cancers, and even early stages of some cancers, there is a need to develop new treatment options to improve patient outcomes.

In the pursuit of new anticancer targets and therapeutics, significant efforts have been invested studying molecular chaperones, also known as heat shock proteins (HSPs). HSPs function to help newly translated and/or unfolded proteins fold to their native, functional states, or assist in targeting them for degradation. HSPs are given their names based on their molecular weight (in kDa) and are divided into five primary families: HSP100, HSP90, HSP70, HSP60, and small HSPs known as sHSP's/α-crystallins. HSPs are often mis-regulated in many different cancers, which aid tumor cells in their differentiation, survival, proliferation, metastasis, invasion, and even being able to be recognized by the immune system. Due to their involvement in tumorigenesis, HSPs have become prime targets for cancer therapeutics. Several HSP inhibitors have been developed within recent decades, primarily against the HSP90 and HSP70 systems, including 17AAG, an HSP90 inhibitor; cmHSP70.1, an HSP70 antibody that can induce antibody-dependent cellular cytotoxicity (ADCC) of tumor cells; and KNK423, an HSP105, HSP70, and HSP40 inhibitor [7-9]. While some agents have progressed into clinical trials, no HSP-targeting chemotherapeutic candidate has yet achieved FDA approval.

HSP90 is a molecular chaperone that aids in the protection of cells against harmful stimuli and functions in the regulation of cell growth and apoptosis via aiding in the folding of at least 200 client proteins that are a part of many signaling pathways. HSP90 has four main isoforms in humans that are located in different cellular compartments: HSP90a and HSP900 are located in the cytoplasm; glucose related protein 94 (GRP94) is located in the endoplasmic reticulum; and tumor necrosis factor (TNF) receptor-associated protein 1 (TRAP1) is located in the mitochondria. In cancer, HSP90 client proteins are shown to play important roles in the proliferation and survival of several different types of cancers, so developing inhibitors that would prevent HSP90's function in cancer cell proliferation is desired. 17AAG, Tanespimycin, and Ganetespib are some of the main HSP90 inhibitors investigated for treating cancers like multiple myeloma, lung adenocarcinoma, and lymphoblastic leukemia. However, these inhibitors have shown to be unsuccessful during clinical trials due to their toxicity to non-cancerous cells and upregulation of the heat-shock response, increasing the expression levels of other HSPs, like HSP70, to compensate for the inhibition of HSP90. While there are inhibitors being developed for HSP70, phase I clinical data shows that there was no response in NSCLC cells or colon cancer cells when treated with inhibitor. Despite these challenges, the strategy of targeting molecular chaperones within the protein homeostasis network continues to hold substantial therapeutic promise. However, success may necessitate broadening the scope of investigation to include other molecular chaperones as potential targets. Notably, there has been little effort to develop inhibitors targeting the HSP60 chaperonin system, despite accruing evidence that underscores its fundamental role in cancer progression.

### SUMMARY

In one aspect, the disclosure relates to treating cancer with an effective amount of an antibacterial compound or an antiparasitic compound.

In one aspect, the disclosure relates to a compound or a pharmaceutically acceptable salt thereof, having the formula I,

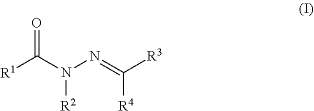

wherein
R¹ is aryl or heteroaryl, wherein aryl or heteroaryl is optionally substituted with halogen, —OR⁵, —NR⁵R⁶, —S(O)₂NR⁵R⁶, or —N(R⁵)SO₂R⁶;
R² is hydrogen;
or R¹ and R² combine with the atoms to which they are attached to form a 3- to 7-membered heterocyclyl;
R³ is aryl or heteroaryl, wherein aryl or heteroaryl is optionally substituted by —OR⁵ or nitro;
R⁴ is hydrogen;
R⁵ and R⁶ are each independently hydrogen, alkyl, aryl, or heteroaryl, wherein each hydrogen atom in alkyl, aryl, and heteroaryl is optionally substituted with halogen or —O alkyl.

In another aspect, the disclosure relates to a compound or a pharmaceutically acceptable salt thereof, having the formula II

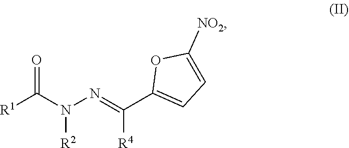

wherein each of R¹, R², and R⁴ is as described herein.

In another aspect, the disclosure relates to a compound or a pharmaceutically acceptable salt thereof, having the formula III,

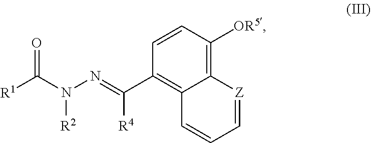

wherein each of R¹, R², R⁴, R^{5′}, and Z is as described herein.

In another aspect, the disclosure relates to a compound or a pharmaceutically acceptable salt thereof, having the formula IV

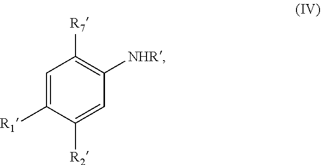

wherein
R′ is C₁-C₆ alkyl or

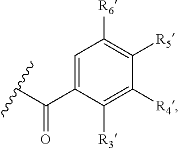

R₁′ is H, —OH, —OC₁-C₆ alkyl, —NHC(O)C₁-C₆ alkyl, —C(O)OC₁-C₆ alkyl, —C(O)OH, C₁-C₆ alkyl, —S-heteroaryl, or

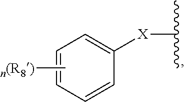

wherein each hydrogen atom in C₁-C₆ alkyl is optionally substituted by —CN,
R₂′ is H or halo,
each of R₃′, R₄′, R₅′, and R₆′ is independently H, —OH, halo, —O—C₁-C₆ alkyl, —NO₂, or —NH₂,
R₇′ is H or C₁-C₆ alkyl,
X is —O—, —S—, —C(R₉′)(R₁₀′)ₘ—, or —C(R₉′)(R₁₀′)ₘO—, optionally X is —O—, —S—, or —C(R₉′)(CN);
R₈′ is halo,
R₉′ is H or —CN,
R₁₀′ is H or —CN,
m is 1 or 2, and
n is 0, 1, or 2.

In another aspect, the disclosure relates to a compound or a pharmaceutically acceptable salt thereof, having the formula V

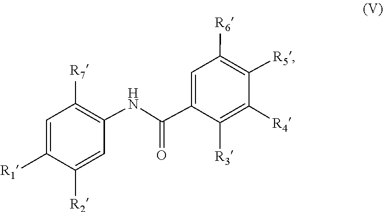

wherein each of R₁′, R₂′, R₃′, R₄′, R₅′, R₆′, and R₇′ is as described herein.

In another aspect, the disclosure relates to a compound or a pharmaceutically acceptable salt thereof, having the formula VI

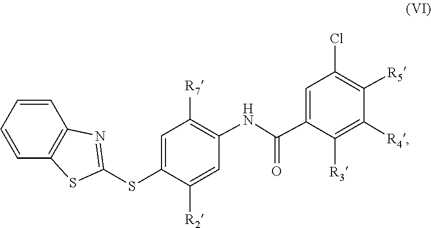

wherein each of R₂′, R₃′, R₄′, R₅′, and R₇′ is as described herein.

In another aspect, the disclosure relates to a compound or a pharmaceutically acceptable salt thereof, having the formula VII

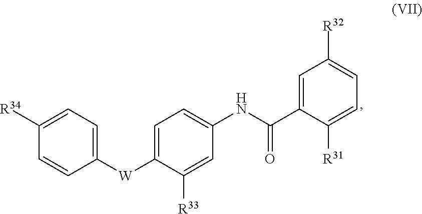

wherein each of R³¹, R³², R³³, R³⁴, and W is as described herein.

In another aspect, the disclosure relates to a compound or a pharmaceutically acceptable salt thereof, having the formula VIII

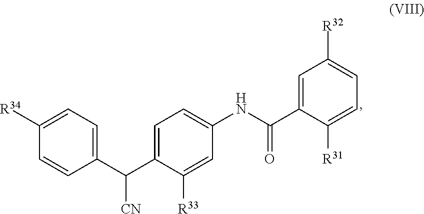

wherein each of R³¹, R³², R³³, and R³⁴ is as described herein.

In another aspect, the disclosure relates to a compound or a pharmaceutically acceptable salt thereof, having the formula IX:

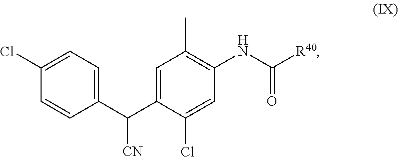

wherein R⁴⁰ is as described herein.

In another aspect, the disclosure relates to a compound or a pharmaceutically acceptable salt thereof, having the formula X,

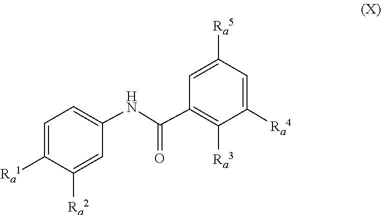

wherein
Rₐ¹ is H or

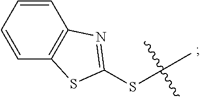 [SMILES: *Sc1nc2ccccc2s1]

Rₐ² is H or Cl;
Rₐ³ is H, OH, or OCH₃;
Rₐ⁴ is H or Br; and
Rₐ⁵ is H or Br;
provided that at least one of Rₐ¹, Rₐ², Rₐ³, Rₐ⁴, and Rₐ⁵ is not H.

In another aspect, the disclosure relates to a compound or a pharmaceutically acceptable salt thereof, having the formula XI,

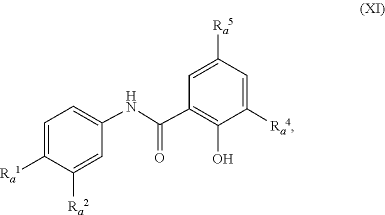

wherein each of Rₐ¹, Rₐ², Rₐ⁴, and Rₐ⁵ is as described herein.

In another aspect, the disclosure relates to a compound or a pharmaceutically acceptable salt thereof, having the formula XII, or a pharmaceutically acceptable salt thereof,

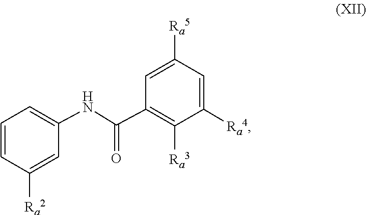

wherein each of Rₐ², Rₐ³, Rₐ⁴, and Rₐ⁵ is as described herein.

In another aspect, the disclosure relates to a compound or a pharmaceutically acceptable salt thereof, having the formula (XIII) or (XIV)

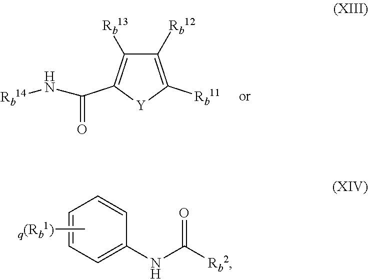

wherein:
Y is O or S;
R_{b}¹ is halo, C₁-C₈ alkyl, aryl (e.g., C₆-C₁₀ aryl or biaryl), 5- to 12-membered heteroaryl, phenoxy, benzyl, —C(O)R^{A}, —OR^{A}, —SR^{A} or —NHR^{A}, or one or more R_{b}¹ in combination with the atoms to which each is attached combine to form a C₉-C₁₄ aryl or C₉-C₁₂ bicyclic heteroaryl, wherein C₆-C₁₀ aryl, 5- to 12-membered heteroaryl, phenoxy, benzyl, C₁-C₈alkyl, —C(O)C₂-C₆ alkenylene-phenyl, C₉-C₁₂ aryl and C₉-C₁₂ bicyclic heteroaryl is optionally substituted by halo, cyano, or —NO₂;
R_{b}² is C₆-C₁₀ aryl or 5- to 12-membered heteroaryl, wherein C₆-C₁₀ aryl and 5- to 12-membered heteroaryl is optionally substituted by halo, hydroxy, or —NO₂;
R_{b}¹¹ is H, nitro, or N(H)COR_{b}³;
R_{b}¹² is H or C(O)OC₁-C₆ alkyl;
R_{b}¹³ is H or C₁-C₆ alkyl;
R_{b}¹⁴ is H, C₆-C₁₀ aryl, or 5 to 12-membered heteroaryl, wherein C₆-C₁₀ aryl and 5 to 12-membered heteroaryl is optionally substituted by halo, 5 to 12-membered heteroaryl, —NHC(O)C₁-C₆ alkyl, C₁-C₆ alkoxy, —C(O)C₁-C₆ alkoxy, —R^{A}, —OR^{A}, —SR^{A}, —C(O)R^{A} or —NHR^{A};
each R^{A} is independently C₁-C₆ alkyl, C₆-C₁₀ aryl, C₂-C₆ alkenyl-C₆-C₁₀ aryl, —C(O)C₁-C₆ alkyl, C₁-C₆ alkoxy, or 5 to 12-membered heteroaryl, wherein C₁-C₆ alkyl, C₆-C₁₀ aryl, C₂-C₆ alkenyl-C₆-C₁₀ aryl, —C(O)C₁-C₆ alkyl, C₁-C₆ alkoxy, or 5 to 12-membered heteroaryl is optionally substituted by nitro or halo; or two R^{A} combine to form C₄-C₆ cycloalkyl optionally substituted by phenyl; and
R_{b}³ is C₆-C₁₀ aryl, 5 to 12-membered heteroaryl, or C₉-C₁₂ bicyclic aryl, wherein C₆-C₁₀ aryl, 5 to 12-membered heteroaryl, and C₉-C₁₂ bicyclic aryl is optionally substituted by halo, C₁-C₆alkyl, or nitro; and
q is 0, 1, 2, or 3.

In another aspect, the disclosure relates to a compound or a pharmaceutically acceptable salt thereof, having the formula (XV) or (XVI)

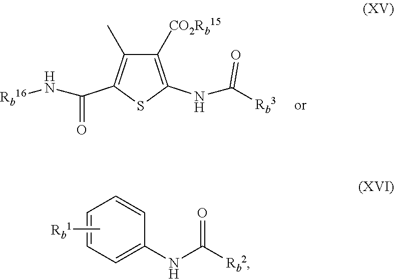

wherein each of R_{b}¹, R_{b}², R_{b}³, R_{b}¹⁵, and R_{b}¹⁶ is as described herein.

In another aspect, the disclosure relates to a compound or a pharmaceutically acceptable salt thereof, having the formula (XVII),

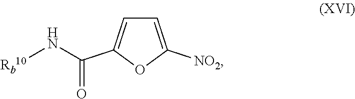

wherein R_{b}¹⁰ is as described herein.

In another aspect, the disclosure relates to a compound or a pharmaceutically acceptable salt thereof, having the formula (XVIII),

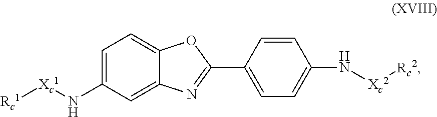

wherein
each of X_{c}¹ and X_{c}² is independently SO₂ or CO; and
each of R_{c}¹ and R_{c}² is independently alkyl, haloalkyl, aryl, or heteroaryl, wherein alkyl, aryl, and heteroaryl are optionally substituted with halogen, hydroxy, —NO₂, amino, —CN, alkyl, aryl, haloalkyl, alkoxy, C(O)OH, or C(O)O-alkyl.

In another aspect, the disclosure relates to a compound or a pharmaceutically acceptable salt thereof, having the formula (XIX),

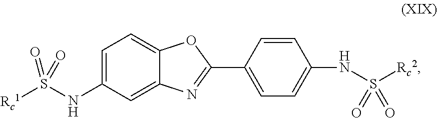

wherein each of R_{c}¹ and R_{c}² is as described herein.

In certain aspects, the present disclosure provides a pharmaceutical composition comprising a compound of the disclosure and a pharmaceutically acceptable excipient.

In certain aspects, the present disclosure provides a method of treating cancer in a patient, comprising administering to a patient in need thereof a compound or composition of the disclosure.

### BRIEF DESCRIPTION OF THE FIGURES

FIG. 1 shows the structures of compounds 1-22 of the chaperonin inhibitor diversity subset from the BMCL screening inhibitor hits.

FIG. 2A shows the structures of compounds 23-42 of the chaperonin inhibitor diversity subset from the LOPAC screening inhibitor hits.

FIG. 2B shows the structures of compounds 43-60 of the chaperonin inhibitor diversity subset from the LOPAC screening inhibitor hits.

FIG. 2C shows the structures of compounds 61-82 of the chaperonin inhibitor diversity subset from the LOPAC screening inhibitor hits.

FIG. 3A shows the structures of compounds 83-99 of the chaperonin inhibitor diversity subset from the MMV screening inhibitor hits.

FIG. 3B shows the structures of compounds 100-116 of the chaperonin inhibitor diversity subset from the MMV screening inhibitor hits.

FIG. 3C shows the structures of compounds 117-119 of the chaperonin inhibitor diversity subset from the MMV screening inhibitor hits.

FIG. 4 shows a correlation plot comparing the inhibition of GroEL/ES-dMDH refolding and mHSP60/10-dMDH refolding of a denature malate dehydrogenase (dMDH) client protein. Each data point indicates an individual compound tested. The grey zones indicate IC50 values that are higher than the maximum concentrations tested. Compounds across the three libraries are nearly equipotent at inhibiting both GroEL/ES and mHSP60/10 refolding activities.

FIG. 5A shows a correlation plot representing HCT-116 cells vs average fold selectivity. The diversity set compounds exhibit a range of potencies and selectivities for the HCT-116 cells over the non-cancerous cells, the most selective of which are annotated.

FIG. 5B shows a correlation plot representing GroEL/ES-dMDH refolding vs average fold selectivity. When examining the ability of the diversity set compounds to inhibit GroEL/ES mHSP60/10 refolding functions, some of the most potent and selective compounds from the HCT-116 cell viability assay were also effective chaperonin inhibitors.

FIG. 5C shows a correlation plot representing mHSP60/10-dMDH refolding vs average fold selectivity. When examining the ability of the diversity set compounds to inhibit GroEL/ES refolding functions, some of the most potent and selective compounds from the HCT-116 cell viability assay were also effective chaperonin inhibitors.

FIG. 6 shows a schematic representation of the refolding assay used in screens to evaluate the three libraries of compounds. Binary complex of GroEL-dMDH (mHSP60-dMDH) is made, followed by the addition of GroES or HSP10. Inhibitors are then added and the chapeornin-mediated refolding cycles are initiated by the addition of ATP. After an incubation period, the folding cycles are quenched using EDTA and reporter reagents, NADH and ketomalonic acid, are added and NADH absorbance is read to determine if the denatured enzyme reporter is folded and functional.

FIG. 7 shows a correlation plot representing HCT-116 cell viability with the Nitrofuran-based (NF) and Hydroxyquinoline-based (HQ) N-acylhydrazone (NAH) compounds vs. average fold selectivity. Each point on this correlation plot represents one inhibitor tested. Many of these inhibitors were selective and potent for the colorectal cancer cells over the noncancerous cells.

FIGS. 8A-8C show correlation plots between cell viability results of the most potent and selective hydroxyquinoline N-acylhydrazone (HQ-NAH) analogs (FIG. 8A), nitrofuran N-acylhydrazone (NF-NAH) (FIG. 8B), and non-grouped N-acylhydrazone (NAH) analogs (FIG. 8C) (SI>5) with relative HSP60 levels in the cancer (gray symbols) and non-cancer (white symbols) cell lines. Spearman correlation coefficients are presented in the insets.

FIG. 9 shows cell growth inhibition results reported in the NCI database for Nifuroxazide. Nifuroxazide exhibits broad-spectrum inhibition of growth across nearly the entire panel of cancer cell lines: 1. KM12; 2. HCT-116; 3. HT29; 4. SW-620; 5. HCT-15; 6. COLO 205; 7. HCC-2998; 8. HOP-92; 9. NCI-H522; 10. A549; 11. NCI-H460; 12. HOP-62; 13. NCI-H23; 14. EKVX; 15. NCI-H322M; 16. NCI-H226; 17. PC-3; 18. DU-145; 19. TK-10; 20. CAKI-1; 21. A498; 22. RXF-393; 23. UO-31; 24. 786-0; 25. SN12C; 26. ACHN; 27. RPMI-8226; 28. K-562; 29. MOLT-4; 30. CCRF-CEM; 31. HL-60(TB); 32. SR; 33. UACC-62; 34. MDA-MB-435; 35. UACC-257; 36. LOX IMVI; 37. SK-MEL-28; 38. SK-MEL-5; 39. SK-MEL-2; 40. MDA-N; 41. MALME-3M; 42. M14; 43. OVCAR-5; 44. OVCAR-3; 45. ADR-RES; 46. IGROV1; 47. OVCAR-8; 48. SK-OV-3; 49. OVCAR-4; 50. MCF7; 51. MDA-MB-231; 52. T-47D; 53. HS 578T; 54. BT-549; 55. U251; 56. SF-295; 57. SNB-19; 58. SF-539; 59. SF-268; 60. SNB-75.

FIGS. 10A-10J show cell growth results for 10 lead candidate inhibitors (compound 162, FIG. 10A; compound 163, FIG. 10B; compound 164, FIG. 10C; compound 4, FIG. 10D; compound 165, FIG. 10E; compound 139, FIG. 10F; compound 132, FIG. 10G; compound 143, FIG. 10H; compound 147, FIG. 10I; compound 128, FIG. 10J) tested at 10 μM against the NCI cancer cell panel. Zero indicates no cells growth/death from what was initially plated (cytostatic inhibition). −100% indicates cell growth inhibition (cytocidal inhibition). +100% indicates cell growth.

FIGS. 11A-11G show cell growth results in dose-response for 7 lead candidate inhibitors (compound 162, FIG. 11A; compound 164, FIG. 11B; compound 128, FIG. 11C compound 165, FIG. 11D; compound 139, FIG. 11E; compound 143, FIG. 11F; compound 147, FIG. 11G) from initial single-dose NCI-60 screen.

FIG. 12 shows detailed cell growth results for each individual cell line for compound 162 tested at 10 μM against the NCI cancer cell panel. Zero indicates no cells growth/death from what was initially plated (cytostatic inhibition). −100% indicates cell growth inhibition (cytocidal inhibition). +100% indicates cell growth.

FIG. 13 shows detailed cell growth results for each individual cell line for compound 163 tested at 10 μM against the NCI cancer cell panel. Zero indicates no cells growth/death from what was initially plated (cytostatic inhibition). −100% indicates cell growth inhibition (cytocidal inhibition). +100% indicates cell growth.

FIG. 14 shows detailed cell growth results for each individual cell line for compound 164 tested at 10 μM against the NCI cancer cell panel. Zero indicates no cells growth/death from what was initially plated (cytostatic inhibition). −100% indicates cell growth inhibition (cytocidal inhibition). +100% indicates cell growth.

FIG. 15 shows detailed cell growth results for each individual cell line for compound 4 tested at 10 μM against the NCI cancer cell panel. Zero indicates no cells growth/death from what was initially plated (cytostatic inhibition). −100% indicates cell growth inhibition (cytocidal inhibition). +100% indicates cell growth.

FIG. 16 shows detailed cell growth results for each individual cell line for compound 165 tested at 10 μM against the NCI cancer cell panel. Zero indicates no cells growth/death from what was initially plated (cytostatic inhibition). −100% indicates cell growth inhibition (cytocidal inhibition). +100% indicates cell growth.

FIG. 17 shows detailed cell growth results for each individual cell line for compound 139 tested at 10 μM against the NCI cancer cell panel. Zero indicates no cells growth/death from what was initially plated (cytostatic inhibition). −100% indicates cell growth inhibition (cytocidal inhibition). +100% indicates cell growth.

FIG. 18 shows detailed cell growth results for each individual cell line for compound 132 tested at 10 μM against the NCI cancer cell panel. Zero indicates no cells growth/death from what was initially plated (cytostatic inhibition). −100% indicates cell growth inhibition (cytocidal inhibition). +100% indicates cell growth.

FIG. 19 shows detailed cell growth results for each individual cell line for compound 143 tested at 10 μM against the NCI cancer cell panel. Zero indicates no cells growth/death from what was initially plated (cytostatic inhibition). −100% indicates cell growth inhibition (cytocidal inhibition). +100% indicates cell growth.

FIG. 20 shows detailed cell growth results for each individual cell line for compound 147 tested at 10 μM against the NCI cancer cell panel. Zero indicates no cells growth/death from what was initially plated (cytostatic inhibition). −100% indicates cell growth inhibition (cytocidal inhibition). +100% indicates cell growth.

FIG. 21 shows detailed cell growth results for each individual cell line for compound 128 tested at 10 μM against the NCI cancer cell panel. Zero indicates no cells growth/death from what was initially plated (cytostatic inhibition). −100% indicates cell growth inhibition (cytocidal inhibition). +100% indicates cell growth.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended clauses.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**”, and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by “A-*”, “A-**”, or “A [symbol] ”, where each of “-*”, “-**”, and “ [symbol] ” represents a bond to A and the point of covalent bond attachment to B. Alternatively, in some embodiments, the portion of A-B defined by the group or chemical structure B can be represented by “*—B”, “**—B”, or “ [symbol] B”, where each of “-*”, “-**”, and “ [symbol] ” represents a bond to B and the point of covalent bond attachment to A.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g., “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted. As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. Coalkyl indicates a hydrogen where the group is in a terminal position, a bond if internal. A C₁₋₆ alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

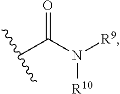

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

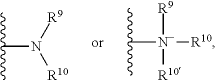

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein includes substituted or unsubstituted aromatic groups in which each atom of the ring is carbon. Preferably the ring is a 5- to 10-membered ring, more preferably a 6-membered or a 10-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include phenyl, phenol, aniline, naphthyl, anthryl, and the like.

In some instances, the term “biaryl”, as used herein, includes a substituted or unsubstituted aryl including two adjoining aromatic rings in which each atom of the ring is carbon. Preferably the ring is a 10-membered ring. Biaryl groups include naphthyl, and the like.

Moreover, the term “aryl” as used throughout the specification, examples, and claims is intended to include both unsubstituted and substituted aryl groups, the latter of which refers to aryl moieties having substituents replacing a hydrogen. In an exemplary embodiment, an aryl or biaryl includes a hydroxynaphthalene, depicted by the structure

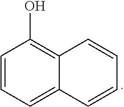 [SMILES: Oc1cccc2ccccc12]

The term “carbamate” is art-recognized and refers to a group

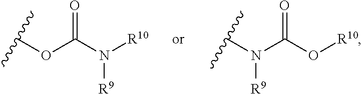

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo [2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁸, wherein R⁸ represents a hydrocarbyl group.

The term “ketone”, as used herein, refers to a group —C(O)R⁷, wherein R⁷ represents a hydrocarbyl group (e.g., alkyl, aryl, heteroaryl).

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic ring structures, preferably 5- to 12-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. The term “monocyclic heteroaryl”, as used herein, includes substituted or unsubstituted aromatic single-ring structures, preferably 5- to 6-membered rings, whose ring structures include at least one heteroatom, preferably one or two heteroatoms. Monocyclic heteroaryl groups include, for example, pyrrole, furan, thiophene, imidazole, oxazole, thiazole, pyrazole, pyridine, pyrazine, pyridazine, and pyrimidine, and the like. The term “bicyclic heteroaryl”, as used herein, includes substituted or unsubstituted aromatic ring including two adjoining aromatic rings, preferably 8- to 12-membered rings, whose ring structures include at least one heteroatom, preferably one or two heteroatoms. Bicyclic heteroaryl groups include, for example, indole, indazole, azaindole, azaindazole, benzofuran, benzothiophene, benzothiazole, benzopyran, benzothiadiazole, benzooxadiazole, quinoline, isoquinoline, quinazoline, thiadiazole, chromone, quinolone, and the like.

Moreover, the terms “heteroaryl” and “hetaryl” as used throughout the specification, examples, and claims is intended to include both unsubstituted and substituted heteroaryl and hetaryl groups, the latter of which refers to heteroaryl and hetaryl moieties having substituents replacing a hydrogen. In an exemplary embodiment, a heteroaryl or bicyclic heteroaryl includes a hydroxyquinoline, depicted by the structure

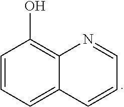 [SMILES: Oc1cccc2cccnc12]

In another exemplary embodiment, a heteroaryl or bicyclic heteroaryl includes a benzothiazole, depicted by the structure

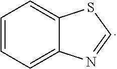

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like. Heterocyclyl groups can contain an oxo group, for example, pyrrolidinone or imidazolidinone, or can contain two oxo groups for example, imidazolidine-dione. Heterocyclyl groups including an oxo group or two oxo groups can be depicted by the structures

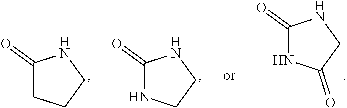 [SMILES: O=C1CCCN1 ; O=C1NCCN1 ; O=C1CNC(=O)N1]

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “hydrazone”, as used herein, refers to a group represented by the structure

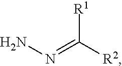

wherein R¹ and R² independently represent hydrogen or a hydrocarbyl group, and at least one of R¹ and R² is a hydrocarbyl group.

The term “hydrazide”, as used herein, refers to a group represented by the structure

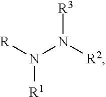

wherein R is acyl (R′—C(═O)—), sulfonyl (R′—S(═O)₂—), phosphoryl ((R′—)2P(═O)—), phosphonyl ((R′—O—)₂P(═O)—, and R¹, R², R³ and R′ independently represent hydrogen or a hydrocarbyl group. For example, an acyl hydrazide can be represented by the structure

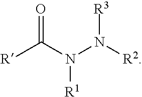

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

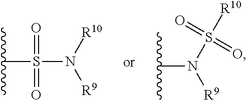

wherein R⁹ and R¹⁰ independently represents hydrogen or hydrocarbyl.

The term “sulfoxide” is art-recognized and refers to the group S(O).

The term “sulfonate” is art-recognized and refers to the group SO₃H, or a pharmaceutically acceptable salt thereof.

The term “sulfone” is art-recognized and refers to the group S(O)₂—R⁹, wherein R⁹ represents hydrocarbyl (e.g., alkyl, aryl, heteroaryl).

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group C(O)SR⁸ or SC(O)R⁸ wherein R⁸ represents a hydrocarbyl.

The term “thioether”, as used herein, is equivalent to an ether, wherein the oxygen is replaced with a sulfur.

The term “urea” is art-recognized and may be represented by the general formula

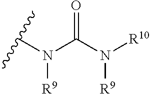

wherein R⁹ and R¹⁰ independently represent hydrogen or a hydrocarbyl.

As used herein, the phrase “R¹ and R² combine with the atoms to which they are attached to form a 3- to 7-membered heterocyclyl optionally substituted by oxo” also means that R¹ and R² are taken together with the carbon or nitrogen atoms to which they are attached to form a 3- to 7-membered heterocyclyl that is optionally substituted. In particular, R¹ and R² can combine with the atoms to which they are attached to form a 3- to 7-membered heterocyclyl optionally substituted by oxo, as represented by the following structures:

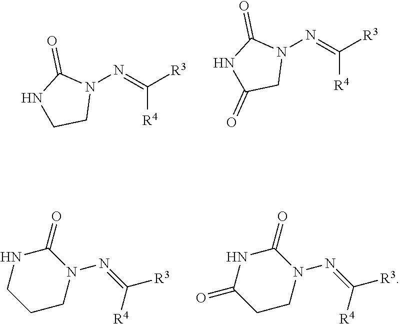

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

As used herein, the term “HSP inhibitor” as used herein refers to a compound that prevents, reverses, slows, or inhibits the activity of a heat shock protein (HSP) or isoform. Examples of HSP inhibitors include HSP100, HSP90, HSP70, HSP60, and small HSPs known as sHSP's/α-crystallins.

In treatment methods according to the disclosure, an “effective amount” means an amount or dose sufficient to generally bring about the desired therapeutic benefit in subjects needing such treatment. Effective amounts or doses of the compounds of the disclosure may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

### REPRESENTATIVE EMBODIMENTS

Compounds of the present disclosure find utility in the treatment and prevention of cancer including colorectal cancer (e.g., colon cancer), lung cancer (e.g., non-small cell lung cancer (NSCLC)), prostate cancer, renal cancer, blood cancer (e.g., leukemia), skin cancer (e.g., melanoma), ovarian cancer, breast cancer, CNS cancer, cervical cancer, and gastric cancer.

In some embodiments, the disclosure relates to treating cancer with an effective amount of an antibacterial or an antiparasitic compound (e.g., a compound of formula I-XIX).

In some embodiments, the disclosure relates to a compound or a pharmaceutically acceptable salt thereof, having the formula I,

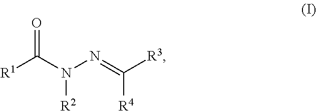

wherein
R¹ is aryl or heteroaryl, wherein aryl or heteroaryl is optionally substituted with halogen, —OR⁵, —NR⁵R⁶, —S(O)₂NR⁵R⁶, or —N(R⁵)SO₂R⁶;
R² is hydrogen;
or R¹ and R² combine with the atoms to which they are attached to form a 3- to 7-membered heterocyclyl;
R³ is aryl or heteroaryl, wherein aryl or heteroaryl is optionally substituted by —OR⁵ or nitro;
R⁴ is hydrogen;
R⁵ and R⁶ are each independently hydrogen, alkyl, aryl or heteroaryl, wherein each hydrogen atom in alkyl, aryl or heteroaryl is optionally substituted with halogen or —O alkyl.

In some embodiments, the disclosure relates to a compound or a pharmaceutically acceptable salt thereof, having the formula I,

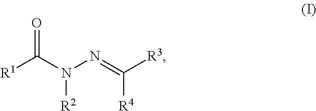

wherein
R¹ is aryl or heteroaryl, wherein aryl or heteroaryl is optionally substituted with halogen, —OR⁵, —NR⁵R⁶, —S(O)₂NR⁵R⁶, or —N(R⁵)SO₂R⁶;
R² is hydrogen;
or R¹ and R² combine with the atoms to which they are attached to form a 3- to 7-membered heterocyclyl optionally substituted by oxo;
R³ is aryl or heteroaryl, wherein aryl or heteroaryl is optionally substituted by —OR^{5′} or nitro;
R⁴ is hydrogen;
R⁵, R^{5′} and R⁶ are each independently hydrogen, alkyl, aryl or heteroaryl, wherein each hydrogen atom in alkyl, aryl or heteroaryl is optionally substituted with halogen or —O alkyl (e.g., C₁-C₆ alkyl).

In some embodiments of compounds of formula I, the compound is not

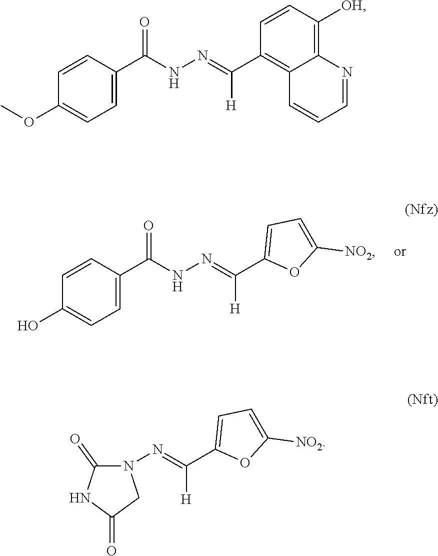 [SMILES: COc1ccc(C(=O)N/N=C/c2ccc(O)c3ncccc23)cc1 ; O=C(N/N=C/c1ccc([N+](=O)[O-])o1)c1ccc(O)cc1 ; "or" ; O=C1CN(/N=C/c2ccc([N+](=O)[O-])o2)C(=O)N1]

In some embodiments of compounds of formula I, II, or III, R¹ is aryl (e.g., C₆-C₁₀ aryl or biaryl), or heteroaryl (e.g., monocyclic or bicyclic heteroaryl). In some embodiments, R¹ is C₆-C₁₀ aryl or biaryl. In some embodiments, R¹ is C₆-C₁₀ aryl or monocyclic heteroaryl. In some embodiments, R¹ is C₆-C₁₀ aryl or biaryl (e.g., phenyl or naphthyl). In some embodiments, R¹ is monocyclic heteroaryl. In some embodiments, R¹ is bicyclic heteroaryl. Illustratively, each aryl, biaryl, monocyclic heteroaryl, or bicyclic heteroaryl of R¹ is optionally substituted with halogen, —OR⁵, —NR⁵R⁶, —S(O)₂NR⁵R⁶, or —N(R⁵)SO₂R⁶. In some embodiments, R¹ is C₆-C₁₀ aryl (e.g., phenyl or naphthyl) optionally substituted with halo, —OR⁵, —NR⁵R⁶, —S(O)₂NR⁵R⁶, or —N(R⁵)SO₂R⁶. In some embodiments, R¹ is C₆-C₁₀ aryl (e.g., phenyl or naphthyl) optionally substituted by —OH, —OC₁-C₆ alkyl, O-aryl, —N(C₁-C₆ alkyl)₂, —S(O)₂N(C₁-C₆ alkyl)₂, —N(H)S(O)₂-aryl, or —N(H)S(O)₂-heteroaryl. In some embodiments, R¹ is phenyl, naphthyl, thienyl, or pyridyl, wherein each phenyl, naphthyl, thienyl, or pyridyl is optionally substituted with halogen, hydroxy, methoxy, phenoxy, or dimethylamino. In some embodiments, R¹ is thienyl optionally substituted with halogen. In some embodiments, R¹ is phenyl optionally substituted with hydroxy, methoxy, phenoxy, or dimethylamino. In some embodiments, R¹ is naphthyl, methoxyphenyl, phenoxyphenyl, ethoxyphenylsulfonamido, or thienylsulfonamido. In some embodiments, R¹ is naphthyl, hydroxyphenyl (e.g., 4-hydroxyphenyl), methoxyphenyl (e.g., 4-methoxyphenyl), phenoxyphenyl (e.g., 2-phenoxyphenyl or 3-phenoxyphenyl), or ethoxyphenylsulfonamidophenyl.

In some embodiments of compounds of formula I, R² is hydrogen.

In some embodiments of compounds of formula I, R¹ and R² combine with the atoms to which they are attached to form a 3- to 7-membered heterocyclyl optionally substituted by oxo. In some embodiments, R¹ and R² combine with the atoms to which they are attached to form a 5- to 6-membered heterocyclyl optionally substituted by oxo.

In some embodiments of compounds of formula I, R³ is aryl (e.g., C₆-C₁₀ aryl or biaryl), or heteroaryl (e.g., monocyclic or bicyclic heteroaryl). In some embodiments, R³ is C₆-C₁₀ aryl or biaryl. In some embodiments, R³ is C₆-C₁₀ aryl or monocyclic heteroaryl. In some embodiments, R³ is C₆-C₁₀ aryl or biaryl (e.g., phenyl or naphthyl). In some embodiments, R³ is phenyl. In some embodiments, R³ is naphthyl. In some embodiments, R³ is monocyclic heteroaryl (e.g., thienyl or furanyl). In some embodiments, R³ is bicyclic heteroaryl (e.g., quinolyl).

In some embodiments of compounds of formula I, wherein R³ is aryl (e.g., C₆-C₁₀ aryl or biaryl), or heteroaryl (e.g., monocyclic or bicyclic heteroaryl), each hydrogen atom in aryl and heteroaryl is optionally substituted by —OR^{5′} or nitro. In some embodiments, each hydrogen atom in aryl, and heteroaryl of R³ is optionally substituted by —OR^{5′}. In some embodiments, each hydrogen atom in aryl, and heteroaryl of R³ is optionally substituted by nitro. In some embodiments, R³ is heteroaryl optionally substituted by nitro. In some embodiments, R³ is quinolyl optionally substituted by —OR^{5′}. In some embodiments, R³ is hydroxyquinolyl. In some embodiments, R³ is furanyl optionally substituted by nitro. In some embodiments, R³ is nitrofuranyl.

In some embodiments of compounds of formula I, R⁴ is hydrogen.

In some embodiments of compounds of formula I, R^{5′} is hydrogen or alkyl (e.g., C₁-C₆ alkyl). In some embodiments, R^{5′} is hydrogen.

In some embodiments of compounds of formula I, II, or III, R⁵ and R⁶ are each independently hydrogen, C₁-C₆ alkyl, C₆-C₁₀ aryl, or heteroaryl, wherein each C₁-C₆ alkyl, aryl, and heteroaryl is optionally substituted with halogen or —OC₁-C₆ alkyl. In some embodiments, R⁵ and R⁶ are each independently hydrogen, methyl, ethyl, phenyl, or thienyl, wherein each ethyl, ethyl, phenyl, or thienyl s optionally substituted with chloro, bromo, methoxy or ethoxy. In some embodiments, R⁵ is hydrogen, methyl, ethyl, or phenyl. In some embodiments, R⁶ is methyl, ethyl, phenyl, bromophenyl, methoxyphenyl, ethoxyphenyl,

In some embodiments of compounds of formula I, II, or III, R⁵ is a hydrogen, C₁-C₆ alkyl, C₆-C₁₀ aryl, or heteroaryl (e.g., monocyclic or bicyclic heteroaryl), wherein each hydrogen atom in C₁-C₆ alkyl, aryl, and heteroaryl is optionally substituted with halogen or —OC₁-C₆ alkyl. In some embodiments, R⁵ is a hydrogen. In some embodiments, R⁵ is a C₁-C₆ alkyl. In some embodiments, each hydrogen atom in C₁-C₆ alkyl is optionally substituted with halogen or —OC₁-C₆ alkyl.

In some embodiments of compounds of formula I, II, or III, R⁵ is C₆-C₁₀ aryl. In some embodiments, R⁵ is aryl is optionally substituted with halogen or —OC₁-C₆ alkyl. In some embodiments, R⁵ is monocyclic heteroaryl. In some embodiments, R⁵ is monocyclic heteroaryl optionally substituted with halogen or —OC₁-C₆ alkyl. In some embodiments, R⁵ is a bicyclic heteroaryl. In some embodiments, R⁵ is bicyclic heteroaryl optionally substituted with halogen or —OC₁-C₆ alkyl.

In some embodiments of compounds of formula I, II, or III, R⁶ is a hydrogen, C₁-C₆ alkyl, C₆-C₁₀ aryl, or heteroaryl (e.g., monocyclic or bicyclic heteroaryl), wherein C₁-C₆ alkyl, aryl, and heteroaryl is optionally substituted with halogen or —OC₁-C₆ alkyl. In some embodiments, R⁶ is a hydrogen. In some embodiments, R⁶ is a C₁-C₆ alkyl. In some embodiments, R⁶ is C₁-C₆ alkyl optionally substituted with halogen or —OC₁-C₆ alkyl.

In some embodiments of compounds of formula I, II, or III, R⁶ is C₆-C₁₀ aryl. In some embodiments, R⁶ is aryl optionally substituted with halogen or —OC₁-C₆ alkyl. In some embodiments, R⁶ is monocyclic heteroaryl. In some embodiments, R⁶ is monocyclic heteroaryl optionally substituted with halogen or —OC₁-C₆ alkyl. In some embodiments, R⁶ is a bicyclic heteroaryl. In some embodiments, R⁶ is bicyclic heteroaryl optionally substituted with halogen or —OC₁-C₆ alkyl.

In some embodiments, compounds of the disclosure may be of the formula II, or a pharmaceutically acceptable salt thereof,

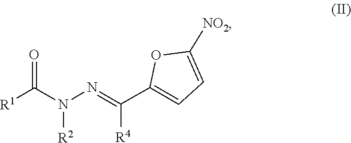

wherein each of R¹, R², and R⁴ is as described herein.

In some embodiments of compounds of formula II, R¹ is aryl (e.g., C₆-C₁₀ aryl or biaryl), or heteroaryl (e.g., monocyclic or bicyclic heteroaryl). In some embodiments, R¹ is aryl, biaryl, or monocyclic or bicyclic heteroaryl. In some embodiments, R¹ is C₆-C₁₀ aryl, biaryl, or monocyclic heteroaryl. In some embodiments, R¹ is C₆-C₁₀ aryl or biaryl. In some embodiments, R¹ is C₆-C₁₀ aryl. In some embodiments, R¹ is C₆-C₁₀ biaryl. In some embodiments, R¹ is monocyclic heteroaryl. In some embodiments, R¹ is bicyclic heteroaryl. In some embodiments, each hydrogen atom in C₆-C₁₀ aryl, biaryl, or mono- or bicyclic heteroaryl of R¹ is optionally substituted with halogen, —OR⁵, —NR⁵R⁶, —S(O)₂NR⁵R⁶, or —NR⁵SO₂R⁶.

In some embodiments of compounds of formula II, R² is hydrogen. In some embodiments, R¹ and R² combine with the atoms to which they are attached to form a 3- to 7-membered heterocyclyl. In some embodiments, the 3- to 7-membered heterocyclyl is optionally substituted by oxo.

In some embodiments of compounds of formula II, R⁴ is hydrogen.

In some embodiments, compounds of the disclosure may be of the formula III, or a pharmaceutically acceptable salt thereof,

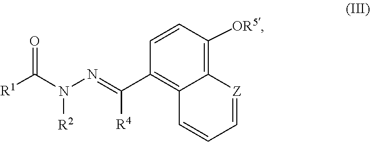

wherein each of R¹, R², R⁴, R^{5′}, and Z is as described herein.

In some embodiments of compounds of formula III, Z is CH or N. In some embodiments, Z is N.

In some embodiments of compounds of formula III, R¹ is aryl (e.g., C₆-C₁₀ aryl or biaryl), or heteroaryl (e.g., monocyclic or bicyclic heteroaryl). In some embodiments, R¹ is C₆-C₁₀ aryl optionally substituted by —OH, —OC₁-C₆ alkyl, O-aryl, —N(C₁-C₆ alkyl)₂, —S(O)₂N(C₁-C₆ alkyl)₂, —NHSO₂-aryl, —NHSO₂-heteroaryl. In some embodiments, the aryl is optionally substituted with halogen or —OC₁-C₆ alkyl.

In some embodiments of compounds of formula III, R² is hydrogen. In some embodiments, R¹ and R² combine with the atoms to which they are attached to form a 3- to 7-membered heterocyclyl. In some embodiments, the 3- to 7-membered heterocyclyl is optionally substituted by oxo.

In some embodiments of compounds of formula III, R⁴ is hydrogen.

In some embodiments, the compound is of formula I, formula II, or formula III or a pharmaceutically acceptable salt thereof, R¹ is monocyclic heteroaryl optionally substituted with halogen. In some embodiments, the compound is of formula I, formula II, or formula III or a pharmaceutically acceptable salt thereof, R¹ is C₆-C₁₀ aryl (e.g., phenyl) optionally substituted with halo, —OR⁵, —NR⁵R⁶, —S(O)₂NR⁵R⁶, or —NR⁵SO₂R⁶.

In some embodiments, the compound of Formula (I), (II), or (III) or a pharmaceutically acceptable salt thereof is selected from Table A.

TABLE A
Hydroxyquinoline-based N-acylhydrazone analogs (HQ-NAH series)
Compound 18

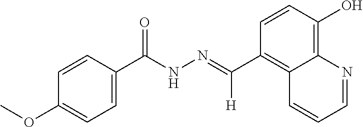 [SMILES: COc1ccc(C(=O)N/N=C/c2ccc(O)c3ncccc23)cc1]

N′-((8-hydroxyquinolin-5-yl)methylene)-4-
methoxybenzohydrazide

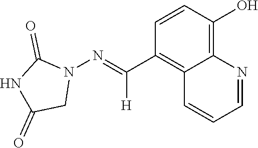 [SMILES: O=C1CN(/N=C/c2ccc(O)c3ncccc23)C(=O)N1]

1-(((8-hydroxyquinolin-5-
yl)methylene)amino)imidazolidine-2,4-dione

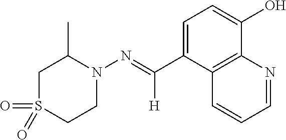 [SMILES: CC1CS(=O)(=O)CCN1/N=C/c1ccc(O)c2ncccc12]

4-(((8-hydroxyquinolin-5-
yl)methylene)amino)-3-methylthiomorpholine
1,1-dioxide

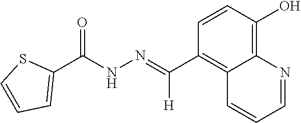 [SMILES: O=C(N/N=C/c1ccc(O)c2ncccc12)c1cccs1]

N′-((8-hydroxyquinolin-5-
yl)methylene)thiophene-2-carbohydrazide

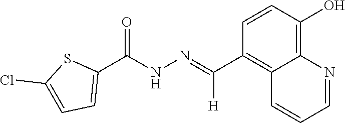 [SMILES: O=C(N/N=C/c1ccc(O)c2ncccc12)c1ccc(Cl)s1]

5-chloro-N′-((8-hydroxyquinolin-5-
yl)methylene)thiophene-2-carbohydrazide

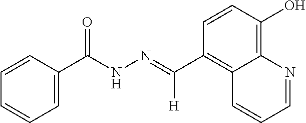 [SMILES: O=C(N/N=C/c1ccc(O)c2ncccc12)c1ccccc1]

N′-((8-hydroxyquinolin-5-
yl)methylene)benzohydrazide

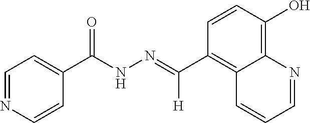 [SMILES: O=C(N/N=C/c1ccc(O)c2ncccc12)c1ccncc1]

N′-((8-hydroxyquinolin-5-
yl)methylene)isonicotinohydrazide
Compound 139

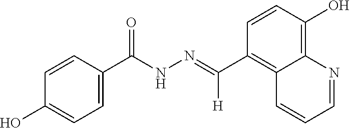 [SMILES: O=C(N/N=C/c1ccc(O)c2ncccc12)c1ccc(O)cc1]

4-hydroxy-N′-((8-hydroxyquinolin-5-
yl)methylene)benzohydrazide

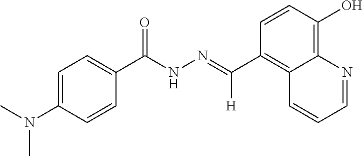 [SMILES: CN(C)c1ccc(C(=O)N/N=C/c2ccc(O)c3ncccc23)cc1]

4-(dimethylamino)-N′-((8-hydroxyquinolin-5-
yl)methylene)benzohydrazide

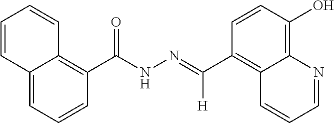 [SMILES: O=C(N/N=C/c1ccc(O)c2ncccc12)c1cccc2ccccc12]

N′-((8-hydroxyquinolin-5-yl)methylene)-1-
naphthohydrazide

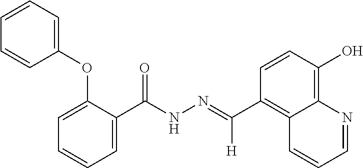 [SMILES: O=C(N/N=C/c1ccc(O)c2ncccc12)c1ccccc1Oc1ccccc1]

N′-((8-hydroxyquinolin-5-yl)methylene)-2-
phenoxybenzohydrazide
Compound 143

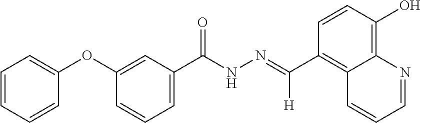 [SMILES: O=C(N/N=C/c1ccc(O)c2ncccc12)c1cccc(Oc2ccccc2)c1]

N′-((8-hydroxyquinolin-5-yl)methylene)-3-
phenoxybenzohydrazide

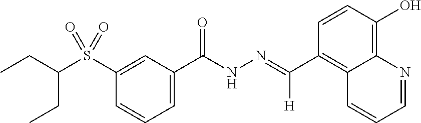 [SMILES: CCC(CC)S(=O)(=O)c1cccc(C(=O)N/N=C/c2ccc(O)c3ncccc23)c1]

N,N-diethyl-3-(2-((8-hydroxyquinolin-5-
yl)methylene)hydrazine-1-
carbonyl)benzenesulfonamide

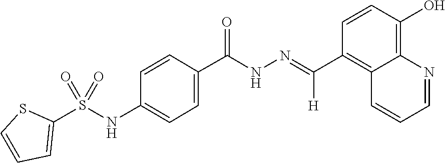 [SMILES: O=C(N/N=C/c1ccc(O)c2ncccc12)c1ccc(NS(=O)(=O)c2cccs2)cc1]

N-(4-(2-((8-hydroxyquinolin-5-
yl)methylene)hydrazine-1-
carbonyl)phenyl)thiophene-2-sulfonamide

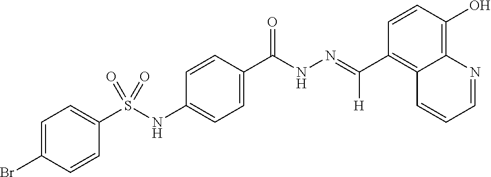 [SMILES: O=C(N/N=C/c1ccc(O)c2ncccc12)c1ccc(NS(=O)(=O)c2ccc(Br)cc2)cc1]

4-bromo-N-(4-(2-((8-hydroxyquinolin-5-
yl)methylene)hydrazine-1-
carbonyl)phenyl)benzenesulfonamide
Compound 147

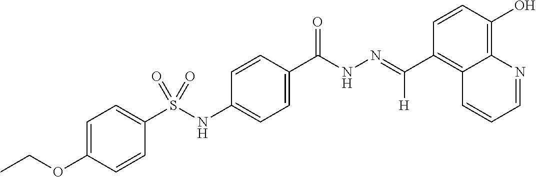 [SMILES: CCOc1ccc(S(=O)(=O)Nc2ccc(C(=O)N/N=C/c3ccc(O)c4ncccc34)cc2)cc1]

4-ethoxy-N-(4-(2-((8-hydroxyquinolin-5-
yl)methylene)hydrazine-1-
carbonyl)phenyl)benzenesulfonamide
Nitrofuran-based N-acylhydrazone analogs (NF-NAH series)

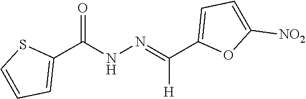 [SMILES: O=C(N/N=C/c1ccc([N+](=O)[O-])o1)c1cccs1]

N′-((5-nitrofuran-2-yl)methylene)thiophene-
2-carbohydrazide

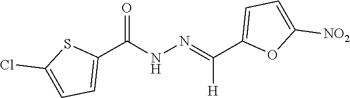 [SMILES: O=C(N/N=C/c1ccc([N+](=O)[O-])o1)c1ccc(Cl)s1]

5-chloro-N′-((5-nitrofuran-2-
yl)methylene)thiophene-2-carbohydrazide

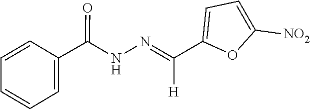 [SMILES: O=C(N/N=C/c1ccc([N+](=O)[O-])o1)c1ccccc1]

N′-((5-nitrofuran-2-
yl)methylene)benzohydrazide

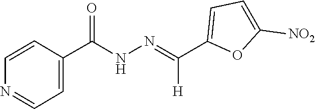 [SMILES: O=C(N/N=C/c1ccc([N+](=O)[O-])o1)c1ccncc1]

N′-((5-nitrofuran-2-
yl)methylene)isonicotinohydrazide

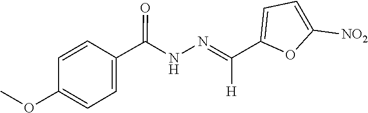 [SMILES: COc1ccc(C(=O)N/N=C/c2ccc([N+](=O)[O-])o2)cc1]

4-methoxy-N′-((5-nitrofuran-2-
yl)methylene)benzohydrazide

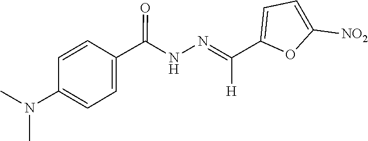 [SMILES: CN(C)c1ccc(C(=O)N/N=C/c2ccc([N+](=O)[O-])o2)cc1]

4-(dimethylamino)-N′-((5-nitrofuran-2-
yl)methylene)benzohydrazide

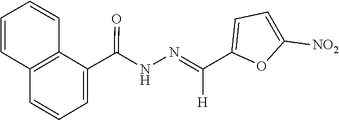 [SMILES: O=C(N/N=C/c1ccc([N+](=O)[O-])o1)c1cccc2ccccc12]

N′-((5-nitrofuran-2-yl)methylene)-1-
naphthohydrazide

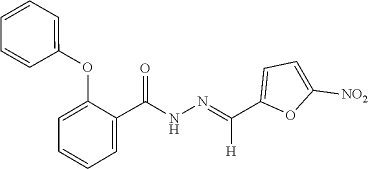 [SMILES: O=C(N/N=C/c1ccc([N+](=O)[O-])o1)c1ccccc1Oc1ccccc1]

N′-((5-nitrofuran-2-yl)methylene)-2-
phenoxybenzohydrazide
Compound 128

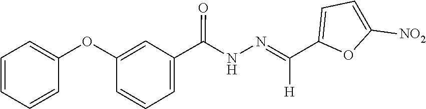 [SMILES: O=C(N/N=C/c1ccc([N+](=O)[O-])o1)c1cccc(Oc2ccccc2)c1]

N′-((5-nitrofuran-2-yl)methylene)-3-
phenoxybenzohydrazide

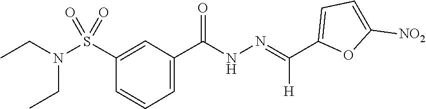 [SMILES: CCN(CC)S(=O)(=O)c1cccc(C(=O)N/N=C/c2ccc([N+](=O)[O-])o2)c1]

N,N-diethyl-3-(2-((5-nitrofuran-2-
yl)methylene)hydrazine-1-
carbonyl)benzenesulfonamide

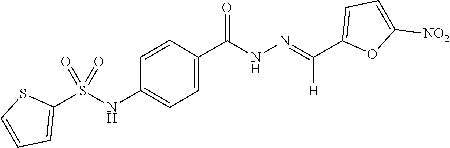 [SMILES: O=C(N/N=C/c1ccc([N+](=O)[O-])o1)c1ccc(NS(=O)(=O)c2cccs2)cc1]

N-(4-(2-((5-nitrofuran-2-
yl)methylene)hydrazine-1-
carbonyl)phenyl)thiophene-2-sulfonamide

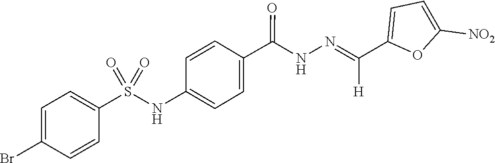 [SMILES: O=C(N/N=C/c1ccc([N+](=O)[O-])o1)c1ccc(NS(=O)(=O)c2ccc(Br)cc2)cc1]

4-bromo-N-(4-(2-((5-nitrofuran-2-
yl)methylene)hydrazine-1-
carbonyl)phenyl)benzenesulfonamide
Compound 132

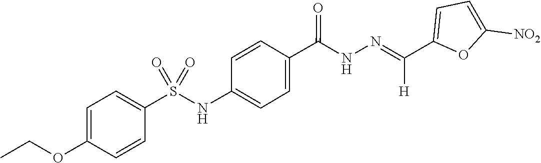 [SMILES: CCOc1ccc(S(=O)(=O)Nc2ccc(C(=O)N/N=C/c3ccc([N+](=O)[O-])o3)cc2)cc1]

4-ethoxy-N-(4-(2-((5-nitrofuran-2-
yl)methylene)hydrazine-1-
carbonyl)phenyl)benzenesulfonamide
Initial N-acylhydrazone analogs (NAH series)

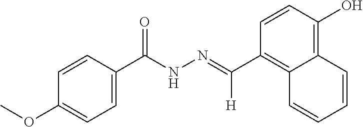 [SMILES: COc1ccc(C(=O)N/N=C/c2ccc(O)c3ccccc23)cc1]

N′-((4-hydroxynaphthalen-1-yl)methylene)-4-
methoxybenzohydrazide

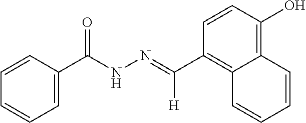 [SMILES: O=C(N/N=C/c1ccc(O)c2ccccc12)c1ccccc1]

N′-((4-hydroxynaphthalen-1-
yl)methylene)benzohydrazide

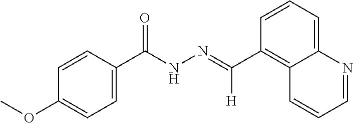 [SMILES: COc1ccc(C(=O)N/N=C/c2cccc3ncccc23)cc1]

4-methoxy-N′-(quinolin-5-
ylmethylene)benzohydrazide

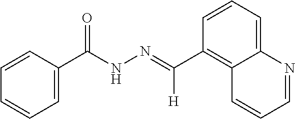 [SMILES: O=C(N/N=C/c1cccc2ncccc12)c1ccccc1]

N′-(quinolin-5-ylmethylene)benzohydrazide

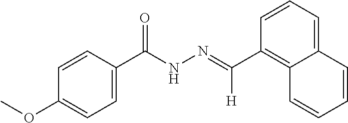 [SMILES: COc1ccc(C(=O)N/N=C/c2cccc3ccccc23)cc1]

4-methoxy-N′-(naphthalen-1-
ylmethylene)benzohydrazide

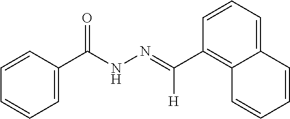 [SMILES: O=C(N/N=C/c1cccc2ccccc12)c1ccccc1]

N′-(naphthalen-1-
ylmethylene)benzohydrazide

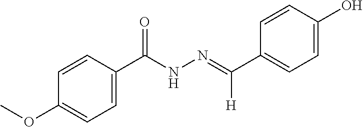 [SMILES: COc1ccc(C(=O)N/N=C/c2ccc(O)cc2)cc1]

N′-(4-hydroxybenzylidene)-4-
methoxybenzohydrazide

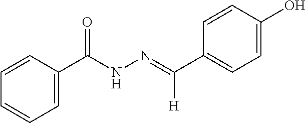 [SMILES: O=C(N/N=C/c1ccc(O)cc1)c1ccccc1]

N′-(4-hydroxybenzylidene)benzohydrazide

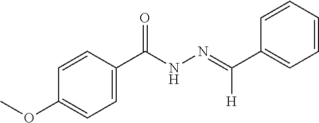 [SMILES: COc1ccc(C(=O)N/N=C/c2ccccc2)cc1]

N′-benzylidene-4-methoxybenzohydrazide

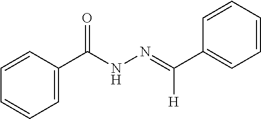 [SMILES: O=C(N/N=C/c1ccccc1)c1ccccc1]

N′-benzylidenebenzohydrazide

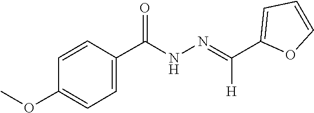 [SMILES: COc1ccc(C(=O)N/N=C/c2ccco2)cc1]

N′-(furan-2-ylmethylene)-4-
methoxybenzohydrazide
8-hydroxyquinoline-5-carbaldehyde
5-nitrofuran-2-carbaldehyde
4-methoxybenzohydrazide

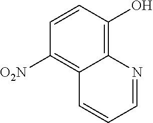 [SMILES: O=[N+]([O-])c1ccc(O)c2ncccc12]

Nitroxoline

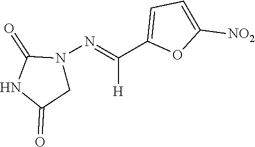 [SMILES: O=C1CN(/N=C/c2ccc([N+](=O)[O-])o2)C(=O)N1]

Nitrofurantoin (Nft)

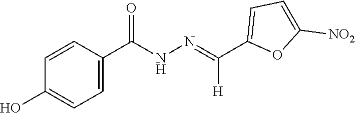 [SMILES: O=C(N/N=C/c1ccc([N+](=O)[O-])o1)c1ccc(O)cc1]

Nifuroxazide (Nfz)
Nifurtimox (Nfx)

In some embodiments, the disclosure relates to a compound or a pharmaceutically acceptable salt thereof, having the formula IV

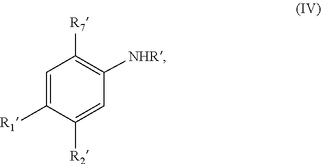

wherein:
R′ is C₁-C₆ alkyl or

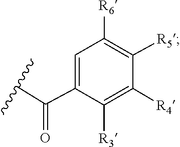

R₁′ is H, —OH, —OC₁-C₆ alkyl, —NHC(O)C₁-C₆ alkyl, —C(O)OC₁-C₆ alkyl, —C(O)OH, C₁-C₆ alkyl, —S-heteroaryl, or

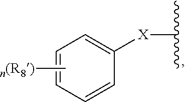

wherein C₁-C₆ alkyl is optionally substituted by —CN;
R₂′ is H or halo;
each of R₃′, R₄′, R^{5′}, and R₆′ is independently H, —OH, halo, —O—C₁-C₆ alkyl, —NO₂, or —NH₂;
R₇′ is H or C₁-C₆ alkyl;
X is —O—, —S—, —C(R₉′)(R₁₀′)ₘ—, or —C(R₉′)(R₁₀′)ₘO—, optionally X is —O—, —S—, or —C(R₉′)(CN);
R₈′ is halo;
R₉′ is H or —CN,
R₁₀′ is H or —CN;
m is 1 or 2; and
n is 0, 1, or 2.

In some embodiments of compounds of formula IV, the compound is not

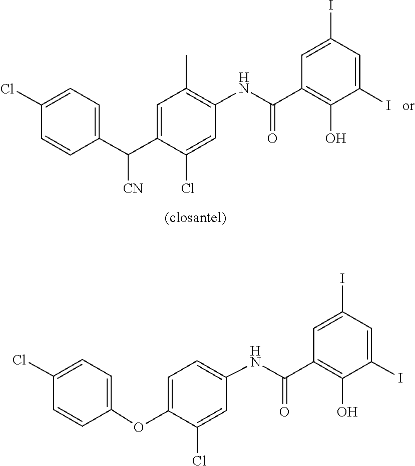 [SMILES: Cc1cc(C(C#N)c2ccc(Cl)cc2)c(Cl)cc1NC(=O)c1cc(I)cc(I)c1O ; O=C(Nc1ccc(Oc2ccc(Cl)cc2)c(Cl)c1)c1cc(I)cc(I)c1O]

rafoxanide).

In some embodiments of compounds of formula IV, R′ is C₁-C₆ alkyl or

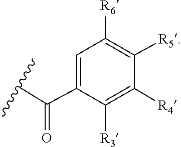

In some embodiments, R′ is C₁-C₆ alkyl. In some embodiments R′ is

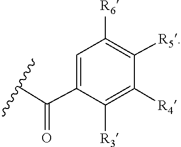

In some embodiments of compounds of formula IV, R₁′ is H, —OH, —OC₁-C₆ alkyl, —NHC(O)C₁-C₆ alkyl, —C(O)OC₁-C₆ alkyl, —C(O)OH, C₁-C₆ alkyl, —S-heteroaryl, or

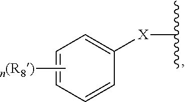

wherein C₁-C₆ alkyl is optionally substituted by —CN. In some embodiments, R₁′ is H. In some embodiments, R₁′ is —OH. In some embodiments, R₁′ is —OC₁-C₆ alkyl. In some embodiments, R₁′ is —NHC(O)C₁-C₆ alkyl. In some embodiments, R₁′ is —C(O)OC₁-C₆ alkyl. In some embodiments, R₁′ is —C(O)OH. In some embodiments, R₁′ is C₁-C₆ alkyl. In some embodiments, R₁′ is —S-heteroaryl. In some embodiments, R₁′ is

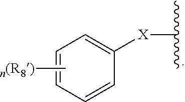

In some embodiments, when R¹ comprises a C₁-C₆ alkyl, each hydrogen atom may be optionally substituted by —CN. In some embodiments, R₁′ is —S-benzothiazole.

In some embodiments of compounds of formula IV, R₂′ is H or a halo. In some embodiments, R₂′ is H. In some embodiments, R₂′ is a halo. In some embodiments, R₂′ is bromo. In some embodiments, R₂′ is chloro. In some embodiments, R₂′ is iodo. In some embodiments, R₂′ is fluoro.

In some embodiments of compounds of formula IV, each of R₃′, R₄′, R₅′, and R₆′ is independently H, —OH, halo, C₁-C₆ alkyl, —O—C₁-C₆ alkyl, —NO₂, or —NH₂. In some embodiments, R₃′ is H. In some embodiments, R₃′ is —OH. In some embodiments, R₃′ is a halo. In some embodiments, R₃′ is selected from the group consisting of fluoro, chloro, bromo, and iodo. In some embodiments, R³ is —O—C₁-C₆ alkyl. In some embodiments, R₃′ is —NO₂. In some embodiments, R₃′ is —NH₂.

In some embodiments of compounds of formula IV, R₄′ is H. In some embodiments, R₄′ is —OH. In some embodiments, R₄′ is a halo. In some embodiments, R₄′ is selected from the group consisting of fluoro, chloro, bromo, and iodo. In some embodiments, R₄′ is —O—C₁-C₆ alkyl. In some embodiments, R₄′ is —NO₂. In some embodiments, R₄′ is —NH₂.

In some embodiments of compounds of formula IV, R₅′ is H. In some embodiments, R₅′ is —OH. In some embodiments, R₅′ is a halo. In some embodiments, R₅′ is selected from the group consisting of fluoro, chloro, bromo, and iodo. In some embodiments, R₅′ is —O—C₁-C₆ alkyl. In some embodiments, R₅′ is —NO₂. In some embodiments, R₅′ is —NH₂.

In some embodiments of compounds of formula IV, R₆′ is H. In some embodiments, R₆′ is —OH. In some embodiments, R₆′ is a halo. In some embodiments, R₆′ is selected from the group consisting of fluoro, chloro, bromo, and iodo. In some embodiments, R₆′ is —O—C₁-C₆ alkyl. In some embodiments, R₆′ is —NO₂. In some embodiments, R₆′ is —NH₂. In some embodiments, R₆′ is chloro.

In some embodiments of compounds of formula IV, R₇′ is H or C₁-C₆ alkyl. In some embodiments, R₇′ is H. In some embodiments, R₇′ is C₁-C₆ alkyl. In some embodiments, R₇′ is methyl.

In some embodiments of compounds of formula IV, X is —O—, —S—, —C(R₉′)(R₁₀′)ₘ—, or —C(R₉′)(R₁₀′)ₘO—. In some embodiments, X is —C(R₉′)(CN), preferably —C(H)(CN)—. In some embodiments, X is —O—. In some embodiments, X is —S—. In some embodiments, X is —C(R₉′)(R₁₀′)ₘ—. In some embodiments, X is —C(R₉′)(R₁₀′)ₘO—. In some embodiments, m is 1 and X is —C(R₉′)(R₁₀′)O—. In some embodiments, m is 1 and X is —C(R₉′)(R₁₀′)—. In some embodiments, m is 1 and X is —C(H)(CN)—O—. In some embodiments, m is 1 and X is —C(H)(CN)—.

In some embodiments of compounds of formula IV, R₈′ is halo. In some embodiments, R₈′ is bromo. In some embodiments, R₈′ is chloro. In some embodiments, R₈′ is fluoro. In some embodiments, R₈′ is iodo.

In some embodiments of compounds of formula IV, R₉′ is H or —CN. In some embodiments, R₉′ is H. In some embodiments, R₉′ is —CN.

In some embodiments of compounds of formula IV, R₁₀′ is H or —CN. In some embodiments, R₁₀′ is H. In some embodiments, R₁₀′ is —CN.

In some embodiments of compounds of formula IV, m is 1 or 2. In some embodiments, m is 1. In some embodiments, m is 2.

In some embodiments of compounds of formula IV, n is 0, 1, or 2. In some embodiments, n is 0. In some embodiments, n is 1. In some embodiments, n is 2.

In some embodiments, the disclosure relates to a compound or a pharmaceutically acceptable salt thereof, having the formula V

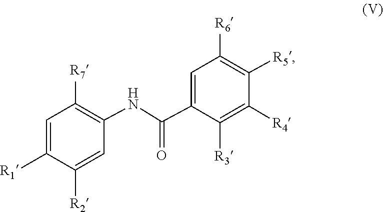

wherein each of R₁′, R₂′, R₃′, R₄′, R₅′, R₆′, and R₇′ is as described herein.

In some embodiments of compounds of formula V, R₃′ is —OH or —O—C₁-C₆ alkyl. In some embodiments, R₃′ is —OH. In some embodiments R₃′ is —O—C₁-C₆ alkyl.

In some embodiments of compounds of formula V, R₄′ is halo. In some embodiments, R₄′ is a bromine. In some embodiments, R₄′ is a chlorine. In some embodiments, R₄′ is a fluorine. In some embodiments, R₄′ is an iodine.

In some embodiments of compounds of formula V, R₆′ is halo. In some embodiments, R₆′ is a bromo. In some embodiments, R₆′ is a chloro. In some embodiments, R₆′ is a fluoro. In some embodiments, R₆′ is an iodo.

In some embodiments, the disclosure relates to a compound or a pharmaceutically acceptable salt thereof, having the formula VI

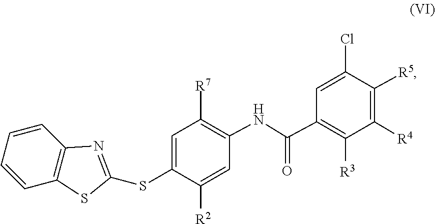

wherein each of R₂′, R₃′, R₄′, R₅′, and R₇′ is as described herein.

In some embodiments, the disclosure relates to a compound or a pharmaceutically acceptable salt thereof, having the formula (VII)

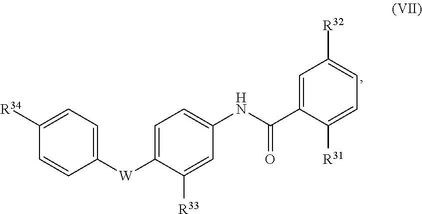

wherein
W is O or CHCN;
R³¹ is OH, or OCH₃;
R³² is halo (e.g., Cl or Br);
R³³ is H, or halo;
R³⁴ is H, or halo.

In some embodiments of compounds of formula VII, W is O or CHCN, R³¹ is OH, R³² is Cl, and R³³ and R³⁴ are independently H, or Cl. In one embodiment W is O, R³¹ is OH, R³² is Cl, and R³³ and R³⁴ are independently H, or Cl.

In some embodiments, the disclosure relates to a compound or a pharmaceutically acceptable salt thereof, having the formula VIII

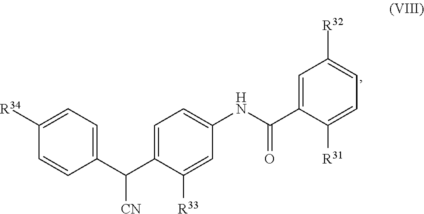

wherein each of R³¹, R³², R³³, and R³⁴ is as described herein.

In some embodiments of compounds of formula VIII, R³¹ is OH, R³² is Cl, and R³³ and R³⁴ are independently H, or Cl.

In some embodiments, the disclosure relates to a compound or a pharmaceutically acceptable salt thereof, having the formula IX:

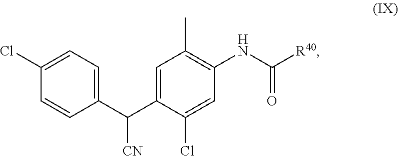

wherein R⁴⁰ is

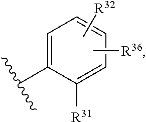

wherein
R³¹ is OH or OCH₃;
R³² and R³⁶ are independently H, Br or Cl, with the proviso that R³² and R³⁶ are not both H.

In some embodiments of compounds of formula IX, R³¹ is OH, R³⁶ is H, and R³² is Br or Cl. In one embodiment R⁴⁰ is

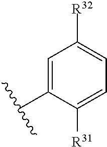

wherein
R³¹ is OH, and R³² is Br or Cl.

In some embodiments, the disclosure relates to a compound or a pharmaceutically acceptable salt thereof, having the formula X,

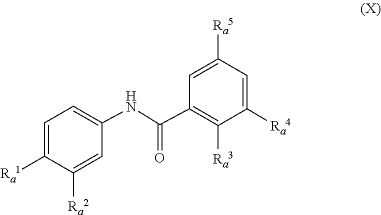

wherein
Rₐ¹ is H or

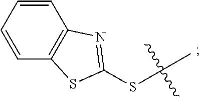 [SMILES: *Sc1nc2ccccc2s1]

Rₐ² is H or Cl;
Rₐ³ is H, OH, or OCH₃;
Rₐ⁴ is H or Br; and
Rₐ⁵ is H or Br;
provided that at least one of Rₐ¹, Rₐ², Rₐ³, Rₐ⁴, and Rₐ⁵ is not H.

In some embodiments, the disclosure relates to a compound or a pharmaceutically acceptable salt thereof, having the formula XI,

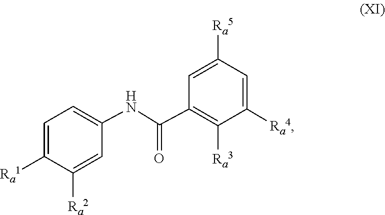

wherein each of Rₐ¹, Rₐ², Rₐ⁴, and Rₐ⁵ is as described herein.

In some embodiments, the disclosure relates to a compound or a pharmaceutically acceptable salt thereof, having the formula XII,

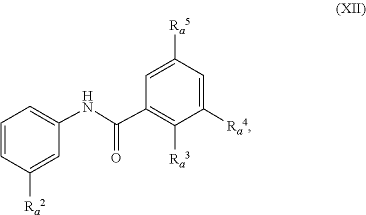

wherein each of Rₐ², Rₐ³, Rₐ⁴, and Rₐ⁵ is as described herein.

In some embodiments of compounds of formula (X) or (XI), Rₐ¹ is H or

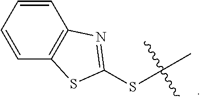 [SMILES: *Sc1nc2ccccc2s1]

In some embodiments Rₐ¹ is H. In some embodiments, Rₐ¹ is

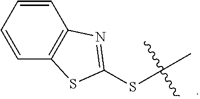 [SMILES: *Sc1nc2ccccc2s1]

In some embodiments of compounds of formula (X), (XI), or (XII), Rₐ² is H or Cl. In some embodiments, Rₐ² is H. In some embodiments, Rₐ² is Cl.

In some embodiments of compounds of formula (X) or (XII), Rₐ³ is H, OH, or OCH₃. In some embodiments, Rₐ³ is H. In some embodiments, Rₐ³ is OH. In some embodiments, Rₐ³ is OCH₃. In some embodiments, Rₐ³ is H or OH. In some embodiments, Rₐ³ is H or OCH₃. In some embodiments, Rₐ³ is OH or OCH₃.

In some embodiments of compounds of formula (X), (XI), or (XII), Rₐ⁴ is H or Br. In some embodiments, Rₐ⁴ is H. In some embodiments, Rₐ⁴ is Br.

In some embodiments of compounds of formula (X), (XI), or (XII), Rₐ⁵ is H or Br. In some embodiments, Rₐ⁵ is H. In some embodiments, Rₐ⁵ is Br.

In some embodiments of compounds of formula (X), (XI), or (XII), at least one of Rₐ¹, Rₐ², Rₐ³, Rₐ⁴, and Rₐ⁵ is not H. In some embodiments, at least two of Rₐ¹, Rₐ², Rₐ³, Rₐ⁴, and Rₐ⁵ are independently not H. In some embodiments, at least three of Rₐ¹, Rₐ², Rₐ³, Rₐ⁴, and Rₐ⁵ are independently not H. In some embodiments, at least four of Rₐ¹, Rₐ², Rₐ³, Rₐ⁴, and Rₐ⁵ are independently not H.

In some embodiments of compounds of formula (X), (XI), or (XII), Rₐ⁴ is Br and Rₐ⁵ is Br.

In some embodiments, the compound of Formula (IV), (V), (VI), (VII), (VIII), (IX), (X), (XI), or (XII) or a pharmaceutically acceptable salt thereof is selected from Table B.

TABLE B
Compound 165

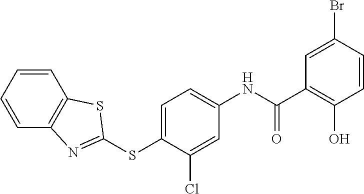 [SMILES: O=C(Nc1ccc(Sc2nc3ccccc3s2)c(Cl)c1)c1cc(Br)ccc1O]

N-(4-(Benzo[d]thiazol-2-ylthio)-3-
chlorophenyl)-5-bromo-2-hydroxybenzamide

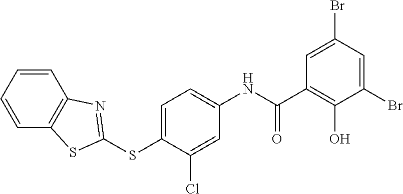 [SMILES: O=C(Nc1ccc(Sc2nc3ccccc3s2)c(Cl)c1)c1cc(Br)cc(Br)c1O]

N-(4-(Benzo[d]thiazol-2-ylthio)-3-
chlorophenyl)-3,5-dibromo-2-
hydroxybenzamide

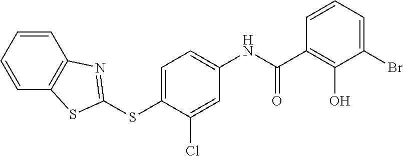 [SMILES: O=C(Nc1ccc(Sc2nc3ccccc3s2)c(Cl)c1)c1cccc(Br)c1O]

N-(4-(Benzo[d]thiazol-2-ylthio)-3-
chlorophenyl)-3-bromo-2-hydroxybenzamide

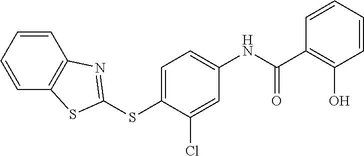 [SMILES: O=C(Nc1ccc(Sc2nc3ccccc3s2)c(Cl)c1)c1ccccc1O]

N-(4-(Benzo[d]thiazol-2-ylthio)-3-
chlorophenyl)-2-hydroxybenzamide

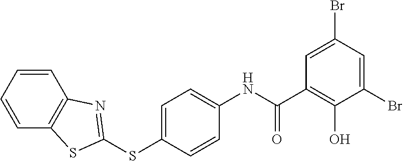 [SMILES: O=C(Nc1ccc(Sc2nc3ccccc3s2)cc1)c1cc(Br)cc(Br)c1O]

N-(4-(Benzo[d]thiazol-2-ylthio)phenyl)-3,5-
dibromo-2-hydroxybenzamide

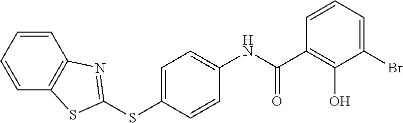 [SMILES: O=C(Nc1ccc(Sc2nc3ccccc3s2)cc1)c1cccc(Br)c1O]

N-(4-(Benzo[d]thiazol-2-ylthio)phenyl)-
3-bromo-2-hydroxybenzamide

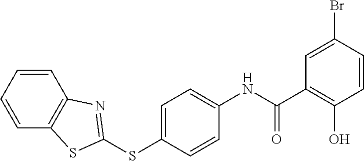 [SMILES: O=C(Nc1ccc(Sc2nc3ccccc3s2)cc1)c1cc(Br)ccc1O]

N-(4-(Benzo[d]thiazol-2-ylthio)phenyl)-5-
bromo-2-hydroxybenzamide

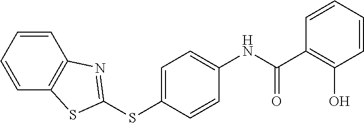 [SMILES: O=C(Nc1ccc(Sc2nc3ccccc3s2)cc1)c1ccccc1O]

N-(4-(Benzo[d]thiazol-2-ylthio)phenyl)-
2-hydroxybenzamide

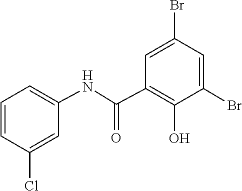 [SMILES: O=C(Nc1cccc(Cl)c1)c1cc(Br)cc(Br)c1O]

3,5-Dibromo-N-(3-chlorophenyl)-2-
hydroxybenzamide

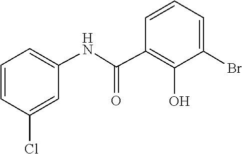 [SMILES: O=C(Nc1cccc(Cl)c1)c1cccc(Br)c1O]

3-Bromo-N-(3-chlorophenyl)-2-
hydroxybenzamide

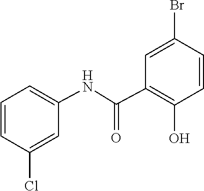 [SMILES: O=C(Nc1cccc(Cl)c1)c1cc(Br)ccc1O]

5-Bromo-N-(3-chlorophenyl)-2-
hydroxybenzamide

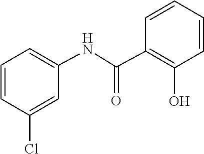 [SMILES: O=C(Nc1cccc(Cl)c1)c1ccccc1O]

N-(3-Chlorophenyl)-2-
hydroxybenzamide

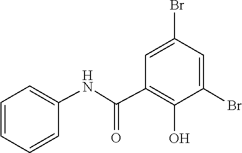 [SMILES: O=C(Nc1ccccc1)c1cc(Br)cc(Br)c1O]

3,5-Dibromo-2-hydroxy-N-phenylbenzamide

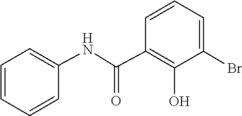 [SMILES: O=C(Nc1ccccc1)c1cccc(Br)c1O]

3-Bromo-2-hydroxy-N-phenylbenzamide

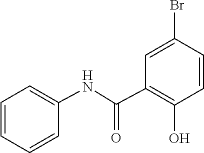 [SMILES: O=C(Nc1ccccc1)c1cc(Br)ccc1O]

5-Bromo-2-hydroxy-N-phenylbenzamide

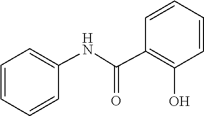 [SMILES: O=C(Nc1ccccc1)c1ccccc1O]

2-Hydroxy-N-phenylbenzamide

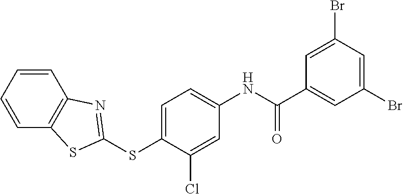 [SMILES: O=C(Nc1ccc(Sc2nc3ccccc3s2)c(Cl)c1)c1cc(Br)cc(Br)c1]

N-(4-(Benzo[d]thiazol-2-ylthio)-3-
chlorophenyl)-3,5-dibromobenzamide

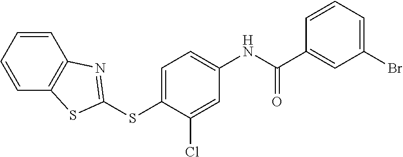 [SMILES: O=C(Nc1ccc(Sc2nc3ccccc3s2)c(Cl)c1)c1cccc(Br)c1]

N-(4-(Benzo[d]thiazol-2-ylthio)-3-
chlorophenyl)-3-bromobenzamide

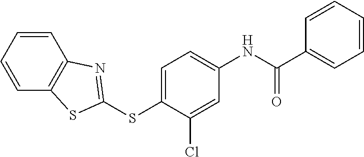 [SMILES: O=C(Nc1ccc(Sc2nc3ccccc3s2)c(Cl)c1)c1ccccc1]

N-(4-(Benzo[d]thiazol-2-ylthio)-3-
chlorophenyl)benzamide

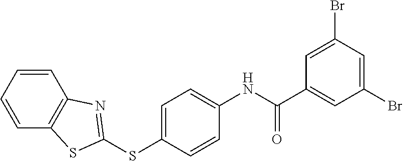 [SMILES: O=C(Nc1ccc(Sc2nc3ccccc3s2)cc1)c1cc(Br)cc(Br)c1]

N-(4-(Benzo[d]thiazol-2-ylthio)phenyl)-
3,5-dibromobenzamide

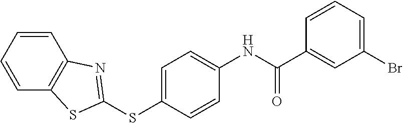 [SMILES: O=C(Nc1ccc(Sc2nc3ccccc3s2)cc1)c1cccc(Br)c1]

N-(4-(Benzo[d]thiazol-2-ylthio)phenyl)-3-
bromobenzamide

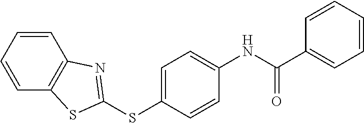 [SMILES: O=C(Nc1ccc(Sc2nc3ccccc3s2)cc1)c1ccccc1]

N-(4-(Benzo[d]thiazol-2-
ylthio)phenyl)benzamide

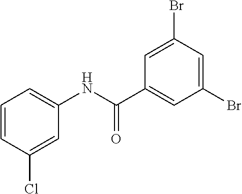 [SMILES: O=C(Nc1cccc(Cl)c1)c1cc(Br)cc(Br)c1]

3,5-Dibromo-N-(3-chlorophenyl)benzamide

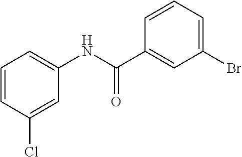 [SMILES: O=C(Nc1cccc(Cl)c1)c1cccc(Br)c1]

3-Bromo-N-(3-chlorophenyl)benzamide

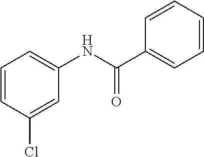 [SMILES: O=C(Nc1cccc(Cl)c1)c1ccccc1]

N-(3-Chlorophenyl)benzamide

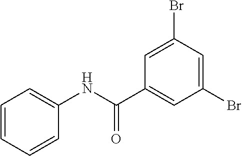 [SMILES: O=C(Nc1ccccc1)c1cc(Br)cc(Br)c1]

3,5-Dibromo-N-phenylbenzamide

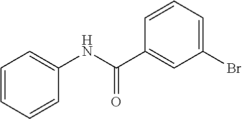 [SMILES: O=C(Nc1ccccc1)c1cccc(Br)c1]

3-Bromo-N-phenylbenzamide

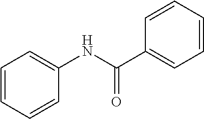 [SMILES: O=C(Nc1ccccc1)c1ccccc1]

N-Phenylbenzamide

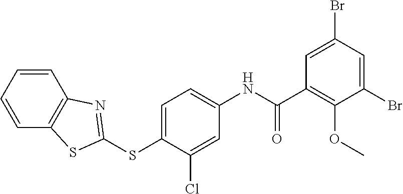 [SMILES: COc1c(Br)cc(Br)cc1C(=O)Nc1ccc(Sc2nc3ccccc3s2)c(Cl)c1]

N-(4-(Benzo[d]thiazol-2-ylthio)-3-
chlorophenyl)-3,5-dibromo-2-
methoxybenzamide

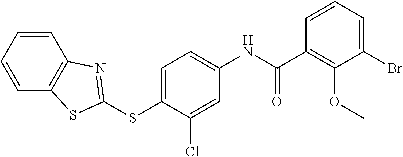 [SMILES: COc1c(Br)cccc1C(=O)Nc1ccc(Sc2nc3ccccc3s2)c(Cl)c1]

N-(4-(Benzo[d]thiazol-2-ylthio)-3-
chlorophenyl)-3-bromo-2-
methoxybenzamide

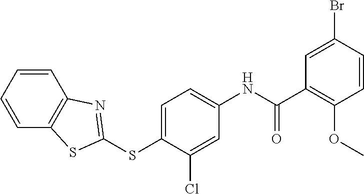 [SMILES: COc1ccc(Br)cc1C(=O)Nc1ccc(Sc2nc3ccccc3s2)c(Cl)c1]

(Benzo[d]thiazol-2-ylthio)-3-chlorophenyl)-5-
bromo-2-methoxybenzamide

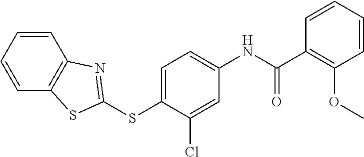 [SMILES: COc1ccccc1C(=O)Nc1ccc(Sc2nc3ccccc3s2)c(Cl)c1]

N-(4-(Benzo[d]thiazol-2-ylthio)-3-
chlorophenyl)-2-methoxybenzamide.

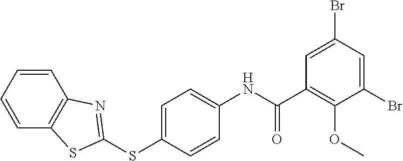 [SMILES: COc1c(Br)cc(Br)cc1C(=O)Nc1ccc(Sc2nc3ccccc3s2)cc1]

N-(4-(Benzo[d]thiazol-2-ylthio)phenyl)-3,5-
dibromo-2-methoxybenzamide

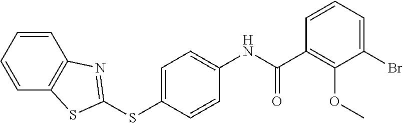 [SMILES: COc1c(Br)cccc1C(=O)Nc1ccc(Sc2nc3ccccc3s2)cc1]

N-(4-(Benzo[d]thiazol-2-ylthio)phenyl)-
3-bromo-2-methoxybenzamide

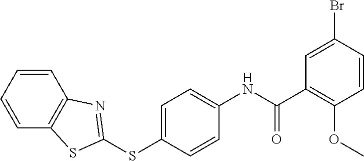 [SMILES: COc1ccc(Br)cc1C(=O)Nc1ccc(Sc2nc3ccccc3s2)cc1]

N-(4-(Benzo[d]thiazol-2-ylthio)phenyl)-5-
bromo-2-methoxybenzamide

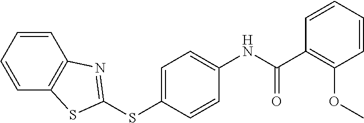 [SMILES: COc1ccccc1C(=O)Nc1ccc(Sc2nc3ccccc3s2)cc1]

(Benzo[d]thiazol-2-ylthio)phenyl)-2-
methoxybenzamide

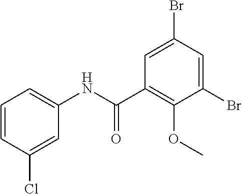 [SMILES: COc1c(Br)cc(Br)cc1C(=O)Nc1cccc(Cl)c1]

3,5-Dibromo-N-(3-chlorophenyl)-2-
methoxybenzamide

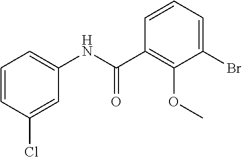 [SMILES: COc1c(Br)cccc1C(=O)Nc1cccc(Cl)c1]

3,5-Dibromo-N-(3-chlorophenyl)-2-
methoxybenzamide

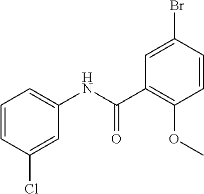 [SMILES: COc1ccc(Br)cc1C(=O)Nc1cccc(Cl)c1]

5-Bromo-N-(3-chlorophenyl)-2-
methoxybenzamide

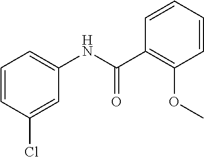 [SMILES: COc1ccccc1C(=O)Nc1cccc(Cl)c1]

N-(3-Chlorophenyl)-2-
methoxybenzamide

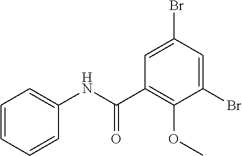 [SMILES: COc1c(Br)cc(Br)cc1C(=O)Nc1ccccc1]

3,5-Dibromo-2-methoxy-N-phenylbenzamide

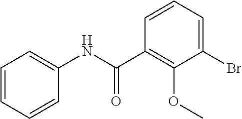 [SMILES: COc1c(Br)cccc1C(=O)Nc1ccccc1]

3-Bromo-2-methoxy-N-phenylbenzamide

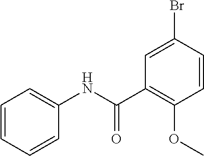 [SMILES: COc1ccc(Br)cc1C(=O)Nc1ccccc1]

5-Bromo-2-methoxy-N-phenylbenzamide

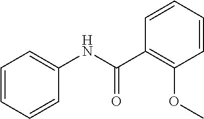 [SMILES: COc1ccccc1C(=O)Nc1ccccc1]

2-Methoxy-N-phenylbenzamide

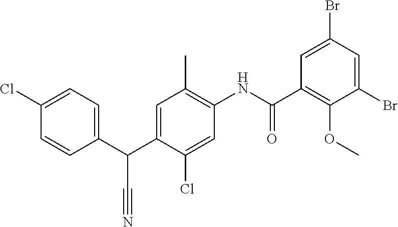 [SMILES: COc1c(Br)cc(Br)cc1C(=O)Nc1cc(Cl)c(C(C#N)c2ccc(Cl)cc2)cc1C]

3,5-dibromo-N-(5-chloro-4-((4-
chlorophenyl)(cyano)methyl)-2-
methylphenyl)-2-methoxybenzamide

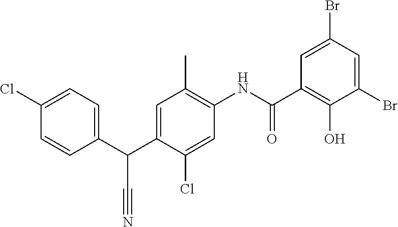 [SMILES: Cc1cc(C(C#N)c2ccc(Cl)cc2)c(Cl)cc1NC(=O)c1cc(Br)cc(Br)c1O]

3,5-dibromo-N-(5-chloro-4-((4-
chlorophenyl)(cyano)methyl)-2-
methylphenyl)-2-hydroxybenzamide

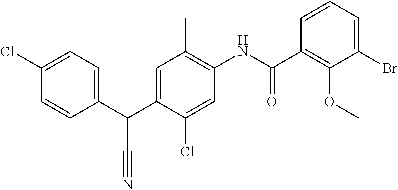 [SMILES: COc1c(Br)cccc1C(=O)Nc1cc(Cl)c(C(C#N)c2ccc(Cl)cc2)cc1C]

3-bromo-N-(5-chloro-4-((4-
chlorophenyl)(cyano)methyl)-2-
methylphenyl)-2-methoxybenzamide

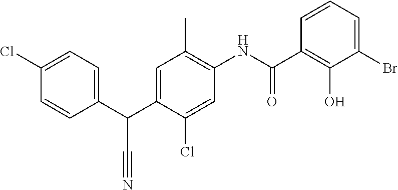 [SMILES: Cc1cc(C(C#N)c2ccc(Cl)cc2)c(Cl)cc1NC(=O)c1cccc(Br)c1O]

3-bromo-N-(5-chloro-4-((4-
chlorophenyl)(cyano)methyl)-2-
methylphenyl)-2-hydroxybenzamide.

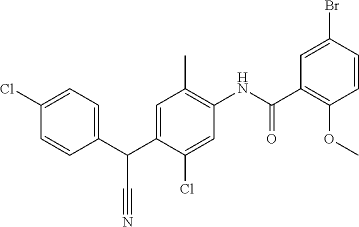 [SMILES: COc1ccc(Br)cc1C(=O)Nc1cc(Cl)c(C(C#N)c2ccc(Cl)cc2)cc1C]

5-bromo-N-(5-chloro-4-((4-
chlorophenyl)(cyano)methyl)-2-
methylphenyl)-2-methoxybenzamide

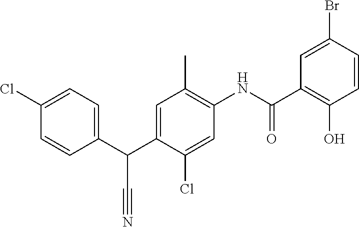 [SMILES: Cc1cc(C(C#N)c2ccc(Cl)cc2)c(Cl)cc1NC(=O)c1cc(Br)ccc1O]

5-bromo-N-(5-chloro-4-((4-
chlorophenyl)(cyano)methyl)-2-
methylphenyl)-2-hydroxybenzamide.

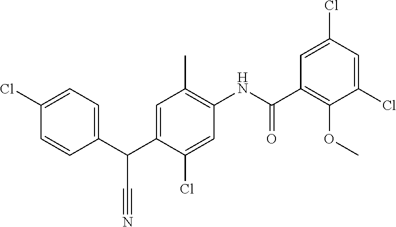 [SMILES: COc1c(Cl)cc(Cl)cc1C(=O)Nc1cc(Cl)c(C(C#N)c2ccc(Cl)cc2)cc1C]

3,5-dichloro-N-(5-chloro-4-((4-
chlorophenyl)(cyano)methyl)-2-
methylphenyl)-2-methoxybenzamide

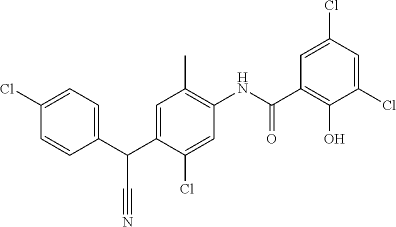 [SMILES: Cc1cc(C(C#N)c2ccc(Cl)cc2)c(Cl)cc1NC(=O)c1cc(Cl)cc(Cl)c1O]

3,5-dichloro-N-(5-chloro-4-((4-
chlorophenyl)(cyano)methyl)-2-
methylphenyl)-2-hydroxybenzamide

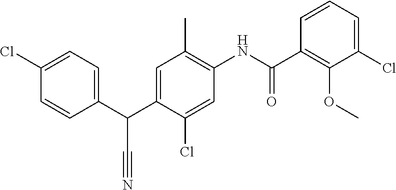 [SMILES: COc1c(Cl)cccc1C(=O)Nc1cc(Cl)c(C(C#N)c2ccc(Cl)cc2)cc1C]

3-chloro-N-(5-chloro-4-((4-
chlorophenyl)(cyano)methyl)-2-
methylphenyl)-2-methoxybenzamide

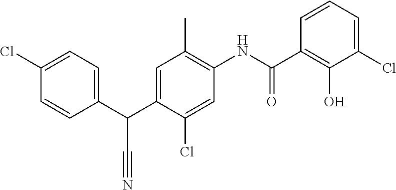 [SMILES: Cc1cc(C(C#N)c2ccc(Cl)cc2)c(Cl)cc1NC(=O)c1cccc(Cl)c1O]

3-chloro-N-(5-chloro-4-((4-
chlorophenyl)(cyano)methyl)-2-
methylphenyl)-2-hydroxybenzamide

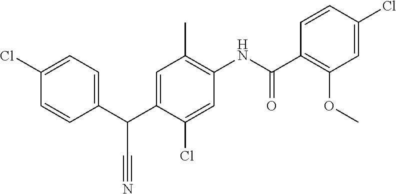 [SMILES: COc1cc(Cl)ccc1C(=O)Nc1cc(Cl)c(C(C#N)c2ccc(Cl)cc2)cc1C]

4-chloro-N-(5-chloro-4-((4-
chlorophenyl)(cyano)methyl)-2-
methylphenyl)-2-methoxybenzamide

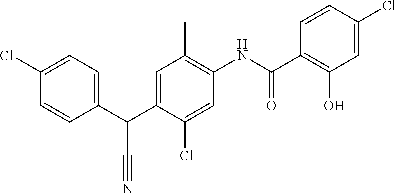 [SMILES: Cc1cc(C(C#N)c2ccc(Cl)cc2)c(Cl)cc1NC(=O)c1ccc(Cl)cc1O]

4-chloro-N-(5-chloro-4-((4-
chlorophenyl)(cyano)methyl)-2-
methylphenyl)-2-hydroxybenzamide

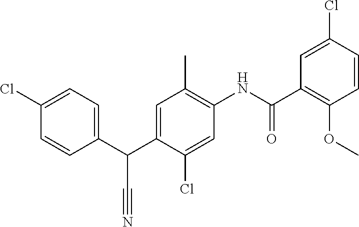 [SMILES: COc1ccc(Cl)cc1C(=O)Nc1cc(Cl)c(C(C#N)c2ccc(Cl)cc2)cc1C]

5-chloro-N-(5-chloro-4-((4-
chlorophenyl)(cyano)methyl)-2-
methylphenyl)-2-methoxybenzamide

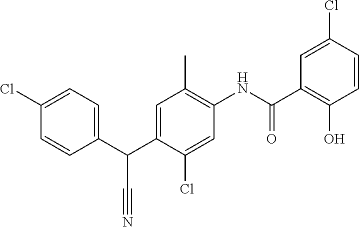 [SMILES: Cc1cc(C(C#N)c2ccc(Cl)cc2)c(Cl)cc1NC(=O)c1cc(Cl)ccc1O]

5-chloro-N-(5-chloro-4-((4-
chlorophenyl)(cyano)methyl)-2-
methylphenyl)-2-hydroxybenzamide

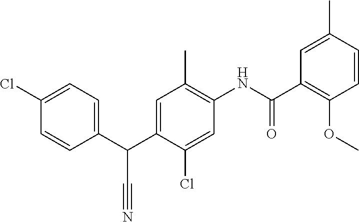 [SMILES: COc1ccc(C)cc1C(=O)Nc1cc(Cl)c(C(C#N)c2ccc(Cl)cc2)cc1C]

N-(5-chloro-4-((4-
chlorophenyl)(cyano)methyl)-2-
methylphenyl)-2-methoxy-5-
methylbenzamide

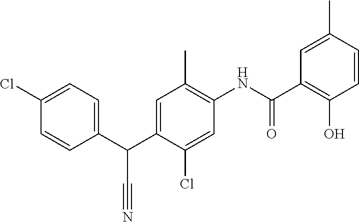 [SMILES: Cc1ccc(O)c(C(=O)Nc2cc(Cl)c(C(C#N)c3ccc(Cl)cc3)cc2C)c1]

N-(5-chloro-4-((4-
chlorophenyl)(cyano)methyl)-2-
methylphenyl)-2-hydroxy-5-
methylbenzamide

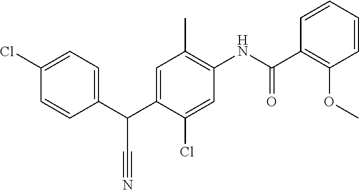 [SMILES: COc1ccccc1C(=O)Nc1cc(Cl)c(C(C#N)c2ccc(Cl)cc2)cc1C]

N-(5-chloro-4-((4-
chlorophenyl)(cyano)methyl)-2-
methylphenyl)-2-methoxybenzamide

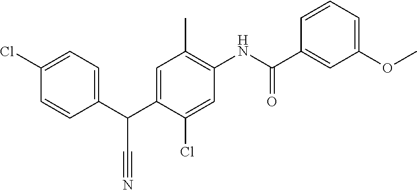 [SMILES: COc1cccc(C(=O)Nc2cc(Cl)c(C(C#N)c3ccc(Cl)cc3)cc2C)c1]

N-(5-chloro-4-((4-
chlorophenyl)(cyano)methyl)-2-
methylphenyl)-3-methoxybenzamide

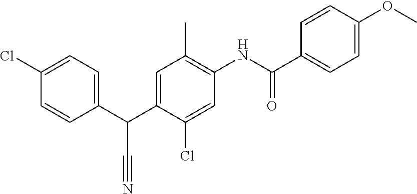 [SMILES: COc1ccc(C(=O)Nc2cc(Cl)c(C(C#N)c3ccc(Cl)cc3)cc2C)cc1]

N-(5-chloro-4-((4-
chlorophenyl)(cyano)methyl)-2-
methylphenyl)-4-methoxybenzamide

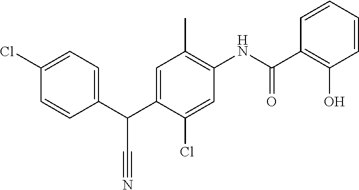 [SMILES: Cc1cc(C(C#N)c2ccc(Cl)cc2)c(Cl)cc1NC(=O)c1ccccc1O]

N-(5-chloro-4-((4-
chlorophenyl)(cyano)methyl)-2-
methylphenyl)-2-hydroxybenzamide

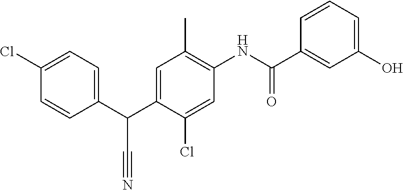 [SMILES: Cc1cc(C(C#N)c2ccc(Cl)cc2)c(Cl)cc1NC(=O)c1cccc(O)c1]

N-(5-chloro-4-((4-
chlorophenyl)(cyano)methyl)-2-
methylphenyl)-3-hydroxybenzamide

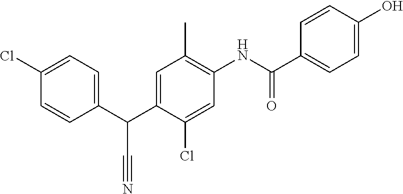 [SMILES: Cc1cc(C(C#N)c2ccc(Cl)cc2)c(Cl)cc1NC(=O)c1ccc(O)cc1]

N-(5-chloro-4-((4-
chlorophenyl)(cyano)methyl)-2-
methylphenyl)-4-hydroxybenzamide

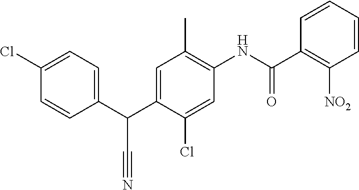 [SMILES: Cc1cc(C(C#N)c2ccc(Cl)cc2)c(Cl)cc1NC(=O)c1ccccc1[N+](=O)[O-]]

N-(5-chloro-4-((4-
chlorophenyl)(cyano)methyl)-2-
methylphenyl)-2-nitrobenzamide

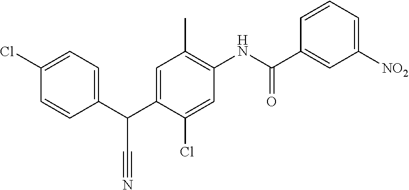 [SMILES: Cc1cc(C(C#N)c2ccc(Cl)cc2)c(Cl)cc1NC(=O)c1cccc([N+](=O)[O-])c1]

N-(5-chloro-4-((4-
chlorophenyl)(cyano)methyl)-2-
methylphenyl)-3-nitrobenzamide.

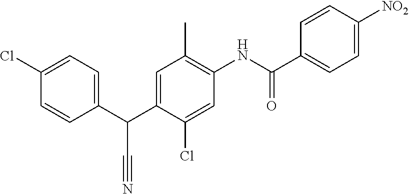 [SMILES: Cc1cc(C(C#N)c2ccc(Cl)cc2)c(Cl)cc1NC(=O)c1ccc([N+](=O)[O-])cc1]

N-(5-chloro-4-((4-
chlorophenyl)(cyano)methyl)-2-
methylphenyl)-4-nitrobenzamide

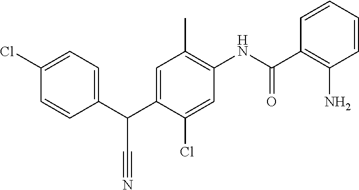 [SMILES: Cc1cc(C(C#N)c2ccc(Cl)cc2)c(Cl)cc1NC(=O)c1ccccc1N]

2-amino-N-(5-chloro-4-((4-
chlorophenyl)(cyano)methyl)-2-
methylphenyl)benzamide

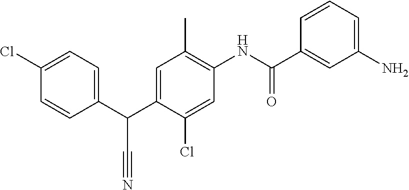 [SMILES: Cc1cc(C(C#N)c2ccc(Cl)cc2)c(Cl)cc1NC(=O)c1cccc(N)c1]

3-amino-N-(5-chloro-4-((4-
chlorophenyl)(cyano)methyl)-2-
methylphenyl)benzamide

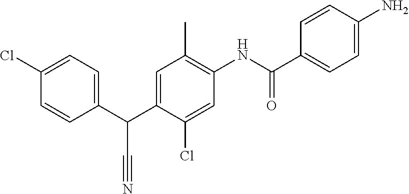 [SMILES: Cc1cc(C(C#N)c2ccc(Cl)cc2)c(Cl)cc1NC(=O)c1ccc(N)cc1]

4-amino-N-(5-chloro-4-((4-
chlorophenyl)(cyano)methyl)-2-
methylphenyl)benzamide

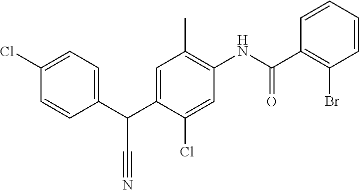 [SMILES: Cc1cc(C(C#N)c2ccc(Cl)cc2)c(Cl)cc1NC(=O)c1ccccc1Br]

2-bromo-N-(5-chloro-4-((4-
chlorophenyl)(cyano)methyl)-2-
methylphenyl)benzamide

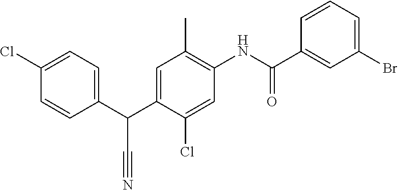 [SMILES: Cc1cc(C(C#N)c2ccc(Cl)cc2)c(Cl)cc1NC(=O)c1cccc(Br)c1]

3-bromo-N-(5-chloro-4-((4-
chlorophenyl)(cyano)methyl)-2-
methylphenyl)benzamide

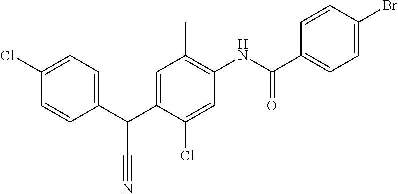 [SMILES: Cc1cc(C(C#N)c2ccc(Cl)cc2)c(Cl)cc1NC(=O)c1ccc(Br)cc1]

4-bromo-N-(5-chloro-4-((4-
chlorophenyl)(cyano)methyl)-2-
methylphenyl)benzamide

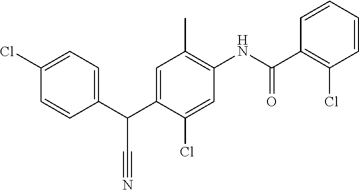 [SMILES: Cc1cc(C(C#N)c2ccc(Cl)cc2)c(Cl)cc1NC(=O)c1ccccc1Cl]

2-chloro-N-(5-chloro-4-((4-
chlorophenyl)(cyano)methyl)-2-
methylphenyl)benzamide

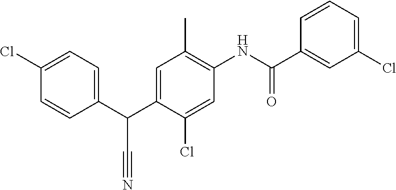 [SMILES: Cc1cc(C(C#N)c2ccc(Cl)cc2)c(Cl)cc1NC(=O)c1cccc(Cl)c1]

3-chloro-N-(5-chloro-4-((4-
chlorophenyl)(cyano)methyl)-2-
methylphenyl)benzamide

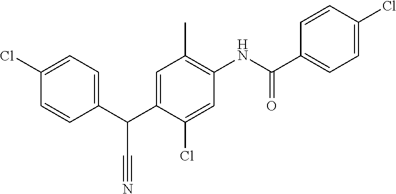 [SMILES: Cc1cc(C(C#N)c2ccc(Cl)cc2)c(Cl)cc1NC(=O)c1ccc(Cl)cc1]

4-chloro-N-(5-chloro-4-((4-
chlorophenyl)(cyano)methyl)-2-
methylphenyl)benzamide

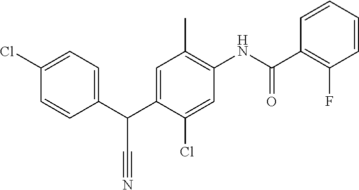 [SMILES: Cc1cc(C(C#N)c2ccc(Cl)cc2)c(Cl)cc1NC(=O)c1ccccc1F]

N-(5-chloro-4-((4-
chlorophenyl)(cyano)methyl)-2-
methylphenyl)-2-fluorobenzamide

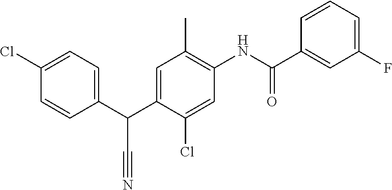 [SMILES: Cc1cc(C(C#N)c2ccc(Cl)cc2)c(Cl)cc1NC(=O)c1cccc(F)c1]

N-(5-chloro-4-((4-
chlorophenyl)(cyano)methyl)-2-
methylphenyl)-3-fluorobenzamide

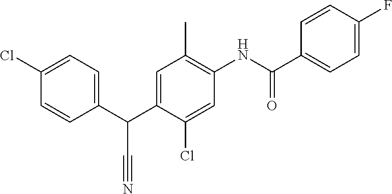 [SMILES: Cc1cc(C(C#N)c2ccc(Cl)cc2)c(Cl)cc1NC(=O)c1ccc(F)cc1]

N-(5-chloro-4-((4-
chlorophenyl)(cyano)methyl)-2-
methylphenyl)-4-fluorobenzamide

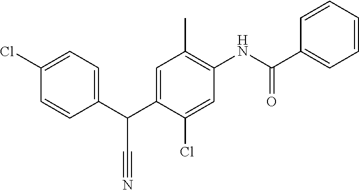 [SMILES: Cc1cc(C(C#N)c2ccc(Cl)cc2)c(Cl)cc1NC(=O)c1ccccc1]

N-(5-chloro-4-((4-
chlorophenyl)(cyano)methyl)-2-
methylphenyl)benzamide

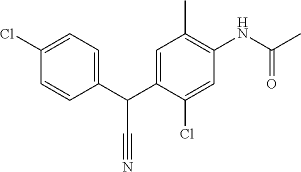 [SMILES: CC(=O)Nc1cc(Cl)c(C(C#N)c2ccc(Cl)cc2)cc1C]

N-(5-chloro-4-((4-
chlorophenyl)(cyano)methyl)-2-
methylphenyl)acetamide

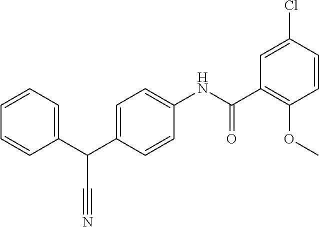 [SMILES: COc1ccc(Cl)cc1C(=O)Nc1ccc(C(C#N)c2ccccc2)cc1]

5-chloro-N-(4-
(cyano(phenyl)methyl)phenyl)-2-
methoxybenzamide

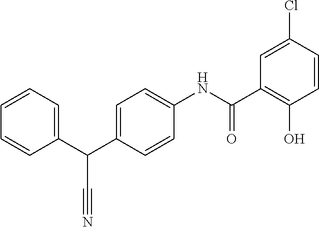 [SMILES: N#CC(c1ccccc1)c1ccc(NC(=O)c2cc(Cl)ccc2O)cc1]

5-chloro-N-(4-(cyano(phenyl)methyl)phenyl)-
2-hydroxybenzamide

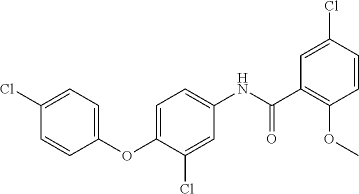 [SMILES: COc1ccc(Cl)cc1C(=O)Nc1ccc(Oc2ccc(Cl)cc2)c(Cl)c1]

5-chloro-N-(3-chloro-4-(4-
chlorophenoxy)phenyl)-2-
methoxybenzamide

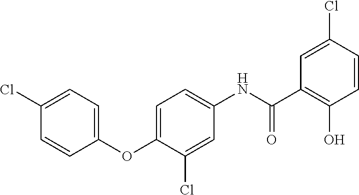 [SMILES: O=C(Nc1ccc(Oc2ccc(Cl)cc2)c(Cl)c1)c1cc(Cl)ccc1O]

5-chloro-N-(3-chloro-4-(4-
chlorophenoxy)phenyl)-2-hydroxybenzamide

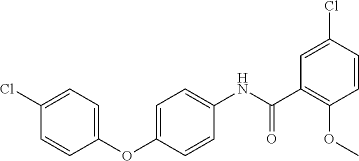 [SMILES: COc1ccc(Cl)cc1C(=O)Nc1ccc(Oc2ccc(Cl)cc2)cc1]

5-chloro-N-(4-(4-
chlorophenoxy)phenyl)-2-
methoxybenzamide

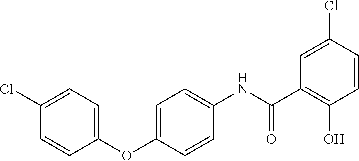 [SMILES: O=C(Nc1ccc(Oc2ccc(Cl)cc2)cc1)c1cc(Cl)ccc1O]

5-chloro-N-(4-(4-chlorophenoxy)phenyl)-2-
hydroxybenzamide

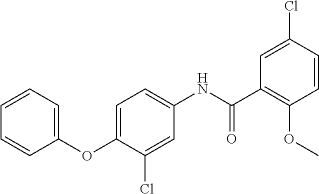 [SMILES: COc1ccc(Cl)cc1C(=O)Nc1ccc(Oc2ccccc2)c(Cl)c1]

5-chloro-N-(3-chloro-4-phenoxyphenyl)-
2-methoxybenzamide

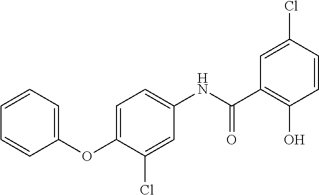 [SMILES: O=C(Nc1ccc(Oc2ccccc2)c(Cl)c1)c1cc(Cl)ccc1O]

5-chloro-N-(3-chloro-4-phenoxyphenyl)-2-
hydroxybenzamide

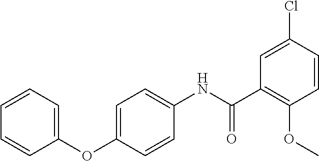 [SMILES: COc1ccc(Cl)cc1C(=O)Nc1ccc(Oc2ccccc2)cc1]

5-chloro-2-methoxy-N-(4-
phenoxyphenyl)benzamide

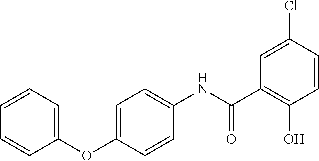 [SMILES: O=C(Nc1ccc(Oc2ccccc2)cc1)c1cc(Cl)ccc1O]

5-chloro-2-hydroxy-N-(4-
phenoxyphenyl)benzamide

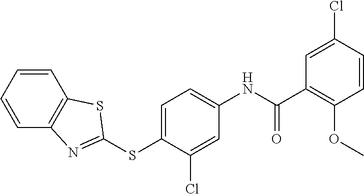 [SMILES: COc1ccc(Cl)cc1C(=O)Nc1ccc(Sc2nc3ccccc3s2)c(Cl)c1]

N-(4-(benzo[d]thiazol-2-ylthio)-3-
chlorophenyl)-5-chloro-2-
methoxybenzamide

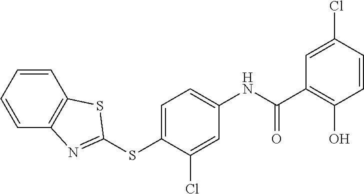 [SMILES: O=C(Nc1ccc(Sc2nc3ccccc3s2)c(Cl)c1)c1cc(Cl)ccc1O]

N-(4-(benzo[d]thiazol-2-ylthio)-3-
chlorophenyl)-5-chloro-2-hydroxybenzamide

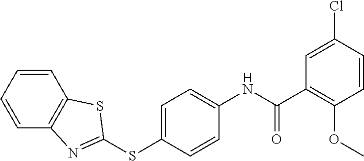 [SMILES: COc1ccc(Cl)cc1C(=O)Nc1ccc(Sc2nc3ccccc3s2)cc1]

N-(4-(benzo[d]thiazol-2-ylthio)phenyl)-
5-chloro-2-methoxybenzamide

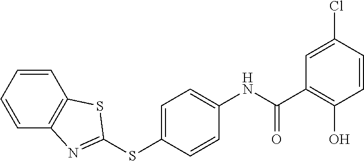 [SMILES: O=C(Nc1ccc(Sc2nc3ccccc3s2)cc1)c1cc(Cl)ccc1O]

N-(4-(benzo[d]thiazol-2-ylthio)phenyl)-5-
chloro-2-hydroxybenzamide

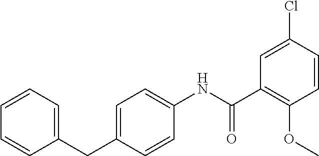 [SMILES: COc1ccc(Cl)cc1C(=O)Nc1ccc(Cc2ccccc2)cc1]

N-(4-benzylphenyl)-5-chloro-2-
methoxybenzamide

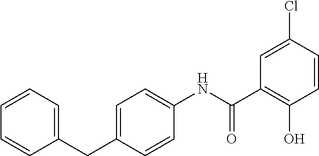 [SMILES: O=C(Nc1ccc(Cc2ccccc2)cc1)c1cc(Cl)ccc1O]

N-(4-benzylphenyl)-5-chloro-2-
hydroxybenzamide

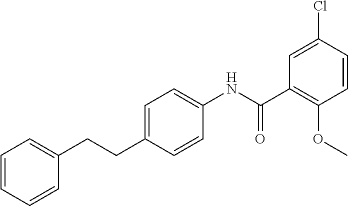 [SMILES: COc1ccc(Cl)cc1C(=O)Nc1ccc(CCc2ccccc2)cc1]

5-chloro-2-methoxy-N-(4-
phenethylphenyl)benzamide

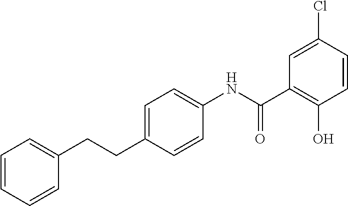 [SMILES: O=C(Nc1ccc(CCc2ccccc2)cc1)c1cc(Cl)ccc1O]

5-chloro-2-methoxy-N-(4-
phenethylphenyl)benzamide

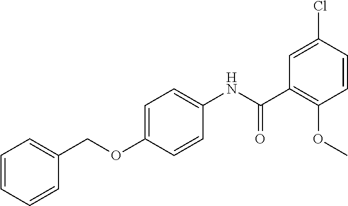 [SMILES: COc1ccc(Cl)cc1C(=O)Nc1ccc(OCc2ccccc2)cc1]

N-(4-(benzyloxy)phenyl)-5-chloro-2-
methoxybenzamide

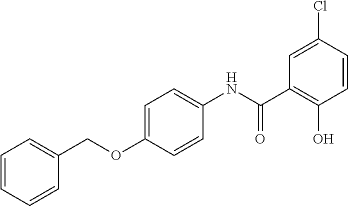 [SMILES: O=C(Nc1ccc(OCc2ccccc2)cc1)c1cc(Cl)ccc1O]

N-(4-(benzyloxy)phenyl)-5-chloro-2-
hydroxybenzamide

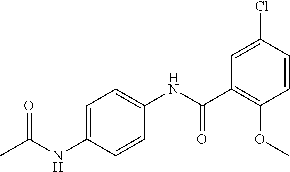 [SMILES: COc1ccc(Cl)cc1C(=O)Nc1ccc(NC(C)=O)cc1]

N-(4-acetamidophenyl)-5-chloro-2-
methoxybenzamide

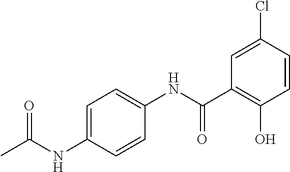 [SMILES: CC(=O)Nc1ccc(NC(=O)c2cc(Cl)ccc2O)cc1]

N-(4-acetamidophenyl)-5-chloro-2-
hydroxybenzamide

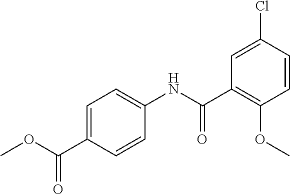

Methyl 4-(5-chloro-2-
methoxybenzamido)benzoate

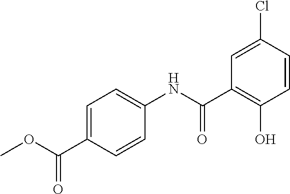 [SMILES: COC(=O)c1ccc(NC(=O)c2cc(Cl)ccc2O)cc1]

methyl 4-(5-chloro-2-
hydroxybenzamido)benzoate

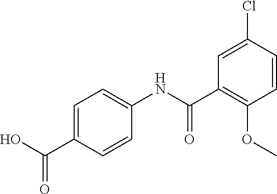 [SMILES: COc1ccc(Cl)cc1C(=O)Nc1ccc(C(=O)O)cc1]

4-(5-chloro-2-
methoxybenzamido)benzoic acid

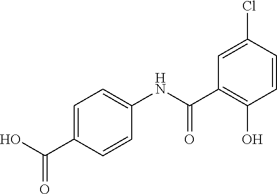 [SMILES: O=C(O)c1ccc(NC(=O)c2cc(Cl)ccc2O)cc1]

4-(5-chloro-2-hydroxybenzamido)benzoic
acid

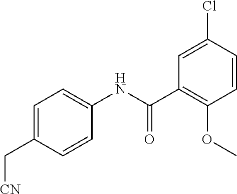 [SMILES: COc1ccc(Cl)cc1C(=O)Nc1ccc(CC#N)cc1]

5-chloro-N-(4-(cyanomethyl)phenyl)-2-
methoxybenzamide

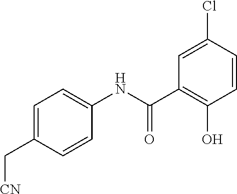 [SMILES: N#CCc1ccc(NC(=O)c2cc(Cl)ccc2O)cc1]

5-chloro-N-(4-(cyanomethyl)phenyl)-2-
hydroxybenzamide

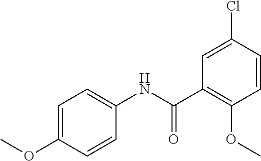

5-chloro-2-methoxy-N-(4-
methoxyphenyl)benzamide

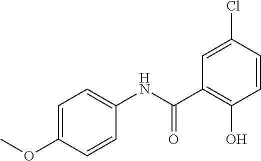 [SMILES: COc1ccc(NC(=O)c2cc(Cl)ccc2O)cc1]

5-chloro-2-hydroxy-N-(4-
methoxyphenyl)benzamide

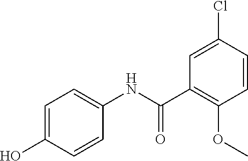 [SMILES: COc1ccc(Cl)cc1C(=O)Nc1ccc(O)cc1]

5-chloro-N-(4-hydroxyphenyl)-2-
methoxybenzamide

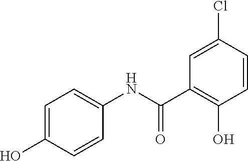 [SMILES: O=C(Nc1ccc(O)cc1)c1cc(Cl)ccc1O]

5-chloro-2-hydroxy-N-(4-
hydroxyphenyl)benzamide

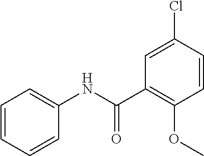 [SMILES: COc1ccc(Cl)cc1C(=O)Nc1ccccc1]

5-chloro-2-methoxy-N-phenylbenzamide

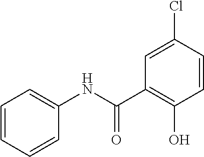 [SMILES: O=C(Nc1ccccc1)c1cc(Cl)ccc1O]

5-chloro-2-hydroxy-N-phenylbenzamide

and pharmaceutically acceptable salts thereof.

In some embodiments, the disclosure relates to a compound or a pharmaceutically acceptable salt thereof, having the formula (XIII) or (XIV),

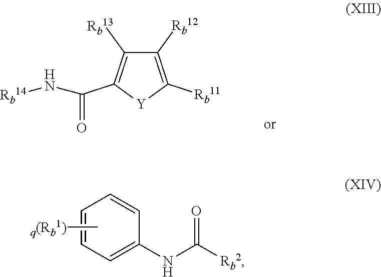

wherein
Y is O or S;
R_{b}¹ is halo, C₁-C₈ alkyl, aryl (e.g., C₆-C₁₀ aryl or biaryl), 5- to 12-membered heteroaryl, phenoxy, benzyl, —C(O)R^{A}, —OR^{A}, —SR^{A} or —NHR^{A}, or one or more R_{b}¹ in combination with the atoms to which each is attached combine to form a C₉-C₁₄ aryl or C₉-C₁₂ bicyclic heteroaryl, wherein C₆-C₁₀ aryl, 5- to 12-membered heteroaryl, phenoxy, benzyl, C₁-C₈alkyl, —C(O)C₂-C₆ alkenylene-phenyl, C₉-C₁₂ aryl and C₉-C₁₂ bicyclic heteroaryl is optionally substituted by halo, cyano, or —NO₂;
R_{b}² is C₆-C₁₀ aryl or 5- to 12-membered heteroaryl, wherein C₆-C₁₀ aryl and 5- to 12-membered heteroaryl is optionally substituted by halo, hydroxy, or —NO₂;
R_{b}¹¹ is H, nitro, or N(H)COR_{b}³;
R_{b}¹² is H or C(O)OC₁-C₆ alkyl;
R_{b}¹³ is H or C₁-C₆ alkyl;
R_{b}¹⁴ is H, C₆-C₁₀ aryl, or 5 to 12-membered heteroaryl, wherein C₆-C₁₀ aryl and 5 to 12-membered heteroaryl is optionally substituted by halo, 5 to 12-membered heteroaryl, —NHC(O)C₁-C₆ alkyl, C₁-C₆ alkoxy, —C(O)C₁-C₆ alkoxy, —R^{A}, —OR^{A}, —SR^{A}, —C(O)R^{A} or —NHR^{A};
each R^{A} is independently C₁-C₆ alkyl, C₆-C₁₀ aryl, C₂-C₆ alkenyl-C₆-C₁₀ aryl, —C(O)C₁-C₆ alkyl, C₁-C₆ alkoxy, or 5 to 12-membered heteroaryl, wherein C₁-C₆ alkyl, C₆-C₁₀ aryl, C₂-C₆ alkenyl-C₆-C₁₀ aryl, —C(O)C₁-C₆ alkyl, C₁-C₆ alkoxy, or 5 to 12-membered heteroaryl is optionally substituted by nitro or halo; or two R^{A} combine to form C₄-C₆ cycloalkyl optionally substituted by phenyl; and
R_{b}³ is C₆-C₁₀ aryl, 5 to 12-membered heteroaryl, or C₉-C₁₂ bicyclic aryl, wherein C₆-C₁₀ aryl, 5 to 12-membered heteroaryl, and C₉-C₁₂ bicyclic aryl is optionally substituted by halo, C₁-C₆alkyl, or nitro; and
q is 0, 1, 2, or 3.

In some embodiments, the disclosure relates to a compound or a pharmaceutically acceptable salt thereof, having the formula (XV) or (XVI)

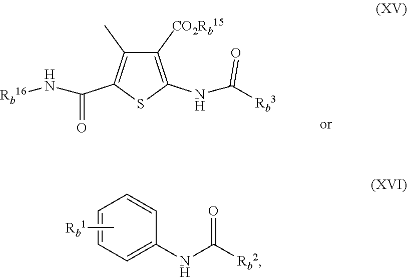

wherein
R_{b}¹ is H, halo, aryl, heteroaryl, substituted aryl, substituted heteroaryl, phenoxy, alkyl, or R_{b}¹ in combination with the atom to which it is attached forms a bicyclic aryl or bicyclic heteroaryl, wherein said substituted aryl and substituted heteroaryl comprise 1 to 5 substituents selected from halo and —NO₂;
R_{b}² is aryl, heteroaryl, substituted aryl, substituted heteroaryl, wherein said substituted aryl and substituted heteroaryl comprise 1 to 5 substituents selected from halo and —NO₂;
R_{b}³ is aryl, heteroaryl, bicyclic aryl, bicyclic heteroaryl, substituted aryl, substituted heteroaryl, substituted bicyclic aryl or substituted bicyclic heteroaryl, wherein said substituted aryl, substituted heteroaryl, substituted bicyclic aryl or substituted bicyclic heteroaryl comprise 1 to 5 substituents selected from halo and —NO₂; and
R_{b}¹⁵ and R_{b}¹⁶ are each independently hydrogen or C₁-C₆ alkyl.

In some embodiments, the disclosure relates to a compound or a pharmaceutically acceptable salt thereof, having the formula (XV), wherein
R_{b}³ is C₅-C₈ aryl, C₅-C₈ heteroaryl, a C₉-C₁₂ bicyclic aryl or C₉-C₁₂ bicyclic heteroaryl, wherein one or more of the hydrogen atoms of the R_{b}³ substituents is optionally substituted with halogen, —OR_{b}⁵, nitro or —NR_{b}⁵R_{b}⁶;
R_{b}⁵ and R_{b}⁶ are each independently hydrogen or —CH₃;
R_{b}¹⁵ and R_{b}¹⁶ are each independently hydrogen, C₁-C₆ alkyl, C₆-C₁₀ aryl, or bicyclic C₆-C₁₀ heteroaryl; optionally wherein R_{b}¹⁵ and R_{b}¹⁶ are each independently hydrogen or —CH₃.

In some embodiments, the disclosure relates to a compound or a pharmaceutically acceptable salt thereof, having the formula (XV), wherein
R_{b}³ is

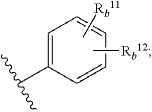

R_{b}¹¹ is halo (e.g., fluoro, chloro, bromo, and iodo);
R_{b}¹² is —NO₂; and
R_{b}¹⁵ and R¹⁶ are each independently hydrogen or C₁-C₆ alkyl.

In some embodiments, the disclosure relates to a compound or a pharmaceutically acceptable salt thereof, having the formula (XVII),

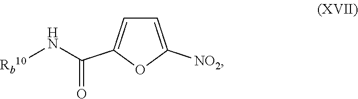

wherein
R_{b}¹⁰ is C₅-C₈ aryl, C₅-C₈ heteroaryl, C₉-C₁₂ bicyclic aryl or C₉-C₁₂ bicyclic heteroaryl, wherein one or more of the hydrogen atoms of the R¹⁰ substituent is optionally substituted with halo, phenoxy, —OR⁵, —NR_{b}⁵R_{b}⁶, —S(O)₂NR_{b}⁵R_{b}⁶, or —NHSO₂R_{b}⁷;
R_{b}⁵ and R_{b}⁶ are each independently hydrogen, C₁-C₆ alkyl, C₆-C₁₀ aryl, or bicyclic C₆-C₁₀ heteroaryl, wherein each hydrogen atom in C₁-C₆ alkyl, aryl, and heteroaryl is optionally substituted with halogen or —OC₁-C₆ alkyl; and
R_{b}⁷ is C₅-C₆ aryl, or C₅-C₁₆ heteroaryl wherein one or more of the hydrogen atoms of the C₅-C₆ aryl, or C₅-C₁₆ heteroaryl is optionally substituted with halo, —OR⁵ or nitro; or a pharmaceutically acceptable salt thereof, optionally wherein R⁵ and R⁶ are each independently hydrogen or —CH₃.

In some embodiments of compounds of formula XIII, Y is O or S. In some embodiments Y is O. In some embodiments, Y is S.

In some embodiments of compounds of formula XIII or XIV, q is 0, 1, 2, or 3. In some embodiments, q is 0 and the phenyl ring is unsubstituted. In some embodiments, q is 1. In certain embodiments when q is 1, R_{b}¹ is optionally substituted phenoxy. In some embodiments, q is 2. In some embodiments, q is 2 and the two R_{b}¹ in combination with the atoms to which each is attached combine to form a C₉-C₁₄ aryl

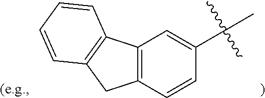 [SMILES: *c1ccc2c(c1)-c1ccccc1C2]

or C₉-C₁₂ bicyclic heteroaryl, each of which may be optionally substituted, for example by a heteroaryl.

In some embodiments of compounds of formula XIII or XIV, R_{b}¹ is halo, C₁-C₈ alkyl, C₆-C₁₀ aryl, 5- to 12-membered heteroaryl, phenoxy, benzyl, —C(O)R^{A}, —OR^{A}, —SR^{A} or —NHR^{A}. In some embodiments, one or more R¹ in combination with the atoms to which each is attached combine to form a C₉-C₁₄ aryl or C₉-C₁₂ bicyclic heteroaryl. In certain embodiments, each hydrogen atom in C₆-C₁₀ aryl, 5- to 12-membered heteroaryl, phenoxy, benzyl, C₁-C₈ alkyl, —C(O)C₂-C₆ alkenylene-phenyl, C₉-C₁₂ aryl and C₉-C₁₂ bicyclic heteroaryl is optionally substituted by halo, cyano, or —NO₂.

In some embodiments of compounds of formula XIII or XIV, R_{b}² is C₆-C₁₀ aryl or 5- to 12-membered heteroaryl, wherein each hydrogen atom in C₆-C₁₀ aryl and 5- to 12-membered heteroaryl is optionally substituted by halo, hydroxy, or —NO₂. For example, R_{b}² may be phenyl optionally substituted by alkoxy, ester, halo such as chloro, etc. In some embodiments, R² is 5- to 12-membered heteroaryl, for example

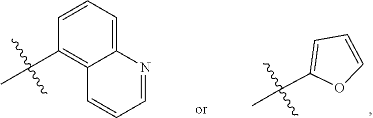 [SMILES: *c1cccc2ncccc12 ; *c1ccco1]

each of which may be optionally substituted.

In some embodiments of compounds of formula XV, R_{b}¹¹ is H, nitro, or —N(H)COR_{b}³. In some embodiments, R_{b}¹¹ is H or nitro. In some embodiments, R_{b}¹¹ is H.

In some embodiments of compounds of formula XV, R_{b}¹² is H or —C(O)OC₁-C₆ alkyl (e.g., —C(O)ethyl).

In some embodiments of compounds of formula XIII or XIV, R_{b}¹³ is H or C₁-C₆ alkyl (e.g., methyl).

In some embodiments of compounds of formula XIII or XIV, R_{b}¹⁴ is H, C₆-C₁₀ aryl (e.g., phenyl), or 5 to 12-membered heteroaryl, wherein each hydrogen atom in C₆-C₁₀ aryl and 5 to 12-membered heteroaryl is optionally substituted by halo, 5 to 12-membered heteroaryl, —NHC(O)C₁-C₆alkyl, C₁-C₆ alkoxy, —C(O)C₁-C₆ alkoxy, —R^{A}, —OR^{A}, —SR^{A}, —C(O)R^{A} or —NHR^{A}. In some embodiments of compounds of formula XIII, R_{b}¹¹ is H or nitro.

In some embodiments of compounds of formula XIII, R_{b}¹² and R_{b}¹³ are each H.

In some embodiments of compounds of formula XIII, R_{b}¹⁴ is C₆-C₁₀ aryl, wherein C₆-C₁₀ aryl is optionally substituted by —C(O)R^{A}; and R^{A} is C₂-C₆ alkenyl-C₆-C₁₀ aryl, wherein C₂-C₆ alkenyl-C₆-C₁₀ aryl is optionally substituted by nitro. In some embodiments, R_{b}¹⁴ is

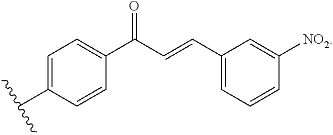 [SMILES: *c1ccc(C(=O)/C=C/c2cccc([N+](=O)[O-])c2)cc1]

In some embodiments of compounds of formula XIII or XIV, each R^{A} is C₁-C₆ alkyl (e.g., methyl), C₆-C₁₀ aryl, C₂-C₆ alkenyl-C₆-C₁₀ aryl, —C(O)C₁-C₆ alkyl, C₁-C₆ alkoxy (e.g., methoxy), or 5 to 12-membered heteroaryl, wherein each hydrogen atom in C₁-C₆ alkyl, C₆-C₁₀ aryl, C₂-C₆ alkenyl-C₆-C₁₀ aryl, —C(O)C₁-C₆ alkyl, C₁-C₆ alkoxy, or 5 to 12-membered heteroaryl is optionally substituted by nitro or halo; or two R^{A} combine to form C₄-C₆ cycloalkyl optionally substituted by phenyl, for example if R_{b}¹⁴ is phenyl, two R^{A} can combine with the phenyl to form

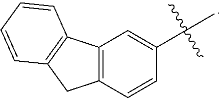 [SMILES: *c1ccc2c(c1)-c1ccccc1C2]

In some embodiments of compounds of formula XIII or XIV, R_{b}³ is C₆-C₁₀ aryl, 5 to 12-membered heteroaryl, or C₉-C₁₂ bicyclic aryl, wherein each hydrogen atom in C₆-C₁₀ aryl, 5 to 12-membered heteroaryl, and C₉-C₁₂ bicyclic aryl is optionally substituted by halo, C₁-C₆ alkyl, or nitro.

In some embodiments, the compound of Formula (XIII), (XIV), (XV), (XVI), or (XVII) or a pharmaceutically acceptable salt thereof is selected from Table C.

TABLE C
Compound 4

[symbol]

(E)-N-(4-(3-(3-
nitrophenyl)acryloyl)phenyl)furan-2-
carboxamide

[symbol]

(E)-5-nitro-N-(4-(3-(3-
nitrophenyl)acryloyl)phenyl)furan-2-
carboxamide.

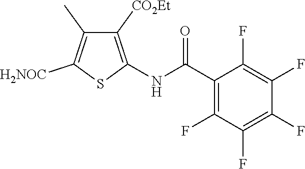 [SMILES: CCOC(=O)c1c(NC(=O)c2c(F)c(F)c(F)c(F)c2F)sc(C(N)=O)c1C]

[symbol]

Ethyl 2-amino-5-carbamoyl-4-
methylthiophene-3-carboxylate.

[symbol]

Ethyl 5-carbamoyl-2-(2-fluorobenzamido)-4-
methylthiophene-3-carboxylate.

[symbol]

Ethyl 5-carbamoyl-2-(4-fluorobenzamido)-4-
methylthiophene-3-carboxylate.

[symbol]

Ethyl 5-carbamoyl-2-(2-chlorobenzamido)-4-
methylthiophene-3-carboxylate.

[symbol]

Ethyl 5-carbamoyl-2-(3-chlorobenzamido)-4-
methylthiophene-3-carboxylate.

[symbol]

Ethyl 5-carbamoyl-2-(4-chlorobenzamido)-4-
methylthiophene-3-carboxylate.

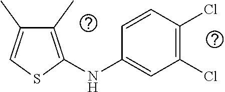 [SMILES: Cc1csc(Nc2ccc(Cl)c(Cl)c2)c1C]

Ethyl 5-carbamoyl-2-(3,4-dichlorobenzamido)-
4-methylthiophene-3-carboxylate.

[symbol]

Ethyl 5-carbamoyl-4-methyl-2-(3-
methylbenzamido)thiophene-3-carboxylate.

[symbol]

Ethyl 5-carbamoyl-4-methyl-2-(4-
methylbenzamido)thiophene-3-carboxylate.

[symbol]

Ethyl 5-carbamoyl-4-methyl-2-(3-
nitrobenzamido)thiophene-3-carboxylate.

[symbol]

Ethyl 5-carbamoyl-4-methyl-2-(4-
nitrobenzamido)thiophene-3-carboxylate.

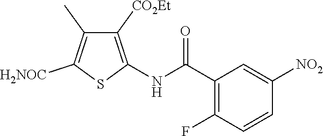 [SMILES: CCOC(=O)c1c(NC(=O)c2cc([N+](=O)[O-])ccc2F)sc(C(N)=O)c1C]

Ethyl 5-carbamoyl-2-(2-fluoro-5-
nitrobenzamido)-4-methylthiophene-3-
carboxylate.

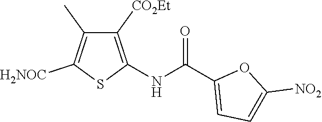 [SMILES: CCOC(=O)c1c(NC(=O)c2ccc([N+](=O)[O-])o2)sc(C(N)=O)c1C]

Ethyl 5-carbamoyl-4-methyl-2-(5-nitrofuran-2-
carboxamido)thiophene-3-carboxylate.

[symbol]

2-fluoro-5-nitro-N-phenylbenzamide.

[symbol]

2,3,4,5,6-pentafluoro-N-phenylbenzamide.

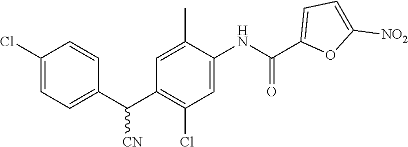 [SMILES: Cc1cc(C(C#N)c2ccc(Cl)cc2)c(Cl)cc1NC(=O)c1ccc([N+](=O)[O-])o1]

N-(5-chloro-4-((4-
chlorophenyl)(cyano)methyl)-2-
methylphenyl)-5-nitrofuran-2-carboxamide.

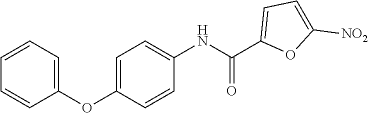 [SMILES: O=C(Nc1ccc(Oc2ccccc2)cc1)c1ccc([N+](=O)[O-])o1]

5-nitro-N-(4-phenoxyphenyl)furan-2-
carboxamide.

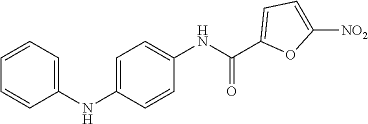 [SMILES: O=C(Nc1ccc(Nc2ccccc2)cc1)c1ccc([N+](=O)[O-])o1]

5-nitro-N-(4-(phenylamino)phenyl)furan-2-
carboxamide.

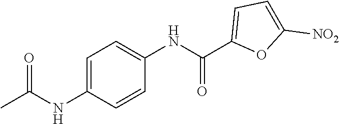 [SMILES: CC(=O)Nc1ccc(NC(=O)c2ccc([N+](=O)[O-])o2)cc1]

N-(4-acetamidophenyl)-5-nitrofuran-2-
carboxamide.

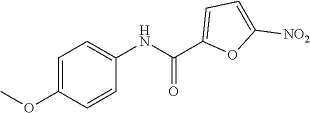 [SMILES: COc1ccc(NC(=O)c2ccc([N+](=O)[O-])o2)cc1]

N-(4-methoxyphenyl)-5-nitrofuran-2-
carboxamide.

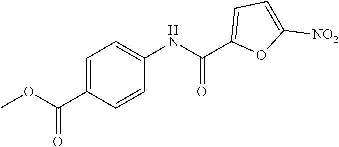 [SMILES: COC(=O)c1ccc(NC(=O)c2ccc([N+](=O)[O-])o2)cc1]

methyl 4-(5-nitrofuran-2-
carboxamido)benzoate.

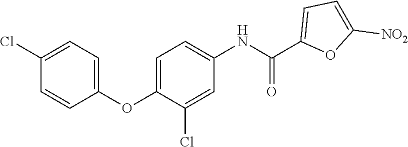 [SMILES: O=C(Nc1ccc(Oc2ccc(Cl)cc2)c(Cl)c1)c1ccc([N+](=O)[O-])o1]

N-(3-chloro-4-(4-chlorophenoxy)phenyl)-5-
nitrofuran-2-carboxamide.

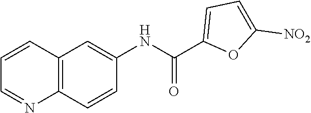 [SMILES: O=C(Nc1ccc2ncccc2c1)c1ccc([N+](=O)[O-])o1]

5-nitro-N-(quinolin-6-yl)furan-2-carboxamide.

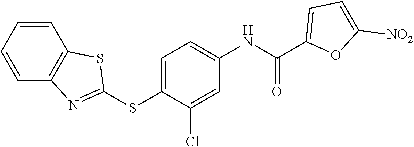 [SMILES: O=C(Nc1ccc(Sc2nc3ccccc3s2)c(Cl)c1)c1ccc([N+](=O)[O-])o1]

N-(4-(benzo[d]thiazol-2-ylthio)-3-
chlorophenyl)-5-nitrofuran-2-carboxamide.

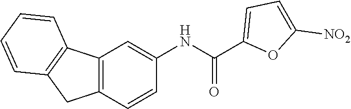 [SMILES: O=C(Nc1ccc2c(c1)-c1ccccc1C2)c1ccc([N+](=O)[O-])o1]

N-(9H-fluoren-3-yl)-5-nitrofuran-2-
carboxamide.

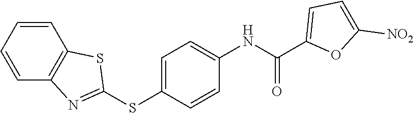 [SMILES: O=C(Nc1ccc(Sc2nc3ccccc3s2)cc1)c1ccc([N+](=O)[O-])o1]

N-(4-(benzo[d]thiazol-2-ylthio)phenyl)-5-
nitrofuran-2-carboxamide.

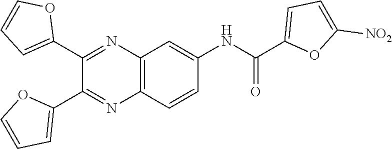 [SMILES: O=C(Nc1ccc2nc(-c3ccco3)c(-c3ccco3)nc2c1)c1ccc([N+](=O)[O-])o1]

N-(2,3-di(furan-2-yl)quinoxalin-6-yl)-5-
nitrofuran-2-carboxamide.

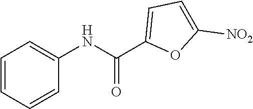 [SMILES: O=C(Nc1ccccc1)c1ccc([N+](=O)[O-])o1]

5-nitro-N-phenylfuran-2-carboxamide.

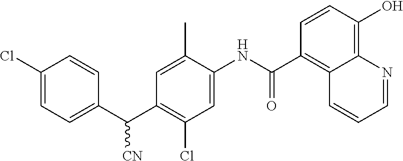 [SMILES: Cc1cc(C(C#N)c2ccc(Cl)cc2)c(Cl)cc1NC(=O)c1ccc(O)c2ncccc12]

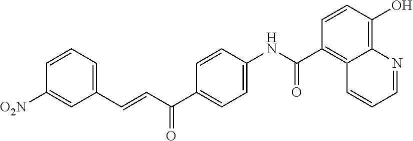 [SMILES: O=C(/C=C/c1cccc([N+](=O)[O-])c1)c1ccc(NC(=O)c2ccc(O)c3ncccc23)cc1]

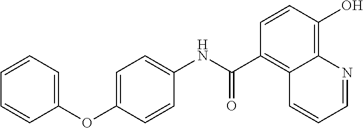 [SMILES: O=C(Nc1ccc(Oc2ccccc2)cc1)c1ccc(O)c2ncccc12]

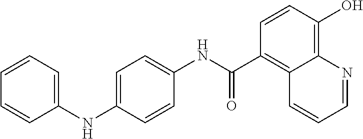 [SMILES: O=C(Nc1ccc(Nc2ccccc2)cc1)c1ccc(O)c2ncccc12]

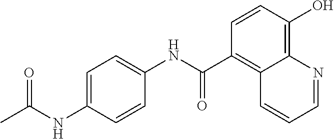 [SMILES: CC(=O)Nc1ccc(NC(=O)c2ccc(O)c3ncccc23)cc1]

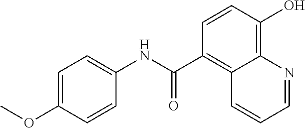 [SMILES: COc1ccc(NC(=O)c2ccc(O)c3ncccc23)cc1]

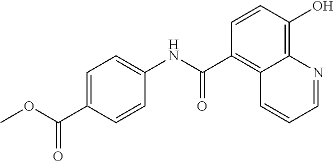 [SMILES: COC(=O)c1ccc(NC(=O)c2ccc(O)c3ncccc23)cc1]

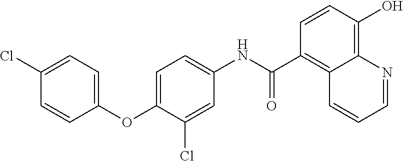 [SMILES: O=C(Nc1ccc(Oc2ccc(Cl)cc2)c(Cl)c1)c1ccc(O)c2ncccc12]

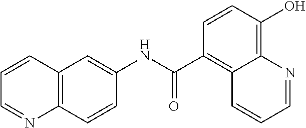 [SMILES: O=C(Nc1ccc2ncccc2c1)c1ccc(O)c2ncccc12]

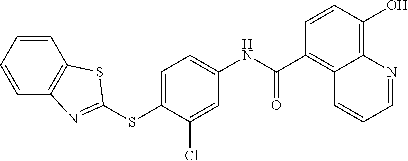 [SMILES: O=C(Nc1ccc(Sc2nc3ccccc3s2)c(Cl)c1)c1ccc(O)c2ncccc12]

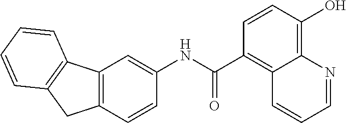 [SMILES: O=C(Nc1ccc2c(c1)-c1ccccc1C2)c1ccc(O)c2ncccc12]

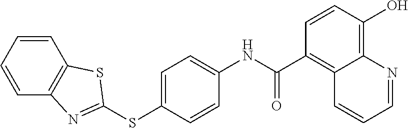 [SMILES: O=C(Nc1ccc(Sc2nc3ccccc3s2)cc1)c1ccc(O)c2ncccc12]

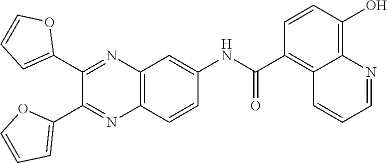 [SMILES: O=C(Nc1ccc2nc(-c3ccco3)c(-c3ccco3)nc2c1)c1ccc(O)c2ncccc12]

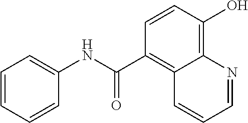 [SMILES: O=C(Nc1ccccc1)c1ccc(O)c2ncccc12]

[symbol]

indicates data missing or illegible when filed

In some embodiments, the disclosure relates to a compound or a pharmaceutically acceptable salt thereof, having the formula (XVIII),

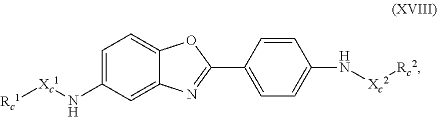

wherein
each of X_{c}¹ and X_{c}² is independently —SO₂— or —C(O)—; and
each of R_{c}¹ and R_{c}² is independently alkyl, haloalkyl, aryl, or heteroaryl, wherein alkyl, aryl, and heteroaryl are optionally substituted with halogen, hydroxy, nitro, amino, cyano, alkyl, aryl, haloalkyl, alkoxy, C(O)OH, or C(O)O-alkyl.

In some embodiments of compounds of formula XVIII, at least one of X_{c}¹ and X_{c}² is —SO₂—. In some embodiments, X_{c}¹ is —SO₂—, and X_{c}² is —SO₂— or —C(O)—. In some embodiments, X_{c}¹ and X_{c}² are the same. In some embodiments, each of X_{c}¹ and X_{c}² is —SO₂—.

In some embodiments, the disclosure relates to a compound or a pharmaceutically acceptable salt thereof, having the formula (XIX),

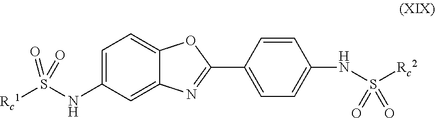

wherein each of R_{c}¹ and R_{c}² is independently alkyl, haloalkyl, aryl, or heteroaryl, wherein alkyl, aryl, and heteroaryl are optionally substituted with halogen, hydroxy, nitro, amino, cyano, alkyl, aryl, haloalkyl, alkoxy, C(O)OH, or C(O)O-alkyl.

In some embodiments of compounds of formula XVIII or XIX, each of R_{c}¹ and R_{c}² is independently C₁-C₆ alkyl, C₁-C₆ haloalkyl, C₆-C₁₀ aryl, 5- to 12-membered heteroaryl optionally substituted with halogen, hydroxy, —NO₂, —NH₂, —CN, —CF₃, C₁-C₆ alkyl, C₁-C₆ alkoxy, phenyl, C(O)OH, or C(O)O—C₁-C₆ alkyl. In some embodiments, R_{c}¹ and R_{c}² are the same.

In some embodiments of compounds of formula XVIII or XIX, each of R_{c}¹ and R_{c}² is independently C₁-C₆ alkyl or C₁-C₆ haloalkyl. In some embodiments, each of R¹ and R² is independently C₁-C₆ alkyl optionally substituted with C₆-C₁₀ aryl (e.g., phenyl). In some embodiments, each of R_{c}¹ and R_{c}² is independently methyl, ethyl, propyl, trifluoromethyl, or benzyl.

In some embodiments of compounds of formula XVIII or XIX, each of R_{c}¹ and R_{c}² is independently C₆-C₁₀ aryl (e.g., phenyl or naphthyl) or 5- to 12-membered heteroaryl (e.g., thienyl, benzothiadiazole, benzooxadiazole) optionally substituted with halogen, hydroxy, —NO₂, —NH₂, —CN, —CF₃, C₁-C₆ alkyl, C₁-C₆ alkoxy, phenyl, C(O)OH, or C(O)O—C₁-C₆ alkyl. In some embodiments, each of R_{c}¹ and R_{c}² is independently phenyl optionally substituted with halogen, hydroxy, methyl, or methoxy. In some embodiments, each of R_{c}¹ and R_{c}² is independently thienyl optionally substituted with halogen.

In some embodiments, the compound of formula (XVIII) or (XIX) or a pharmaceutically acceptable salt thereof is selected from Table D.

TABLE D
Compound 162

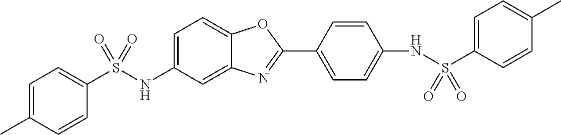

4-methyl-N-(4-(5-((4-methylphenyl)sulfonamido)benzo[d]oxazol-2-
yl)phenyl)benzenesulfonamide
Compound 163

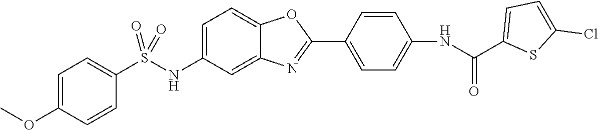 [SMILES: COc1ccc(S(=O)(=O)Nc2ccc3oc(-c4ccc(NC(=O)c5ccc(Cl)s5)cc4)nc3c2)cc1]

5-chloro-N-(4-(5-((4-methoxyphenyl)sulfonamido)benzo[d]oxazol-2-
yl)phenyl)thiophene-2-carboxamide
Compound 164

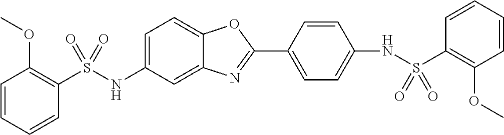 [SMILES: COc1ccccc1S(=O)(=O)Nc1ccc(-c2nc3cc(NS(=O)(=O)c4ccccc4OC)ccc3o2)cc1]

2-methoxy-N-(4-(5-((2-methoxyphenyl)sulfonamido)benzo[d]oxazol-2-
yl)phenyl)benzenesulfonamide

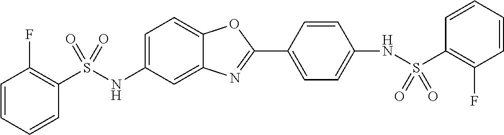 [SMILES: O=S(=O)(Nc1ccc(-c2nc3cc(NS(=O)(=O)c4ccccc4F)ccc3o2)cc1)c1ccccc1F]

2-fluoro-N-(4-(5-((2-fluorophenyl)sulfonamido)benzo[d]oxazol-2-
yl)phenyl)benzenesulfonamide

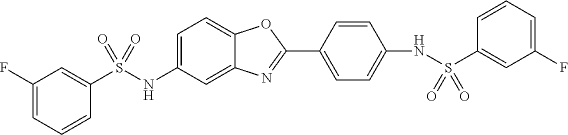 [SMILES: O=S(=O)(Nc1ccc(-c2nc3cc(NS(=O)(=O)c4cccc(F)c4)ccc3o2)cc1)c1cccc(F)c1]

3-fluoro-N-(4-(5-((2-fluorophenyl)sulfonamido)benzo[d]oxazol-2-
yl)phenyl)benzenesulfonamide

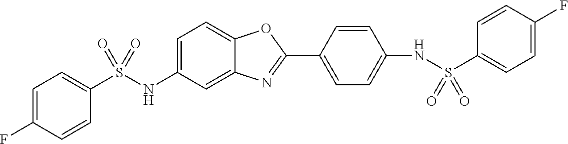 [SMILES: O=S(=O)(Nc1ccc(-c2nc3cc(NS(=O)(=O)c4ccc(F)cc4)ccc3o2)cc1)c1ccc(F)cc1]

4-fluoro-N-(4-(5-((4-fluorophenyl)sulfonamido)benzo[d]oxazol-2-
yl)phenyl)benzenesulfonamide

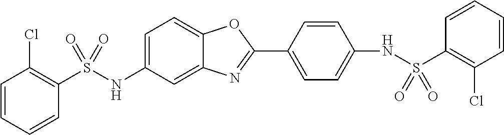 [SMILES: O=S(=O)(Nc1ccc(-c2nc3cc(NS(=O)(=O)c4ccccc4Cl)ccc3o2)cc1)c1ccccc1Cl]

2-chloro-N-(4-(5-((2-chlorophenyl)sulfonamido)benzo[d]oxazol-2-
yl)phenyl)benzenesulfonamide

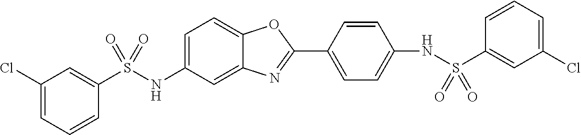 [SMILES: O=S(=O)(Nc1ccc(-c2nc3cc(NS(=O)(=O)c4cccc(Cl)c4)ccc3o2)cc1)c1cccc(Cl)c1]

3-chloro-N-(4-(5-((3-chlorophenyl)sulfonamido)benzo[d]oxazol-2-
yl)phenyl)benzenesulfonamide

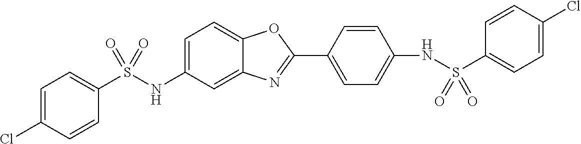 [SMILES: O=S(=O)(Nc1ccc(-c2nc3cc(NS(=O)(=O)c4ccc(Cl)cc4)ccc3o2)cc1)c1ccc(Cl)cc1]

4-chloro-N-(4-(5-((4-chlorophenyl)sulfonamido)benzo[d]oxazol-2-
yl)phenyl)benzenesulfonamide

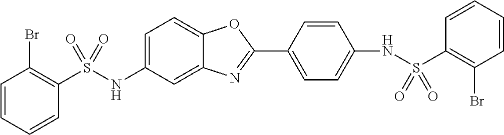 [SMILES: O=S(=O)(Nc1ccc(-c2nc3cc(NS(=O)(=O)c4ccccc4Br)ccc3o2)cc1)c1ccccc1Br]

2-bromo-N-(4-(5-((2-bromophenyl)sulfonamido)benzo[d]oxazol-2-
yl)phenyl)benzenesulfonamide

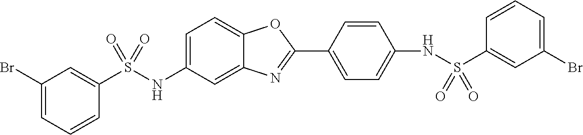 [SMILES: O=S(=O)(Nc1ccc(-c2nc3cc(NS(=O)(=O)c4cccc(Br)c4)ccc3o2)cc1)c1cccc(Br)c1]

3-bromo-N-(4-(5-((3-bromophenyl)sulfonamido)benzo[d]oxazol-2-
yl)phenyl)benzenesulfonamide

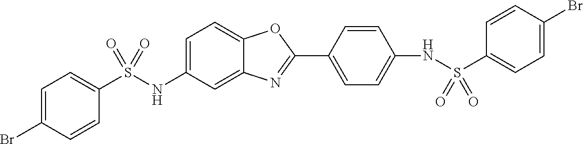 [SMILES: O=S(=O)(Nc1ccc(-c2nc3cc(NS(=O)(=O)c4ccc(Br)cc4)ccc3o2)cc1)c1ccc(Br)cc1]

4-bromo-N-(4-(5-((4-bromophenyl)sulfonamido)benzo[d]oxazol-2-
yl)phenyl)benzenesulfonamide

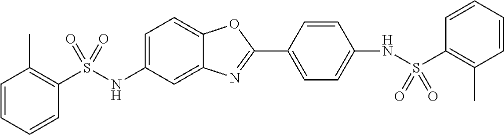 [SMILES: Cc1ccccc1S(=O)(=O)Nc1ccc(-c2nc3cc(NS(=O)(=O)c4ccccc4C)ccc3o2)cc1]

2-methyl-N-(4-(5-((2-methylphenyl)sulfonamido)benzo[d]oxazol-2-
yl)phenyl)benzenesulfonamide

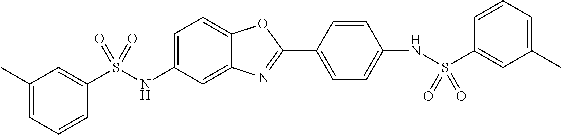

3-methyl-N-(4-(5-((2-methylphenyl)sulfonamido)benzo[d]oxazol-2-
yl)phenyl)benzenesulfonamide

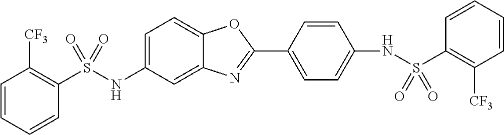 [SMILES: O=S(=O)(Nc1ccc(-c2nc3cc(NS(=O)(=O)c4ccccc4C(F)(F)F)ccc3o2)cc1)c1ccccc1C(F)(F)F]

2-(trifluoromethyl)-N-(4-(5-((2-(trifluoromethyl)phenyl)sulfonamido)benzo[d]oxazol-2-
yl)phenyl)benzenesulfonamide

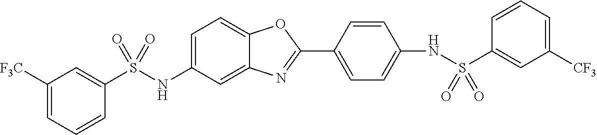 [SMILES: O=S(=O)(Nc1ccc(-c2nc3cc(NS(=O)(=O)c4cccc(C(F)(F)F)c4)ccc3o2)cc1)c1cccc(C(F)(F)F)c1]

3-(trifluoromethyl)-N-(4-(5-((3-(trifluoromethyl)phenyl)sulfonamido)benzo[d]oxazol-2-
yl)phenyl)benzenesulfonamide

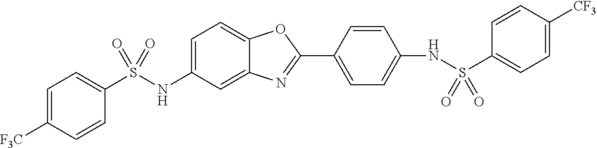 [SMILES: O=S(=O)(Nc1ccc(-c2nc3cc(NS(=O)(=O)c4ccc(C(F)(F)F)cc4)ccc3o2)cc1)c1ccc(C(F)(F)F)cc1]

4-(trifluoromethyl)-N-(4-(5-((4-(trifluoromethyl)phenyl)sulfonamido)benzo[d]oxazol-2-
yl)phenyl)benzenesulfonamide

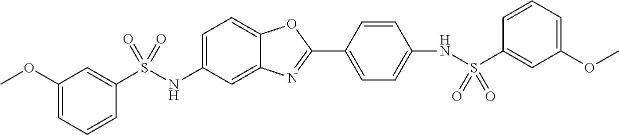

3-methoxy-N-(4-(5-((3-methoxyphenyl)sulfonamido)benzo[d]oxazol-2-
yl)phenyl)benzenesulfonamide

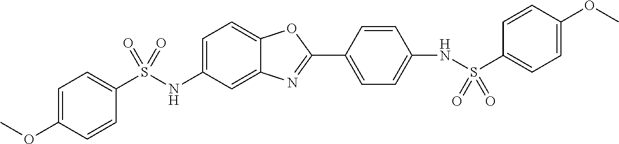

4-methoxy-N-(4-(5-((4-methoxyphenyl)sulfonamido)benzo[d]oxazol-2-
yl)phenyl)benzenesulfonamide

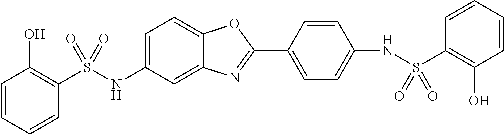 [SMILES: O=S(=O)(Nc1ccc(-c2nc3cc(NS(=O)(=O)c4ccccc4O)ccc3o2)cc1)c1ccccc1O]

2-hydroxy-N-(4-(5-((2-hydroxyphenyl)sulfonamido)benzo[d]oxazol-2-
yl)phenyl)benzenesulfonamide

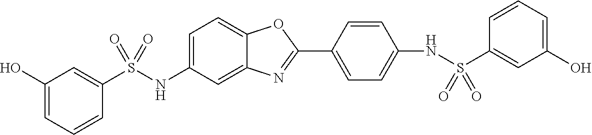 [SMILES: O=S(=O)(Nc1ccc(-c2nc3cc(NS(=O)(=O)c4cccc(O)c4)ccc3o2)cc1)c1cccc(O)c1]

3-hydroxy-N-(4-(5-((3-hydroxyphenyl)sulfonamido)benzo[d]oxazol-2-
yl)phenyl)benzenesulfonamide

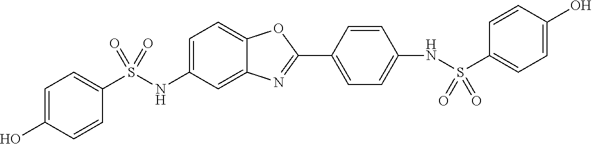 [SMILES: O=S(=O)(Nc1ccc(-c2nc3cc(NS(=O)(=O)c4ccc(O)cc4)ccc3o2)cc1)c1ccc(O)cc1]

4-hydroxy-N-(4-(5-((4-hydroxyphenyl)sulfonamido)benzo[d]oxazol-2-
yl)phenyl)benzenesulfonamide

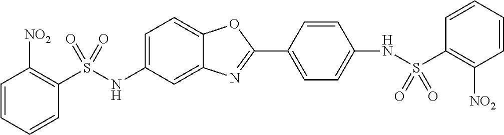 [SMILES: O=[N+]([O-])c1ccccc1S(=O)(=O)Nc1ccc(-c2nc3cc(NS(=O)(=O)c4ccccc4[N+](=O)[O-])ccc3o2)cc1]

2-nitro-N-(4-(5-((2-nitrophenyl)sulfonamido)benzo[d]oxazol-2-
yl)phenyl)benzenesulfonamide

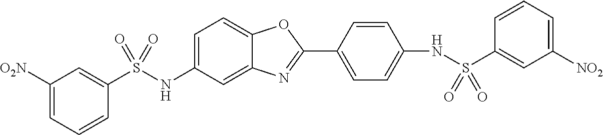 [SMILES: O=[N+]([O-])c1cccc(S(=O)(=O)Nc2ccc(-c3nc4cc(NS(=O)(=O)c5cccc([N+](=O)[O-])c5)ccc4o3)cc2)c1]

3-nitro-N-(4-(5-((3-nitrophenyl)sulfonamido)benzo[d]oxazol-2-
yl)phenyl)benzenesulfonamide

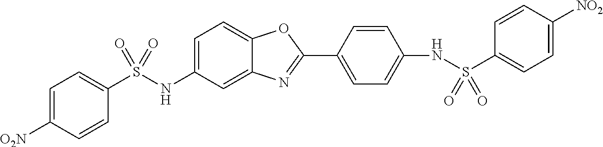 [SMILES: O=[N+]([O-])c1ccc(S(=O)(=O)Nc2ccc(-c3nc4cc(NS(=O)(=O)c5ccc([N+](=O)[O-])cc5)ccc4o3)cc2)cc1]

4-nitro-N-(4-(5-((4-nitrophenyl)sulfonamido)benzo[d]oxazol-2-
yl)phenyl)benzenesulfonamide

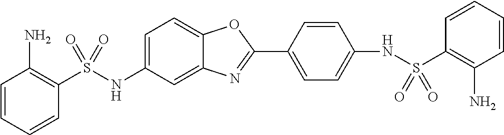 [SMILES: Nc1ccccc1S(=O)(=O)Nc1ccc(-c2nc3cc(NS(=O)(=O)c4ccccc4N)ccc3o2)cc1]

2-amino-N-(4-(5-((2-aminophenyl)sulfonamido)benzo[d]oxazol-2-
yl)phenyl)benzenesulfonamide

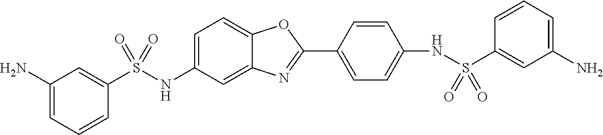 [SMILES: Nc1cccc(S(=O)(=O)Nc2ccc(-c3nc4cc(NS(=O)(=O)c5cccc(N)c5)ccc4o3)cc2)c1]

3-amino-N-(4-(5-((3-aminophenyl)sulfonamido)benzo[d]oxazol-2-
yl)phenyl)benzenesulfonamide

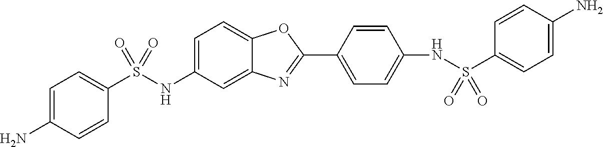 [SMILES: Nc1ccc(S(=O)(=O)Nc2ccc(-c3nc4cc(NS(=O)(=O)c5ccc(N)cc5)ccc4o3)cc2)cc1]

4-amino-N-(4-(5-((4-aminophenyl)sulfonamido)benzo[d]oxazol-2-
yl)phenyl)benzenesulfonamide

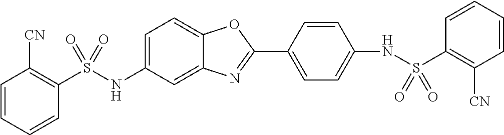 [SMILES: N#Cc1ccccc1S(=O)(=O)Nc1ccc(-c2nc3cc(NS(=O)(=O)c4ccccc4C#N)ccc3o2)cc1]

2-cyano-N-(4-(5-((2-cyanophenyl)sulfonamido)benzo[d]oxazol-2-
yl)phenyl)benzenesulfonamide

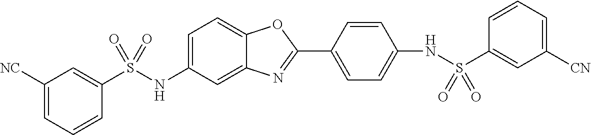 [SMILES: N#Cc1cccc(S(=O)(=O)Nc2ccc(-c3nc4cc(NS(=O)(=O)c5cccc(C#N)c5)ccc4o3)cc2)c1]

3-cyano-N-(4-(5-((3-cyanophenyl)sulfonamido)benzo[d]oxazol-2-
yl)phenyl)benzenesulfonamide

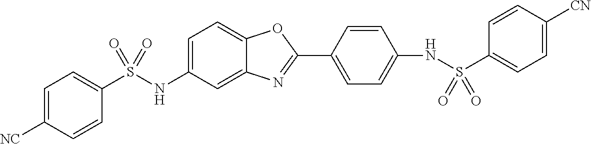 [SMILES: N#Cc1ccc(S(=O)(=O)Nc2ccc(-c3nc4cc(NS(=O)(=O)c5ccc(C#N)cc5)ccc4o3)cc2)cc1]

4-cyano-N-(4-(5-((4-cyanophenyl)sulfonamido)benzo[d]oxazol-2-
yl)phenyl)benzenesulfonamide

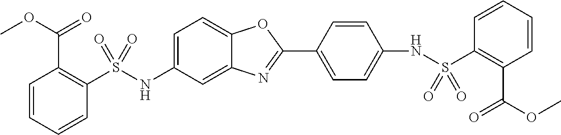

methyl 2-(N-(4-(5-((2-(methoxycarbonyl)phenyl)sulfonamido)benzo[d]oxazol-2-
yl)phenyl)sulfamoyl)benzoate

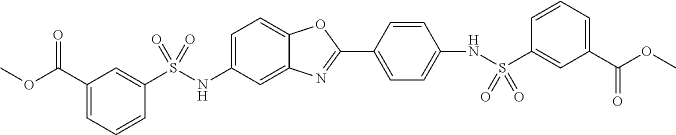

methyl 3-(N-(4-(5-((3-(methoxycarbonyl)phenyl)sulfonamido)benzo[d]oxazol-2-
yl)phenyl)sulfamoyl)benzoate

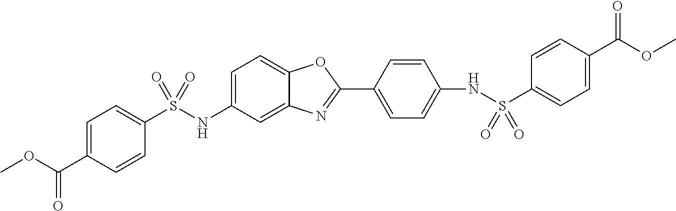

methyl 4-(N-(4-(5-((4-(methoxycarbonyl)phenyl)sulfonamido)benzo[d]oxazol-2-
9yl)phenyl)sulfamoyl)benzoate

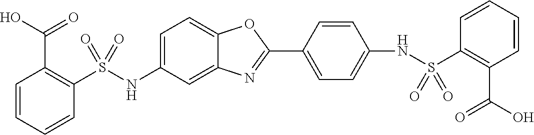 [SMILES: O=C(O)c1ccccc1S(=O)(=O)Nc1ccc(-c2nc3cc(NS(=O)(=O)c4ccccc4C(=O)O)ccc3o2)cc1]

2-(N-(4-(5-((2-carboxyphenyl)sulfonamido)benzo[d]oxazol-2-
yl)phenyl)sulfamoyl)benzoic acid

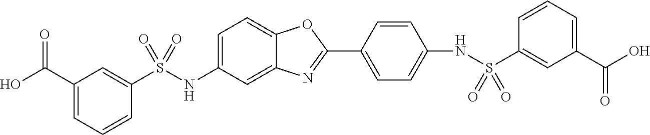 [SMILES: O=C(O)c1cccc(S(=O)(=O)Nc2ccc(-c3nc4cc(NS(=O)(=O)c5cccc(C(=O)O)c5)ccc4o3)cc2)c1]

3-(N-(4-(5-((3-carboxyphenyl)sulfonamido)benzo[d]oxazol-2-
yl)phenyl)sulfamoyl)benzoic acid

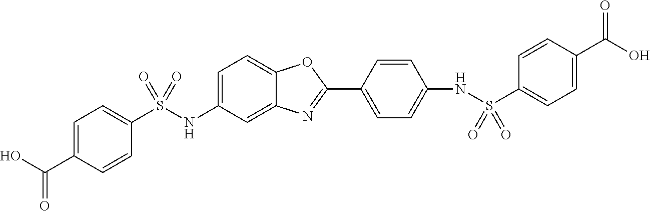 [SMILES: O=C(O)c1ccc(S(=O)(=O)Nc2ccc(-c3nc4cc(NS(=O)(=O)c5ccc(C(=O)O)cc5)ccc4o3)cc2)cc1]

4-(N-(4-(5-((4-carboxyphenyl)sulfonamido)benzo[d]oxazol-2-
yl)phenyl)sulfamoyl)benzoic acid

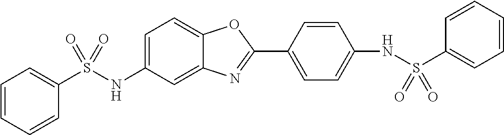 [SMILES: O=S(=O)(Nc1ccc(-c2nc3cc(NS(=O)(=O)c4ccccc4)ccc3o2)cc1)c1ccccc1]

N-(4-(5-(phenylsulfonamido)benzo[d]oxazol-2-yl)phenyl)benzenesulfonamide

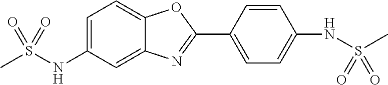

N-(4-(5-(methylsulfonamido)benzo[d]oxazol-2-yl)phenyl)methanesulfonamide

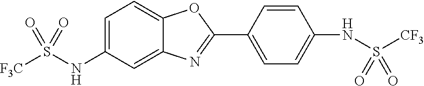 [SMILES: O=S(=O)(Nc1ccc(-c2nc3cc(NS(=O)(=O)C(F)(F)F)ccc3o2)cc1)C(F)(F)F]

1,1,1-trifluoro-N-(4-(5-((trifluoromethyl)sulfonamido)benzo[d]oxazol-2-
yl)phenyl)methanesulfonamide

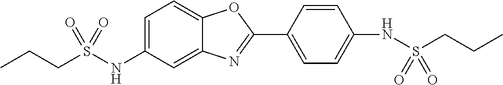 [SMILES: CCCS(=O)(=O)Nc1ccc(-c2nc3cc(NS(=O)(=O)CCC)ccc3o2)cc1]

N-(4-(5-(propylsulfonamido)benzo[d]oxazol-2-yl)phenyl)propane-1-sulfonamide

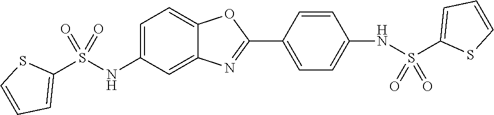 [SMILES: O=S(=O)(Nc1ccc(-c2nc3cc(NS(=O)(=O)c4cccs4)ccc3o2)cc1)c1cccs1]

N-(4-(5-(thiophene-2-sulfonamido)benzo[d]oxazol-2-yl)phenyl)thiophene-2-sulfonamide

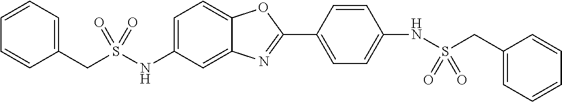 [SMILES: O=S(=O)(Cc1ccccc1)Nc1ccc(-c2nc3cc(NS(=O)(=O)Cc4ccccc4)ccc3o2)cc1]

1-phenyl-N-(4-(5-((phenylmethyl)sulfonamido)benzo[d]oxazol-2-
yl)phenyl)methanesulfonamide

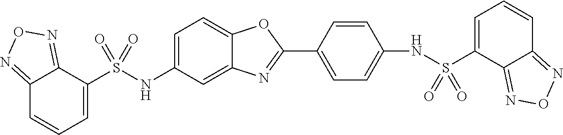 [SMILES: O=S(=O)(Nc1ccc(-c2nc3cc(NS(=O)(=O)c4cccc5nonc45)ccc3o2)cc1)c1cccc2nonc12]

N-(4-(5-(benzo[c][1,2,5]oxadiazole-4-sulfonamido)benzo[d]oxazol-2-
yl)phenyl)benzo[c][1,2,5]oxadiazole-4-sulfonamide

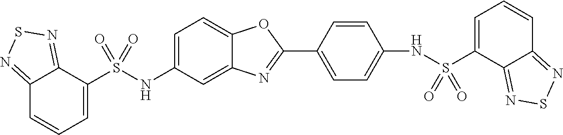 [SMILES: O=S(=O)(Nc1ccc(-c2nc3cc(NS(=O)(=O)c4cccc5nsnc45)ccc3o2)cc1)c1cccc2nsnc12]

N-(4-(5-(benzo[c][1,2,5]thiadiazole-4-sulfonamido)benzo[d]oxazol-2-
yl)phenyl)benzo[c][1,2,5]thiadiazole-4-sulfonamide

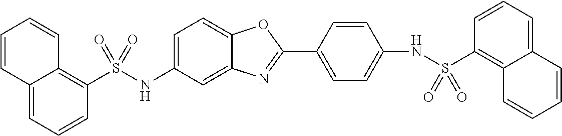 [SMILES: O=S(=O)(Nc1ccc(-c2nc3cc(NS(=O)(=O)c4cccc5ccccc45)ccc3o2)cc1)c1cccc2ccccc12]

N-(4-(5-(naphthalene-1-sulfonamido)benzo[d]oxazol-2-yl)phenyl)naphthalene-1-
sulfonamide

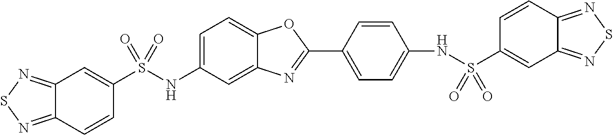 [SMILES: O=S(=O)(Nc1ccc(-c2nc3cc(NS(=O)(=O)c4ccc5nsnc5c4)ccc3o2)cc1)c1ccc2nsnc2c1]

N-(4-(5-(benzo[c][1,2,5]thiadiazole-5-sulfonamido)benzo[d]oxazol-2-
yl)phenyl)benzo[c][1,2,5]thiadiazole-5-sulfonamide

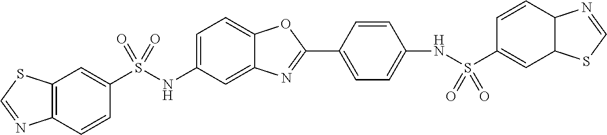 [SMILES: O=S(=O)(Nc1ccc(-c2nc3cc(NS(=O)(=O)c4ccc5ncsc5c4)ccc3o2)cc1)C1=CC2SC=NC2C=C1]

N-(4-(5-(benzo[d]thiazole-6-sulfonamido)benzo[d]oxazol-2-yl)phenyl)benzo[d]thiazole-
6-sulfonamide

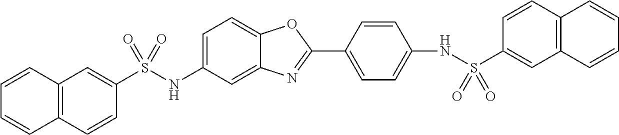 [SMILES: O=S(=O)(Nc1ccc(-c2nc3cc(NS(=O)(=O)c4ccc5ccccc5c4)ccc3o2)cc1)c1ccc2ccccc2c1]

N-(4-(5-(naphthalene-2-sulfonamido)benzo[d]oxazol-2-yl)phenyl)naphthalene-2-
sulfonamide

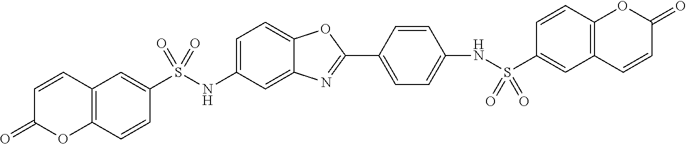 [SMILES: O=c1ccc2cc(S(=O)(=O)Nc3ccc(-c4nc5cc(NS(=O)(=O)c6ccc7oc(=O)ccc7c6)ccc5o4)cc3)ccc2o1]

2-oxo-N-(4-(5-((2-oxo-2H-chromene)-6-sulfonamido)benzo[d]oxazol-2-yl)phenyl)-2H-
chromene-6-sulfonamide

Those skilled in the art will recognize that the species listed or illustrated herein are not exhaustive, and that additional species within the scope of these defined terms may also be selected.

### Pharmaceutical Compositions

The compositions and methods of the present disclosure may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (or preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with a compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the disclosure may be used alone or conjointly administered with another type of therapeutic agent.

The present disclosure includes the use of pharmaceutically acceptable salts of compounds of the disclosure in the compositions and methods of the present disclosure. In certain embodiments, contemplated salts of the disclosure include, but are not limited to, alkyl, dialkyl, trialkyl or tetra-alkyl ammonium salts. In certain embodiments, contemplated salts of the disclosure include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the disclosure include, but are not limited to, Na, Ca, K, Mg, Zn or other metal salts. In certain embodiments, contemplated salts of the disclosure include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like. In certain embodiments, the present invention provides pharmaceutical compositions comprising a compound described herein, such as a compound of Formula I-IV. In certain embodiments, the pharmaceutical compositions further comprise a pharmaceutically acceptable excipient.

In certain embodiments, the pharmaceutical compositions may be for use in treating or preventing a condition or disease as described herein.

The compounds described herein are useful, for example, as therapeutics for the treatment of diseases, such as cancer. In certain aspects, the present disclosure provides a pharmaceutical composition comprising a compound of the disclosure and a pharmaceutically acceptable excipient.

In certain aspects, the present disclosure provides a method of treating a disease in a patient, the method comprising administering to the patient in need thereof an effective amount of a compound or composition of the disclosure. In certain aspects, the present disclosure provides a method of treating cancer in a patient, comprising administering to a patient in need thereof an effective amount of a compound or composition of the disclosure.

In certain aspects, the compounds and pharmaceutical compositions of the disclosure specifically target a heat shock protein (HSP) (e.g., HSP60, such as cHSP60 and/or mHSP60). Thus, these compounds and pharmaceutical compositions can be used to prevent, reverse, slow, or inhibit the activity of HSP (e.g., HSP60). In preferred embodiments, methods of treating a target cancer are described.

In certain aspects, the present disclosure provides methods of inhibiting a heat shock protein (HSP) (e.g., HSP60). In certain aspects, the present disclosure provides methods of inhibiting cHSP60, mHSP60, or both.

In certain aspects, the present disclosure provides methods of treating cancer. In certain aspects, the present disclosure provides methods of treating colorectal cancer (e.g., colon cancer), lung cancer (e.g., non-small cell lung cancer (NSCLC)), prostate cancer, renal cancer, blood cancer (e.g., leukemia), skin cancer (e.g., melanoma), ovarian cancer, breast cancer, CNS cancer, cervical cancer, and gastric cancer. In certain aspects, the present disclosure provides methods of treating colorectal cancer (e.g., colon cancer).

In certain aspects, the compounds of the present disclosure are for use in inhibiting a heat shock protein (HSP) (e.g., HSP60). In certain aspects, the compounds of the present disclosure are for use in inhibiting cHSP60, mHSP60, or both.

In certain aspects, the compounds of the present disclosure are for use in treating cancer. In certain aspects, the compounds of the present disclosure are for use in treating colorectal cancer (e.g., colon cancer), lung cancer (e.g., non-small cell lung cancer (NSCLC)), prostate cancer, renal cancer, blood cancer (e.g., leukemia), skin cancer (e.g., melanoma), ovarian cancer, breast cancer, CNS cancer, cervical cancer, and gastric cancer. In certain aspects, the compounds of the present disclosure are for use in treating colorectal cancer (e.g., colon cancer).

In certain aspects, the present disclosure provides methods of treating a mammal suffering from cancer. In certain aspects, the present disclosure provides methods of treating a mammal suffering from colorectal cancer (e.g., colon cancer), lung cancer (e.g., non-small cell lung cancer (NSCLC)), prostate cancer, renal cancer, blood cancer (e.g., leukemia), skin cancer (e.g., melanoma), ovarian cancer, breast cancer, CNS cancer, cervical cancer, and gastric cancer. In certain aspects, the present disclosure provides methods of treating a mammal suffering from colorectal cancer (e.g., colon cancer).

In certain embodiments, compounds of the disclosure are prodrugs of the compounds described herein. For example, wherein a hydroxyl in the parent compound is presented as an ester or a carbonate, or a carboxylic acid present in the parent compound is presented as an ester. In certain such embodiments, the prodrug is metabolized to the active parent compound in vivo (e.g., the ester is hydrolyzed to the corresponding hydroxyl or carboxylic acid).

In certain embodiments, compounds of the disclosure may be racemic. In certain embodiments, compounds of the disclosure may be enriched in one enantiomer. For example, a compound of the invention may have greater than 30% ee, 40% ee, 50% ee, 60% ee, 70% ee, 80% ee, 90% ee, or even 95% or greater ee. In certain embodiments, compounds of the disclosure may have more than one stereocenter. In certain such embodiments, compounds of the disclosure may be enriched in one or more diastereomers. For example, a compound of the disclosure may have greater than 30% de, 40% de, 50% de, 60% de, 70% de, 80% de, 90% de, or even 95% or greater de.

### Chemical Synthesis Methods

The following examples are offered to illustrate but not to limit the disclosure. One of skill in the art will recognize that the following synthetic reactions and schemes may be modified by choice of suitable starting materials and reagents in order to access other compounds of Formula I-XIX.

Abbreviations: The examples described herein use materials, including but not limited to, those described by the following abbreviations known to those skilled in the art:

| | |
|---|---|
| g | grams |
| eq | equivalents |
| mmol | millimoles |
| mL | milliliters |
| EtOAc | ethyl acetate |
| MHz | megahertz |
| ppm | parts per million |
| CDCl₃ | deuterated chloroform |
| δ | chemical shift |
| s | singlet |
| d | doublet |
| t | triplet |
| q | quartet |
| quin | quintet |
| br | broad |
| m | multiplet |
| Hz | hertz |
| THF | tetrahydrofuran |
| ° C. | degrees Celsius |
| PE | petroleum ether |
| EA | ethyl acetate |
| R_{f} | retardation factor |
| N | normal |
| J | coupling constant |
| DMSO-d₆ | deuterated dimethyl sulfoxide |
| n-BuOH | n-butanol |
| DIEA | n,n-diisopropylethylamine |
| TMSCl | trimethylsilyl chloride |
| min | minutes |
| hr | hours |
| Me | methyl |
| Et | ethyl |
| i-Pr | isopropyl |
| TLC | thin layer chromatography |
| M | molar |
| Compd# | compound number |
| MS | mass spectrum |
| ESI | Electrospray ionization |
| m/z | mass-to-charge ratio |
| Ms | methanesulfonyl |
| Boc | tert-butyloxycarbonyl |
| TFA | trifluoroacetic acid |
| Tos | toluenesulfonyl |
| DMAP | 4-(dimethylamino)pyridine |
| mM | millimolar |
| uM | micromolar |
| ATP | adenosine triphosphate |
| IC₅₀ | half maximal inhibitory concentration |
| U/mL | units of activity per milliliter |
| DIAD | diisopropyl azodicarboxylate |
| DMSO | dimethyl sulfoxide |
| MeTHF | 2-methyltetrahydrofuran |
| MOM | methoxymethyl |
| DCM | dichloromethane |
| DMF | N,N-dimethylformamide |
| DBU | 1,8-diazabicyclo[5.4.0]undec-7-ene |
| DIPEA | N,N-diisopropylethylamine |
| Hex | hexanes |
| Pd(dppf)Cl₂ | [1,1′- |
| | Bis(diphenylphosphino)ferrocene]dichloropalladium(II) |
| MeCN | Acetonitrile |
| (ACN) | |
| Hunig's | N,N-diisopropylethylamine |
| Base | |
| TBAF | Tert butyl ammonium fluoride |
| HBTU | (2-(1H-benzotriazol-1-yl)-1,1,3,3- |
| | tetramethyluronium hexafluorophosphate |
| PPh₃ | Triphenyl phosphine |
| RT | Room Temperature |
| p-TSA | Para-Tolylsulfonic acid |
| t-BuOH | Tert-Butanol |
| mCPBA | Meta-Chloroperoxy benzoic acid |
| AcOH | Acetic Acid |
| DMAc | N, N-Dimethylformamide |
| MDH | Malate dehydrogenase |
| Rho | Rhodanese |
| IC₅₀ | Inhibitory concentration for half- |
| | maximal signal in biochemical assay |
| EC₅₀ | Effective concentration for half- |
| | maximal signal in proliferation assays |
| CC₅₀ | Cytotoxicity concentration for half- |
| | maximal signal in cell viability assays. |

The proposed targets can be prepared via the conventional chemistry or following the general schemes as shown below.

Unless otherwise stated, all chemicals were purchased from commercial suppliers and used without further purification. Reaction progress was monitored by thin-layer chromatography on silica gel 60 F254 coated glass plates (EM Sciences). Flash chromatography was performed using a Biotage Isolera One flash chromatography system and eluting through Biotage KP-Sil Zip or Snap silica gel columns for normal-phase separations (hexanes:EtOAc gradients), or Snap KP-C18-HS columns for reverse-phase separations (H₂O:MeOH gradients). Reverse-phase high-performance liquid chromatography (RP-HPLC) was performed using a Waters 1525 binary pump, 2489 tunable UV/Vis detector (254 and 280 nm detection), and 2707 autosampler. For preparatory HPLC purification, samples were chromatographically separated using a Waters XSelect CSH C18 OBD prep column (part number 186005422, 130 Å pore size, 5 μm particle size, 19×150 mm), eluting with a H₂O:CH₃CN gradient solvent system. Linear gradients were run from either 100:0, 80:20, or 60:40 A:B to 0:100 A:B (A=95:5 H₂O:CH₃CN, 0.05% TFA; B=5:95 H₂O:CH₃CN, 0.05% TFA). Products from normal-phase separations were concentrated directly, and reverse-phase separations were concentrated, diluted with H₂O, frozen, and lyophilized. For primary compound purity analyses (HPLC-1), samples were chromatographically separated using a Waters XSelect CSH C18 column (part number 186005282, 130 Å pore size, 5 μm particle size, 3.0×150 mm), eluting with the above H₂O:CH₃CN gradient solvent systems. For secondary purity analyses (HPLC-2) of final test compounds, samples were chromatographically separated using a Waters XBridge C18 column (part number 186003132, 130 Å pore size, 5.0 μm particle size, 3.0×100 mm), eluting with a H₂O:MeOH gradient solvent system. Linear gradients were run from either 100:0, 80:20, 60:40, or 20:80 A:B to 0:100 A:B (A=95:5 H₂O:MeOH, 0.05% TFA; B=5:95 H₂O:MeOH, 0.05% TFA). Test compounds were found to be >95% in purity from both RP-HPLC analyses. Mass spectrometry data were collected using either Agilent LC 1200-MS 6130 or Agilent LC 1290-MS 6545 Q-TOF analytical LC-MS instruments at the IU Chemical Genomics Core Facility (CGCF). ¹H-NMR spectra were recorded on a Bruker 300 MHz spectrometer at the IU CGCF. Chemical shifts are reported in parts per million and calibrated to the d₆-DMSO solvent peaks at 2.50 ppm.

The compounds of formula (I), (II), and (III) were synthesized according to the synthetic procedures and methods found in U.S. Patent Application Publications US 2022-0009914 and US 2023-0295136, the contents of which are incorporated by reference in their entirety.

The compounds of formula (IV), (V), (VI), (VII), (VIII), (IX), (X), (XI) and (XII) were synthesized according to the synthetic procedures and methods found in U.S. Patent Application Publications US 2021-0395210 and US 2023-0406831, the contents of which are incorporated by reference in their entirety.

### General Synthetic Method A

The general amide coupling procedure to give parent inhibitor and analogs is presented below, followed by characterization data for each analog.

### General Procedure A-1 for the Amide Coupling.

To stirring mixtures of either aniline or ethyl 2-amino-5-carbamoyl-4-methylthiophene-3-carboxylate (1 eq.) in anhydrous acetonitrile were added the respective R—COCl reagents (1 eq.) and Cs₂CO₃ (1.3 eq.). Note that for any analogs where the R—CO₂H starting materials were only commercially available, the acids were first converted to the acid chlorides by stirring in thionyl chloride at 60° C. for 1 h, then concentrating. The reactions were allowed to stir at room temperature overnight, then diluted with water/DMSO to solubilize, and flash chromatographic purification (reverse-phase with a water:MeOH gradients) afforded the products as solids after lyophilization. If necessary, products were further purified by preparatory RP-HPLC (water:CH₃CN and/or water:MeOH gradients), concentrated, and lyophilized.

### General Procedure A-2 for the Amide Coupling.

To stirring mixture of the respective arylamine (1 eq.) in dichloromethane was added 5-nitro2-furoyl chloride (1.1 eq.) followed by pyridine (1.2 eq.), then the reactions were left to stir at RT overnight. The following day, the reactions were concentrated and flash chromatographic purification over silica (hexanes:EtOAc gradient) afforded the products as solids. If necessary, products were further purified by preparatory RP-HPLC (water:CH₃CN gradient), concentrated, and lyophilized.

The following examples of compounds of formula (XIII), (XIV), (XV), (XVI) and (XVII) were synthesized as indicated in the methods above and via conventional chemistry:

| Structure | Characterization |
|---|---|
| Compound 4 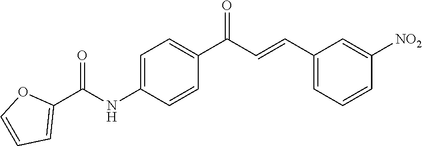 [SMILES: O=C(/C=C/c1cccc([N+](=O)[O-])c1)c1ccc(NC(=O)c2ccco2)cc1] (E)-N-(4-(3-(3- nitrophenyl)acryloyl)phenyl)furan-2- carboxamide. | Commercially available. Synthesized Characterization: Yield = 76%. ¹H NMR (300 MHz, d6-DMSO) δ 10.56 (s, 1H), 8.79 (t, J = 1.7 Hz, 1H), 8.31-8.38 (m, 1H), 8.14-8.30 (m, 4H), 7.96-8.03 (m, 3H), 7.84 (d, J = 15.7 Hz, 1H), 7.75 (t, J = 8.0 Hz, 1H), 7.43 (dd, J = 3.5, 0.7 Hz, 1H), 6.74 (dd, J = 3.5, 1.8 Hz, 1H). ¹³C NMR (75 MHz, d6- DMSO) δ 187.45, 156.46, 148.45, 147.12, 146.26, 143.45, 140.92, 136.72, 135.13, 132.34, 130.36, 130.00, 124.74, 124.60, 122.96, 119.54, 115.61, 112.37. MS (Q-TOF ESI) C₂₀H₁₃N₂O₅ [M − H]⁻ m/z expected = 361.0830, observed = 361.0791. HPLC-1 = 99%; HPLC-2 = 99% |
| 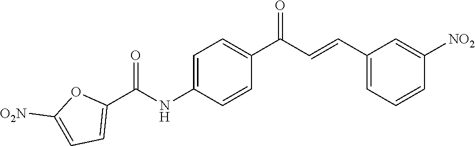 [SMILES: O=C(/C=C/c1cccc([N+](=O)[O-])c1)c1ccc(NC(=O)c2ccc([N+](=O)[O-])o2)cc1] (E)-5-nitro-N-(4-(3-(3- nitrophenyl)acryloyl)phenyl)furan-2- carboxamide. | Yield = 10%. ¹H NMR (600 MHz, DMSO- d₆) δ ppm 7.71 (d, J = 2.9 Hz, 1 H), 7.76 (t, J = 7.9 Hz, 1 H), 7.82-7.89 (m, 2 H), 7.98 (d, J = 8.1 Hz, 2 H), 8.19 (d, J = 15.4 Hz, 1 H), 8.28 (d, J = 8.1 Hz, 3 H), 8.35 (d, J = 7.7 Hz, 1 H), 8.80 (br. s., 1 H), 10.95 (s, 1 H); ¹³C-NMR (150 MHz, d₆-DMSO) δ 113.45, 117.18, 119.98, 122.95, 124.67, 130.04, 130.37, 133.03, 135.13, 136.67, 141.11, 142.57, 147.45, 148.45, 151.92, 154.92, 187.52; MS (ESI) C₂₀H₁₃N₃O₇ [M − H]⁻ m/z expected = 406.0681, observed = 406.0649. HPLC-1 = 98%, HPLC-2 = 97%. |
| 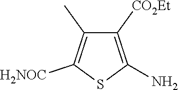 Ethyl 2-amino-5-carbamoyl-4- methylthiophene-3-carboxylate. | Commercially obtained starting material. ¹H NMR (600 MHz, DMSO-d₆) δ ppm 1.27 (t, J = 7.2 Hz, 3 H), 2.47 (s, 3 H), 4.20 (q, J = 7.2 Hz, 2 H) 7.07 (br. s., 2 H) 7.64 (s, 2 H); ¹³C- NMR (150 MHz, d₆-DMSO) δ 14.29, 16.05, 59.18, 105.46, 113.06, 139.82, 164.38, 164.83, 164.99; MS (ESI) C₉H₁₁N₂O₃S [M − H]⁻ m/z expected = 227.1, observed = 226.9. HPLC-1 = 98%, HPLC-2 = >99%. |
| 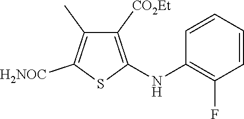 Ethyl 5-carbamoyl-2-(2-fluorobenzamido)-4- methylthiophene-3-carboxylate. | Yield = 22%. ¹H NMR (600 MHz, DMSO- d₆) δ ppm 1.34 (t, J = 7.2 Hz, 3 H), 2.56 (s, 3 H), 4.36 (q, J = 7.0 Hz, 2 H), 7.41-7.60 (m, 4 H), 7.70-7.79 (m, 1 H), 8.06 (t, J = 7.5 Hz, 1 H), 12.28 (d, J = 10.6 Hz, 1 H); ¹³C-NMR (150 MHz, d₆-DMSO) δ 14.00, 15.47, 61.01, 114.41, 116.69, 116.85, 118.96, 119.03, 124.05, 125.52, 131.64, 135.44, 135.49, 136.94, 148.05, 159.18, 159.94, 160.48, 164.22, 165.01; MS (ESI) C₁₆H₁₄FN₂O₄S [M − H]⁻ m/z expected = 349.1, observed = 349.0. |
| | HPLC-1 = >99%, HPLC-2 = >99%. |
| 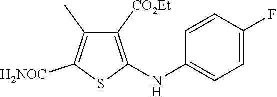 [SMILES: CCOC(=O)c1c(Nc2ccc(F)cc2)sc(C(N)=O)c1C] Ethyl 5-carbamoyl-2-(4-fluorobenzamido)-4- methylthiophene-3-carboxylate | Yield = 11%. ¹H NMR (600 MHz, DMSO- d₆) δ ppm 1.36 (t, J = 7.0 Hz, 3 H), 2.56 (s, 3 H), 4.37 (q, J = 7.0 Hz, 2 H), 7.41-7.61 (m, 4 H), 8.00 (dd, J = 7.7, 5.5 Hz, 2 H), 12.07 (br. s., 1 H); ¹³C-NMR (150 MHz, d₆-DMSO) δ 14.01, 15.41, 61.14, 114.36, 116.30, 116.44, 123.72, 128.26, 130.13, 130.20, 136.94, 148.62, 162.36, 164.02, 164.22, 165.48, 165.69; MS (ESI) C₁₆H₁₄FN₂O₄S [M − H]⁻ m/z |
| | expected = 349.1, observed = 349.0. HPLC-1 = |
| | >99%, HPLC-2 = >99%. |
| 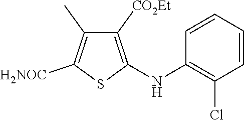 Ethyl 5-carbamoyl-2-(2-chlorobenzamido)- 4-methylthiophene-3-carboxylate. | Yield = 6%. ¹H NMR (600 MHz, DMSO-d₆) δ ppm 1.30 (t, J = 7.2 Hz, 3 H), 2.55 (s, 3 H), 4.30 (q, J = 7.2 Hz, 2 H), 7.47-7.59 (m, 3 H), 7.59-7.69 (m, 2 H), 7.79 (d, J = 7.3 Hz, 1 H), 11.71 (s, 1 H); ¹³C-NMR (150 MHz, d₆- DMSO) δ 13.92, 15.24, 61.02, 115.20, 123.81, 127.82, 130.04, 130.29, 130.51, 132.94, 133.05, 137.05, 146.99, 163.16, 164.13, 164.71; MS (ESI) C₁₆H₁₄ClN₂O₄S [M − H]⁻ m/z expected = 365.0, observed = 365.0. HPLC-1 = >99%, HPLC-2 = >99%. |
| 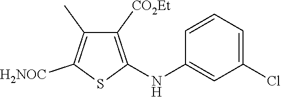 Ethyl 5-carbamoyl-2-(3-chlorobenzamido)- 4-methylthiophene-3-carboxylate. | Yield = 29%. ¹H NMR (600 MHz, DMSO- d₆) δ ppm 1.37 (t, J = 7.2 Hz, 3 H), 2.56 (s, 3 H), 4.37 (q, J = 7.1 Hz, 2 H), 7.52 (br. s., 2 H), 7.64-7.69 (m, 1 H), 7.76-7.80 (m, 1 H), 7.86- 7.89 (m, 1 H), 7.93 (t, J = 1.8 Hz, 1 H), 12.00 (s, 1 H); ¹³C-NMR (150 MHz, d₆-DMSO) δ 13.98, 15.35, 61.16, 114.86, 123.95, 125.91, 127.21, 131.23, 132.83, 133.82, 133.93, 137.04, 147.99, 162.14, 164.16, 165.17; MS |
| | (ESI) C₁₆H₁₄ClN₂O₄S [M − H]⁻ m/z expected = |
| | 365.0, observed = 365.0. HPLC-1 = >99%, |
| | HPLC-2 = 99%. |
| 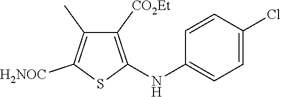 Ethyl 5-carbamoyl-2-(4-chlorobenzamido)- 4-methylthiophene-3-carboxylate. | Yield = 11%. ¹H NMR (600 MHz, DMSO- d₆) δ ppm 1.36 (t, J = 7.2 Hz, 3 H), 2.56 (s, H), 4.37 (q, J = 7.0 Hz, 2 H), 7.52 (br. s., 2 H), 7.71 (d, J = 8.1 Hz, 2 H), 7.94 (d, J = 8.1 Hz, 2 H), 12.08 (s, 1 H); ¹³C-NMR (150 MHz, d₆- DMSO) δ 14.01, 15.41, 61.16, 114.50, 123.84, 129.19, 129.37, 130.45, 136.96, 137.98, 148.45, 162.40, 164.19, 165.45; MS (ESI) C₁₆H₁₄ClN₂O₄S [M − H]⁻ m/z expected = |
| | 365.0, observed = 365.0. HPLC-1 = >99%, |
| | HPLC-2 = >99%. |
| 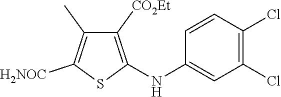 Ethyl 5-carbamoyl-2-(3,4- dichlorobenzamido)-4-methylthiophene-3- carboxylate. | Yield = 9%. ¹H NMR (600 MHz, DMSO-d₆) δ ppm 1.37 (t, J = 7.0 Hz, 3 H), 2.56 (s, 3 H), 4.38 (q, J = 7.0 Hz, 2 H), 7.53 (br. s., 2 H), 7.88 (dd, J = 8.4, 2.2 Hz, 1H), 7.93 (d, J = 8.4 Hz, 1 H), 8.13 (d, J = 1.8 Hz, 1 H), 11.97 (s, 1 H); ¹³C-NMR (150 MHz, d₆-DMSO) δ 14.46, 15.82, 61.86, 115.65, 124.61, 127.88, 129.95, 132.02, 132.54, 132.80, 136.32, 137.51, 148.18, 161.97, 164.60, 165.52; MS (ESI) C₁₆H₁₃Cl₂N₂O₄S [M − H]⁻ m/z expected = |
| | 399.0, observed = 398.9. HPLC-1 = 97%, |
| | HPLC-2 = 96%. |
| 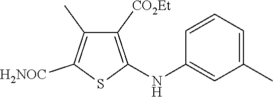 Ethyl 5-carbamoyl-4-methyl-2-(3- methylbenzamido)thiophene-3-carboxylate. | Yield = 6%. ¹H NMR (600 MHz, DMSO-d₆) δ ppm 1.37 (t, J = 67.0 Hz, 3 H), 2.42 (s, 3 H), 2.56 (s, 3 H), 4.37 (q, J = 7.1 Hz, 2 H), 7.45- 7.57 (m, 4 H), 7.72 (br. s., 1 H), 7.75 (s, 1 H), 12.04 (s, 1 H); ¹³C-NMR (150 MHz, d₆- DMSO) δ 14.00, 15.42, 20.93, 61.12, 114.18, 123.82, 124.29, 127.76, 129.16, 131.66, 133.75, 137.00, 138.75, ,148.67, 163.45, 164.25, 165.39; MS (ESI) C₁₇H₁₇N₂O₄S [M − |
| | H]⁻ m/z expected = 345.1, observed = 345.0. |
| | HPLC-1 = >99%, HPLC-2 = >99%. |
| 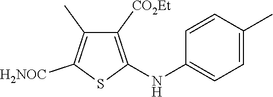 Ethyl 5-carbamoyl-4-methyl-2-(4- methylbenzamido)thiophene-3-carboxylate. | Yield = 6%. ¹H NMR (600 MHz, DMSO-d₆) δ ppm 1.36 (t, J = 7.2 Hz, 3 H), 2.41 (s, 3 H), 2.56 (s, 3 H), 4.37 (q, J = 7.2 Hz, 2 H), 7.43 (m, J = 8.1 Hz, 2 H), 7.50 (br. s., 2 H), 7.83 (m, J = 8.1 Hz, 2 H), 12.07 (s, 1 H); ¹³C-NMR (150 MHz, d₆-DMSO) δ 14.01, 15.44, 21.12, 61.11, 113.9, 123.54, 127.25, 128.80, 129.79, 136.96, 143.55, 148.92, 163.19, 164.26, 165.55; MS (ESI) C₁₇H₁₇N₂O₄S [M − H]⁻ m/z |
| | expected = 345.1, observed = 345.0. HPLC-1 = |
| | >99%, HPLC-2 = >99%. |
| 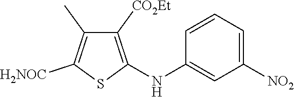 [SMILES: CCOC(=O)c1c(Nc2cccc([N+](=O)[O-])c2)sc(C(N)=O)c1C] Ethyl 5-carbamoyl-4-methyl-2-(3- nitrobenzamido)thiophene-3-carboxylate. | Yield = 4%. ¹H NMR (600 MHz, DMSO-d₆) δ ppm 1.38 (t, J = 7.2 Hz, 3 H), 2.56 (s, 3 H), 4.39 (q, J = 7.1 Hz, 2 H), 7.54 (br. s., 2 H), 7.94 (t, J = 8.1 Hz, 1 H), 8.34 (d, J = 7.7 Hz, 1 H), 8.53 (d, J = 8.4 Hz, 1 H), 8.68 (s, 1 H), 12.11 (s, 1 H); ¹³C-NMR (150 MHz, d₆- DMSO) δ 13.97, 15.33, 61.20, 115.35, 122.23, 124.25, 127.37, 131.03, 133.30, 133.53, 137.05, 147.80, 148.03, 161.85, |
| | 164.12, 165.04; MS (ESI) C₁₆H₁₄N₃O₆S [M − |
| | H]⁻ m/z expected = 376.1, observed = 375.9. |
| | HPLC-1 = >99%, HPLC-2 = >99%. |
| 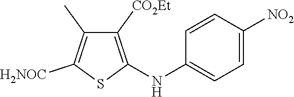 Ethyl 5-carbamoyl-4-methyl-2-(4- nitrobenzamido)thiophene-3-carboxylate. | Yield = 3%. ¹H NMR (600 MHz, DMSO-d₆) δ ppm 1.37 (t, J = 7.0 Hz, 3 H), 2.57 (s, 3 H), 4.39 (q, J = 7.2 Hz, 3 H), 7.55 (br. s., 2 H), 8.18 (d, J = 8.8 Hz, 2 H), 8.46 (d, J = 8.8 Hz, 2 H), 12.16 (s, 1 H); ¹³C-NMR (150 MHz, d₆- DMSO) δ 14.02, 15.38, 61.22, 115.21, 124.23, 124.32, 128.97, 137.00, 137.24, 147.82, 149.92, 161.96, 164.11, 165.25; MS (ESI) C₁₆H₁₄N₃O₆S [M − H]⁻ m/z expected = |
| | 376.1, observed = 376.0. HPLC-1 = >99%, |
| | HPLC-2 = 99%. |
| 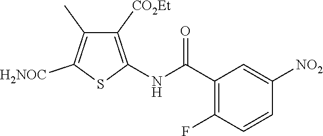 [SMILES: CCOC(=O)c1c(NC(=O)c2cc([N+](=O)[O-])ccc2F)sc(C(N)=O)c1C] Ethyl 5-carbamoyl-2-(2-fluoro-5- nitrobenzamido)-4-methylthiophene-3- carboxylate. | Yield = 68%. ¹H NMR (600 MHz, DMSO- d₆) δ ppm 1.35 (t, J = 7.2 Hz, 3 H), 2.55 (s, 3 H), 4.36 (q, J = 7.0 Hz, 2 H), 7.54 (br. s., 2 H), 7.77 (t, J = 9.9 Hz, 1 H), 8.51-8.60 (m, 1 H), 8.74 (dd, J = 6.1, 2.8 Hz, 1 H), 12.33 (d, J = 9.9 Hz, 1 H); ¹³C-NMR (150 MHz, d₆-DMSO) δ 13.98, 15.42, 61.14, 115.02, 118.75, 118.92, 120.53, 120.44, 124.54, 127.06, 130.15, 130.22, 136.97, 14.36, 147.44, 158.18, 162.18, 163.91, 164.07, 164.95; MS (ESI) C₁₆H₁₃FN₃O₆S [M − H]⁻ m/z expected = 394.1, observed = 393.9. HPLC-1 = >99%, HPLC-2 = 97%. |
| 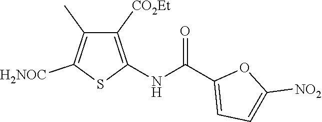 [SMILES: CCOC(=O)c1c(NC(=O)c2ccc([N+](=O)[O-])o2)sc(C(N)=O)c1C] Ethyl 5-carbamoyl-4-methyl-2-(5-nitrofuran- 2-carboxamido)thiophene-3-carboxylate. | Yield = 22%. ¹H NMR (600 MHz, DMSO- d₆) δ ppm 1.39 (t, J = 7.0 Hz, 3 H), 2.56 (s, 3 H), 4.40 (q, J = 7.0 Hz, 2 H), 7.57 (br. s., 2 H), 7.68 (d, J = 3.7 Hz, 1 H), 7.87 (d, J = 4.0 Hz, 1 H), 12.08 (s, 1H); ¹³C-NMR (150 MHz, d₆- DMSO) δ 13.96, 15.38, 61.33, 113.84, 115.24, 118.65, 124.82, 137.00, 145.66, 146.79, 151.71, 153.17, 163.97, 164.85; MS (ESI) C₁₄H₁₂N₃O₇S [M − H]⁻ m/z expected = 366.0, observed = 366.0. HPLC-1 = 97%, HPLC-2 = 98%. |
| [symbol] | Yield = 89%. ¹H NMR (300 MHz, DMSO- |
| 2-fluoro-5-nitro-N-phenylbenzamide. | d₆) δ ppm 7.08-7.21 (m, 1 H), 7.38 (t, J = 7.9 |
| | Hz, 2 H), 7.63-7.78 (m, 3 H), 8.46 (ddd, |
| | J = 9.1, 4.3, 3.0 Hz, 1 H), 8.54 (dd, J = 5.9, 2.9 |
| | Hz, 1 H), 10.68 (s, 1 H); ¹³C-NMR (75 MHz, |
| | d₆-DMSO) δ 117.89, 118.22, 119.89, 124.30, |
| | 125.75, 125.82, 126.03, 127.94, 128.08, |
| | 138.45, 143.78, 143.81, 160.56, 160.65, |
| | 184.09; MS (ESI) C₁₃H₈FN₂O₃ [M − H]⁻ m/z |
| | expected = 259.1, observed = 259.0. HPLC-1 = |
| | >99%, HPLC-2 = 97%. |
| [symbol] | Yield = 79%. ¹H NMR (300 MHz, DMSO- |
| 2,3,4,5,6-pentafluoro-N-phenylbenzamide. | d₆) δ ppm 7.12-7.25 (m, 1 H), 7.40 (t, J = 7.9 |
| | Hz, 2 H), 7.60-7.74 (m, 2 H), 11.00 (s, 1 H); |
| | ¹³C-NMR (75 MHz, d₆-DMSO) δ 119.62, |
| | 124.79, 129.12, 137.90, 154.87; MS (ESI) |
| | C₁₃H₅F₅NO [M − H]⁻ m/z 286.0, |
| | observed = 286.0. HPLC-1 = >99%, HPLC-2 = |
| | >99%. |
| 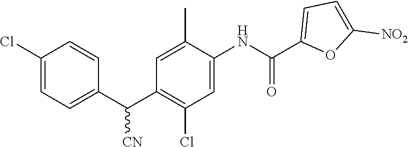 [SMILES: Cc1cc(C(C#N)c2ccc(Cl)cc2)c(Cl)cc1NC(=O)c1ccc([N+](=O)[O-])o1] N-(5-chloro-4-((4-chlorophenyl)(cyano)methyl)-2- methylphenyl)-5-nitrofuran-2-carboxamide. | Yield = 86%. ¹H NMR (600 MHz, DMSO- d₆) δ ppm 2.27 (s, 3 H), 6.04 (s, 1 H), 7.41 (d, J = 8.1 Hz, 2 H), 7.51 (d, J = 7.7 Hz, 2 H), 7.56 (d, J = 9.5 Hz, 2 H), 7.61 (d, J = 3.7 Hz, 1 H), 7.83 (d, J = 3.3 Hz, 1 H), 10.45 (s, 1 H); ¹³C- NMR (150 MHz, d₆-DMSO) δ 17.48, 38.09, 113.43, 116.94, 119.00, 127.67, 129.18, 129.61, 130.83, 131.73, 133.00, 133.63, 133.93, 136.45, 147.48, 151.77, 154.94; MS (ESI) C₂₀H₁₃Cl₂N₃O₄ [M − H]⁻ m/z expected = 428.0210, observed = 428.0172. HPLC-1 = 98%, HPLC-2 = 97%. |
| 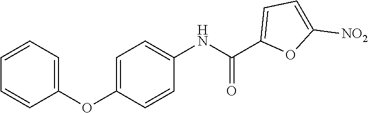 [SMILES: O=C(Nc1ccc(Oc2ccccc2)cc1)c1ccc([N+](=O)[O-])o1] 5-nitro-N-(4-phenoxyphenyl)furan-2- carboxamide. | Yield = 92%. ¹H NMR (600 MHz, DMSO- d₆) δ ppm 7.01 (d, J = 8.1 Hz, 2 H), 7.06 (m, J = 8.4 Hz, 2 H), 7.13 (t, J = 7.3 Hz, 1 H), 7.39 (t, J = 7.5 Hz, 2 H), 7.63 (d, J = 3.3 Hz, 1 H), 7.75 (m, J = 8.4 Hz, 2 H), 7.82 (d, J = 3.3 Hz, 1 H), 10.67 (s, 1 H); ¹³C-NMR (150 MHz, d₆- DMSO) δ 113.49, 116.45, 118.25, 119.16, 122.47, 123.29, 130.04, 133.46, 147.96, 151.74, 152.98, 154.44, 156.95; MS (ESI) C₁₇H₁₂N₂O₅ [M − H]⁻ m/z expected = 323.0673, |
| | observed = 323.0652. HPLC-1 = >99%, |
| | HPLC-2 = >99%. |
| 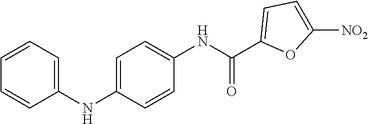 [SMILES: O=C(Nc1ccc(Nc2ccccc2)cc1)c1ccc([N+](=O)[O-])o1] 5-nitro-N-(4-(phenylamino)phenyl)furan-2- carboxamide. | Yield = 83%. ¹H NMR (600 MHz, DMSO- d₆) δ ppm 6.81 (t, J = 7.2 Hz, 1 H), 7.06 (d, J = 7.7 Hz, 2 H), 7.09 (d, J = 8.4 Hz, 2 H), 7.22 (t, J = 7.5 Hz, 2 H), 7.60 (d, J = 6.2 Hz, 3 H), 7.77-7.85 (m, 1 H), 8.18 (s, 1 H), 10.50 (s, 1 H); ¹³C-NMR (150 MHz, d₆-DMSO) δ 113.54 (s, 1 C) 116.03, 116.38, 117.03, 119.49, 122.06, 129.18, 130.11, 140.20, 143.51, 148.32, 151.66, 154.08; MS (ESI) C₁₇H₁₃N₃O₄ [MH]⁺ m/z expected = 324.0979, observed = 324.0914. HPLC-1 = 98%, HPLC- |
| | 2 = >99%. |
| 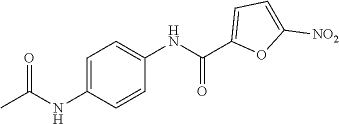 [SMILES: CC(=O)Nc1ccc(NC(=O)c2ccc([N+](=O)[O-])o2)cc1] N-(4-acetamidophenyl)-5-nitrofuran-2- carboxamide. | Yield = 48%. ¹H NMR (600 MHz, DMSO- d₆) δ ppm 2.03 (s, 3 H), 7.55-7.60 (m, 2 H), 7.60-7.63 (m, 1 H), 7.64 (d, J = 8.1 Hz, 2 H), 7.76-7.85 (m, 1 H), 9.96 (s, 1 H), 10.57 (s, 1 H); ¹³C-NMR (150 MHz, d₆-DMSO) δ 23.92, 113.49, 116.28, 119.24, 121.18, 132.83, 136.01, 148.08, 151.71, 154.31, 168.11; MS (ESI) C₁₃H₁₁N₃O₅ [M − H]⁻ m/z expected = 288.0626, observed = 288.0609. HPLC-1 = >99%, HPLC-2 = >99%. |
| 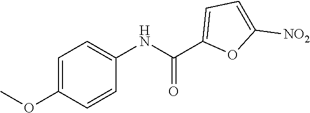 [SMILES: COc1ccc(NC(=O)c2ccc([N+](=O)[O-])o2)cc1] N-(4-methoxyphenyl)-5-nitrofuran-2- carboxamide. | Yield = 78%. ¹H NMR (600 MHz, DMSO- d₆) δ ppm 3.75 (s, 3 H), 6.96 (d, J = 8.4 Hz, 2 H), 7.59 (d, J = 3.7 Hz, 1 H), 7.64 (d, J = 8.4 Hz, 2 H), 7.81 (d, J = 3.7 Hz, 1 H), 10.52 (s, 1 H); ¹³C-NMR (150 MHz, d₆-DMSO) δ 55.21, 113.51, 113.94, 116.17, 122.32, 130.73, 148.18, 151.66, 154.25, 156.15; MS (ESI) C₁₂H₁₀N₂O₅ [M − H]⁻ m/z expected = 261.0517, observed = 261.0496. HPLC-1 = 98%, HPLC- 2 = >99%. |
| 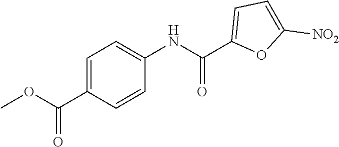 [SMILES: COC(=O)c1ccc(NC(=O)c2ccc([N+](=O)[O-])o2)cc1] methyl 4-(5-nitrofuran-2-carboxamido)benzoate. | Yield = 21%. ¹H NMR (600 MHz, DMSO- d₆) δ ppm 3.86 (s, 3 H), 7.66-7.72 (m, 1 H), 7.81-7.85 (m, 1 H), 7.92 (d, J = 8.1 Hz, 2 H), 8.00 (d, J = 8.1 Hz, 2 H), 10.91 (s, 1 H); ¹³C- NMR (150 MHz, d₆-DMSO) δ 52.01, 113.41, 117.10, 119.99, 125.14, 130.23, 142.31, 147.44, 151.91, 154.87, 165.72; MS (ESI) C₁₃H₁₀N₂O₆ [M − H]⁻ m/z expected = 289.0466, observed = 289.0460. HPLC-1 = 97%, HPLC- 2 = >99%. |
| 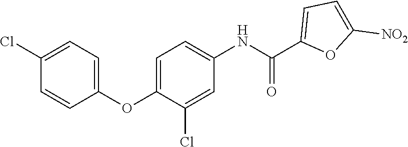 [SMILES: O=C(Nc1ccc(Oc2ccc(Cl)cc2)c(Cl)c1)c1ccc([N+](=O)[O-])o1] N-(3-chloro-4-(4-chlorophenoxy)phenyl)-5- nitrofuran-2-carboxamide. | Yield = 93%. ¹H NMR (600 MHz, DMSO- d₆) δ ppm 6.97 (m, J = 8.4 Hz, 2 H), 7.24 (d, J = 8.8 Hz, 1 H), 7.42 (m, J = 8.4 Hz, 2 H), 7.64 (d, J = 3.7 Hz, 1 H), 7.74 (d, J = 9.2 Hz, 1 H), 7.83 (d, J = 2.9 Hz, 1 H), 8.06 (s, 1 H), 10.83 (s, 1 H); ¹³C-NMR (150 MHz, d₆-DMSO) δ 113.47, 116.94, 118.58, 120.97, 122.20, 122.29, 124.89, 126.97, 129.88, 135.48, 147.16, 147.47, 151.83, 154.67, 155.77; MS (ESI) C₁₇H₁₀Cl₂N₂O₅ [M − H]⁻ m/z expected = 390.9894, observed = 390.9882. HPLC-1 = 97%, HPLC-2 = >99%. |
| 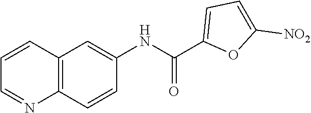 [SMILES: O=C(Nc1ccc2ncccc2c1)c1ccc([N+](=O)[O-])o1] 5-nitro-N-(quinolin-6-yl)furan-2-carboxamide. | Yield = 41%. ¹H NMR (600 MHz, DMSO- d₆) δ ppm 7.52 (dd, J = 8.1, 4.0 Hz, 1 H), 7.71 (d, J = 3.3 Hz, 1 H), 7.85 (d, J = 3.3 Hz, 1 H), 7.99-8.09 (m, 2 H), 8.36 (d, J = 8.4 Hz, 1 H), 8.46 (s, 1 H), 8.79-8.87 (m, 1 H), 10.94 (s, 1 H); ¹³C-NMR (150 MHz, d₆-DMSO) δ 113.49, 116.85, 117.14, 121.94, 124.13, 128.09, 129.60, 135.77, 145.15, 147.72, 149.73, 151.88, 154.91; MS (ESI) C₁₄H₉N₃O₄ |
| | [M − H]⁻ m/z expected = 282.0520, observed = |
| | 282.0514. HPLC-1 = >99%, HPLC-2 = 99%. |
| 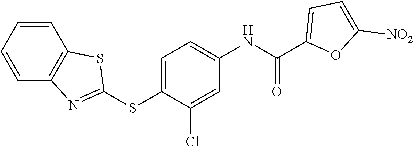 [SMILES: O=C(Nc1ccc(Sc2nc3ccccc3s2)c(Cl)c1)c1ccc([N+](=O)[O-])o1] N-(4-(benzo[d]thiazol-2-ylthio)-3- chlorophenyl)-5-nitrofuran- 2-carboxamide. | Yield = 74%. ¹H NMR (600 MHz, DMSO- d₆) δ ppm 7.52 (dd, J = 8.1, 4.0 Hz, 1 H), 7.71 (d, J = 3.3 Hz, 1 H), 7.85 (d, J = 3.3 Hz, 1 H), 8.00-8.08 (m, 2 H), 8.36 (d, J = 8.4 Hz, 1 H), 8.46 (s, 1 H), 8.80-8.89 (m, 1 H), 10.94 (s, 1 H); ¹³C-NMR (150 MHz, d₆-DMSO) δ 113.46, 117.49, 120.19, 121.49, 121.73, 121.87, 122.30, 124.62, 126.53, 134.89, 138.75, 138.88, 141.94, 147.12, 151.96, 153.39, 155.06, 167.61; MS (ESI) C₁₈H₁₀ClN₃O₄S₂ [M − H]⁻ m/z expected = 429.9728, observed = 429.9709. HPLC-1 = >99%, HPLC-2 = >99%. |
| 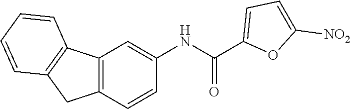 [SMILES: O=C(Nc1ccc2c(c1)-c1ccccc1C2)c1ccc([N+](=O)[O-])o1] N-(9H-fluoren-3-yl)-5-nitrofuran-2-carboxamide. | Yield = 46%. ¹H NMR (600 MHz, DMSO-d₆) δ ppm 3.96 (s, 2 H), 7.30 (t, J = 7.3 Hz, 1 H), 7.38 (t, J = 7.3 Hz, 1 H), 7.57 (d, J = 7.3 Hz, 1 H), 7.67 (d, J = 3.3 Hz, 1 H), 7.73 (d, J = 8.4 Hz, 1 H), 7.83 (d, J = 3.3 Hz, 1 H), 7.86 (d, J = 7.3 Hz, 1 H), 7.90 (d, J = 8.1 Hz, 1 H), 8.05 (s, 1 H), 10.70 (s, 1 H); ¹³C-NMR (150 MHz, d₆-DMSO) δ 36.54, 113.51, 116.45, 117.48, 119.48, 119.73, 120.19, 125.08, 126.45, 126.80, 136.76, 137.62, 140.75, 143.01, |
| | 143.73, 148.03, 151.79, 154.50; MS (ESI) |
| | C₁₈H₁₂N₂O₄ [M − H]⁻ m/z expected = 319.0724, |
| | observed = 319.0718. HPLC-1 = 99%, HPLC-2 = 98%. |
| 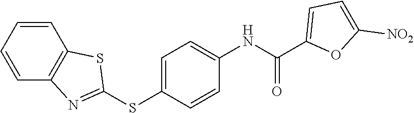 [SMILES: O=C(Nc1ccc(Sc2nc3ccccc3s2)cc1)c1ccc([N+](=O)[O-])o1] N-(4-(benzo[d]thiazol-2-ylthio)phenyl)-5- nitrofuran-2-carboxamide. | Yield = 69%. ¹H NMR (600 MHz, DMSO- d₆) δ ppm 7.34 (t, J = 7.5 Hz, 1 H), 7.45 (t, J = 7.7 Hz, 1 H), 7.70 (d, J = 4.0 Hz, 1 H), 7.84 (d, J = 8.4 Hz, 4 H), 7.93 (d, J = 7.7 Hz, 1 H), 7.97 (d, J = 7.7 Hz, 2 H), 10.92 (s, 1 H); ¹³C- NMR (150 MHz, d₆-DMSO) δ 113.46, 117.10, 121.34, 121.77, 121.82, 123.45, 124.45, 126.45, 134.78, 136.42, 140.47, 147.49, 151.90, 153.49, 154.89, 169.75; MS (ESI) C₁₈H₁₁N₃O₄S₂ [M − H]⁻ m/z expected = |
| | 396.0118, observed = 396.0095. HPLC-1 = |
| | >99%, HPLC-2 = >99%. |
| 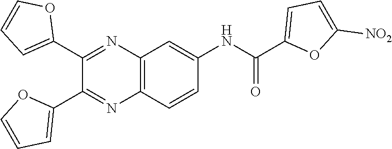 [SMILES: O=C(Nc1ccc2nc(-c3ccco3)c(-c3ccco3)nc2c1)c1ccc([N+](=O)[O-])o1] N-(2,3-di(furan-2-yl)quinoxalin-6-yl)-5- nitrofuran-2-carboxamide. | Yield = 60%. ¹H NMR (600 MHz, DMSO-d₆) δ ppm 6.69-6.77 (m, 4 H), 7.74 (d, J = 3.7 Hz, 1 H), 7.86 (d, J = 3.7 Hz, 2 H), 7.90 (d, J = 8.4 Hz, 1 H), 8.06-8.17 (m, 2 H), 8.63 (s, 1 H), 11.12 (s, 1 H); ¹³C-NMR (150 MHz, d₆-DMSO) δ 112.15, 112.23, 112.54, 113.02, 113.49, 116.88, 117.29, 125.12, 129.25, 137.30, 139.80, 140.49, 141.03, 142.52, 144.75, 145.01, 147.45, 150.38, 151.94, 155.14; MS (ESI) C₂₁H₁₂N₄O₆ [M − H]⁻ m/z expected = 416.0684, observed = 415.0672. HPLC-1 = >99%, HPLC-2 = 96%. |
| 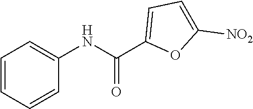 [SMILES: O=C(Nc1ccccc1)c1ccc([N+](=O)[O-])o1] 5-nitro-N-phenylfuran-2-carboxamide. | Yield = 86%. ¹H NMR (600 MHz, DMSO-d₆) δ ppm 7.16 (t, J = 7.3 Hz, 1 H), 7.39 (t, J = 7.7 Hz, 2 H), 7.64 (d, J = 3.7 Hz, 1 H), 7.73 (d, J = 7.7 Hz, 2 H), 7.82 (d, J = 3.7 Hz, 1 H), 10.62 (s, 1 H); ¹³C-NMR (7150 MHz, d₆-DMSO) δ 113.46, 116.50, 120.71, 124.57, 128.82, 137.79, 147.93, 151.76, 154.59; MS (ESI) C₁₁H₈N₂O₄ [M − H]⁻ m/z expected = 231.0411, observed = 231.0395. HPLC-1 = >99%, HPLC-2 = >99%. |

[symbol]

indicates data missing or illegible when filed

### General Synthetic Method B

The general protocols for the sulfonamide coupling, methoxy-to-hydroxy deprotection, nitro-to-amine reduction, and methyl ester-to-carboxyl deprotection reactions are presented below, with compound characterizations for each analog following.

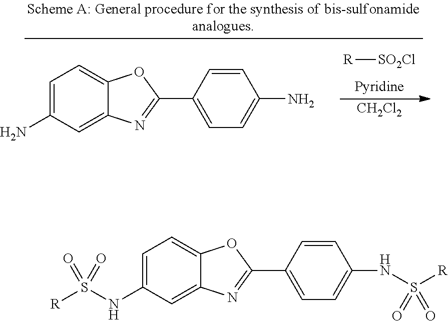

To a stirring mixture of 5-amino-2-(4-aminophenyl) benzoxazole (1 eq.) in anhydrous CH₂Cl₂ (5 mL) was added the respective sulfonyl chloride (2.1 eq.) followed by anhydrous pyridine (2.1 eq.). The reaction was allowed to stir at room temperature for 18 h and was then chromatographed over silica and concentrated. If necessary, the product was further purified by preparatory RP-HPLC (H₂O:CH₃CN gradient), concentrated, and lyophilized. Refer below for individual compound synthesis and characterization data.

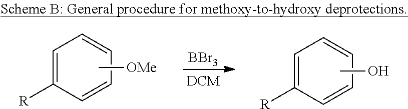

To a stirring mixture of the respective bis-sulfonamide (1 eq.) or mono-sulfonamide (1 eq.) in anhydrous CH₂Cl₂ (5 mL), was added BBr₃ (6 eq. or 3 eq., respectively, in CH₂Cl₂). The reaction was allowed to stir at room temperature for 18 h and then diluted drop-wise with MeOH (2 mL). The reaction was then washed with brine and extracted into EtOAc. The organics were dried over Na₂SO₄, filtered, and concentrated. The crude product was chromatographed over silica and concentrated. If necessary, the product was further purified by preparatory RP-HPLC (H₂O:CH₃CN gradient), concentrated, and lyophilized. Refer below for individual compound synthesis and characterization data.

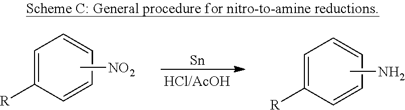

To the respective bis-sulfonamide (1 eq.) or mono-sulfonamide (1 eq.) was added tin powder (6 eq. or 3 eq., respectively), followed by a 1:10 mixture of HCl:AcOH (generally 0.2:2.0 mL). The reaction was allowed to stir at room temperature for 18 h, then diluted with EtOAc and H₂O, neutralized with NaHCO₃, and filtered. The filtrate was extracted with EtOAc and the organics dried over Na₂SO₄, filtered, and concentrated. The crude product was then chromatographed over silica and concentrated. If necessary, the product was further purified by preparatory RP-HPLC (H₂O:CH₃CN gradient), concentrated, and lyophilized. Refer below for individual compound synthesis and characterization data.

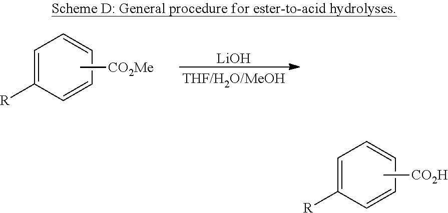

To a stirring mixture of the respective methyl ester compound (1 eq.) in THF (1.5 mL), MeOH (0.5 mL), and H₂O (0.5 mL), was added LiOH·H₂O (˜6-10 eq.). The reaction was allowed to stir at room temperature for 18 h and then was diluted with H₂O (10 mL) and acidified with 1M HCl. The precipitate was filtered, washed with H₂O, and dried. If necessary, the product was further purified by preparatory RP-HPLC (H₂O:CH₃CN gradient), concentrated, and lyophilized. Refer below for individual compound synthesis and characterization data.

The following examples of compounds of formula (XVIII), were synthesized as indicated in the methods above and via conventional chemistry:

| Structure | Characterization |
|---|---|
| Compound 162 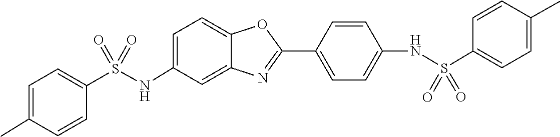 4-methyl-N-(4-(5-((4- methylphenyl)sulfonamido)benzo[d]oxazol-2- yl)phenyl)benzenesulfonamide | ¹H-NMR (500 MHz, d₆- DMSO) δ 10.82 (br s, 1H), 10.26 (br s, 1H), 7.76-8.02 (m, 2H), 7.70-7.75 (m, 2H), 7.57- 7.63 (m, 3H), 7.34-7.39 (m, 3H), 7.26-7.34 (m, 4H), 7.07 (dd, J = 8.5, 2.2 Hz, 1H), 2.32 (s, 3H), 2.30 (s, 3H); MS (ESI) 531.9 m/z [M − H⁺]⁻, C₂₇H₂₂N₃O₅S₂ requires 532.1; HPLC-1 = >99%; HPLC-2 = 98%. |
| Compound 163 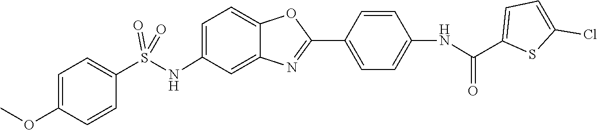 [SMILES: COc1ccc(S(=O)(=O)Nc2ccc3oc(-c4ccc(NC(=O)c5ccc(Cl)s5)cc4)nc3c2)cc1] 5-chloro-N-(4-(5-((4- methoxyphenyl)sulfonamido)benzo[d]oxazol-2- yl)phenyl)thiophene-2-carboxamide | ¹H-NMR (300 MHz, d₆-DMSO) δ 10.61 (s, 1H), 10.22 (br s, 1H), 8.12-8.17 (m, 2H), 7.92- 7.99 (m, 3H), 7.65-7.71 (m, 2H), 7.63 (d, J = 8.7 Hz, 1H), 7.42 (d, J = 2.0 Hz, 1H), 7.30 (d, J = 4.1 Hz, 1H), 7.09 (dd, J = 8.8, 2.1 Hz, 1H), 7.01-7.07 (m, 2H), 3.77 (s, 3H); MS (ESI) C₂₅H₁₈ClN₃O₅S₂ [M + H]⁺ m/z expected = 540.0, observed = 539.9; HPLC-1 = 98%; HPLC-2 = >99%. |
| Compound 164 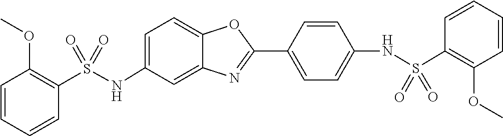 [SMILES: COc1ccccc1S(=O)(=O)Nc1ccc(-c2nc3cc(NS(=O)(=O)c4ccccc4OC)ccc3o2)cc1] 2-methoxy-N-(4-(5-((2- methoxyphenyl)sulfonamido)benzo[d]oxazol-2- yl)phenyl)benzenesulfonamide | ¹H-NMR (500 MHz, d₆- DMSO) δ 10.61 (br s, 1H), 10.04 (s, 1H), 7.92-7.96 (m, 2H), 7.85 (dd, J = 7.7, 1.7 Hz, 1H), 7.73 (dd, J = 7.7, 1.7 Hz, 1H), 7.50-7.58 (m, 3H), 7.36 (d, J = 1.9 Hz, 1H), 7.23-7.27 (m, 2H), 7.15 (dd, J = 7.9, .4.4 Hz, 2H), 7.10 (dd, J = 8.8, 2.2 Hz, 1H), 7.04-7.07 (m, 1H), 6.96-7.00 (m, 1H) 3.89 (s, 3H), 3.84 (s, 3H); MS (ESI) 566.1 m/z [MH⁺], C₂₇H₂₄N₃O₇S₂ requires 566.1; HPLC-1 = 97%; HPLC-2 = 96%. |
| 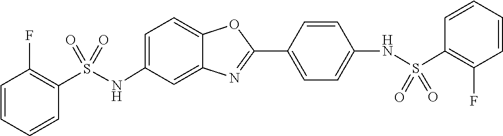 [SMILES: O=S(=O)(Nc1ccc(-c2nc3cc(NS(=O)(=O)c4ccccc4F)ccc3o2)cc1)c1ccccc1F] 2-fluoro-N-(4-(5-((2- fluorophenyl)sulfonamido)benzo[d]oxazol-2- yl)phenyl)benzenesulfonamide | ¹H-NMR (500 MHz, d₆- DMSO) δ 11.21 (br s, 1H), 10.66 (br s, 1H), 7.97 (d, J = 8.5 Hz, 2H), 7.92 (t, J = 6.9 Hz, 1H), 7.76-7.84 (m, 1H), 7.63-7.70 (m, 2H), 7.60 (d, J = 8.8 Hz, 1H), 7.35-7.44 (m, 4H), 7.32 (td, J = 7.6, 1.1 Hz, 1H), 7.26 (d, J = 8.2 Hz, 2H), 7.10 (dd, J = 8.7, 2.0 Hz, 1H); MS (ESI) 539.8 m/z [M − H⁺], C₂₅H₁₆F₂N₃O₅S₂ requires |
| | 540.0; HPLC-1 = >99%; |
| | HPLC-2 = 97%. |
| 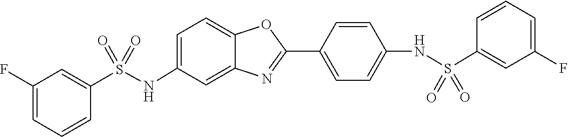 [SMILES: O=S(=O)(Nc1ccc(-c2nc3cc(NS(=O)(=O)c4cccc(F)c4)ccc3o2)cc1)c1cccc(F)c1] 3-fluoro-N-(4-(5-((2- fluorophenyl)sulfonamido)benzo[d]oxazol-2- yl)phenyl)benzenesulfonamide | ¹H-NMR (300 MHz, d₆- DMSO) δ 10.99 (br s, 1H), 10.47 (br s, 1H), 8.03 (d, J = 8.8 Hz, 2H), 7.40-7.77 (m, 10H), 7.31 (d, J = 8.8 Hz, 2H), 7.08 (dd, J = 8.7, 2.1 Hz, 1H); MS (ESI) 542.0 m/z [MH⁺], C₂₅H₁₈F₂N₃O₅S₂ requires 542.1; HPLC-1 = 100%; HPLC-2 = 98%. |
| 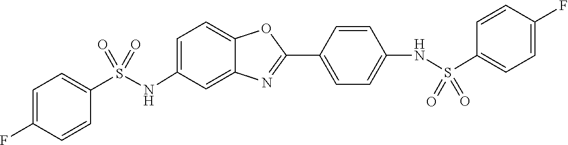 [SMILES: O=S(=O)(Nc1ccc(-c2nc3cc(NS(=O)(=O)c4ccc(F)cc4)ccc3o2)cc1)c1ccc(F)cc1] 4-fluoro-N-(4-(5-((4- fluorophenyl)sulfonamido)benzo[d]oxazol-2- yl)phenyl)benzenesulfonamide | ¹H-NMR (550 MHz, d₆- DMSO) δ 10.93 (br s, 1H), 10.35 (br s, 1H), 8.00 (d, J = 8.8 Hz, 2H), 7.83-7.92 (m, 2H), 7.75-7.80 (m, 2H), 7.61 (d, J = 8.8 Hz, 1H), 7.33-7.43 (m, 5H), 7.27 (d, J = 8.8 Hz, 2H), 7.06 (dd, J = 8.8, 2.2 Hz, 1H); MS (ESI) 542.0 m/z [MH⁺], C₂₅H₁₈F₂N₃O₅S₂ requires 542.1; HPLC-1 = 99%; HPLC-2 = 98%. |
| 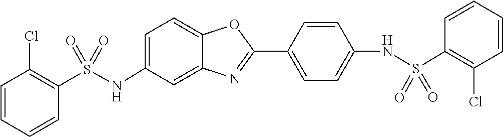 [SMILES: O=S(=O)(Nc1ccc(-c2nc3cc(NS(=O)(=O)c4ccccc4Cl)ccc3o2)cc1)c1ccccc1Cl] 2-chloro-N-(4-(5-((2- chlorophenyl)sulfonamido)benzo[d]oxazol-2- yl)phenyl)benzenesulfonamide | ¹H-NMR (500 MHz, d₆- DMSO) δ 11.21 (s, 1H), 10.67 (s, 1H), 8.11-8.15 (m, 1H), 8.02 (dd, J = 7.7, 1.4 Hz, 1H), 7.97 (d, J = 8.8 Hz, 2H), 7.53- 7.65 (m, 6H), 7.46-7.50 (m, 1H), 7.39 (d, J = 2.2 Hz, 1H), 7.26 (d, J = 8.5 Hz, 2H), 7.11 (dd, J = 8.8, 2.2 Hz, 1H); MS (ESI) 571.8 m/z [M − H]⁺]⁻; C₂₅H₁₆Cl₂N₃O₅S₂ requires 572.0; HPLC-1 = 99%; HPLC- 2 = 98%. |
| 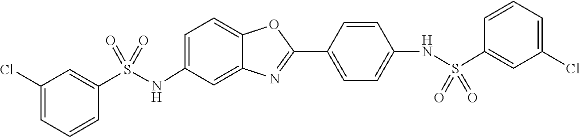 [SMILES: O=S(=O)(Nc1ccc(-c2nc3cc(NS(=O)(=O)c4cccc(Cl)c4)ccc3o2)cc1)c1cccc(Cl)c1] 3-chloro-N-(4-(5-((3- chlorophenyl)sulfonamido)benzo[d]oxazol-2- yl)phenyl)benzenesulfonamide | ¹H-NMR (500 MHz, d₆- DMSO) δ 10.98 (br s, 1H), 10.45 (br s, 1H), 8.03 (d, J = 8.8 Hz, 2H), 7.83 (t, J = 1.9 Hz, 1H), 7.53-7.80 (m, 8H), 7.41 (d, J = 2.2 Hz, 1H), 7.31 (d, J = 8.8 Hz, 2H), 7.07 (dd, J = 8.5, 2.2 Hz, 1H); MS (ESI) 571.7 m/z [M − H⁺]⁻;^{,} C₂₅H₁₆Cl₂N₃O₅S₂ requires 572.0; HPLC-1 = 99%; HPLC- 2 = 99%. |
| 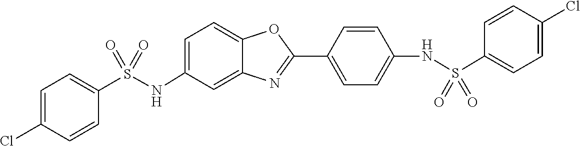 [SMILES: O=S(=O)(Nc1ccc(-c2nc3cc(NS(=O)(=O)c4ccc(Cl)cc4)ccc3o2)cc1)c1ccc(Cl)cc1] 4-chloro-N-(4-(5-((4- chlorophenyl)sulfonamido)benzo[d]oxazol-2- yl)phenyl)benzenesulfonamide | ¹H-NMR (500 MHz, d₆- DMSO) δ 10.95 (br s, 1H), 10.42 (br s, 1H), 8.00-8.04 (m, 2H), 7.81-7.85 (m, 2H), 7.70- 7.74 (m, 2H), 7.59-7.67 (m, 5H), 7.40 (d, J = 1.9 Hz, 1H), 7.27-7.32 (m, 2H), 7.07 (dd, J = 8.7, 2.0 Hz, 1H); MS (ESI) 571.8 m/z [M − H⁺]⁻, C₂₅H₁₆Cl₂N₃O₅S₂ requires 572.0; HPLC-1 = 98%; HPLC- 2 = 97%_{.} |
| 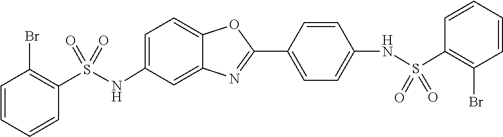 [SMILES: O=S(=O)(Nc1ccc(-c2nc3cc(NS(=O)(=O)c4ccccc4Br)ccc3o2)cc1)c1ccccc1Br] 2-bromo-N-(4-(5-((2- bromophenyl)sulfonamido)benzo[d]oxazol-2- yl)phenyl)benzenesulfonamide | ¹H-NMR (500 MHz, d₆- DMSO) δ 11.22 (s, 1H), 10.60 (s, 1H), 8.16 (dd, J = 7.9, 1.6 Hz, 1H), 8.07 (dd, J = 7.6, 1.9 Hz, 1H), 7.98 (d, J = 8.5 Hz, 2H), 7.82 (td, J = 8.0, 1.1 Hz, 2H), 7.57-7.63 (m, 2H), 7.54 (td, J = 7.7, 1.3 Hz, 2H), 7.47- 7.51 (m, 1H), 7.28 (d, J = 2.2 Hz, 1H), 7.26 (d, J = 8.5 Hz, 2H), 7.12 (dd, J = 8.8, 2.2 Hz, 1H); MS (ESI) 659.7 m/z [M − H⁺]⁻, C₂₅H₁₆Br₂N₃O₅S₂ |
| | requires 659.9; HPLC-1 = |
| | >99%; HPLC-2 = >99%. |
| 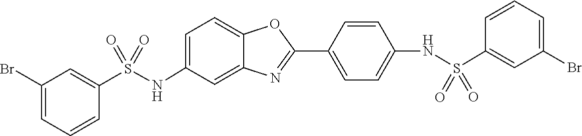 [SMILES: O=S(=O)(Nc1ccc(-c2nc3cc(NS(=O)(=O)c4cccc(Br)c4)ccc3o2)cc1)c1cccc(Br)c1] 3-bromo-N-(4-(5-((3- bromophenyl)sulfonamido)benzo[d]oxazol-2- yl)phenyl)benzenesulfonamide | ¹H-NMR (500 MHz, d₆- DMSO) δ 10.97 (br s, 1H), 10.43 (br s, 1H), 8.03 (d, J = 8.8 Hz, 2H), 7.98 (s, 1H), 7.88 (t, J = 1.7 Hz, 1H), 7.80-7.86 (m, 3H), 7.69 (dq, J = 7.9, 0.8 Hz, 1H), 7.64 (d, J = 8.5 Hz, 1H), 7.53 (t, J = 8.0 Hz, 1H), 7.48 (t, J = 8.0 Hz, 1H), 7.40 (d, J = 1.9 Hz, 1H), 7.30 (d, J = 7.9 Hz, 2H), 7.07 (dd, J = 8.7, 2.0 Hz, 1H); MS (ESI) 659.7 m/z [M − H⁺]⁻, |
| | C₂₅H₁₆Br₂N₃O₅S₂ requires |
| | 659.9; HPLC-1 = 98%; HPLC- |
| | 2 = 98%. |
| 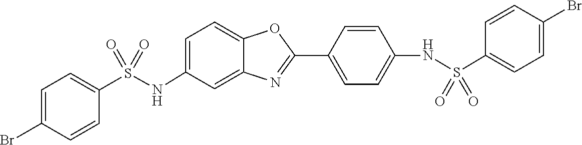 [SMILES: O=S(=O)(Nc1ccc(-c2nc3cc(NS(=O)(=O)c4ccc(Br)cc4)ccc3o2)cc1)c1ccc(Br)cc1] 4-bromo-N-(4-(5-((4- bromophenyl)sulfonamido)benzo[d]oxazol-2- yl)phenyl)benzenesulfonamide | ¹H-NMR (500 MHz, d₆- DMSO) δ 10.95 (br s, 1H), 10.42 (br s, 1H), 8.02 (d, J = 8.8 Hz, 2H), 7.77-7.82 (m, 2H), 7.73-7.77 (m, 4H), 7.61- 7.65 (m, 3H), 7.40 (d, J = 1.9 Hz, 1H), 7.29 (d, J = 8.8 Hz, 2H), 7.06 (dd, J = 8.8, 2.2 Hz, 1H); MS (ESI) 661.8 m/z [MH⁺], C₂₅H₁₈Br₂N₃O₅S₂ requires 661.9; HPLC-1 = 98%; HPLC-2 = 98%. |
| 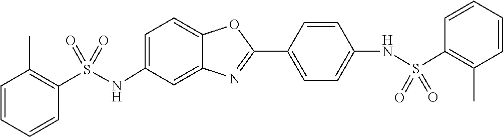 [SMILES: Cc1ccccc1S(=O)(=O)Nc1ccc(-c2nc3cc(NS(=O)(=O)c4ccccc4C)ccc3o2)cc1] 2-methyl-N-(4-(5-((2- methylphenyl)sulfonamido)benzo[d]oxazol-2- yl)phenyl)benzenesulfonamide | ¹H-NMR (300 MHz, d₆- DMSO) δ 11.03 (s, 1H), 10.47 (s, 1H), 7.94-8.00 (m, 3H), 7.86 (d, J = 7.6 Hz, 1H), 7.58 (d, J = 8.8 Hz, 1H), 7.30-7.55 (m, 7H), 7.24 (d, J = 8.8 Hz, 2H), 7.06 (dd, J = 8.8, 2.1 Hz, 1H), 2.60 (s, 3H), 2.59 (s, 3H); MS (ESI) 534.0 m/z [MH⁺], C₂₇H₂₄N₃O₅S₂ requires 534.1; HPLC-1 = 99%; HPLC-2 = 99%. |
| 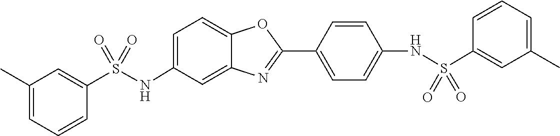 3-methyl-N-(4-(5-((2- methylphenyl)sulfonamido)benzo[d]oxazol-2- yl)phenyl)benzenesulfonamide | ¹H-NMR (300 MHz, d₆- DMSO) δ 10.86 (br s, 1H), 10.32 (br s, 1H), 8.00 (d, J = 8.8 Hz, 2H), 7.38-7.69 (m, 10H), 7.30 (d, J = 8.8 Hz, 2H), 7.07 (dd, J = 8.7, 2.1 Hz, 1H), 2.34 (s, 3H), 2.31 (s, 3H); MS (ESI) 534.0 m/z [MH⁺], C₂₇H₂₄N₃O₅S₂ requires 534.1; HPLC-1 = 98%; HPLC-2 = 98%. |
| 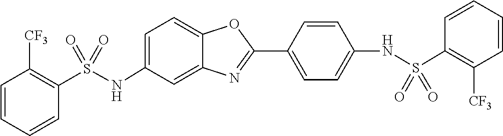 [SMILES: O=S(=O)(Nc1ccc(-c2nc3cc(NS(=O)(=O)c4ccccc4C(F)(F)F)ccc3o2)cc1)c1ccccc1C(F)(F)F] 2-(trifluoromethyl)-N-(4-(5-((2- (trifluoromethyl)phenyl)sulfonamido)benzo[d]oxazol-2- yl)phenyl)benzenesulfonamide | ¹H-NMR (500 MHz, d₆- DMSO) δ 11.24 (br s, 1H), 10.73 (br s, 1H), 8.13-8.17 (m, 1H), 8.10 (d, J = 7.6 Hz, 1H), 8.00-8.05 (m, 3H), 7.98 (dd, J = 7.4, 1.4 Hz, 1H), 7.78-7.90 (m, 4H), 7.64 (d, J = 8.8 Hz, 1H), 7.41 (d, J = 1.9 Hz, 1H), 7.29 (d, J = 8.8 Hz, 2H), 7.12 (dd, J = 8.8, 2.2 Hz, 1H); MS (ESI) 641.9 m/z [MH⁺], C₂₇H₁₈F₆N₃O₅S₂ requires 642.1; HPLC-1 = >99%; |
| | HPLC-2 = 99%. |
| 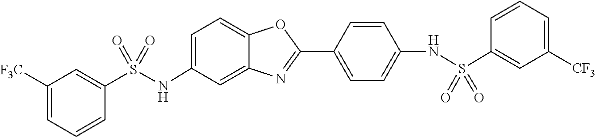 [SMILES: O=S(=O)(Nc1ccc(-c2nc3cc(NS(=O)(=O)c4cccc(C(F)(F)F)c4)ccc3o2)cc1)c1cccc(C(F)(F)F)c1] 3-(trifluoromethyl)-N-(4-(5-((3- (trifluoromethyl)phenyl)sulfonamido)benzo[d]oxazol-2- yl)phenyl)benzenesulfonamide | ¹H-NMR (500 MHz, d₆- DMSO) δ 11.01 (br s, 1H), 10.48 (br s, 1H), 8.05-8.13 (m, 2H), 7.95-8.05 (m, 6H), 7.83 (t, J = 7.9 Hz, 1H), 7.77 (t, J = 7.9 Hz, 1H), 7.64 (d, J = 8.8 Hz, 1H), 7.40 (d, J = 1.9 Hz, 1H), 7.31 (d, J = 8.8 Hz, 2H), 7.06 (dd, J = 8.7, 2.0 Hz, 1H); MS (ESI) 639.9 m/z [M − H⁺]⁻; C₂₇H₁₆F₆N₃O₅S₂ requires 640.0; HPLC-1 = >99%; HPLC-2 = 98%. |
| 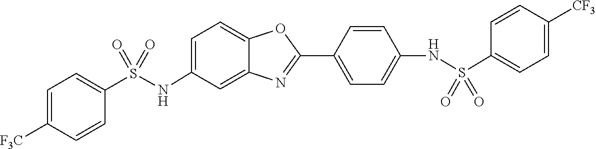 [SMILES: O=S(=O)(Nc1ccc(-c2nc3cc(NS(=O)(=O)c4ccc(C(F)(F)F)cc4)ccc3o2)cc1)c1ccc(C(F)(F)F)cc1] 4-(trifluoromethyl)-N-(4-(5-((4- (trifluoromethyl)phenyl)sulfonamido)benzo[d]oxazol-2- yl)phenyl)benzenesulfonamide | ¹H-NMR (500 MHz, d₆- DMSO) δ 11.09 (br s, 1H), 10.60 (br s, 1H), 8.03 (dd, J = 8.5, 6.0 Hz, 4H), 7.97 (d, J = 8.5 Hz, 2H), 7.93 (s, 4H), 7.63 (d, J = 8.8 Hz, 1H), 7.43 (d, J = 2.2 Hz, 1H), 7.31 (d, J = 8.8 Hz, 2H), 7.07 (dd, J = 8.7, 2.0 Hz, 1H); MS (ESI) 641.9 m/z [MH⁺], C₂₇H₁₈F₆N₃O₅S₂ requires 642.1; HPLC-1 = 98%; HPLC-2 = 98%. |
| 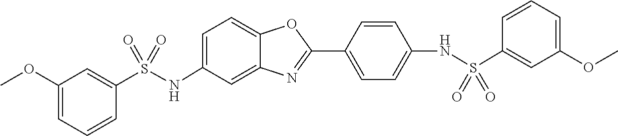 3-methoxy-N-(4-(5-((3- methoxyphenyl)sulfonamido)benzo[d]oxazol-2- yl)phenyl)benzenesulfonamide | ¹H-NMR (500 MHz, d₆- DMSO) δ 10.84 (br s, 1H), 10.32 (s, 1H), 7.99-8.04 (m, 2H), 7.61 (d, J = 8.8 Hz, 1H), 7.45-7.50 (m, 1H), 7.37-7.44 (m, 3H), 7.26-7.33 (m, 4H), 7.23-7.25 (m, 1H), 7.19 (ddd, J = 8.3, 2.6, 0.8 Hz, 1H), 7.13- 7.16 (m, 1H), 7.09 (dd, J = 8.8, 2.2 Hz, 1H), 3.77 (s, 3H), 3.74 (s, 3H); MS (ESI) 566.0 m/z [MH⁺], C₂₇H₂₄N₃O₇S₂ requires 566.1; HPLC-1 = >99%; |
| | HPLC-2 = >99%. |
| 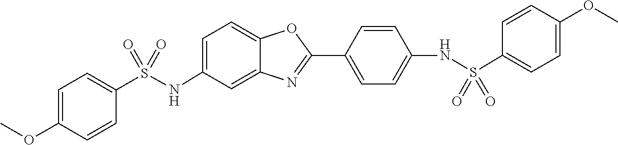 4-methoxy-N-(4-(5-((4- methoxyphenyl)sulfonamido)benzo[d]oxazol-2- yl)phenyl)benzenesulfonamide | ¹H-NMR (500 MHz, d₆- DMSO) δ 10.73 (br s, 1H), 10.20 (s, 1H), 7.97-8.02 (m, 2H), 7.75-7.80 (m, 2H), 7.64- 7.68 (m, 2H), 7.60 (d, J = 8.8 Hz, 1H), 7.38 (d, J = 2.2 Hz, 1H), 7.26-7.31 (m, 2H), 7.05- 7.10 (m, 3H), 7.00-7.05 (m, 2H), 3.78 (s, 3H), 3.76 (s, 3H); MS (ESI) 566.1 m/z [MH⁺], C₂₇H₂₄N₃O₇S₂ requires 566.1; HPLC-1 = 98%; HPLC-2 = 97%. |
| 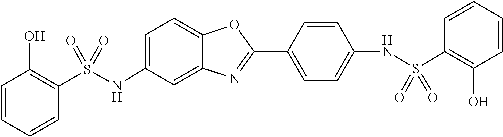 [SMILES: O=S(=O)(Nc1ccc(-c2nc3cc(NS(=O)(=O)c4ccccc4O)ccc3o2)cc1)c1ccccc1O] 2-hydroxy-N-(4-(5-((2- hydroxyphenyl)sulfonamido)benzo[d]oxazol-2- yl)phenyl)benzenesulfonamide | ¹H-NMR (300 MHz, d₆- DMSO) δ 10.91 (br s, 1H), 10.70 (br s, 1H), 9.99 (br s, 1H), 7.93 (d, J = 8.8 Hz, 2H), 7.78 (dd, J = 8.1, 1.6 Hz, 1H), 7.65 (dd, J = 7.9, 1.5 Hz, 1H), 7.54 (d, J = 8.8 Hz, 1H), 7.31- 7.44 (m, 3H), 7.26 (d, J = 8.8 Hz, 2H), 7.13 (dd, J = 8.8, 2.0 Hz, 1H), 6.87-6.96 (m, 3H), 6.79-6.86 (m, 1H); MS (ESI) 538.0 m/z [MH⁺], C₂₅H₂₀N₃O₇S₂ requires 538.1; |
| | HPLC-1 = 95%; HPLC-2 = |
| | 96%. |
| 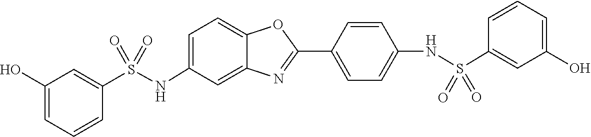 [SMILES: O=S(=O)(Nc1ccc(-c2nc3cc(NS(=O)(=O)c4cccc(O)c4)ccc3o2)cc1)c1cccc(O)c1] 3-hydroxy-N-(4-(5-((3- hydroxyphenyl)sulfonamido)benzo[d]oxazol-2- yl)phenyl)benzenesulfonamide | ¹H-NMR (300 MHz, d₆- DMSO) δ 10.84 (s, 1H), 10.30 (s, 1H), 10.19 (br s, 1H), 10.09 (br s, 1H), 8.02 (d, J = 88 Hz, 2H), 7.62 (d, J = 8.8 Hz, 1H), 7.23-7.42 (m, 6H), 7.04-7.21 (m, 4H), 6.90-7.01 (m, 2H); MS (ESI) 538.0 m/z [MH⁺], C₂₅H₂₀N₃O₇S₂ requires 538.1; HPLC-1 = 97%; HPLC-2 = 97%. |
| 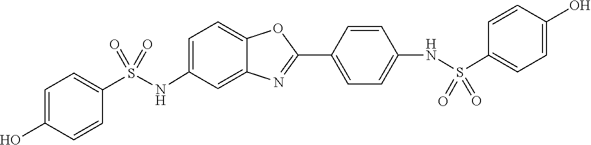 [SMILES: O=S(=O)(Nc1ccc(-c2nc3cc(NS(=O)(=O)c4ccc(O)cc4)ccc3o2)cc1)c1ccc(O)cc1] 4-hydroxy-N-(4-(5-((4- hydroxyphenyl)sulfonamido)benzo[d]oxazol-2- yl)phenyl)benzenesulfonamide | ¹H-NMR (500 MHz, d₆- DMSO) δ 10.41 (br s, 2H), 10.07 (br s, 1H), 7.96 (d, J = 8.2 Hz, 2H), 7.65 (d, J = 8.8 Hz, 2H), 7.53-7.60 (m, 3H), 7.35 (d, J = 1.9 Hz, 1H), 7.22 (d, J = 8.2 Hz, 2H), 7.04 (dd, J = 8.7, 2.0 Hz, 1H), 6.79-6.86 (m, 4H); MS (ESI) 535.8 m/z [M − H⁺]⁻, C₂₅H₁₈N₃O₇S₂ requires 536.1; HPLC-1 = 95%; HPLC-2 = 95%. |
| 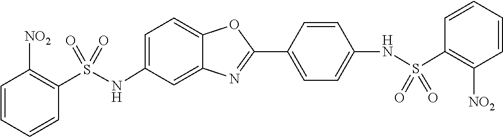 [SMILES: O=[N+]([O-])c1ccccc1S(=O)(=O)Nc1ccc(-c2nc3cc(NS(=O)(=O)c4ccccc4[N+](=O)[O-])ccc3o2)cc1] 2-nitro-N-(4-(5-((2- nitrophenyl)sulfonamido)benzo[d]oxazol-2- yl)phenyl)benzenesulfonamide | ¹H-NMR (500 MHz, d6- DMSO) δ 11.28 (br s, 1H), 10.78 (br s, 1H), 8.03-8.07 (m, 3H), 7.99 (d, J = 7.6 Hz, 1H), 7.96 (td, J = 7.5, 1.4 Hz, 2H), 7.77-7.87 (m, 4H), 7.67 (d, J = 8.8 Hz, 1H), 7.43 (d, J = 2.2 Hz, 1H), 7.31 (d, J = 8.5 Hz, 2H), 7.13 (dd, J = 8.8, 2.2 Hz, 1H); MS (ESI) 593.8 m/z [M − H⁺]⁻, C₂₅H₁₆N₅O₉S₂ requires 594.0; HPLC-1 = 98%; HPLC- 2 = 99%. |
| 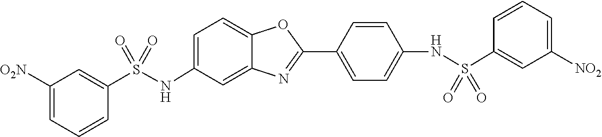 [SMILES: O=[N+]([O-])c1cccc(S(=O)(=O)Nc2ccc(-c3nc4cc(NS(=O)(=O)c5cccc([N+](=O)[O-])c5)ccc4o3)cc2)c1] 3-nitro-N-(4-(5-((3- nitrophenyl)sulfonamido)benzo[d]oxazol-2- yl)phenyl)benzenesulfonamide | ¹H-NMR (500 MHz, d₆- DMSO) δ 11.11 (br s, 1H), 10.63 (br s, 1H), 8.54 (t, J = 1.9 Hz, 1H), 8.49 (t, J = 1.9 Hz, 1H), 8.40-8.45 (m, 2H), 8.21 (dq, J = 7.9, 0.8 Hz, 1H), 8.07 (dq, J = 7.9, 0.8 Hz, 1H), 8.00 (d, J = 8.5 Hz, 2H), 7.77- 7.87 (m, 2H), 7.63 (d, J = 8.5 Hz, 1H), 7.42 (d, J = 2.2 Hz, 1H), 7.29 (d, J = 8.5 Hz, 2H), 7.08 (dd, J = 8.8, 2.2 Hz, 1H); MS (ESI) 596.1 m/z [MH⁺], |
| | C₂₅H₁₈N₅O₉S₂ requires 596.1; |
| | HPLC-1 = 95%; HPLC-2 = |
| | 95%. |
| 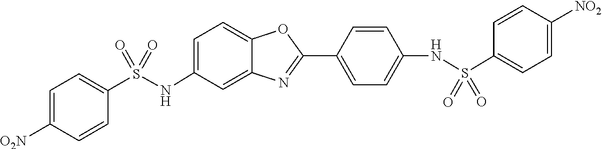 [SMILES: O=[N+]([O-])c1ccc(S(=O)(=O)Nc2ccc(-c3nc4cc(NS(=O)(=O)c5ccc([N+](=O)[O-])cc5)ccc4o3)cc2)cc1] 4-nitro-N-(4-(5-((4- nitrophenyl)sulfonamido)benzo[d]oxazol-2- yl)phenyl)benzenesulfonamide | ¹H-NMR (500 MHz, d₆- DMSO) δ 11.16 (br s, 1H), 10.67 (br s, 1H), 8.36-8.40 (m, 2H), 8.32-8.36 (m, 2H), 8.06- 8.10 (m, 2H), 8.01-8.05 (m, 2H), 7.94-7.98 (m, 2H), 7.64 (d, J = 8.8 Hz, 1H), 7.43 (d, J = 1.9 Hz, 1H), 7.30-7.34 (m, 2H), 7.08 (dd, J = 8.8, 2.2 Hz, 1H); MS (ESI) 593.9 m/z [M − H⁺], C₂₅H₁₆N₅O₉S₂ requires 594.0; HPLC-1 = 99%; HPLC- 2 = >99%. |
| 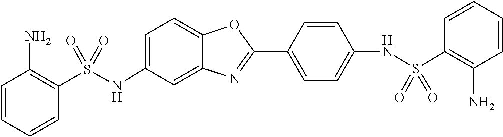 [SMILES: Nc1ccccc1S(=O)(=O)Nc1ccc(-c2nc3cc(NS(=O)(=O)c4ccccc4N)ccc3o2)cc1] 2-amino-N-(4-(5-((2- aminophenyl)sulfonamido)benzo[d]oxazol-2- | ¹H-NMR (300 MHz, d₆- DMSO) δ 10.80 (br s, 1H), 10.29 (br s, 1H), 7.98 (d, J = 8.8 Hz, 2H), 7.55-7.64 (m, 2H), 7.43-7.48 (m, 1H), 7.32- 7.36 (m, 1H), 7.15-7.25 (m, 4H), 7.04 (dd, J = 8.7, 2.1 Hz, 1H), 6.70-6.78 (m, 2H), 6.48- 6.60 (m, 2H), 6.00 (br s, 4H); MS (ESI) 536.0 m/z [MH⁺], C₂₅H₂₂N₅O₅S₂ requires 536.1; |
| yl)phenyl)benzenesulfonamide | HPLC-1 = >99%; HPLC-2 = |
| | 98%. |
| 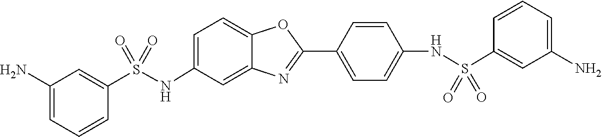 [SMILES: Nc1cccc(S(=O)(=O)Nc2ccc(-c3nc4cc(NS(=O)(=O)c5cccc(N)c5)ccc4o3)cc2)c1] 3-amino-N-(4-(5-((3- aminophenyl)sulfonamido)benzo[d]oxazol-2- yl)phenyl)benzenesulfonamide | ¹H-NMR (300 MHz, d₆- DMSO) δ 10.76 (s, 1H), 10.23 (s, 1H), 7.97-8.04 (m, 2H), 7.57-7.64 (m, 1H), 7.39 (d, J = 2.0 Hz, 1H), 7.25-7.32 (m, 2H), 7.12-7.22 (m, 2H), 7.04- 7.10 (m, 2H), 6.88-7.00 (m, 3H), 6.72-7.79 (m, 2H); MS (ESI) 536.1 m/z [MH⁺], C₂₅H₂₂N₅O₅S₂ requires 536.0; HPLC-1 = >99%; HPLC-2 = 98%. |
| 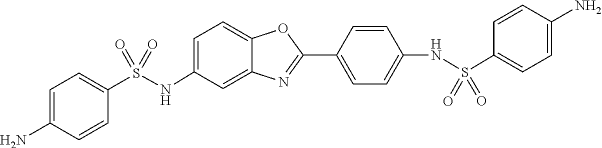 [SMILES: Nc1ccc(S(=O)(=O)Nc2ccc(-c3nc4cc(NS(=O)(=O)c5ccc(N)cc5)ccc4o3)cc2)cc1] 4-amino-N-(4-(5-((4- aminophenyl)sulfonamido)benzo[d]oxazol-2- yl)phenyl)benzenesulfonamide | ¹H-NMR (500 MHz, d₆- DMSO) δ 10.50 (br s, 1H), 9.88 (s, 1H), 7.97 (d, J = 8.5 Hz, 2H), 7.56 (d, J = 8.8 Hz, 1H), 7.46 (d, J = 8.8 Hz, 2H), 7.34-7.37 (m, 3H), 7.23 (d, J = 8.5 Hz, 2H), 7.04 (dd, J = 8.7, 2.0 Hz, 1H), 6.54 (d, J = 8.8 Hz, 2H), 6.50 (d, J = 8.8 Hz, 2H), 6.00 (br s, 2H), 5.95 (br s, 2H); MS (ESI) 536.0 m/z [MH⁺], C₂₅H₂₂N₅O₅S₂ requires 536.1; HPLC-1 = 98%; HPLC- |
| | 2 = 97%. |
| 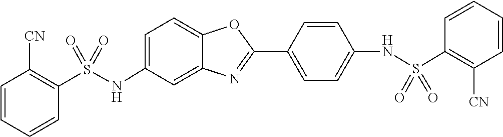 [SMILES: N#Cc1ccccc1S(=O)(=O)Nc1ccc(-c2nc3cc(NS(=O)(=O)c4ccccc4C#N)ccc3o2)cc1] 2-cyano-N-(4-(5-((2- cyanophenyl)sulfonamido)benzo[d]oxazol-2- yl)phenyl)benzenesulfonamide | ^{l}H-NMR (500 MHz, d₆- DMSO) δ 11.04 (s, 1H), 10.81 (s, 1H), 8.07-8.14 (m, 2H), 8.01-8.06 (m, 4H), 7.77-7.94 (m, 4H), 7.65 (d, J = 8.8 Hz, 1H), 7.40 (d, J = 2.2 Hz, 1H), 7.29 (d, J = 8.5 Hz, 2H), 7.08 (dd, J = 8.7, 2.0 Hz, 1H); MS (ESI) 556.1 m/z [MH⁺], C₂₇H₁₈N₅O₅S₂ requires 556.1; HPLC-1 = 95%; HPLC-2 = 95%. |
| 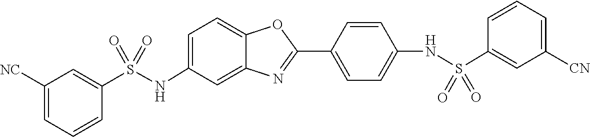 [SMILES: N#Cc1cccc(S(=O)(=O)Nc2ccc(-c3nc4cc(NS(=O)(=O)c5cccc(C#N)c5)ccc4o3)cc2)c1] 3-cyano-N-(4-(5-((3- cyanophenyl)sulfonamido)benzo[d]oxazol-2- yl)phenyl)benzenesulfonamide | ¹H-NMR (500 MHz, d₆- DMSO) δ 11.05 (br s, 1H), 10.52 (br s, 1H), 8.28 (s, 1H), 8.16 (s, 1H), 8.07-8.14 (m, 3H), 8.03 (d, J = 8.8 Hz, 2H), 7.98 (dd, J = 8.0, 1.1 Hz, 1H), 7.79 (t, J = 8.0 Hz, 1H), 7.74 (t, J = 7.9 Hz, 1H), 7.64 (d, J = 8.8 Hz, 1H), 7.42 (d, J = 2.2 Hz, 1H), 7.32 (d, J = 8.8 Hz, 2H), 7.07 (dd, J = 8.8, 2.2 Hz, 1H); MS (ESI) 556.0 m/z [MH⁺], C₂₇H₁₈N₅O₅S₂ requires |
| | 556.1; HPLC-1 = >99%; |
| | HPLC-2 = >99%. |
| 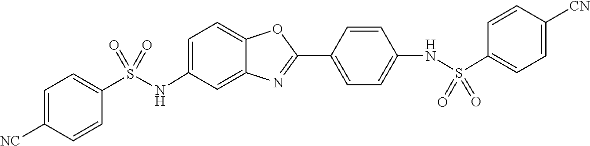 [SMILES: N#Cc1ccc(S(=O)(=O)Nc2ccc(-c3nc4cc(NS(=O)(=O)c5ccc(C#N)cc5)ccc4o3)cc2)cc1] 4-cyano-N-(4-(5-((4- cyanophenyl)sulfonamido)benzo[d]oxazol-2- yl)phenyl)benzenesulfonamide | ¹H-NMR (500 MHz, d₆- DMSO) δ 11.11 (br s, 1H), 10.60 (br s, 1H), 8.05-8.09 (m, 2H), 8.00-8.05 (m, 4H), 7.97- 8.00 (m, 2H), 7.87 (d, J = 8.5 Hz, 2H), 7.64 (d, J = 8.5 Hz, 1H), 7.41 (d, J = 1.9 Hz, 1H), 7.31 (d, J = 8.8 Hz, 2H), 7.07 (dd, J = 8.8, 2.2 Hz, 1H); MS (ESI) 553.9 m/z [M − H⁺]⁻, C₂₇H₁₆N₅O₅S₂ requires 554.1; HPLC-1 = >99%; HPLC-2 = 98%. |
| 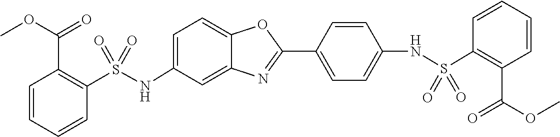 methyl 2-(N-(4-(5-((2- (methoxycarbonyl)phenyl)sulfonamido)benzo[d]oxazol-2- yl)phenyl)sulfamoyl)benzoate | ¹H-NMR (500 MHz, d₆- DMSO) δ 10.84 (br s, 1H), 10.29 (br s, 1H), 8.00 (d, J = 8.8 Hz, 2H), 7.91-7.94 (m, 1H), 7.84-7.87 (m, 1H), 7.58- 7.72 (m, 7H), 7.38 (d, J = 2.2 Hz, 1H), 7.26 (d, J = 8.5 Hz, 2H), 7.08 (dd, J = 8.7, 2.0 Hz, 1H), 3.87 (s, 3H), 3.83 (s, 3H); MS (ESI) 622.0 m/z [MH⁺], C₂₉H₂₄N₃O₉S₂ requires 622.1; HPLC-1 = 99%; HPLC-2 = 99%. |
| 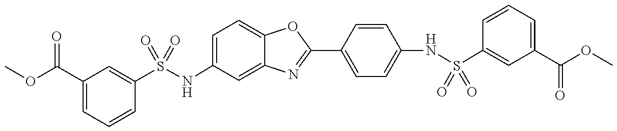 methyl 3-(N-(4-(5-((3- (methoxycarbonyl)phenyl)sulfonamido)benzo[d]oxazol-2- yl)phenyl)sulfamoyl)benzoate | 1H-NMR (500 MHz, d₆- DMSO) δ 11.00 (br s, 1H), 10.48 (br s, 1H), 8.37 (d, J = 1.6 Hz, 1H), 8.30 (d, J = 1.3 Hz, 1H), 8.10-8.18 (m, 2H), 8.07 (dd, J = 7.9, 0.9 Hz, 1H), 8.00 (d, J = 8.5 Hz, 2H), 7.91- 7.95 (m, 1H), 7.73 (t, J = 7.9 Hz, 1H), 7.67 (t, J = 7.9 Hz, 1H), 7.61 (d, J = 8.8 Hz, 1H), 7.38 (s, 1H), 7.29 (d, J = 8.5 Hz, 2H), 7.05 (dd, J = 8.7, 2.0 Hz, 1H), 3.87 (s, 3H), 3.85 (s, |
| | 3H); MS (ESI) 622.0 m/z |
| | [MH⁺], C₂₉H₂₄N₃O₉S₂ requires |
| | 622.1; HPLC-1 = 98%; HPLC- |
| | 2 = 99%. |
| 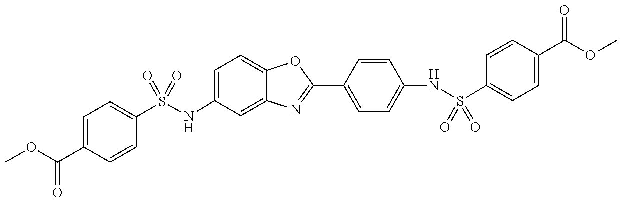 methyl 4-(N-(4-(5-((4- (methoxycarbonyl)phenyl)sulfonamido)benzo[d]oxazol-2- 9yl)phenyl)sulfamoyl)benzoate | ¹H-NMR (300 MHz, d₆- DMSO) δ 11.05 (s, 1H), 10.51 (s, 1H), 7.93-8.13 (m, 8H), 7.82-7.87 (m, 2H), 7.62 (d, J = 8.8 Hz, 1H), 7.39 (d, J = 2.0 Hz, 2H), 7.30 (d, J = 8.8 Hz, 1H), 7.06 (dd, J = 8.8, 2.1 Hz, 1H), 3.84 (s, 3H), 3.83 (s, 3H); MS (ESI) 622.2 m/z [MH⁺], C₂₉H₂₄N₃O₉S₂ requires 622.1; HPLC-1 = 95%; HPLC-2 = 95%. |
| 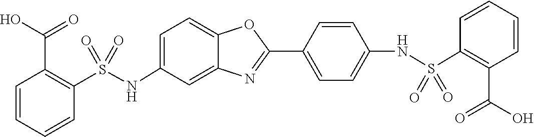 [SMILES: O=C(O)c1ccccc1S(=O)(=O)Nc1ccc(-c2nc3cc(NS(=O)(=O)c4ccccc4C(=O)O)ccc3o2)cc1] 2-(N-(4-(5-((2-carboxyphenyl)sulfonamido)benzo[d]oxazol- 2-yl)phenyl)sulfamoyl)benzoic acid | ¹H-NMR (300 MHz, d₆- DMSO) δ 13.65 (br s, 1H), 10.66 (br s, 1H), 10.05 (s, 1H), 8.00 (m, J = 8.8 Hz, 2H), 7.89 (d, J = 7.5 Hz, 1H), 7.78 (d, J = 7.5 Hz, 1H), 7.53-7.70 (m, 7H), 7.41 (d, J = 2.0 Hz, 1H), 7.30 (m, J = 8.8 Hz, 2H), 7.10 (dd, J = 8.8, 2.1 Hz, 1H); MS (ESI) 591.8 m/z [M − H⁺]^{-,} C₂₇H₁₈N₃O₉S₂ requires 592.1; HPLC-1 = >99%; HPLC-2 = |
| | 95%. |
| 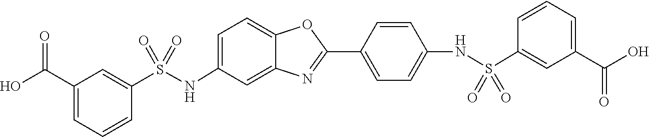 [SMILES: O=C(O)c1cccc(S(=O)(=O)Nc2ccc(-c3nc4cc(NS(=O)(=O)c5cccc(C(=O)O)c5)ccc4o3)cc2)c1] 3-(N-(4-(5-((3-carboxyphenyl)sulfonamido)benzo[d]oxazol- 2-yl)phenyl)sulfamoyl)benzoic acid | ¹H-NMR (500 MHz, d₆- DMSO) δ 13.52 (br s, 2H), 10.99 (br s, 1H), 10.45 (s, 1H), 8.36 (t, J = 1.6 Hz, 1H), 8.29 (t, J = 0.9 Hz, 1H), 8.15 (d, J = 7.9 Hz, 1H), 8.11 (d, J = 7.9 Hz, 1H), 8.00-8.07 (m, 3H), 7.91 (d, J = 8.5 Hz, 1H), 7.71 (t, J = 7.9 Hz, 1H), 7.60-7.67 (m, 2H), 7.39 (d, J = 2.2 Hz, 1H), 7.30 (d, J = 8.8 Hz, 2H), 7.06 (dd, J = 8.8, 1.9 Hz, 1H); |
| | MS (ESI) 594.0 m/z [MH⁺], |
| | C₂₇H₂₀N₃O₉S₂ requires 594.1; |
| | HPLC-1 = 99%; HPLC-2 = |
| | 98%. |
| 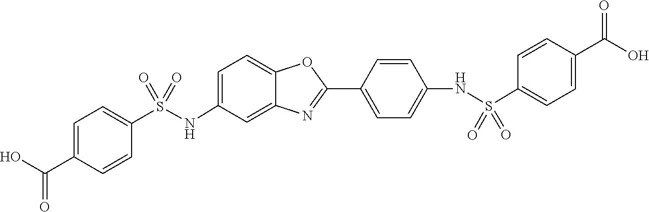 [SMILES: O=C(O)c1ccc(S(=O)(=O)Nc2ccc(-c3nc4cc(NS(=O)(=O)c5ccc(C(=O)O)cc5)ccc4o3)cc2)cc1] 4-(N-(4-(5-((4-carboxyphenyl)sulfonamido)benzo[d]oxazol- 2-yl)phenyl)sulfamoyl)benzoic acid | ¹H-NMR (300 MHz, d₆- DMSO) δ 13.44 (br s, 2H), 11.04 (s, 1H), 10.50 (s, 1H), 8.00-8.12 (m, 6H), 7.94 (d, J = 8.6 Hz, 2H), 7.83 (d, J = 8.5 Hz, 2H), 7.62 (d, J = 8.8 Hz, 1H), 7.40 (d, J = 2.0 Hz, 1H), 7.30 (d, J = 8.8 Hz, 2H), 7.07 (dd, J = 8.8, 2.0 Hz, 1H); MS (ESI) 591.8 m/z [M − H⁺]⁻; C₂₇H₂₀N₃O₉S₂ requires 592.1; HPLC-1 = 96%; HPLC-2 = 96%. |
| 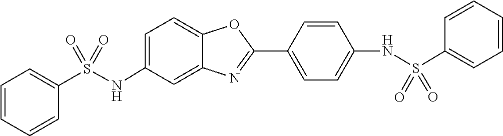 [SMILES: O=S(=O)(Nc1ccc(-c2nc3cc(NS(=O)(=O)c4ccccc4)ccc3o2)cc1)c1ccccc1] N-(4-(5-(phenylsulfonamido)benzo[d]oxazol-2- yl)phenyl)benzenesulfonamide | ¹H-NMR (500 MHz, d₆- DMSO) δ 10.90 (br s, 1H), 10.34 (br s, 1H), 8.00 (d, J = 8.8 Hz, 2H), 7.82-7.86 (m, 2H), 7.72-7.76 (m, 2H), 7.50- 7.65 (m, 7H), 7.39 (d, J = 2.2 Hz, 1H), 7.29 (d, J = 8.5 Hz, 2H), 7.07 (dd, J = 8.8, 2.2 Hz, 1H); MS (ESI) 506.0 m/z [MH⁺], C₂₅H₂₀N₃O₅S₂ requires 506.1; HPLC-1 = 100%; HPLC-2 = 99%. |
| 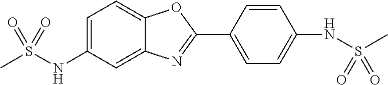 N-(4-(5-(methylsulfonamido)benzo[d]oxazol-2- yl)phenyl)methanesulfonamide | ¹H-NMR (500 MHz, d₆- DMSO) δ 10.38 (s, 1H), 9.79 (s, 1H), 8.15 (d, J = 8.9 Hz, 1H), 7.85 (d, J = 8.7, 1H), 7.75 (d, J = 9.1 Hz, 1H), 7.60 (d, J = 2.2 Hz, 1H), 7.40 (d, J = 8.9 Hz, 1H), 7.26 (dd, J = 8.6, 2.1 Hz, 2H), 3.33 (s, 6H); MS |
| | (ESI) 382.0 m/z [MH⁺], |
| | C₁₅H₁₆N₃O₅S₂ requires 382.1; |
| | HPLC-1 = 100%; HPLC-2 = |
| | 99%. |
| 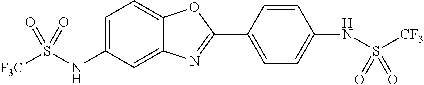 [SMILES: O=S(=O)(Nc1ccc(-c2nc3cc(NS(=O)(=O)C(F)(F)F)ccc3o2)cc1)C(F)(F)F] 1,1,1-trifluoro-N-(4-(5- ((trifluoromethyl)sulfonamido)benzo[d]oxazol-2- yl)phenyl)methanesulfonamide | ¹H-NMR (500 MHz, d₆- DMSO) δ 8.19 (d, J = 8.5 Hz, 1H), 7.85 (d, J = 8.6 Hz, 1H), 7.67 (d, J = 2.1, 1H), 7.46 (d, J = 8.7 Hz, 2H), 7.31 (dd, J = 8.7, 2.1 Hz, 2H); MS (ESI) 490.1 m/z [MH⁺], C₁₅H₁₀F₆N₃O₅S₂ requires 490.0; HPLC-1 = 97%; HPLC- 2 = 97%. |
| 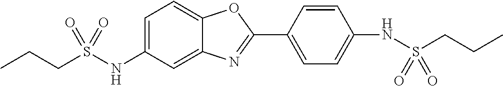 [SMILES: CCCS(=O)(=O)Nc1ccc(-c2nc3cc(NS(=O)(=O)CCC)ccc3o2)cc1] N-(4-(5-(propylsulfonamido)benzo[d]oxazol-2- yl)phenyl)propane-1-sulfonamide | ¹H-NMR (500 MHz, d₆- DMSO) δ 10.41 (br s, 1H), 9.85 (br s, 1H), 8.12 (d, J = 8.2 Hz, 2H), 7.73 (d, J = 8.8 Hz, 1H), 7.58 (d, J = 2.2 Hz, 1H), 7.39 (d, J = 8.8 Hz, 2H), 7.25 (dd, J = 8.8, 2.2 Hz, 1H), 3.16-3.24 (m, 2H), 3.04-3.09 (m, 2H), 1.66-1.75 (m, 4H), 0.90-0.98 |
| | (m, 6H); MS (ESI) 438.2 m/z |
| | [MH⁺], C₁₉H₂₄N₃O₅S₂ requires |
| | 438.1; HPLC-1 = 98%; HPLC- |
| | 2 = 99%. |
| 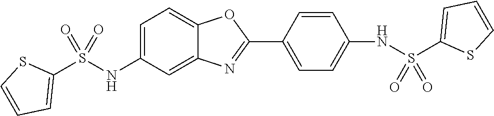 [SMILES: O=S(=O)(Nc1ccc(-c2nc3cc(NS(=O)(=O)c4cccs4)ccc3o2)cc1)c1cccs1] N-(4-(5-(thiophene-2-sulfonamido)benzo[d]oxazol-2- yl)phenyl)thiophene-2-sulfonamide | ¹H-NMR (500 MHz, d₆- DMSO) δ 11.01 (s, 1H), 10.46 (s, 1H), 8.06 (d, J = 8.2 Hz, 2H), 7.92 (d, J = 4.7 Hz, 1H), 7.88 (dd, J = 4.9, 1.4 Hz, 1H), 7.65-7.68 (m, 2H), 7.51 (dd, J = 3.8, 1.6 Hz, 1H), 7.46 (d, J = 1.9 Hz, 1H), 7.35 (d, J = 8.5 Hz, 2H), 7.07-7.15 (m, 3H); MS (ESI) 517.9 m/z [MH⁺], C₂₁H₁₆N₃O₅S₄ requires 518.0; |
| | HPLC-1 = 98%; HPLC-2 = |
| | 99%. |
| 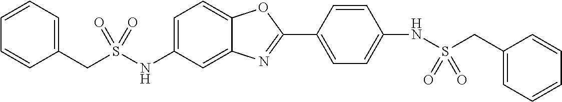 [SMILES: O=S(=O)(Cc1ccccc1)Nc1ccc(-c2nc3cc(NS(=O)(=O)Cc4ccccc4)ccc3o2)cc1] 1-phenyl-N-(4-(5- ((phenylmethyl)sulfonamido)benzo[d]oxazol-2- yl)phenyl)methanesulfonamide | ¹H-NMR (500 MHz, d₆- DMSO) δ 10.42 (br s, 1H), 9.92 (br s, 1H), 8.10-8.14 (m, 2 H), 7.73 (d, J = 8.8 Hz, 1H), 7.59 (d, J = 1.9 Hz, 1H), 7.33- 7.38 (m, 8H), 7.27-7.31 (m, 4H), 7.23 (dd, J = 8.8, 2.2 Hz, 1H), 4.60 (s, 2H), 4.49 (s, 2H); MS (ESI) 534.0 m/z [MH⁺], C₂₇H₂₄N₃O₅S₂ requires 534.1; HPLC-1 = 99%; HPLC-2 = |
| | 97%. |
| 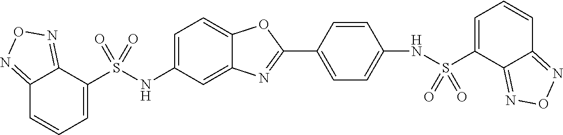 [SMILES: O=S(=O)(Nc1ccc(-c2nc3cc(NS(=O)(=O)c4cccc5nonc45)ccc3o2)cc1)c1cccc2nonc12] N-(4-(5-(benzo[c][1,2,5]oxadiazole-4- sulfonamido)benzo[d]oxazol-2- yl)phenyl)benzo[c][1,2,5]oxadiazole-4-sulfonamide | ¹H-NMR (500 MHz, d₆- DMSO) δ 11.56 (br s, 1H), 11.00 (br s, 1H), 8.31-8.36 (m, 2H), 8.23 (d, J = 5.0 Hz, 1H), 8.09-8.14 (m, 1H), 7.94 (d, J = 8.5 Hz, 2H), 7.70-7.75 (m, 1H), 7.65-7.69 (m, 1H), 7.56 (d, J = 8.8 Hz, 1H), 7.40 (d, J = 1.9 Hz, 1H), 7.27 (d, J = 7.9 Hz, 2H), 7.04 (dd, J = 8.7, 2.0 Hz, 1H); MS (ESI) 590.1 m/z [MH⁺], C₁₅H₁₆N₇O₇S₂ requires 590.1; HPLC-1 = 95; HPLC-2 = |
| | 95%. |
| 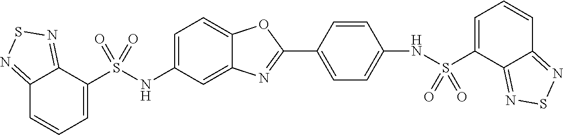 [SMILES: O=S(=O)(Nc1ccc(-c2nc3cc(NS(=O)(=O)c4cccc5nsnc45)ccc3o2)cc1)c1cccc2nsnc12] N-(4-(5-(benzo[c][1,2,5]thiadiazole-4- sulfonamido)benzo[d]oxazol-2- yl)phenyl)benzo[c][1,2,5]thiadiazole-4-sulfonamide | ¹H-NMR (500 MHz, d₆- DMSO) δ 11.34 (br s, 1H), 10.75 (br s, 1H), 8.33-8.38 (m, 3H), 8.24 (dd, J = 7.1, 1.1 Hz, 1H), 7.83-7.89 (m, 3H), 7.79 (dd, J = 8.8, 6.9 Hz, 1H), 7.47 (dd, J = 8.8 Hz, 1H), 7.33 (d, J = 1.9 Hz, 1H), 7.22-7.27 (m, 2H), 7.01 (dd, J = 8.8, 2.2 Hz, 1H); MS (ESI) 621.9 m/z [MH⁺], C₂₃H₁₆N₇O₅S₄ requires 622.0; HPLC-1 = 98; HPLC-2 = 97%. |
| 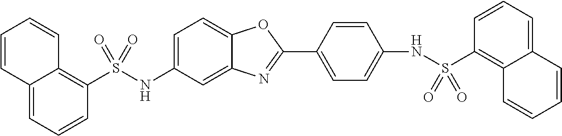 [SMILES: O=S(=O)(Nc1ccc(-c2nc3cc(NS(=O)(=O)c4cccc5ccccc45)ccc3o2)cc1)c1cccc2ccccc12] N-(4-(5-(naphthalene-1-sulfonamido)benzo[d]oxazol-2- yl)phenyl)naphthalene-1-sulfonamide | ¹H NMR (500 MHz, DMSO) δ 11.26 (br s, 1H), 10.70 (br s, 1H), 8.73 (d, J = 8.5 Hz, 2H), 8.29 (d, J = 7.3 Hz, 1H), 8.15- 8.23 (m, 3H), 8.03-8.07 (m, 2H), 7.86 (d, J = 8.5 Hz, 2H), 7.70-7.76 (m, 2H), 7.61-7.67 (m, 3H), 7.57 (t, J = 7.7 Hz, 1H), 7.47 (d, J = 8.8 Hz, 1H), 7.26 (d, J = 1.9 Hz, 1H), 7.18 (d, J = 8.5 Hz, 2H), 6.95 (dd, J = 8.8, 2.2 Hz, 1H); MS (ESI) |
| | 606.0 m/z [MH⁺], |
| | C₃₃H₂₄N₃O₅S₂ requires 606.1 |
| | HPLC-1 = 99; HPLC-2 = 99%. |
| 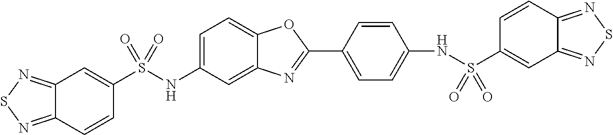 [SMILES: O=S(=O)(Nc1ccc(-c2nc3cc(NS(=O)(=O)c4ccc5nsnc5c4)ccc3o2)cc1)c1ccc2nsnc2c1] N-(4-(5-(benzo[c][1,2,5]thiadiazole-5- sulfonamido)benzo[d]oxazol-2- yl)phenyl)benzo[c][1,2,5]thiadiazole-5-sulfonamide | ¹H-NMR (500 MHz, d₆- DMSO) δ 11.18 (br s, 1H), 10.68 (br s, 1H), 8.59 (s, 1H), 8.45 (dd, J = 1.9, 0.6 Hz, 1H), 8.27-8.30 (m, 2H), 7.93-7.99 (m, 4H), 7.58 (d, J = 8.8 Hz, 1H), 7.44 (d, J = 1.9 Hz, 1H), 7.32 (d, J = 8.8 Hz, 2H), 7.10 (dd, J = 8.8, 2.2 Hz, 1H); MS (ESI) 621.9 m/z [MH⁺], C₂₅H₁₆N₇O₅S₄ requires 622.1; HPLC-1 = 98; HPLC-2 = 98%. |
| 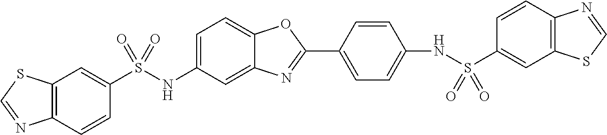 [SMILES: O=S(=O)(Nc1ccc(-c2nc3cc(NS(=O)(=O)c4ccc5ncsc5c4)ccc3o2)cc1)c1ccc2ncsc2c1] N-(4-(5-(benzo[d]thiazole-6-sulfonamido)benzo[d]oxazol-2- yl)phenyl)benzo[d]thiazole-6-sulfonamide | ¹H-NMR (500 MHz, d₆- DMSO) δ 11.01 (br s, 1H), 10.45 (br s, 1H), 9.60 (s, 1H), 9.57 (s, 1H), 8.80 (d, J = 1.9 Hz, 1H), 8.67 (d, J = 1.6 Hz, 1H), 8.23 (d, J = 8.5 Hz, 1H), 8.21 (d, J = 9.1 Hz, 1H), 7.95- 7.98 (m, 2H), 7.92-7.95 (m, 1H), 7.84-7.88 (m, 1H), 7.57 (d, J = 8.8 Hz, 1H), 7.41 (d, J = 1.9 Hz, 1H), 7.30 (d, J = 8.8 Hz, 2H), 7.06 (dd, J = 8.8, 2.2 |
| | Hz, 1H); MS (ESI) 619.8 m/z |
| | [MH⁺], C₂₇H₁₈N₅O₅S₄ requires |
| | 620.0; HPLC-1 = 99; HPLC-2 = |
| | 98%. |
| 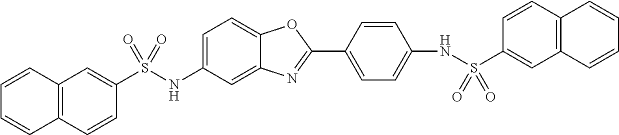 [SMILES: O=S(=O)(Nc1ccc(-c2nc3cc(NS(=O)(=O)c4ccc5ccccc5c4)ccc3o2)cc1)c1ccc2ccccc2c1] N-(4-(5-(naphthalene-2-sulfonamido)benzo[d]oxazol-2- yl)phenyl)naphthalene-2-sulfonamide | ¹H NMR (500 MHz, DMSO) δ 11.01 (br s, 1H), 10.43 (br s, 1H), 8.53 (s, 1H), 8.39 (s, 1H), 8.12-8.16 (m, 1H), 8.04-8.10 (m, 3H), 7.97 (t, J = 7.9 Hz, 2H), 7.92 (d, J = 8.8 Hz, 2H), 7.80 (dd, J = 8.8, 1.9 Hz, 1H), 7.75 (dd, J = 8.5, 1.9 Hz, 1H), 7.58-7.69 (m, 4H), 7.53 (d, J = 8.8 Hz, 1H), 7.40 (d, J = 2.2 Hz, 1H), 7.29 (d, J = 8.8 Hz, 2H), 7.06 (dd, J = 8.8, 2.2 Hz, |
| | 1H); MS (ESI) 606.2 m/z |
| | [MH⁺], C₃₃H₂₄N₃O₅S₂ requires |
| | 606.1; HPLC-1 = 99%; HPLC- |
| | 2 = 99%. |
| 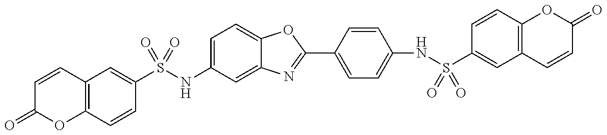 [SMILES: O=c1ccc2cc(S(=O)(=O)Nc3ccc(-c4nc5cc(NS(=O)(=O)c6ccc7oc(=O)ccc7c6)ccc5o4)cc3)ccc2o1] 2-oxo-N-(4-(5-((2-oxo-2H-chromene)-6- sulfonamido)benzo[d]oxazol-2-yl)phenyl)-2H-chromene-6- sulfonamide | ¹H-NMR (500 MHz, d₆- DMSO) δ 11.04 (br s, 1H), 10.47 (br s, 1H), 8.28 (d, J = 2.2 Hz, 1H), 8.12-8.18 (m, 3H), 7.95-7.98 (m, 3H), 7.88 (dd, J = 8.8, 2.2 Hz, 1H), 7.60 (d, J = 8.8 Hz, 1H), 7.54 (dd, J = 8.5, 6.6 Hz, 2H), 7.42 (d, J = 1.9 Hz, 1H), 7.28 (d, J = 8.8 Hz, 2H), 7.06 (dd, J = 8.7, 2.0 Hz, 1H), 6.55-6.61 (m, 2H); MS (ESI) 641.9 m/z [MH⁺], C₃₁H₂₀N₃O₉S₂ requires 642.1; |
| | HPLC-1 = 97%; HPLC-2 = |
| | 97%. |

### Biological Assay Methods

The following examples are offered to illustrate but not to limit the disclosure. One of skill in the art will recognize that the following assays and methods may be modified by choice of suitable materials and methods.

### General Materials and Methods for Biochemical and Cell-Based Experiments.

DH5α and BL21 (DE3) E. coli cells were purchased from New England Biolabs, and Rosetta™ 2 (DE3) E. coli cells from EMD Millipore. The human cell viability assays were performed using FHC (CRL-1831), FHs-74Int (CCL-241), DLD-1 (CCL-221), HT-29 (HTB-38), and HCT-116 (CCL-247) cell lines that were obtained from the ATCC. Antibiotics were used in the following concentrations when appropriate: Kanamycin (34 μg/mL), Ampicillin (50 μg/mL), Chloramphenicol (30 μg/mL) and Streptomycin (100 μg/mL).

### Evaluating the Compound Diversity Set for Effects on the Viability of Colorectal Cancer Cells (HCT-116, DLD-1, and HT-29) and Non-Cancerous Colon (FHC) and Intestine (FHs-74Int) Cell Lines.

Evaluation of test compounds for their cytotoxicity to the colorectal cancer (HCT-116, DLD-1, and HT-29) and non-cancerous colon (FHC) and intestine (FHs-74Int) cell lines was performed using AlamarBlue-based cell viability assays as we previously described [30,40]. The HCT-116, HT-29 and DLD-1 cells were maintained in RPMI-1640 medium supplemented with 100 units/ml penicillin, 100 μg/ml streptomycin, and 10% FBS. FHC cells were maintained in DMEM F12 medium (CC-1831) supplemented with 10 mM HEPES, 10 ng/ml cholera toxin, 5 μg/ml insulin, 5 μg/ml transferrin, 100 ng/ml hydrocortisone, 20 ng/ml human recombinant EGF, 100 units/ml penicillin, 100 μg/ml streptomycin, and 10% FBS. FHs-74Int cells were maintained in Hybri-Care medium 46-X supplemented with 100 units/ml penicillin, 100 μg/ml streptomycin, and 10% FBS. Compound testing was carried out in 384-well plates (BRAND cell culture grade plates, 781980). Cells at 80% confluence were harvested and diluted in growth medium, then 45 μL of the respective cells were dispensed per well (1,500 cells/well), and plates were sealed with “Breathe Easy” oxygen permeable membranes (Diversified Biotech) and incubated at 37° C., 5% CO₂, for 24 h. The following day, 1 μL of the compound stocks (10 mM to 4.6 μM, 3-fold dilutions in DMSO) were pre-diluted by pin-transfer into 25 μL of the relevant growth media. Then, 15 μL aliquots of the diluted compounds were added to the cell assay plates to give inhibitor concentration ranges of 10 μM to 46 nM during the assay (final DMSO concentration of 1% was maintained during the assay). Plates were sealed with “Breathe Easy” oxygen permeable membranes and incubated for an additional 48 h at 37° C. and 5% CO₂. The AlamarBlue reporter reagents were then added to a final concentration of 10%, the plates incubated at 37° C. and 5% CO₂, and sample absorbance (570 and 600 nm) was read using a Molecular Devices SpectraMax Plus 384 microplate reader (readings taken between 4-24 h of incubation so as to achieve signals in the 30-60% range for conversion of resazurin to resorufin). Cell viability was calculated as per vendor instructions (Thermo Fisher—AlamarBlue cell viability assay manual). Inhibitor efficacy against cancer cells (EC₅₀) and cytotoxicity to non-cancer cells (CC₅₀) for the test compounds were obtained by plotting the % resazurin reduction results in GraphPad Prism and analyzing by non-linear regression using the log(inhibitor) vs. response (variable slope) equation. Results presented represent the averages of EC₅₀ or CC₅₀ values obtained from at least four replicates for all the cell lines.

Purification of Human HSP60 with and without the 26 Amino Acid MTS.

Processed human HSP60 (mHSP60—that would be in mitochondria) was expressed in Rosetta™ 2 (DE3) E. coli using a previously reported pET21-HSP60 plasmid with an N-terminal octa-Histidine tag [13,30,40]. Naïve HSP60 was expressed and purified similarly using a plasmid that has the N-terminal 26 amino acid mitochondrial targeting sequence still intact, preceded by the His-tag. Transformed cells were grown at 37° C. in LB/ampicillin/chloramphenicol medium until reaching an OD₆₀₀ of 0.5, then cultures were induced with 0.8 mM IPTG and continued to grow for 2-3 h at 25° C. Cells were centrifuged at 14,000 rpm, then the cell pellets were suspended in 50 mL of lysis buffer composed of 100 mM Tris-HCl (pH 7.7), 10 mM MgSO₄, 1 mM β-ME, 5% glycerol, 0.1% triton X-100, 50 mg/ml lysozyme, and one tablet of EDTA-free complete protease inhibitor cocktail (Roche). Cells were homogenized and passed through a microfluidizer, washing with buffer containing 10 mM Tris-HCl (pH 7.7), 5% glycerol, 1 mM β-ME, and 0.1% triton X-100.

1st Nickel column purification and His-tag cleavage: The lysates were centrifuged at 14,000 rpm and the clarified lysates supplemented with 10 mM imidazole, passed through 0.2 m filters, and loaded onto a nickel-agarose resin column equilibrated with 3 column volumes of 20 mM Tris-HCl (pH 7.7), 5% glycerol, 200 mM NaCl, 1 mM R-ME, and 10 mM imidazole. The loaded column was washed with 6 column volumes of 10% 20 mM Tris-HCl (pH 7.7), 5% glycerol, 200 mM NaCl, 1 mM j-ME, and 500 mM Imidazole. Protein was then eluted with 10 column volumes of 100% 20 mM Tris-HCl (pH 7.7), 5% glycerol, 200 mM NaCl, 1 mM β-ME, and 500 mM Imidazole. Fractions that were enriched with the His-tagged HSP60 were collected, concentrated, dialyzed at 4° C. for 2 h in 4 L of 20 mM Tris-HCl (pH 7.7), 200 mM NaCl, 1 mM j-ME, and 5% glycerol. Proteolytic cleavage of the His-tag was next performed by addition of a 10-fold (w/w) excess of His-tagged TEV protease, while dialyzing over night at 4° C. against 4 L of 20 mM Tris-HCl (pH 7.7), 200 mM NaCl, 1 mM j-ME, and 5% glycerol buffer.

2nd Nickel column purification: The protein samples were loaded onto a second nickel-agarose resin column equilibrated with 20 mM Tris-HCl (pH 7.7), 5% glycerol, and 1 mM j-ME. With this column, undigested His-tagged HSP60 could be separated from digested HSP60 with the His-tag removed. The column was washed with 5 column volumes of 20 mM Tris-HCl (pH 7.7), 5% glycerol, and 1 mM β-ME. The unbound fractions enriched with tag-less HSP60 were collected, and the column was then eluted with 4 column volumes of 20 mM Tris-HCl (pH 7.7), 200 mM NaCl, 500 mM Imidazole, 1 mM β-ME, and 5% glycerol to elute any undigested His-tag HSP60 from the column. Fractions enriched with tag-less HSP60 were collected, concentrated, and dialyzed in storage buffer (20 mM Tris-HCl (pH 7.7), 300 mM NaCl, 5% glycerol, 10 mM MgCl₂, and 1 mM β-ME) using 0.5-3 mL capacity Slide-A-Lyzer Dialysis Cassette (Extra Strength) (Thermo Scientific). Protein concentrations were determined using a Coomassie Protein Assay Kit (Thermo Scientific). Batches of mHSP60 and cHSP60 were stored at 4° C. for up to one week, then discarded.

### Human HSP10 Purification.

Human HSP10 was expressed from a T7-promoted (pET3a-HSP10) plasmid in Rosetta™ 2 (DE3) pLysS cells. Cells were grown at 37° C. in LB/ampicillin/chloramphenicol medium until an OD₆₀₀ of 0.5 was reached, then were induced with 0.8 mM IPTG and continued to grow for 2-3 h at 37° C. The culture was centrifuged at 14,000 rpm, and the cell pellet was re-suspended in Buffer A (50 mM sodium acetate (pH 4.5) and 20 mM NaCl), supplemented with EDTA-free complete protease inhibitor cocktail (Roche), and lysed by sonication. The cell lysate was centrifuged at 14,000 rpm and the clarified lysate was passed through a 0.45 um filter. Clarified and filtered cell lysate was loaded on a cation exchange column (SP Sepharose fast flow resin, GE) and eluted with a linear NaCl gradient using Buffer B (50 mM sodium acetate (pH 4.5) and 1 M NaCl). Fractions containing HSP10 were concentrated, dialyzed overnight in 50 mM Tris-HCl (pH 7.4) and 150 mM NaCl using 10 kDa SnakeSkin™ dialysis tubing (Thermo Scientific), and re-purified on a Superdex 200 column (HiLoad 26/600, GE) with 50 mM Tris-HCl (pH 7.4) and 150 mM NaCl. Protein concentration was determined using a Coomassie Protein Assay Kit (Thermo Scientific). Protein was stored at 4° C. in 50 mM Tris-HCl (pH 7.4) and 150 mM NaCl. Batches of HSP10 for testing were stored at 4° C. for up to three weeks, then discarded.

E. coli GroEL, SR1, and GroES Purification.

E. coli GroEL and SR1 were expressed from a trc-promoted and Amp(+) resistance marker plasmid in NEB 5-alpha E. coli cells. GroES was expressed from a T7-promoted and Amp(+) resistance plasmid in E. coli BL21 (DE3) cells. Transformed colonies were plated onto Ampicillin-treated LB agar and incubated overnight at 37° C. A single colony was selected and grown in LB media treated with Ampicillin for 16 hours at 37° C. at 200 rpm. Cells were then sub-cultured in scaled-up Ampicillin-treated LB medium and grown at 37° C. at 230 rpm until reaching an OD₆₀₀ of 0.5, then were induced with 0.8 mM IPTG and continued to grow for ˜2.5 h at 37° C. The cultures were centrifuged at 8,000 rpm at 4° C. and the cell pellets were collected and re-suspended in Buffer A (50 mM Tris-HCl (pH 7.4) and 20 mM NaCl) supplemented with EDTA-free complete protease inhibitor cocktail (Roche). The suspension was lysed by sonication, the lysate was centrifuged at 14,000 rpm at 4° C., and the clarified lysate was passed through a 0.45 m filter (Millipore).

Anion exchange purification of GroEL and SR1: The filtered lysate was loaded onto a GE HiScale Anion exchange column (Q Sepharose fast flow anion exchange resin) that was equilibrated with 2 column volumes of Buffer A. The loaded column was washed with 3 column volumes of Buffer A containing 30% of Buffer B (50 mM Tris-HCl (pH 7.4) and 1 M NaCl), then bound protein was eluted with a 30-60% gradient elution of Buffer B over 3 column volumes. Protein-containing fractions, as identified by SDS-PAGE, were collected, spin-concentrated using a 10 kDa Amicon Ultra-15 centrifugal filter (EMD Millipore) and dialyzed overnight with 10 kDa SnakeSkin™ dialysis tubing (Thermo Scientific) at 4° C. in 50 mM Tris-HCl (pH 7.4) and 150 mM NaCl solution.

Size exclusion chromatography of GroEL: The dialyzed protein was loaded onto a Superdex 200 size exclusion column (HiLoad 26/600, GE) that was equilibrated with 2 column volumes of 50 mM Tris-HCl (pH 7.4) and 150 mM NaCl solution. The loaded column was eluted with 1 column volume of 50 mM Tris-HCl (pH 7.4) and 150 mM NaCl solution. Protein-containing fractions, as identified by SDS-PAGE, were collected and spin-concentrated using a 10 kDa Amicon Ultra-15 centrifugal filter (EMD Millipore). The final protein concentration was determined using a Coomassie Protein Assay Kit (Thermo Scientific). Batches of GroEL protein for testing were stored at 4° C. for up to one month, then discarded.

Size exclusion chromatography of SR1: The dialyzed protein was loaded onto a Superose 6 size exclusion column (HiLoad 16/600 prep grade, GE) that was equilibrated with 2 column volumes of 50 mM Tris-HCl (pH 7.4) and 150 mM NaCl solution. The loaded column was eluted with 1 column volume of 50 mM Tris-HCl (pH 7.4) and 150 mM NaCl solution. Protein-containing fractions, as identified by SDS-PAGE, were collected and spin-concentrated using a 10 kDa Amicon Ultra-15 centrifugal filter (EMD Millipore. Final protein concentration was determined using a Coomassie Protein Assay Kit (Thermo Scientific). Both SR1 and GroEL protein concentrations were calculated using the molecular weight for a tetradecameric complex (800 kDA) to ensure an equal concentration of subunits, thus IC₅₀ shifts are attributable to structural differences alone. Batches of SR1 protein for testing were stored at 4° C. for up to one month, then discarded.

Anion exchange purification of GroES: The filtered lysate was loaded onto a GE HiScale anion exchange column (Q Sepharose fast flow anion exchange resin) that was equilibrated with 2 column volumes of Buffer A. The loaded column was washed with 3 column volumes of Buffer A containing 10% of Buffer B (50 mM Tris-HCl (pH 7.4) and 1 M NaCl), then bound protein was eluted with a 10-50% gradient elution of Buffer B over 3 column volumes. Protein-containing fractions, as identified by SDS-PAGE, were collected, spin-concentrated using a 10 kDa Amicon Ultra-15 centrifugal filter (EMD Millipore), and dialyzed overnight with 10 kDa SnakeSkin™ dialysis tubing (Thermo Scientific) at 4° C. in 50 mM Tris-HCl (pH 7.4) and 150 mM NaCl solution.

Size exclusion chromatography of GroES: The dialyzed protein was loaded onto a Superdex 200 size exclusion column (HiLoad 26/600, GE) that was equilibrated with 2 column volumes of 50 mM Tris-HCl (pH 7.4) and 150 mM NaCl solution. The loaded column was eluted with 1 column volume of 50 mM Tris-HCl (pH 7.4) and 150 mM NaCl solution. Protein-containing fractions, as identified by SDS-PAGE, were collected and spin-concentrated using a 10 kDa Amicon Ultra-15 centrifugal filter (EMD Millipore). Final protein concentration was determined using a Coomassie Protein Assay Kit (Thermo Scientific). Batches of GroES protein for testing were stored at 4° C. for up to one month, then discarded.

Size Exclusion Chromatographic Analyses of Double and Single-Ring GroEL, BSA, cHSP60, and mHSP60 Proteins.

The proteins were loaded onto a Superose 6 column (10/300, GL) that was equilibrated with 1 column volume of 50 mM Tris-HCl, pH 7.4, and 150 mM NaCl solution. The loaded column was eluted with 1 column volumes of 50 mM Tris-HCl, pH 7.4, and 150 mM NaCl solution. Protein-containing fractions, as identified by SDS-PAGE, were collected to confirm the protein contained in those fractions was the correct protein of interest.

Evaluating Compounds for Inhibition in the mHSP60/10-Mediated dMDH Refolding Assay.

Reagent preparation: For this assay, four primary reagent stocks were prepared: 1) mHSP60/10-dMDH binary complex stock; 2) ATP initiation stock; 3) EDTA quench stock; 4) MDH enzymatic assay stock. Denatured MDH (dMDH) was prepared by 2-fold dilution of MDH (5 mg/ml, porcine heart MDH in glycerol solution from Sigma-Aldrich, product #M2634-5KU) with denaturant buffer (7 M guanidine-HCl, 200 mM Tris, pH 7.4, and 50 mM DTT). MDH was completely denatured by incubating at room temperature for 1 hour. The binary complex solutions were prepared by adding the dMDH stock to a stock with mHSP60 in folding buffer (50 mM Tris-HCl, pH 7.4, 50 mM KCl, 10 mM MgCl2, and 1 mM DTT), inverting several times and allowing to trap at room temperature for 10 min. Next, HSP10 was added to the binary complex stock, inverted, and then incubated at room temperature for 5 min. The binary complex stocks were prepared immediately prior to dispensing into 384-well polystyrene plates and had final protein concentrations of 416.5 nM mHSP60, 500 nM HSP10, and 20 nM dMDH in folding buffer. For the ATP initiation stock, ATP solid was diluted into folding buffer to a final concentration of 2.5 mM. Quench solution contained 600 mM EDTA (pH 8.0). The MDH enzymatic assay stock consisted of 20 mM sodium mesoxalate and 2.4 mM NADH in reaction buffer (50 mM Tris-HCl, pH 7.4, 50 mM KCl, and 1 mM DTT).

Assay protocol: First, 30 μL aliquots of the mHSP60/10-dMDH binary complex stocks were dispensed into clear, 384-well polystyrene plates. Next, 0.5 μL of the compound stocks (10 mM to 4.6 μM, 3-fold dilutions series in DMSO) were added by pin-transfer (V&P Scientific). The chaperonin-mediated refolding cycles were initiated by addition of 20 μL of ATP stock (reagent concentrations during refolding cycle: 250 nM mHSP60, 260 nM HSP10, 12 nM dMDH, 1 mM ATP, and compounds of 100 μM to 46 nM, 3-fold dilution series). The refolding reactions were incubated at 37° C. The incubation time was determined from refolding time-course control experiments until they reached ˜80% completion of refolding cycle—generally ˜30-40 min for mHSP60/10). Next, the assay was quenched by addition of 10 μL of the EDTA to final concentration of 100 mM. Enzymatic activity of the refolded MDH was initiated by addition of 20 μL MDH enzymatic assay stock (20 mM sodium mesoxalate and 2.4 mM NADH in reaction buffer, 50 mM Tris pH 7.4, 50 mM KCl, 1 mM DTT), and followed by measuring the NADH absorbance in each well at 340 nm using a Molecular Devices SpectraMax Plus384 microplate reader (NADH absorbs at 340 nm, while NAD+ does not). A340 nm measurements were recorded at 0.5 minutes (start point) and at successive time points until the amount of NADH consumed reached ˜80% (end point, generally between 20-35 minutes). The differences between the start and end point A₃₄₀ values were used to calculate the % inhibition of the mHSP60/10 machinery by the compounds. IC₅₀ values for the test compounds were obtained by plotting the % inhibition results in GraphPad Prism and analyzing by non-linear regression using the log (inhibitor) vs. response (variable slope) equation. Results presented represent the averages of IC₅₀ values obtained from at least four replicates.

Evaluating the ATPase Activity of cHSP60 and mHSP60.

Reagent preparation: For this assay, five primary reagent stocks were prepared: 1-2) cHSP60 and mHSP60 protein stocks, with and without HSP10; 3) ATP solution stock; 4) EDTA quench stock; 5) Malachite green stock. The protein stocks were prepared by adding either cHSP60 or mHSP60 in folding buffer (50 mM Tris-HCl, pH 7.4, 50 mM KCl, 10 mM MgCl₂, and 1 mM DTT), followed by addition of HSP10, if specified. The stocks were prepared immediately prior to dispensing into the assay plates and had final protein concentrations of 500 nM c/mHSP60 and 1500 nM HSP10 in folding buffer. For the ATP initiation stock, ATP solid was diluted into folding buffer to a final concentration of 2 mM. The quench solution contained 300 mM EDTA (pH 8.0). The Malachite green stock consisted of 0.034% malachite green and 1.04% ammonium molybdate tetrahydrate in 1 M HCl with 0.02% Tween-20.

Assay protocol: The assays were performed in time course, allowing times of 60, 50, 30, 20, 12, 6, 3, and 0 min for ATP hydrolysis to occur. First, 10 μL aliquots of the HSP60 (with or without HSP10) protein stocks were dispensed into clear, 384-well polystyrene plates. Chaperonin-mediated ATPase cycling was initiated by addition of 10 μL of ATP stock at the respective time-points, then the samples were incubated at 37° C. At completion of the last time point, the wells were quenched simultaneously by addition of 10 μL of the EDTA solution to final concentration of 100 mM, with the EDTA solution being added prior to ATP at time 0 min. After quenching, 60 μL of the malachite green assay stock was added, followed by incubation at room temperature for 10 min. Next, the absorbance in each well was measured at 600 nm using a Molecular Devices SpectraMax Plus384 microplate reader. Following the same protocol as for the assays (without protein or ATP in the samples), standard curves (0-50 μM sodium phosphate) were created to determine the total phosphate in each well, corresponding to the amount of ATP hydrolyzed.

Evaluating the Ability of cHSP60 and mHSP60 to Trap and Refold Denature Malate Dehydrogenase (dMDH) Client Protein.

Reagent preparation: For these assays, five primary reagent stocks were prepared: 1) separate cHSP60-dMDH and mHSP60-dMDH protein stocks; 2) separate cHSP60-dMDH and mHSP60-dMDH protein stocks with HSP10; 3) ATP initiation stock; 4) EDTA quench stock; 5) MDH enzymatic assay stock. Denatured MDH (dMDH) was prepared by 2-fold dilution of MDH (5 mg/ml, porcine heart MDH in glycerol solution from Sigma-Aldrich, product #M2634) with denaturant buffer (7 M guanidine-HCl, 200 mM Tris, pH 7.4, and 50 mM DTT). MDH was completely denatured by incubating at room temperature for 1 hour. The protein stocks were prepared by adding the dMDH stock to cHSP60 or mHSP60 in folding buffer (50 mM Tris-HCl, pH 7.4, 50 mM KCl, 10 mM MgCl₂, and 1 mM DTT) while mixing to allow the dMDH to bind to the chaperonin rings, and the stocks were incubated at room temp for 10 min. Next, HSP10 was added and the stocks were mixed and incubated at room temp for an additional 5 min. The protein stocks were prepared immediately prior to dispensing into the assay plates and had final protein concentrations of 416.5 nM c/mHSP60, 500 nM HSP10, and 20 nM dMDH in folding buffer. For the ATP initiation stock, ATP solid was diluted into folding buffer to a final concentration of 2.5 mM. The quench solution contained 600 mM EDTA (pH 8.0). The MDH enzymatic assay stock consisted of 20 mM sodium mesoxalate and 1.6 mM NADH in reaction buffer (50 mM Tris-HCl, pH 7.4, 50 mM KCl, and 1 mM DTT).

Assay protocol: The assays were performed in time course, allowing times of 60, 50, 30, 20, 12, 6, 3, and 0 min for refolding of MDH to occur. First, 30 μL of the four protein stocks were dispensed into clear, 384-well polystyrene plates. The chaperonin-mediated refolding cycles were initiated by addition of 20 μL of ATP stock at the respective time points in the time course, and the samples were incubated at 37° C. At the final time point, the wells were quenched simultaneously by addition of 10 μL of the EDTA to final concentration of 100 mM, with EDTA having been added prior to ATP for the 0 min time point. Enzymatic activity of the refolded MDH was initiated by addition of 20 μL MDH enzymatic assay stock (20 mM sodium mesoxalate and 1.6 mM NADH in reaction buffer, 50 mM Tris pH 7.4, 50 mM KCl, 1 mM DTT), and followed by measuring the NADH absorbance in each well at 340 nm using a Molecular Devices SpectraMax Plus384 microplate reader (NADH absorbs at 340 nm, while NAD+ does not), using the kinetic time point analysis option with a fixed temperature of 37° C. to avoid temperature fluctuations between readings. Readings were done every 5 min over the course of 60 min to follow NADH consumption over time. Results were then analyzed at the point where 80% NADH consumption had occurred in the mHSP60/10-dMDH sample, which was normalized as the 100% refolding activity, to which the other protein samples were compared.

### Example 1: Chaperonin Inhibitor Diversity Subset

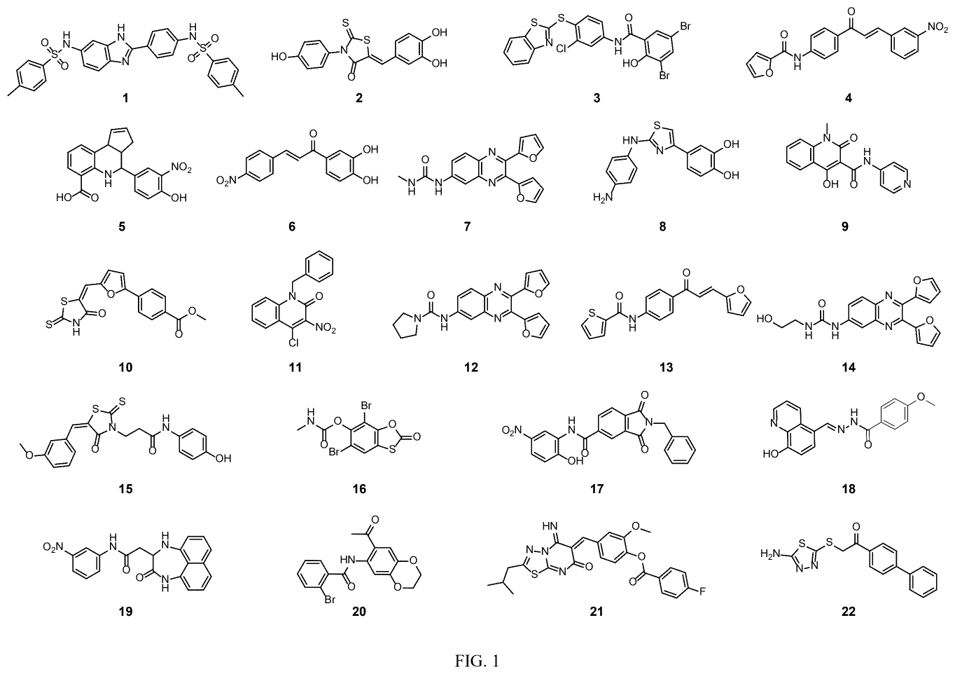

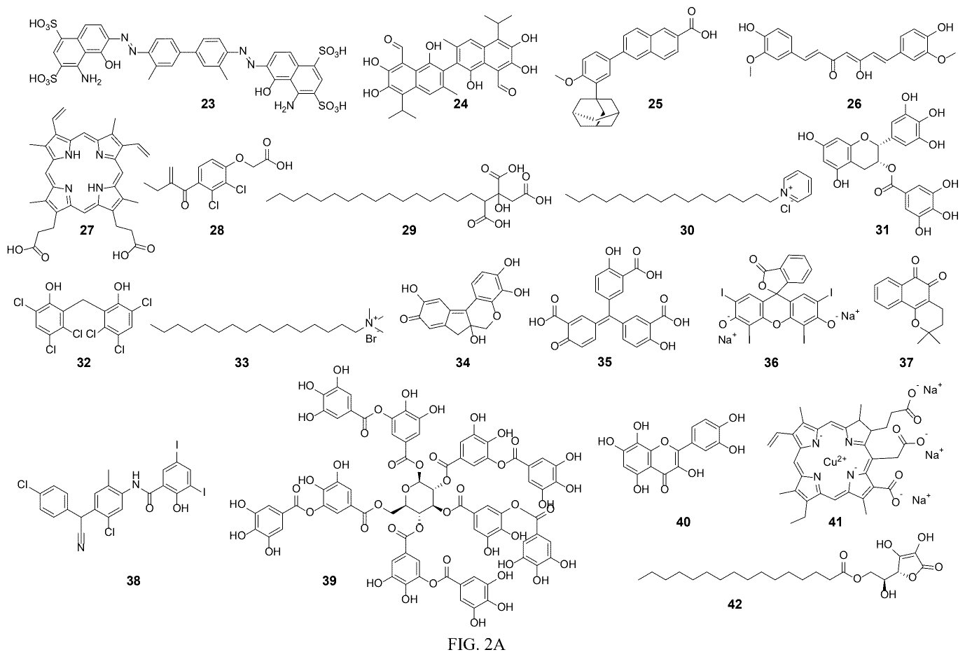

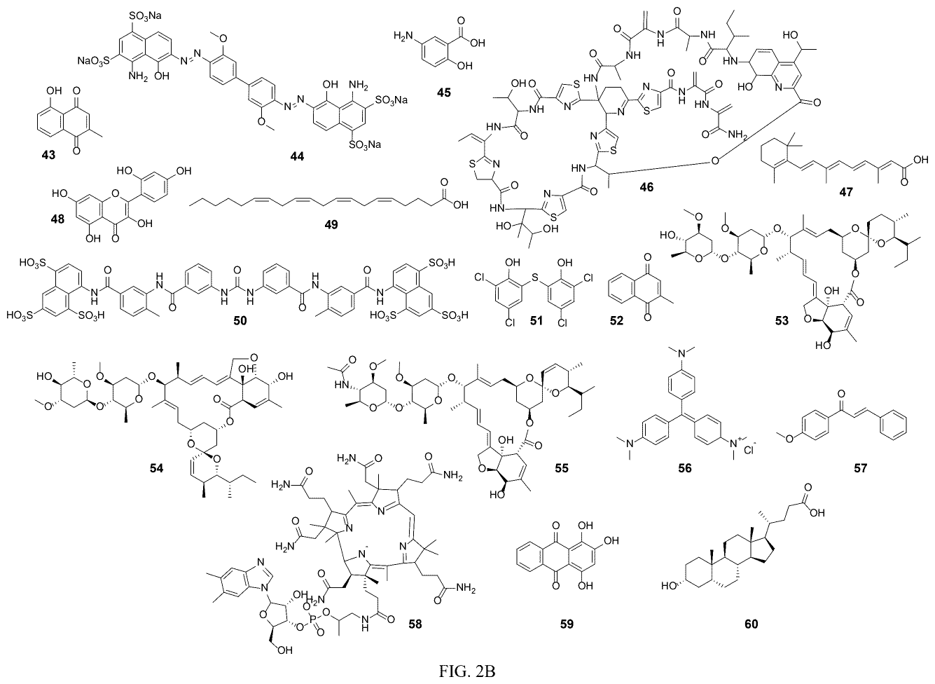

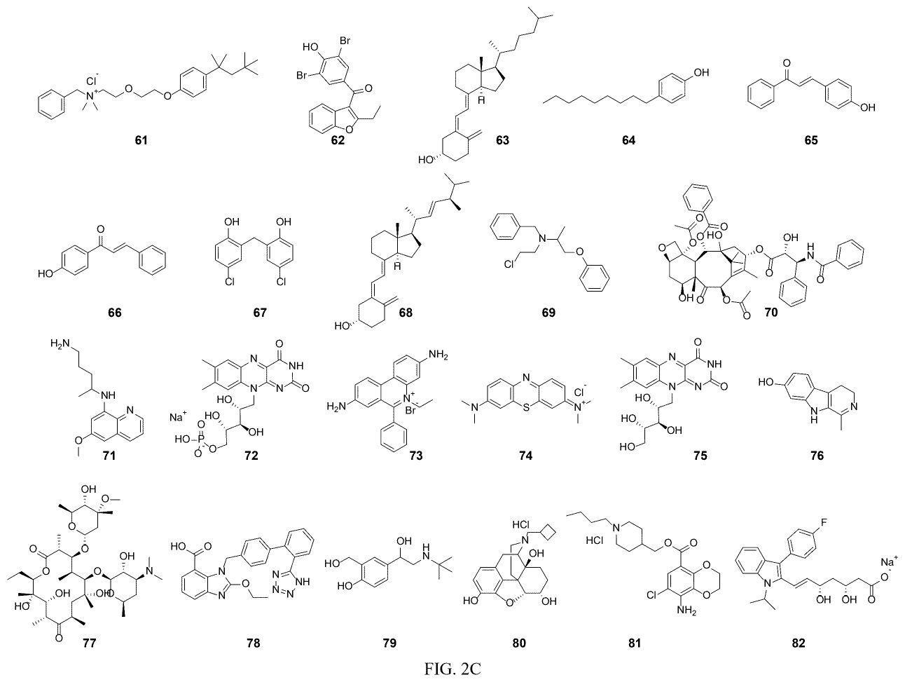

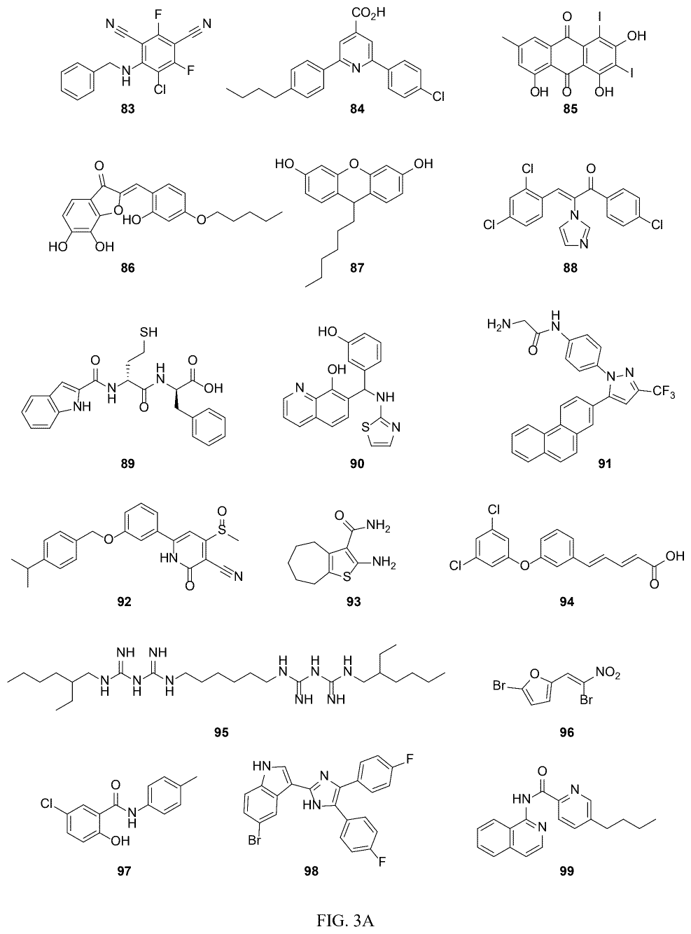

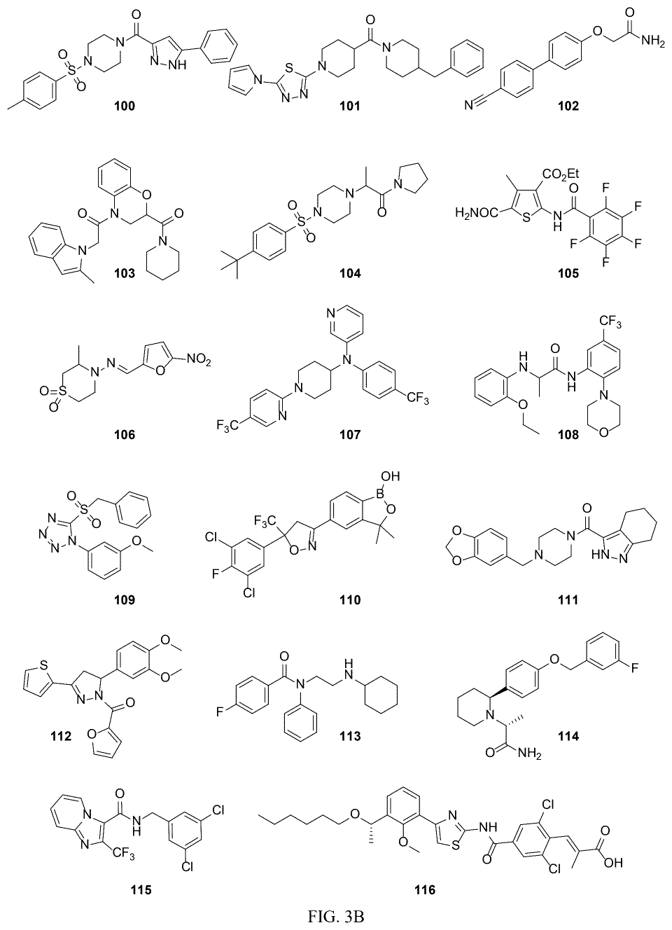

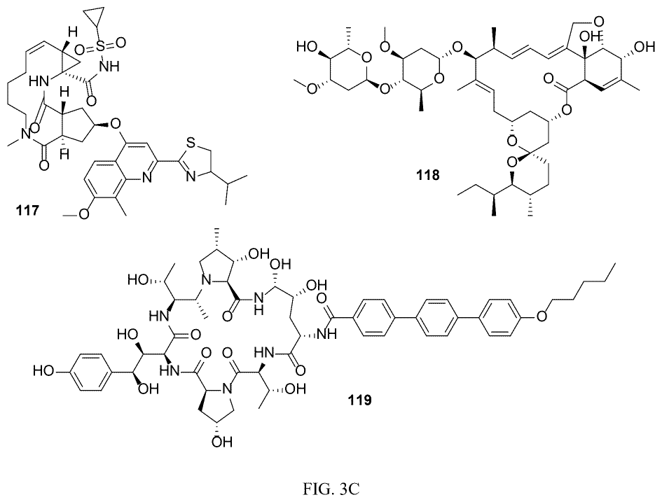

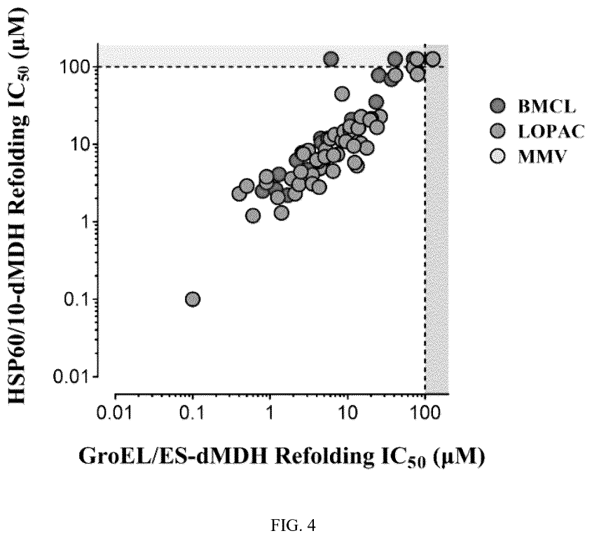

In an effort to identify new classes of chaperonin inhibitors, three high-throughput screens that employed E. coli GroEL were performed, with counter-screening conducted to identify hits that could also target mHSP60. In a first study, screening of a library of ˜700,000 small molecules identified 235 GroEL inhibitors—termed the “BMCL inhibitor set” (FIG. 1). A subsequent study involved screening of a library of 3,680 approved drugs, natural products, and known bioactive molecules known for their diverse biological effects, which identified 161 GroEL inhibitors—termed the “LOPAC inhibitor set” (FIGS. 2A-2C). Most recently, a library of 960 compounds from the Medicines for Malaria Venture (MMV) identified an additional 25 GroEL inhibitors-termed the “MMV inhibitor set” (FIGS. 3A-3C). Subsequent counter-screening of many of these identified inhibitors demonstrated that nearly all of them could also inhibit mHSP60 in vitro (FIG. 4); thus, these candidates were selected to explore their ability to selectively target cancer over non-cancer cells.

An initial investigation into targeting HSP60 as a chemotherapeutic strategy centered on two inhibitor series based on the bis-sulfonamido-2-phenylbenzoxazole (PBZ) and salicylanilide (SCA) scaffolds (Table 1). Furthermore, from the high-throughput screens, it was noted that some of the most potent inhibitors of E. coli GroEL and human mHSP60 contained an α,β-unsaturated ketone (ABK) linking adjacent aromatic rings—a primary example of which are chalcones (Table 1). Considering naturally-derived chalcones are reported to exhibit a wide range of bioactivities including anti-allergy, -ulcer, -tumor, -bacterial, -parasitic, and -inflammatory effects, a small panel of ABK analogs were examined for their ability to inhibit mHSP60 and selectively target colorectal cancer cells in vitro. Notably, it was found that lead analogs across each of these series induced the selective apoptosis of colorectal cancer over non-cancer cells and inhibit cancer cell migration and clonogenicity in vitro, with lead ABK analogs exhibiting the greatest potency and selectivity. To better understand HSP60 targeting, fractionation of colorectal cancer and non-cancer cell lines was performed to investigate correlations between inhibition of cell viability versus relative HSP60 levels in the mitochondria and cytosol. Without being bound by any theory, the findings suggested that the ABK series may target aberrant naïve cHSP60 in the cytosol, the PBZ series may target mitochondrial mHSP60, and that the SCA series may target both.

While the previous findings underscore the potential of a chemotherapeutic approach targeting HSP60, concerns are noted from the initial studies on the PBZ, SCA, and ABK inhibitor series. It is widely acknowledged, for instance, that α,β-unsaturated ketone linkages can potentially covalently modify proteins, such as through Michael additions. Without being bound by any theory, this capability could result in target promiscuity; hence, developing such an inhibitor class must be approached cautiously. In addition, the PBZ and SCA series were initially designed to study their antibiotic effects against bacteria and parasites, meaning there was no presupposed rationale as to why these analogs would be the most suitable candidates for chemotherapeutic effects.

TABLE 1
Representative compounds from SCA, PBZ, and ABK Scaffold Series.

| Series | Structure |
|---|---|
| SCA | 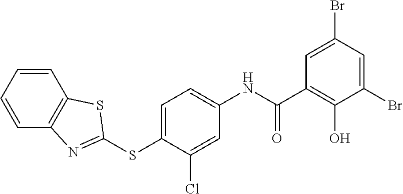 [SMILES: O=C(Nc1ccc(Sc2nc3ccccc3s2)c(Cl)c1)c1cc(Br)cc(Br)c1O] |
| PBZ | 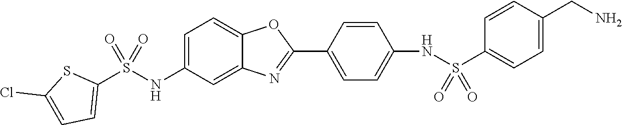 [SMILES: NCc1ccc(S(=O)(=O)Nc2ccc(-c3nc4cc(NS(=O)(=O)c5ccc(Cl)s5)ccc4o3)cc2)cc1] |
| ABK | 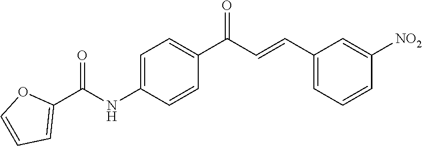 [SMILES: O=C(/C=C/c1cccc([N+](=O)[O-])c1)c1ccc(NC(=O)c2ccco2)cc1] |

In light of these considerations, the present study focused on screening a subset of 119 compounds from our chaperonin inhibitor diversity set to initially identify the most potent and selective inhibitors against colorectal cancer over non-cancerous cells. From this screen, several lead analogs were identified surpassing the previous PBZ, SCA, and ABK lead analogs. These included two parallel series of hydroxyquinoline and nitrofuran-based N-acylhydrazones (HQ-NAH and NF-NAH series, respectively), which had also been previously investigated as anti-bacterial and anti-parasitic agents. Following this, a small panel of lead candidates were submitted to the National Cancer Institute (NCI) for cytotoxicity testing against their panel of cancer cell lines of various origins. The objective was to examine for potential broad-spectrum effects across tumors from different organs. Lastly, the in vitro structure, function, and inhibition of naïve cHSP60 that is putatively in the cytosol of cancer cells was investigated, examining for similarities/differences with mHSP60 that could help enhance the mechanistic understanding of an HSP60-targeting chemotherapeutic strategy.

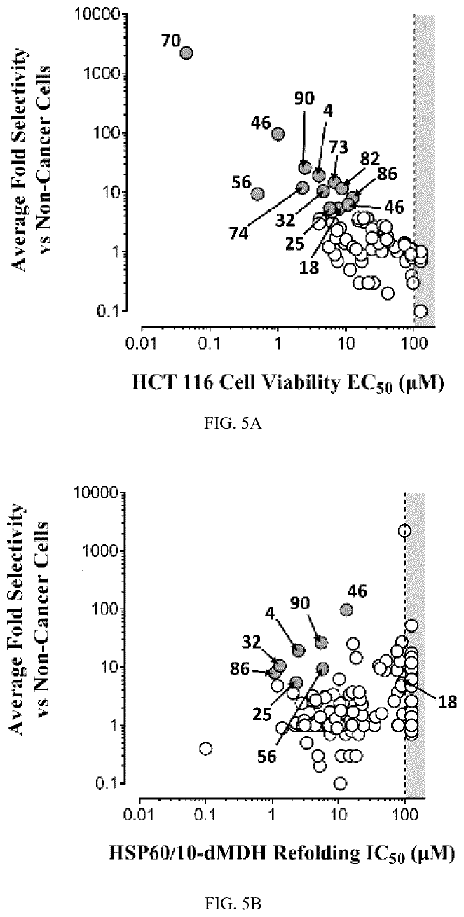

In an effort to identify the most potent and selective compounds suitable for anticancer therapeutic development, the 119 member chaperonin inhibitor diversity subset was evaluated for their cytotoxicity against a small panel of cancerous and noncancerous cells in liquid culture, utilizing an established AlamarBlue-based cell viability assays. To start, both cancerous and non-cancerous cells were grown in flasks to ˜80% confluency, then dispensed into 384-well microplates and incubated for 24 h at 37° C. and 5% CO₂. Next, inhibitors were added and the plates were incubated for a further 48 h, after which the AlamarBlue reagents were added to the wells. Absorbance readings were then measured over time to determine the amount of resazurin reduced by the live cells, with EC₅₀ and CC₅₀ values obtained from 8-point, 3-fold dose-response curves. The compounds were initially screened against HCT-116 colon cancer cells, followed by counter-screening against non-cancerous FHC colon and FHs-741nt intestine cells. These analyses determined inhibitor potencies against the HCT-116 colon cancer cells (EC₅₀ values) and selectivity indices (SI) compared to cytotoxicity (CC₅₀ values) against the non-cancer cell lines: results are plotted in FIG. 5A with data tabulated in Tables 2, 3, and 4.

TABLE 2
BMCL 700k screening inhibitor cell viability results.

| | Human Cell Viability EC₅₀ or CC₅₀ (μM) | | | | | |
|---|---|---|---|---|---|---|
| | | | | | Intestine | Average |
| | Colon Cancer | | | Colon | (FHs | Selectivity |
| # | (HCT 116) | (DLD1) | (HT-29) | (FHC) | 74Int) | Index |
| 1 | 20.5 | 24.9 | 21.3 | >100 | 53.5 | 3.7 |
| 2 | 18.3 | | | 70.3 | 47.6 | 3.2 |
| 3 | 25.9 | 46.5 | 37.3 | 56.0 | 29.5 | 1.7 |
| 4 | 4.0 | 39.7 | 3.6 | >100 | 54.0 | 19.1 |
| 5 | >100 | | | >100 | >100 | 1.0 |
| 6 | 6.3 | 9.6 | 8.2 | 28.3 | 11.4 | 3.1 |
| 7 | 94.7 | | | >100 | >100 | 1.1 |
| 8 | >100 | | | >100 | >100 | 1.0 |
| 9 | >100 | | | >100 | >100 | 1.0 |
| 10 | 28.6 | | | >100 | 42.2 | 2.5 |
| 11 | 8.2 | | | 31.5 | 9.4 | 2.5 |
| 12 | 98.2 | | | >100 | >100 | 1.0 |
| 13 | 35.1 | 60.0 | 53.9 | 88.5 | 78.8 | 2.4 |
| 14 | >100 | | | >100 | >100 | 1.0 |
| 15 | >100 | | | >100 | >100 | 1.0 |
| 16 | >100 | | | >100 | >100 | 1.0 |
| 17 | >100 | | | >100 | >100 | 1.0 |
| 18 | 7.8 | 3.8 | 3.5 | 28.4 | 54.9 | 5.3 |
| 19 | 26.8 | | | >100 | 37.4 | 2.6 |
| 20 | >100 | | | >100 | >100 | 1.0 |
| 21 | 92.1 | | | >100 | >100 | 1.1 |
| 22 | >100 | | | >100 | >100 | 1.0 |

TABLE 3
LOPAC & MicroSource Spectrum screening
inhibitor cell viability results.

| | Human Cell Viability EC₅₀ or CC₅₀ (μM) | | | | | |
|---|---|---|---|---|---|---|
| | | | | | Intestine | Average |
| | Colon Cancer | | | Colon | (FHs | Selectivity |
| # | (HCT 116) | (DLD1) | (HT-29) | (FHC) | 74Int) | Index |
| 23 | 92.6 | | | 32.5 | 45.1 | 0.4 |
| 24 | 6.2 | | | 48.7 | 10.6 | 4.8 |
| 25 | 5.8 | 7.9 | 5.3 | 43.7 | 18.5 | 5.4 |
| 26 | 15.4 | | | 71.0 | 35.4 | 3.4 |
| 27 | 96.6 | | | 17.7 | 42.9 | 0.3 |
| 28 | >100 | | | >100 | >100 | 1.0 |
| 29 | >100 | | | >100 | 97.4 | 1.0 |
| 30 | 5.6 | | | 8.5 | 5.1 | 1.2 |
| 31 | 89.3 | | | >100 | >100 | 1.1 |
| 32 | 4.6 | | | 56.6 | 40.2 | 10.5 |
| 33 | 7.1 | | | 17.0 | 7.5 | 1.7 |
| 34 | >100 | | | >100 | >100 | 1.0 |
| 35 | >100 | | | >100 | >100 | 1.0 |
| 36 | >100 | | | 94.8 | >100 | 1.0 |
| 37 | 4.1 | | | 16.4 | 13.0 | 3.6 |
| 38 | 51.5 | | | 78.7 | 47.6 | 1.2 |
| 39 | >100 | | | >100 | >100 | 1.0 |
| 40 | >100 | | | >100 | >100 | 1.0 |
| 41 | >100 | | | >100 | >100 | 1.0 |
| 42 | >100 | | | >100 | >100 | 1.0 |
| 43 | 4.0 | | | 16.8 | 7.8 | 3.0 |
| 44 | >100 | | | >100 | >100 | 1.0 |
| 45 | >100 | | | >100 | >100 | 1.0 |
| 46 | 1.0 | 16^{BP} | 1.2 | >100 | 93.8 | 96.5 |
| 47 | 71.8 | | | >100 | >100 | 1.4 |
| 48 | >100 | | | >100 | >100 | 1.0 |
| 49 | 37.5 | | | >100 | >100 | 2.7 |
| 50 | >100 | | | >100 | >100 | 1.0 |
| 51 | 24.2 | | | 80.1 | 36.2 | 2.4 |
| 52 | 15.1 | | | 56.2 | 46.0 | 3.4 |
| 53 | 10.0 | | | 20.2 | 12.2 | 1.6 |
| 54 | 12.8 | | | 16.4 | 14.1 | 1.2 |
| 55 | 14.1 | | | 16.2 | 15.1 | 1.1 |
| 56 | 0.5 | 0.2 | 0.1 | 8.6 | 1.6 | 9.4 |
| 57 | 15.4 | 35^{BP} | 23.2 | 67.8 | 45.3 | 3.7 |
| 58 | >100 | | | >100 | >100 | 1.0 |
| 59 | 41.6 | | | 68.4 | 57.3 | 1.5 |
| 60 | >100 | | | >100 | >100 | 1.0 |
| 61 | 7.4 | | | 19.4 | 14.8 | 2.3 |
| 62 | 72.6 | | | >100 | 90.8 | 1.3 |
| 63 | 40.4 | | | >100 | 48.9 | 1.8 |
| 64 | 90.4 | | | 82.2 | 88.5 | 0.9 |
| 65 | 35.2 | 50.5 | 39.3 | >100 | 98.1 | 2.8 |
| 66 | 18.2 | 33.6 | 29.8 | 76.8 | 59.3 | 3.7 |
| 67 | 24.2 | | | 47.2 | 35.0 | 1.7 |
| 68 | 39.3 | | | 77.5 | 49.6 | 1.6 |
| 69 | >100 | | | >100 | >100 | 1.0 |
| 70 | <0.045 | | | >100 | >100 | 2222.2 |
| 71 | 10.8 | | | 77.4 | 56.6 | 6.2 |
| 72 | >100 | | | >100 | >100 | 1.0 |
| 73 | 6.8 | | | >100 | >100 | 14.7 |
| 74 | 2.3 | | | 31.3 | 23.4 | 11.9 |
| 75 | 38.67 | | | >100 | >100 | 2.6 |
| 76 | >100 | | | >100 | >100 | 1.0 |
| 77 | >100 | | | >100 | >100 | 1.0 |
| 78 | >100 | | | >100 | >100 | 1.0 |
| 79 | >100 | | | >100 | >100 | 1.0 |
| 80 | >100 | | | >100 | >100 | 1.0 |
| 81 | >100 | | | >100 | >100 | 1.0 |
| 82 | 8.7 | | | >100 | >100 | 11.6 |

^{BP}indicates that the baselines of the dose-response curves did not reach 0% activity at the highest compound concentrations but plateaued between ~10-20%.

TABLE 4
MMV screening set inhibitor cell viability results.

| | | Human Cell Viability EC₅₀ or CC₅₀ (μM) | | | | | |
|---|---|---|---|---|---|---|---|
| | | | | | | Intestine | Average |
| | | Colon Cancer | | | Colon | (FHs | Selectivity |
| | # | (HCT 116) | (DLD1) | (HT-29) | (FHC) | 74Int) | Index |
| MMV | 83 | 41.0 | | | 11.5 | 7.6 | 0.2 |
| Pandemic | 84 | 75.9 | | | 64.4 | 40.0 | 0.7 |
| Response | 85 | 31.4 | | | 66.1 | 16.9 | 1.3 |
| | 86 | 12.5 | 23^{BP} | 5.9^{BP} | >100 | >100 | 8.0 |
| | 87 | 56.5 | | | 56.7 | 51.7 | 1.0 |
| | 88 | 11.5 | | | 4.6 | 5.8 | 0.5 |
| | 89 | >100 | | | >100 | >100 | 1.0 |
| | 90 | 2.5 | 15^{BP} | 8.3 | 66.6 | 64.6 | 26.2 |
| | 91 | 15.8 | | | 4.9 | 4.5 | 0.3 |
| | 92 | 7.5 | | | 5.3 | 5.1 | 0.7 |
| | 93 | >100 | | | >100 | >100 | 1.0 |
| | 94 | >100 | | | 6.7 | 5.3 | 0.1 |
| | 95 | 94.2 | | | 97.6 | 60.0 | 0.8 |
| | 96 | 25.7 | | | 7.8 | 6.4 | 0.3 |
| | 97 | 9.5 | | | 17.3 | 8.5 | 1.4 |
| | 98 | 17.1 | | | 17.4 | 7.7 | 0.7 |
| MMV | 99 | >100 | | | 70.2 | 73.6 | 0.7 |
| Pathogens | 100 | >100 | | | >100 | >100 | 1.0 |
| | 101 | >100 | | | >100 | >100 | 1.0 |
| | 102 | >100 | | | >100 | >100 | 1.0 |
| | 103 | >100 | | | >100 | 64.2 | 1.0 |
| | 104 | >100 | | | >100 | >100 | 1.0 |
| | 105 | 6.8 | | | 8.4 | 3.9 | 0.9 |
| | 106 | >100 | | | >100 | >100 | 1.0 |
| | 107 | >100 | | | >100 | 93.4 | 1.0 |
| | 108 | >100 | | | >100 | 94.9 | 1.0 |
| | 109 | 21.2 | | | 6.1 | 5.7 | 0.3 |
| | 110 | 24.1 | | | 30.5 | 22.1 | 1.1 |
| | 111 | 73.4 | | | >100 | >100 | 1.4 |
| | 112 | >100 | | | >100 | >100 | 1.0 |
| | 113 | 93.9 | | | >100 | 52.2 | 0.8 |
| | 114 | >100 | | | >100 | 68.7 | 0.8 |
| | 115 | >100 | | | >100 | >100 | 1.0 |
| MMV | 116 | 33.5 | | | 47.8 | 20.3 | 1.0 |
| Covid | 117 | 16.8 | | | 21.7 | 9.8 | 0.9 |
| | 118 | 10.0 | | | 20.2 | 12.2 | 1.6 |
| | 119 | 30.4 | | | 59.0 | 24.6 | 1.4 |

^{BP}indicates that the baselines of the dose-response curves did not reach 0% activity at the highest compound concentrations but plateaued between ~10-20%.

Examining the results revealed that several inhibitors exhibited notable potency and selectivity for the colorectal cancer cells over the non-cancer cells (FIG. 5A). These inhibitors were compounds 4, 18, 25, 32, 46, 56, 70, 71, 73, 74, 82, 86, and 90, each exhibiting a greater than 5-fold selectivity index (SI). Structures of these can be found in FIGS. 1, 2A-2C, and 3A-3C. Compounds 4 (EC₅₀=4 μM) and 18 (EC₅₀=7.8 μM) from the BMCL set exhibit SIs of 5.3-19-fold for colorectal cancer cells. Compounds 25, 32, 46, 70, 71, 73, 74, and 82 are from the LOPAC set, which means that each of these compounds were already known and had established bioactivities. Compound 25 (Adapalene) is a widely used topical retinoid for acne vulgaris treatment and 32 (Hexachlorophene) is an antibacterial cleanser that is mainly used to clean the skin before surgery. Both demonstrated moderate potency and selectivity for colorectal cancer cells over non-cancer cells, with EC₅₀ values of 5.8 μM and 4.6 μM, respectively, SIs ranging from 5.4-fold to 11-fold. Compound 46 (Thiostrepton), a well-known natural antibiotic approved by the FDA for animal use as a translational blocker, exhibited exceptional potency against colorectal cancer cells (EC₅₀=1.0 μM), with a 96-fold selectivity over non-cancer cells. Compound 56 (Crystal violet), a common cellular DNA stain [48], displayed potent inhibition of colorectal cancer cells (EC₅₀=0.54 μM) with an SI of 9.4. Compound 70, Paclitaxel, a known chemotherapeutic agent used for several cancer types, demonstrated high potency and selectivity (EC₅₀<0.045 μM; >2222-fold SI). Compounds 71 (Primaquine; used to treat malaria), 73 (Ethidium bromide; used for DNA staining), 74 (Methylene blue trihydrate; used as a bacteriologic stain and to treat cyanide poisoning), and 82 (Fluvastatin sodium; used to treat high cholesterol), exhibited moderate potency and selectivity, with EC₅₀ values ranging from 2.3-11 μM, and SIs ranging from 6.2-15. Compounds 86 and 90 are both a part of the Medicines of Malaria Venture (MMV) set. Compound 86 bears a cyclical α,β-unsaturated ketone, the core scaffold of our ABK series, so it is perhaps not surprising to see this as a lead hit. Compound 90 is among the most potent and selective compounds (EC₅₀=2.5 μM) and has a similar structure to another inhibitor series reported for antibacterial applications, the N-acylhydrazones (NAH).

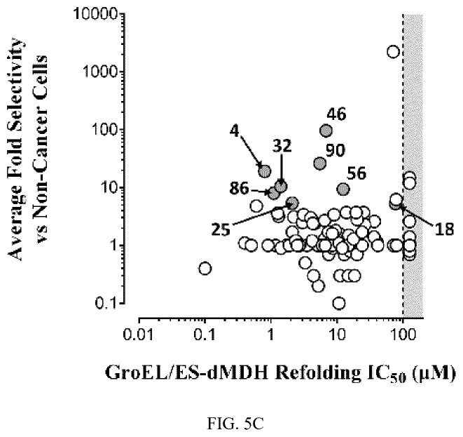

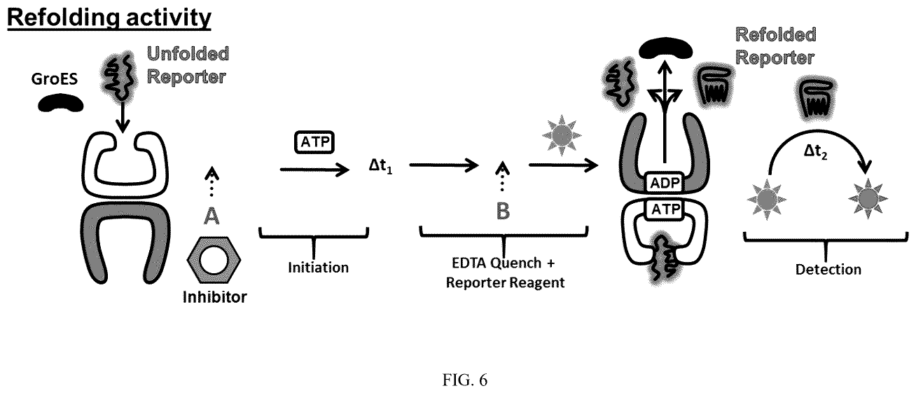

As these compounds were previously reported for their ability to inhibit the client protein folding capabilities of human mHSP60 and E. coli GroEL, the results for which the lead hits were also chaperonin inhibitors were examined to identify those with the highest potential to act on-target. The inhibition results are presented in FIGS. 5B and 5C for biochemical assays where the denatured malate dehydrogenase (dMDH) reporter enzyme is refolded by either mHSP60 or GroEL (FIG. 6). The enzymatic activity of the refolded MDH served as an indicator of chaperonin refolding abilities; thus, without being bound by any theory, a lack of activity may signify either inhibition of chaperonin-mediated folding functions (positive hit) or inhibition of native MDH enzymatic activity (false positive). To distinguish between the two, counter-screening against refolded MDH was performed to identify false positives that may merely inhibit the MDH enzymatic reporter reaction. In these assays, binary complexes of dMDH bound to either GroEL or mHSP60 were prepared. GroES or HSP10 co-chaperones were then added and the solutions were dispensed into 384-well microplates. Following the addition of inhibitors, ATP was added to the plates to initiate refolding of dMDH. The plates were incubated at 37° C. until ˜90% of the dMDH was refolded, at which point the reactions were quenched with EDTA. An enzyme assay solution containing NADH and ketomalonic acid was then added, with successful MDH refolding indicated by the conversion of NADH to NAD+. Of the thirteen lead hits, compounds 4, 18, 25, 32, 46, 56, 86, and 90 inhibited both mHSP60 and GroEL, exhibiting IC₅₀ values ranging from 0.8-9 μM (FIGS. 5B, 5C and Table 5). Conversely, the remaining five hits were inactive and thus false-positives identified in the initial high-throughput screens. Two of the most potent compounds were analogs 4 and 86. Compound 90 being a potent HSP60 inhibitor was an intriguing finding as it is structurally similar to a related series of antibacterial candidates bearing a similar hydroxyquinoline group, as well as a parallel series of nitrofuran-containing analogs similar to known antibacterials nifuroxazide and nitrofurantoin. Because these hydroxyquinoline and nitrofuran-based N-acylhydrazone analogs (HQ-NAH and NF-NAH series) were previously developed, they were selected to be examined in greater detail for chemotherapeutic potential.

TABLE 5
Structures and names of the most selective and potent molecules across the diversity
screen.

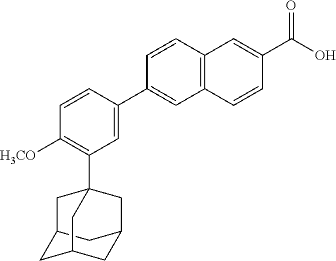 [SMILES: COc1ccc(-c2ccc3cc(C(=O)O)ccc3c2)cc1C12CC3CC(CC(C3)C1)C2]

25 - Adapalene

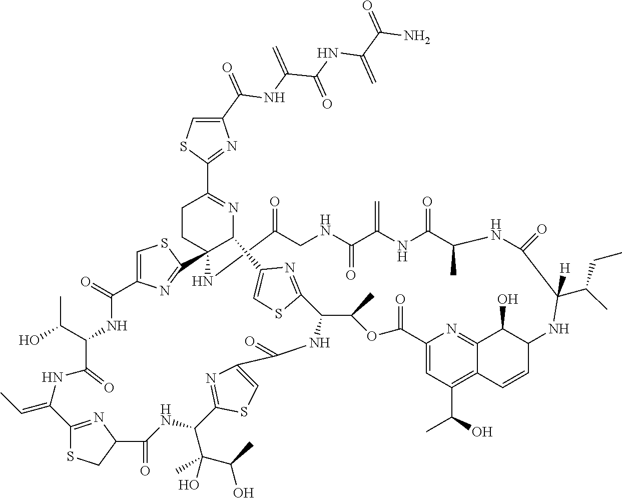 [SMILES: C=C(NC(=O)C(=C)NC(=O)c1csc(C2=N[C@@H]3c4csc(n4)[C@H]4NC(=O)c5csc(n5)[C@H]([C@](C)(O)[C@@H](C)O)NC(=O)C5CSC(=N5)/C(=C/C)NC(=O)[C@H]([C@@H](C)O)NC(=O)c5csc(n5)[C@]3(CC2)NC(=O)CNC(=O)C(=C)NC(=O)[C@H](C)NC(=O)[C@H]([C@@H](C)CC)NC2C=Cc3c([C@H](C)O)cc(nc3[C@H]2O)C(=O)O[C@@H]4C)n1)C(N)=O]

46 - Thiostrepton

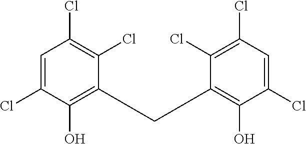 [SMILES: Oc1c(Cl)cc(Cl)c(Cl)c1Cc1c(O)c(Cl)cc(Cl)c1Cl]

32 - Hexachlorophene

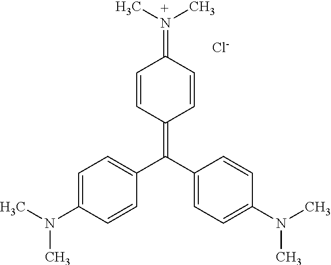

56 - Crystal Violet

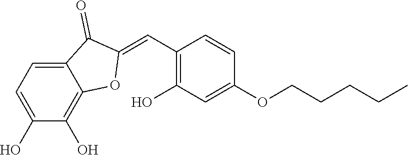 [SMILES: CCCCCOc1ccc(/C=C2\Oc3c(ccc(O)c3O)C2=O)c(O)c1]

Compound 86

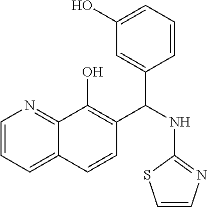 [SMILES: Oc1cccc(C(Nc2nccs2)c2ccc3cccnc3c2O)c1]

Compound 90

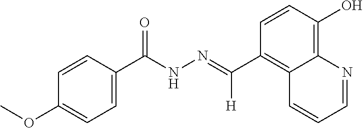 [SMILES: COc1ccc(C(=O)N/N=C/c2ccc(O)c3ncccc23)cc1]

Compound 18

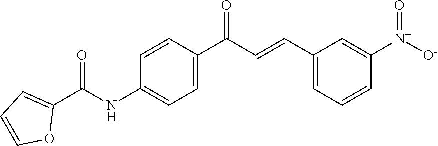 [SMILES: O=C(/C=C/c1cccc([N+](=O)[O-])c1)c1ccc(NC(=O)c2ccco2)cc1]

Compound 4

### Example 2: Hydroxyquinoline (HQ) and Nitrofuran-Based (NF) N-Acylhydrazone (NAH) Analogs

In a previous study, analogs of compound 18 were investigated due to structural similarities with established antibacterials, namely nifuroxazide and nitrofurantoin. Through this, SAR was established across two parallel inhibitor series: the hydroxyquinoline and the nitrofuran-based N-acylhydrazone series (HQ-NAH and NF-NAH, respectively). Each series was designed with a constant right-side substructure featuring either the HQ or NF moiety, while the left-side of the molecule was varied to facilitate the SAR analysis. Following the synthesis of these analogs, they were evaluated in a panel of assays to assess their ability to inhibit GroEL/ES folding functions, bacterial proliferation, and preliminary toxicity profiles in non-cancerous human cells. Results of that study indicated that the parent HQ-based analogs exhibited the highest potency in terms of inhibiting GroEL/ES refolding functions. However, it was evident that their NF-based counterparts were even more potent, with the caveat of operating through a pro-drug mechanism in the presence of E. coli NfsB nitroreductase. Interestingly, only the NF-based analogs demonstrated the capacity to selectively inhibit bacterial proliferation, potentially because of their pro-drug capabilities and ability to selectively inhibit bacterial proliferation with low toxicity to human cells, potentially owing to their reliance of NfsB activation, which human cells lack.

Through screening in the present study, these inhibitors exhibited substantial potency and selectivity for the colon cancer cells over the non-cancer cells, with most of these inhibitors having selectivity indices >5 (FIG. 7). The compounds displaying the highest selectivities were 124, 128, 131, 132, 135, and 147 (>15-fold SI) (Table 6 and 7). Furthermore, it was evident that the complete hydroxyquinoline and nitrofuran substructures were necessary for potent and selective activity, as modification of these substructures (e.g., removal of the nitro, hydroxyl, or altering of the quinoline ring) resulted in the inactivation of the compounds (see compounds 148-158 in Table 8). It is intriguing to note that while the nitrofuran analogs act as pro-drugs in the presence of E. coli NfsB, the mechanism activating these compounds in cancer cells is not clear as human cells do not have nitroreductases. Without being bound by any theory, activation of these compounds, however, may occur through aldehyde dehydrogenases. However, the potency and selectivity of the hydroxyquinoline analogs may indicate a different mechanism in human cells. Future studies will need to investigate the precise mechanisms of each of these series in colorectal cancer cells.

TABLE 6
Nitrofuran-based N-acylhydrazone screening set inhibitor cell viability results.

| | | Human Cell Viability EC₅₀ or CC₅₀ (μM) | | | | | Average |
|---|---|---|---|---|---|---|---|
| | | Colon Cancer | | | | Intestine | Selec- |
| | | (HCT | | (HT- | Colon | (FHs | tivity |
| Structure | # | 116) | (DLD1) | 29) | (FHC) | 74Int) | Index |
| 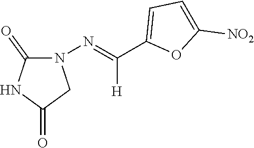 [SMILES: O=C1CN(/N=C/c2ccc([N+](=O)[O-])o2)C(=O)N1] | Nitro- furantoin (Nft) | 95 | | | >100 | >100 | 1.1 |
| 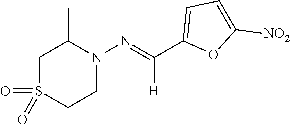 [SMILES: CC1CS(=O)(=O)CCN1/N=C/c1ccc([N+](=O)[O-])o1] | Nifurtimox (Nfx - 106) | >100 | | | >100 | >100 | 1.0 |
| 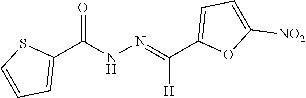 [SMILES: O=C(N/N=C/c1ccc([N+](=O)[O-])o1)c1cccs1] | 120 | 9.1 | 14 | 3.6 | 84 | 50 | 7.4 |
| 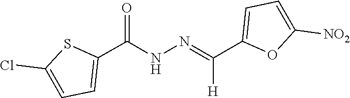 [SMILES: O=C(N/N=C/c1ccc([N+](=O)[O-])o1)c1ccc(Cl)s1] | 121 | 8.2 | 8.0 | 5.7 | 51 | 49 | 6.2 |
| 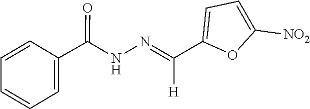 [SMILES: O=C(N/N=C/c1ccc([N+](=O)[O-])o1)c1ccccc1] | 122 | 13 | 18 | 4.9 | >100 | 71 | 6.5 |
| 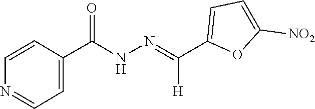 [SMILES: O=C(N/N=C/c1ccc([N+](=O)[O-])o1)c1ccncc1] | 123 | 28 | | | 70 | 75 | 2.6 |
| 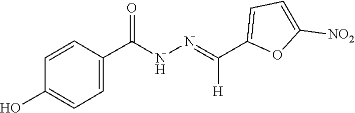 [SMILES: O=C(N/N=C/c1ccc([N+](=O)[O-])o1)c1ccc(O)cc1] | Nifur- oxazide (Nfz) | 8.3 | 1.5 | 3.5 | 66 | 62 | 7.7 |
| 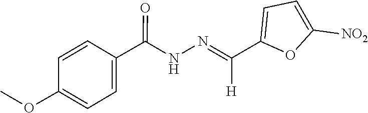 [SMILES: COc1ccc(C(=O)N/N=C/c2ccc([N+](=O)[O-])o2)cc1] | 124 | 4.6 | 7.6 | 2.7 | >100 | 47 | 16.1 |
| 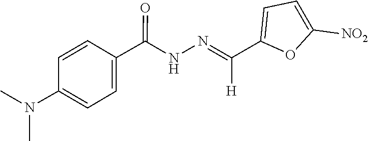 [SMILES: CN(C)c1ccc(C(=O)N/N=C/c2ccc([N+](=O)[O-])o2)cc1] | 125 | 8.5 | >100 | 0.29 | >100 | >100 | 11.8 |
| 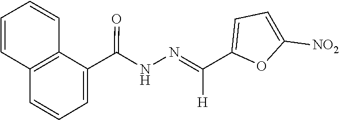 [SMILES: O=C(N/N=C/c1ccc([N+](=O)[O-])o1)c1cccc2ccccc12] | 126 | 9.6 | 11 | 4.4 | 58 | 52 | 5.7 |
| 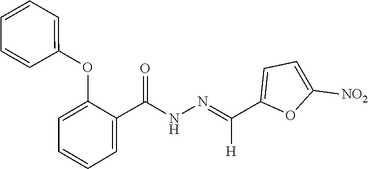 [SMILES: O=C(N/N=C/c1ccc([N+](=O)[O-])o1)c1ccccc1Oc1ccccc1] | 127 | 11 | 26 | 8.3 | 49 | 55 | 4.7 |
| 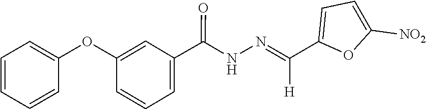 [SMILES: O=C(N/N=C/c1ccc([N+](=O)[O-])o1)c1cccc(Oc2ccccc2)c1] | 128 | 3.5 | 7.3 | 1.6 | 61 | 60 | 17.4 |
| 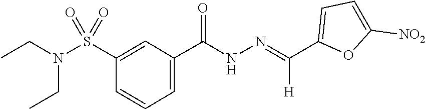 [SMILES: CCN(CC)S(=O)(=O)c1cccc(C(=O)N/N=C/c2ccc([N+](=O)[O-])o2)c1] | 129 | 7.8 | 8.9 | 4.2 | >100 | 8.7 | 12.0 |
| 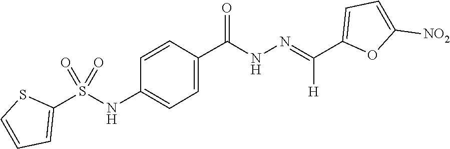 [SMILES: O=C(N/N=C/c1ccc([N+](=O)[O-])o1)c1ccc(NS(=O)(=O)c2cccs2)cc1] | 130 | 9.1 | 57 | 8.1 | >100 | 69 | 9.3 |
| 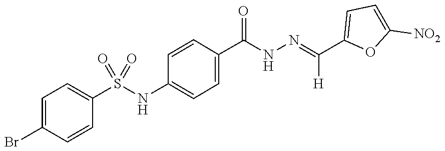 [SMILES: O=C(N/N=C/c1ccc([N+](=O)[O-])o1)c1ccc(NS(=O)(=O)c2ccc(Br)cc2)cc1] | 131 | 5.3 | 29 | 3.6 | >100 | >100 | 18.8 |
| 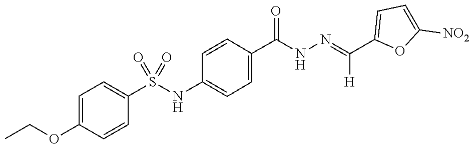 [SMILES: CCOc1ccc(S(=O)(=O)Nc2ccc(C(=O)N/N=C/c3ccc([N+](=O)[O-])o3)cc2)cc1] | 132 | 1.8 | 18 | 2.3 | 89 | >100 | 52.0 |

TABLE 7
Hydroxyquinoline-based N-acylhydrazone screening set inhibitor cell viability results.

| | | Human Cell Viability EC₅₀ or CC₅₀ (μM) | | | | | |
|---|---|---|---|---|---|---|---|
| | | Colon Cancer | | | | | Average |
| | | (HCT | | | Colon | Intestine | Selectivity |
| Structure | # | 116) | (DLD1) | (HT-29) | (FHC) | (FHs 74Int) | Index |
| 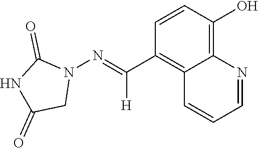 [SMILES: O=C1CN(/N=C/c2ccc(O)c3ncccc23)C(=O)N1] | 133 | 39 | | | >100 | >100 | 2.6 |
| 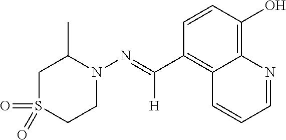 [SMILES: CC1CS(=O)(=O)CCN1/N=C/c1ccc(O)c2ncccc12] | 134 | 7.0 | 6.3 | 8.1 | >100 | 87 | 13 |
| 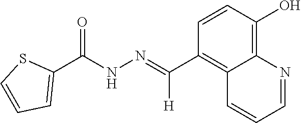 [SMILES: O=C(N/N=C/c1ccc(O)c2ncccc12)c1cccs1] | 135 | 3.2 | 2.3 | 3.6 | 85 | 86 | 27 |
| 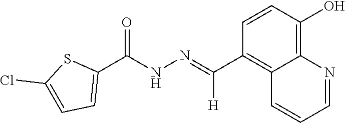 [SMILES: O=C(N/N=C/c1ccc(O)c2ncccc12)c1ccc(Cl)s1] | 136 | 3.8 | 3.9 | 3.2 | 25 | 42 | 8.8 |
| 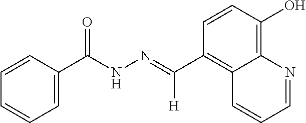 [SMILES: O=C(N/N=C/c1ccc(O)c2ncccc12)c1ccccc1] | 137 | 6.1 | 2.8 | 2.5 | 74 | 57 | 11 |
| 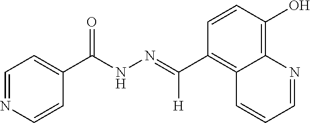 [SMILES: O=C(N/N=C/c1ccc(O)c2ncccc12)c1ccncc1] | 138 | 48 | | | >100 | 69 | 1.8 |
| 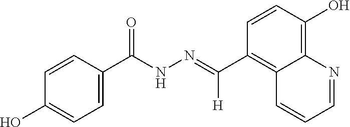 [SMILES: O=C(N/N=C/c1ccc(O)c2ncccc12)c1ccc(O)cc1] | 139 | 7.4 | 19 | 6.3 | >100 | 56 | 11 |
| 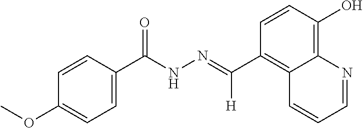 [SMILES: COc1ccc(C(=O)N/N=C/c2ccc(O)c3ncccc23)cc1] | 18 | 7.8 | 3.8 | 3.5 | 28 | 55 | 5.3 |
| 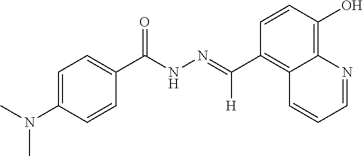 [SMILES: CN(C)c1ccc(C(=O)N/N=C/c2ccc(O)c3ncccc23)cc1] | 140 | 4.4 | | 2.4 | 35 | 42 | 8.8 |
| 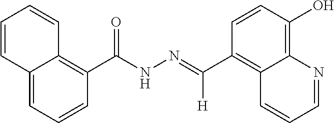 [SMILES: O=C(N/N=C/c1ccc(O)c2ncccc12)c1cccc2ccccc12] | 141 | 4.1 | 1.6 | 1.5 | 46 | 60 | 13 |
| 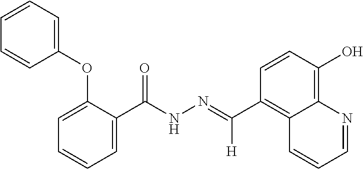 [SMILES: O=C(N/N=C/c1ccc(O)c2ncccc12)c1ccccc1Oc1ccccc1] | 142 | 6.3 | 1.6 | 3.7 | 71 | 87 | 13 |
| 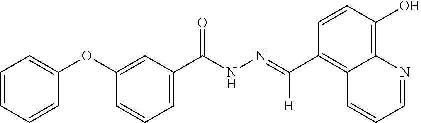 [SMILES: O=C(N/N=C/c1ccc(O)c2ncccc12)c1cccc(Oc2ccccc2)c1] | 143 | 6.5 | | | 41 | 21 | 4.8 |
| 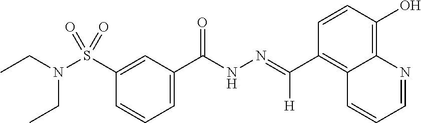 [SMILES: CCN(CC)S(=O)(=O)c1cccc(C(=O)N/N=C/c2ccc(O)c3ncccc23)c1] | 144 | 4.9 | 1.3 | 4.4 | 34 | 59 | 9.5 |
| 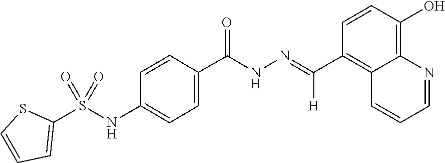 [SMILES: O=C(N/N=C/c1ccc(O)c2ncccc12)c1ccc(NS(=O)(=O)c2cccs2)cc1] | 145 | 2.4 | 5.7 | 2.9 | 24 | 6.1 | 6.2 |
| 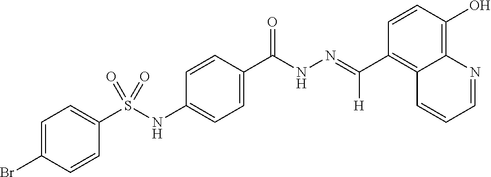 [SMILES: O=C(N/N=C/c1ccc(O)c2ncccc12)c1ccc(NS(=O)(=O)c2ccc(Br)cc2)cc1] | 146 | 5.2 | 6.0 | 1.4 | 59 | 91 | 14 |
| 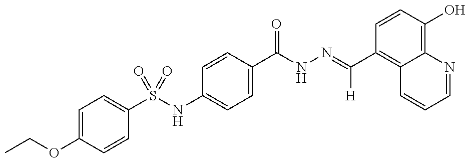 [SMILES: CCOc1ccc(S(=O)(=O)Nc2ccc(C(=O)N/N=C/c3ccc(O)c4ncccc34)cc2)cc1] | 147 | 1.8 | 6.2 | 2.9 | 52 | 40 | 25 |

TABLE 8
Initial N-acylhydrazones screening inhibitor cell viability results.

| | | | Human Cell Viability EC₅₀ or CC₅₀ (μM) | | | | | Average |
|---|---|---|---|---|---|---|---|---|
| | R | | Colon Cancer | | | Colon | Intestine | Selectivity |
| Structure | Group | # | (HCT 116) | (DLD1) | (HT-29) | (FHC) | (FHs 74Int) | Index |
| 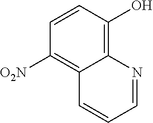 [SMILES: O=[N+]([O-])c1ccc(O)c2ncccc12] | Nitroxoline | | 22 | | | 28 | 41 | 1.6 |
| 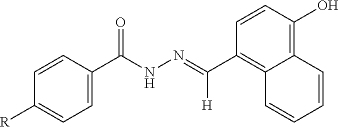 | OCH₃ H | 148 149 | >100 >100 | | | >100 >100 | >100 >100 | 1.0 1.0 |
| 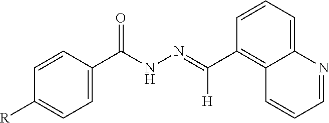 | OCH₃ H | 150 151 | >100 >100 | | | >100 >100 | >100 >100 | 1.0 1.0 |
| 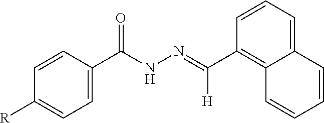 | OCH₃ H | 152 153 | >100 >100 | | | >100 100 | >100 >100 | 1.0 1.0 |
| 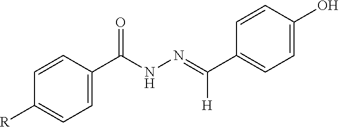 | OCH₃ H | 154 155 | >100 >100 | | | >100 >100 | >100 >100 | 1.0 1.0 |
| 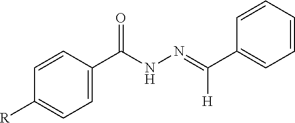 | OCH₃ H | 156 157 | >100 >100 | | | >100 >100 | >100 >100 | 1.0 1.0 |
| 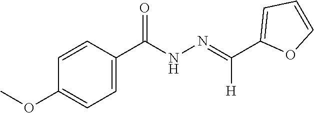 [SMILES: COc1ccc(C(=O)N/N=C/c2ccco2)cc1] | | 158 | >100 | | | >100 | >100 | 1.0 |
| 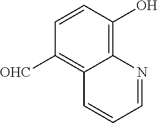 [SMILES: O=Cc1ccc(O)c2ncccc12] | | 159 | 6.2 | | | 4.4 | 15 | 1.6 |
| 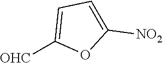 | | 160 | 29 | | | 53 | 53 | 1.9 |
| 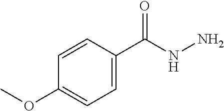 | | 161 | >100 | | | >100 | >100 | 1.0 |

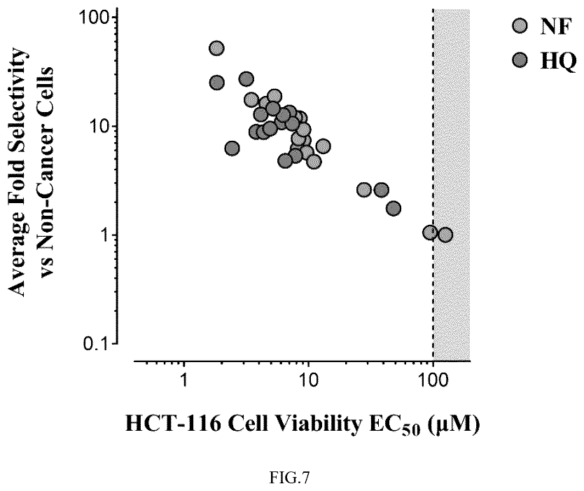

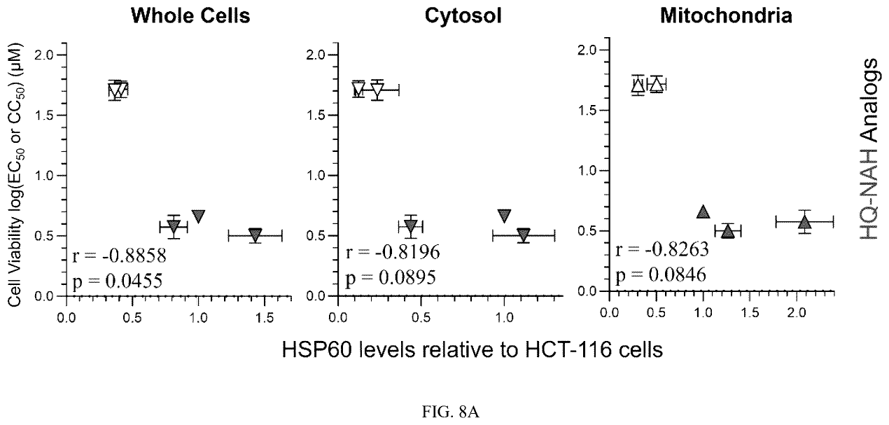

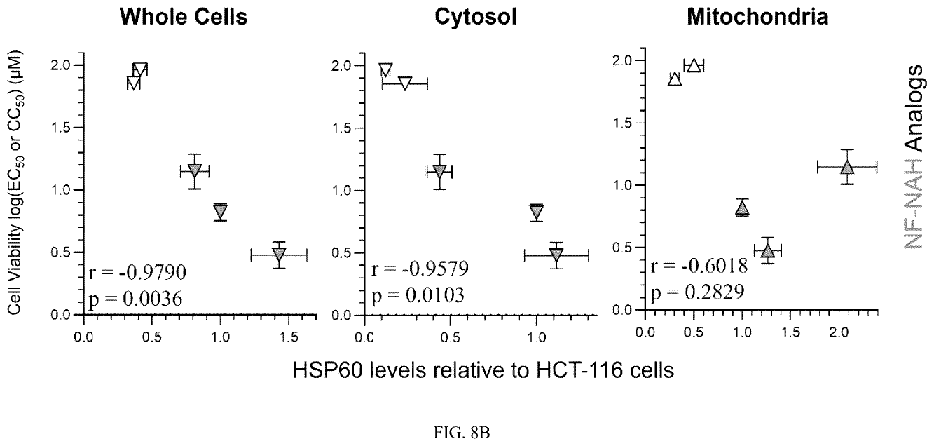

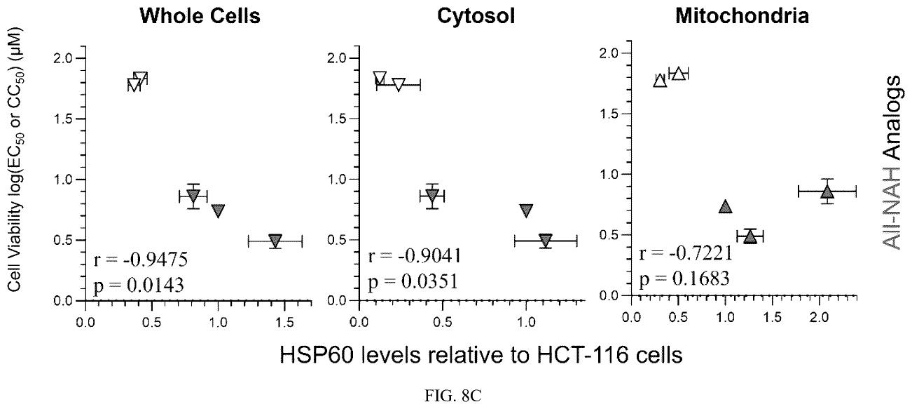

As a previous study performed cell fractionation experiments to evaluate HSP60 levels in the mitochondria, cytosol, and the whole cell, it was similarly examined for correlations between those results and the cytotoxicities of the NF-NAH and HQ-NAH. Results showed that cytotoxicities of the most potent and selective NF-NAH analogs showed higher correlation with HSP60 levels in the cytosol, whereas the HQ-NAH analogs showed similar correlation with HSP60 levels in the mitochondria and cytosol (FIGS. 8A-8Q). While these results provide insights on how the inhibitors may be targeting the two pools of HSP60 in cells, further experiments are warranted to conclusively establish inhibition mechanisms and whether either series is on-target against cHSP60 or mHSP60.

### Example 3: Lead Candidate Cytotoxicity Across Cancer Cell Lines from Different Organs

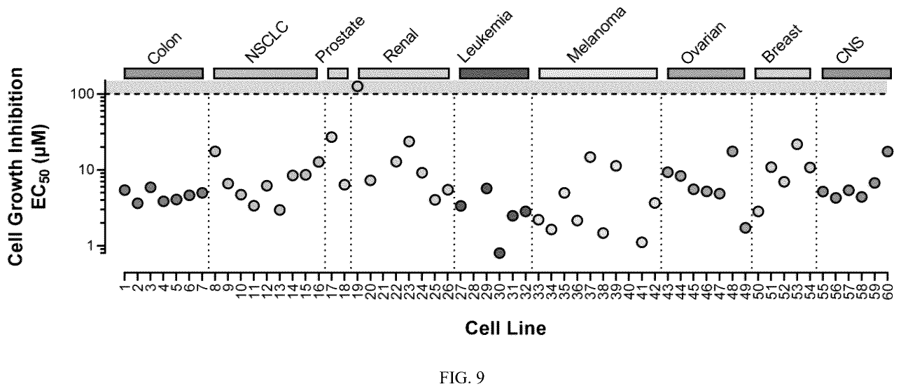

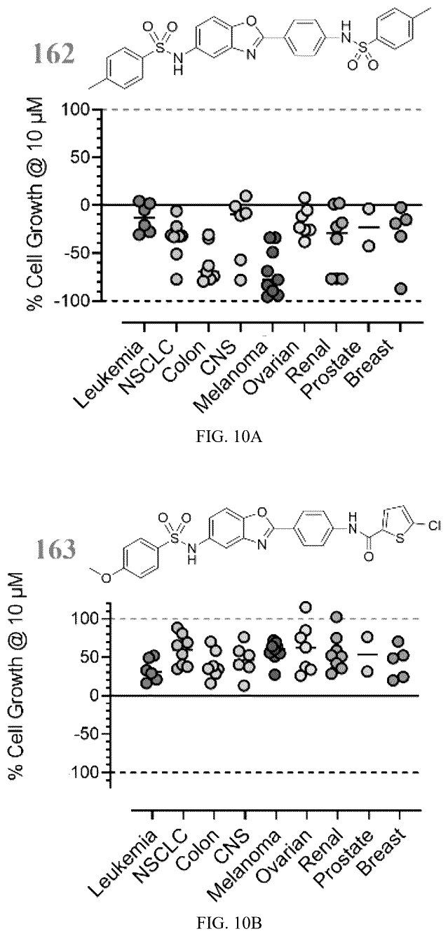

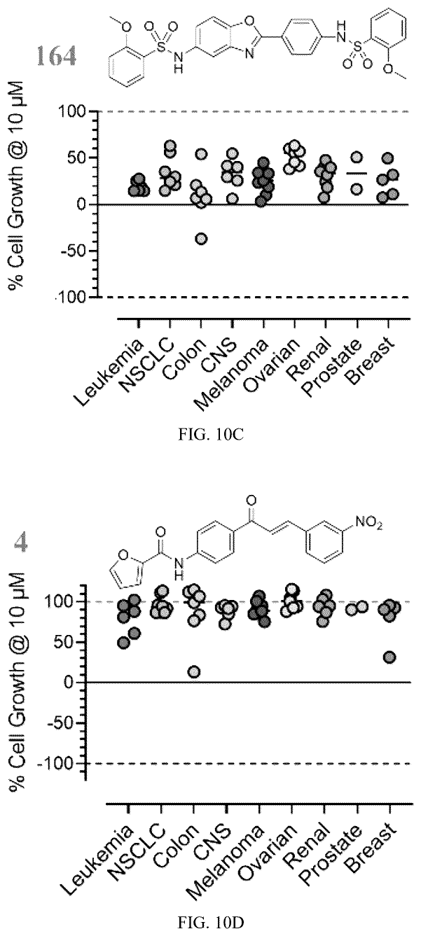

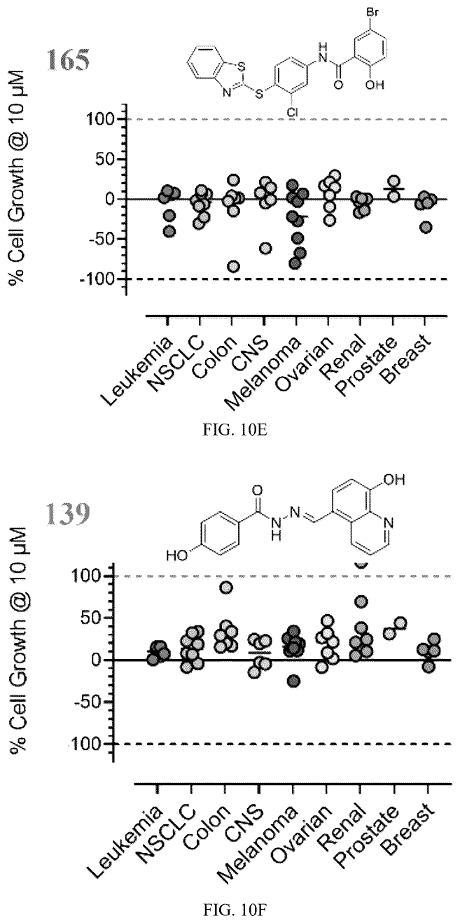

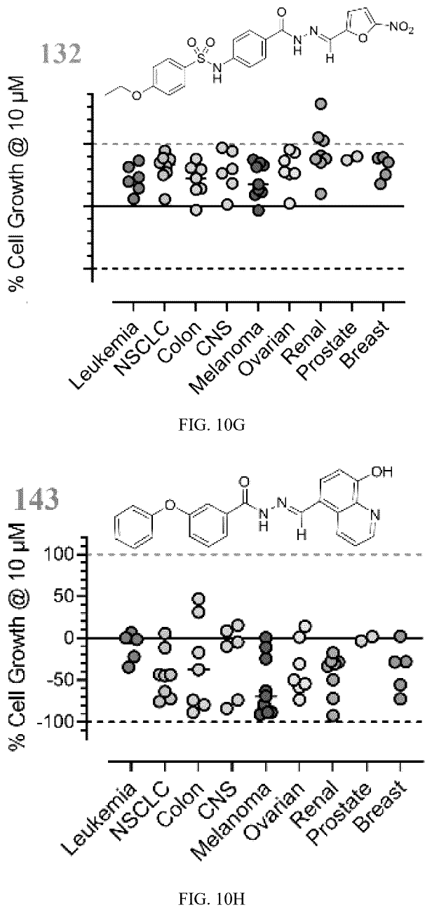

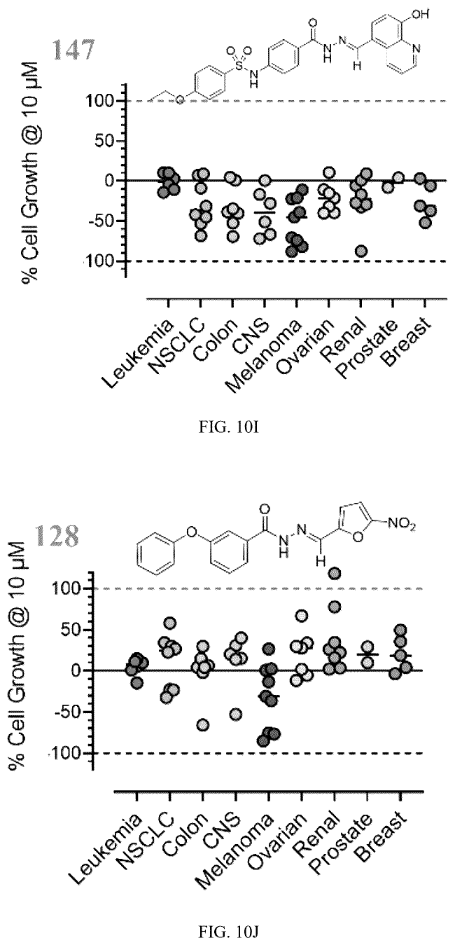

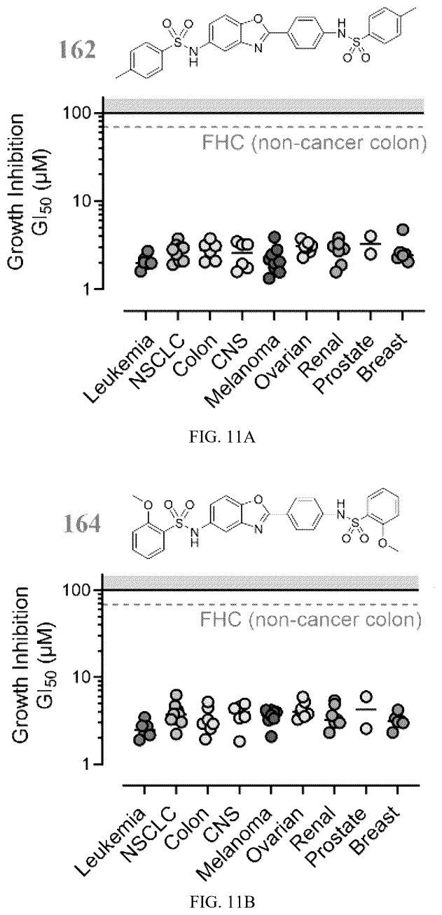

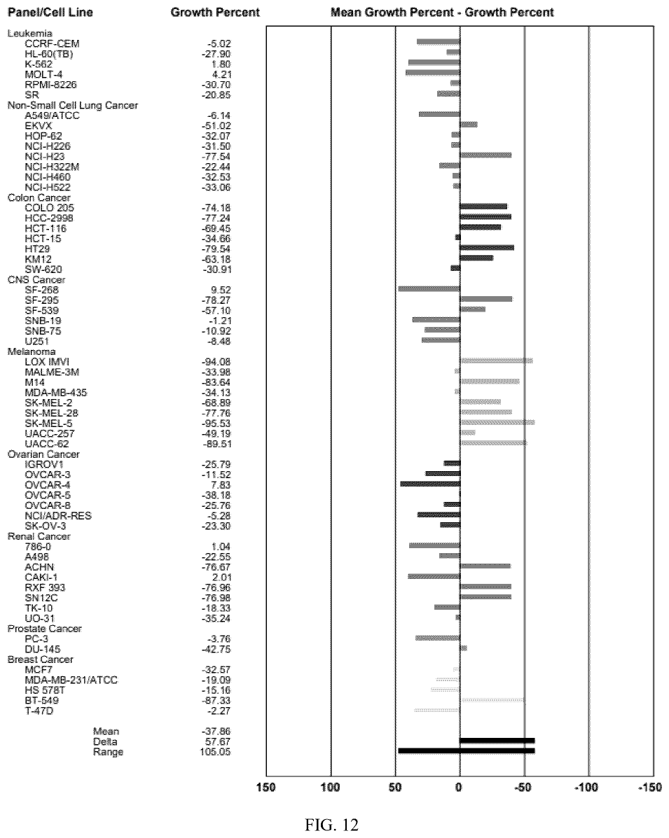

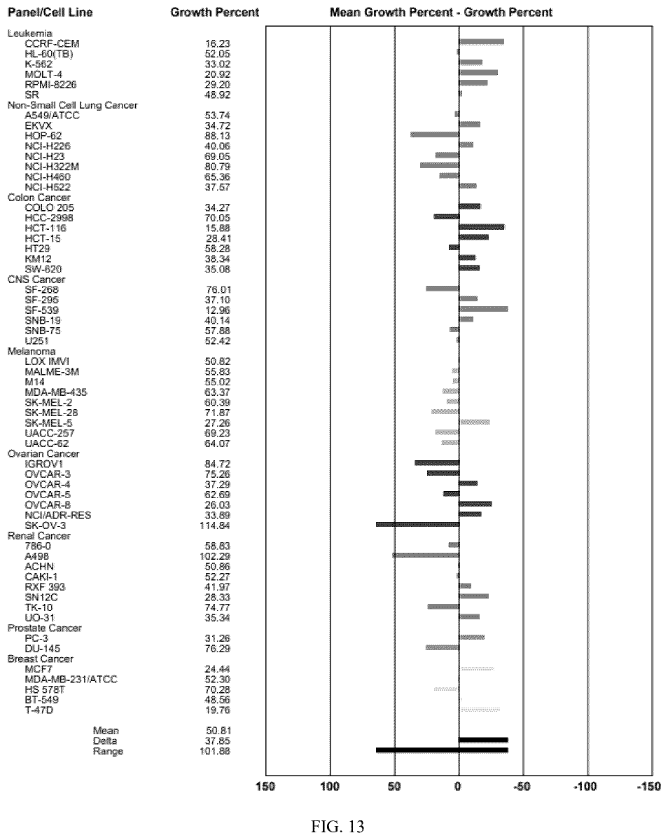

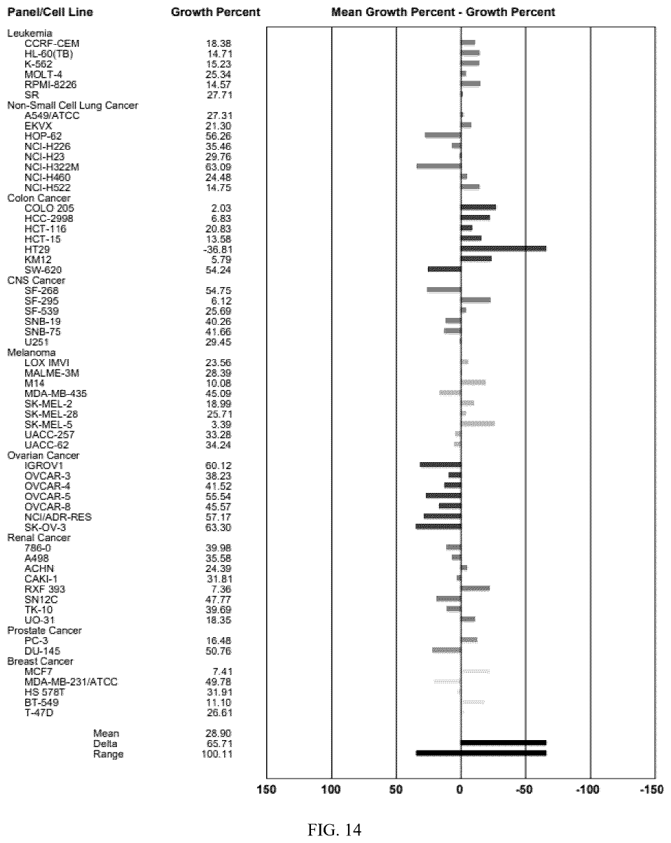

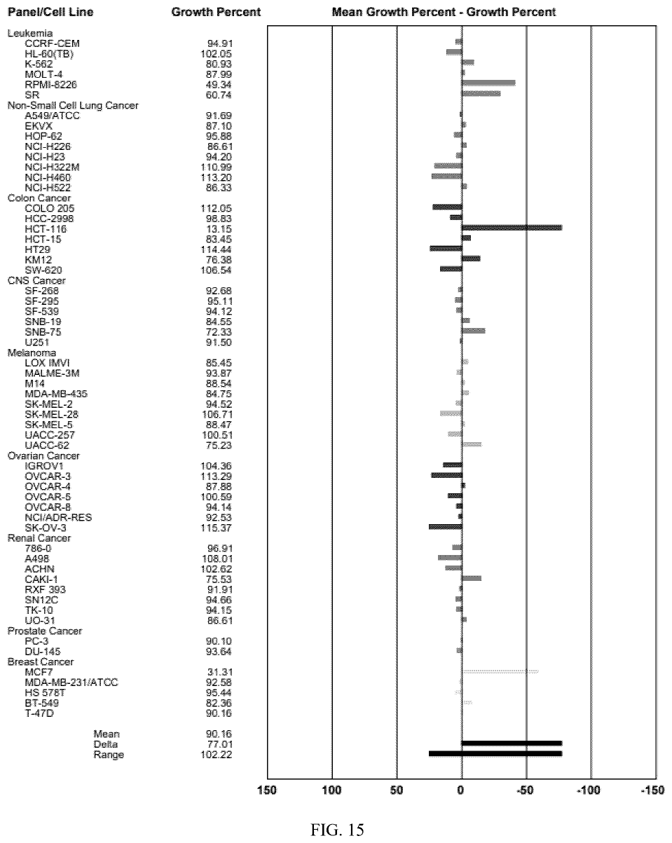

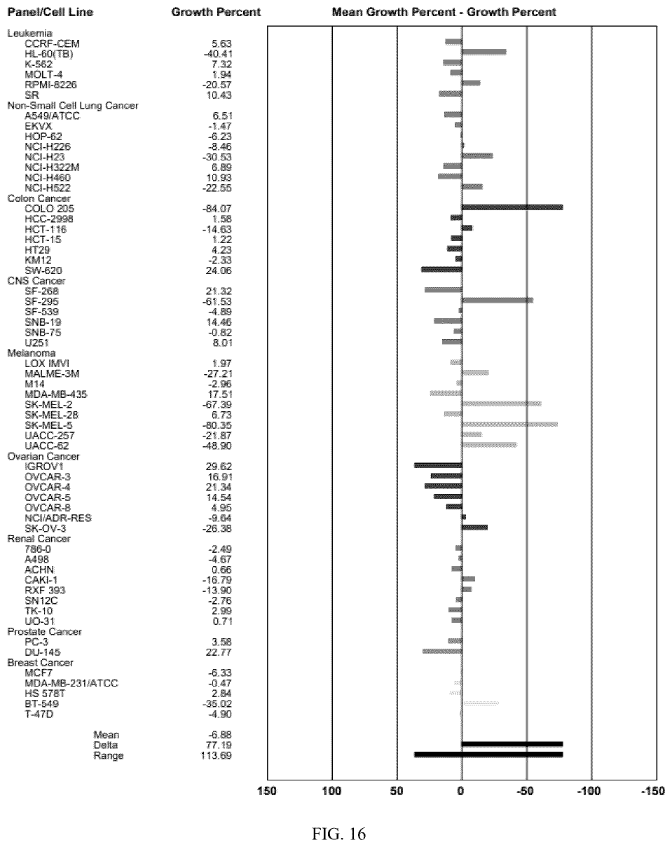

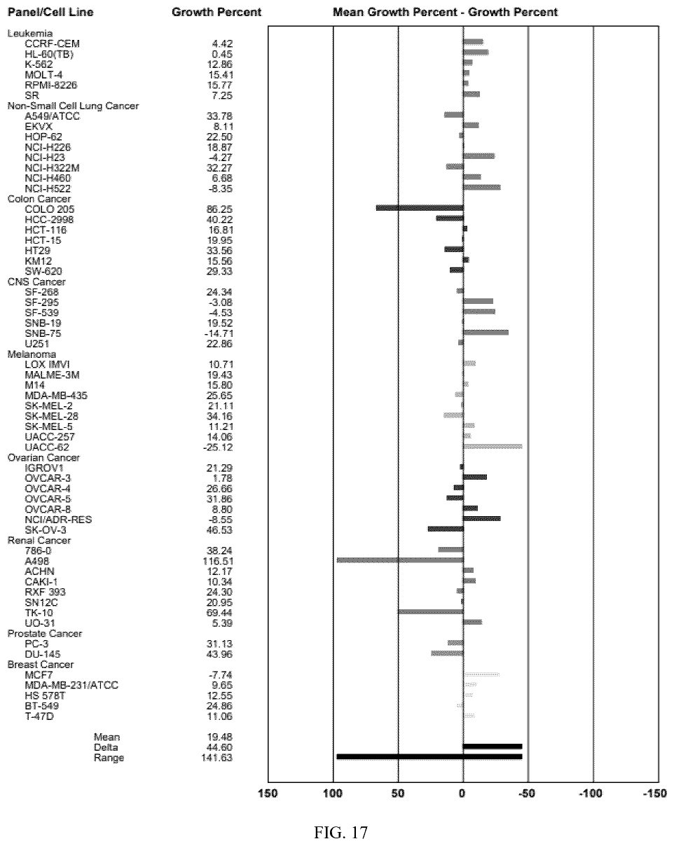

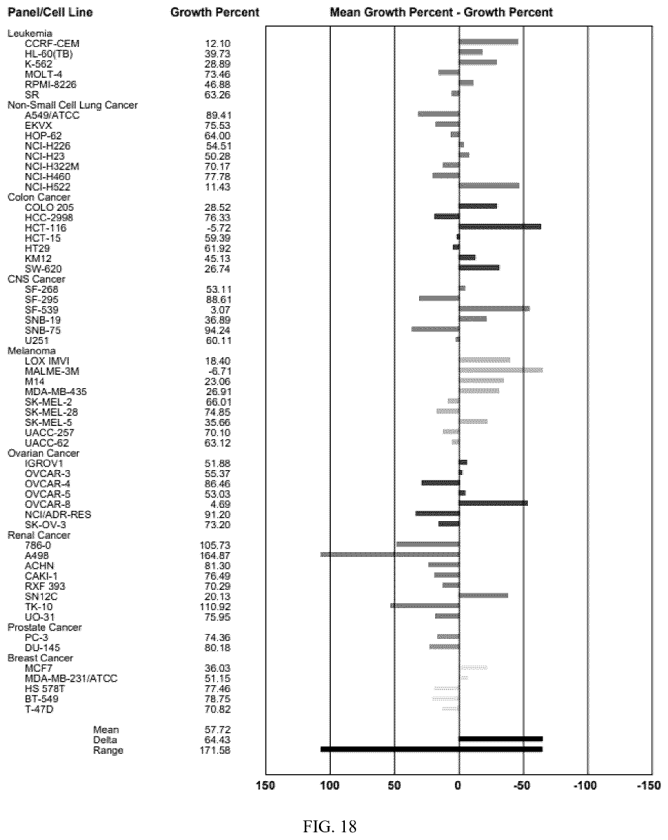

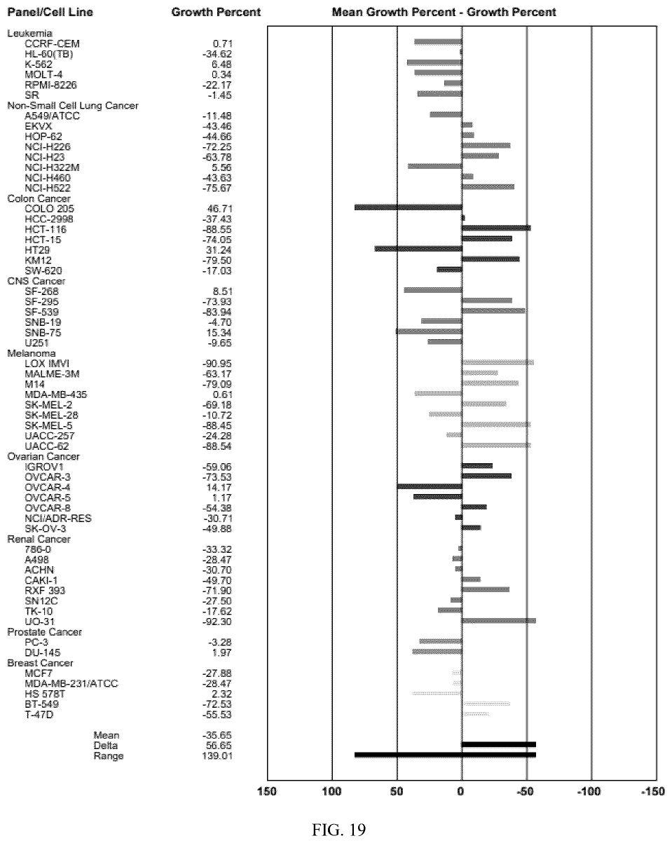

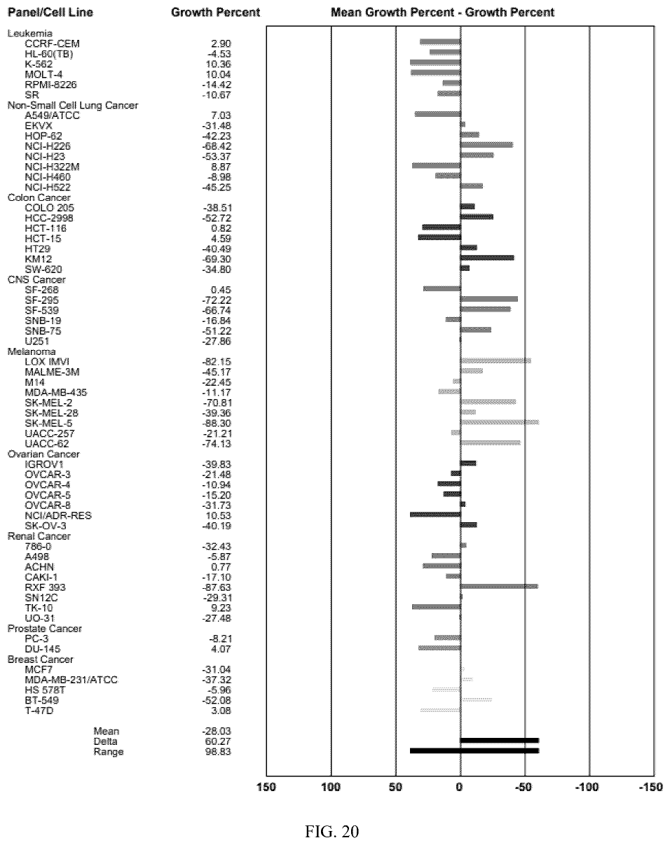

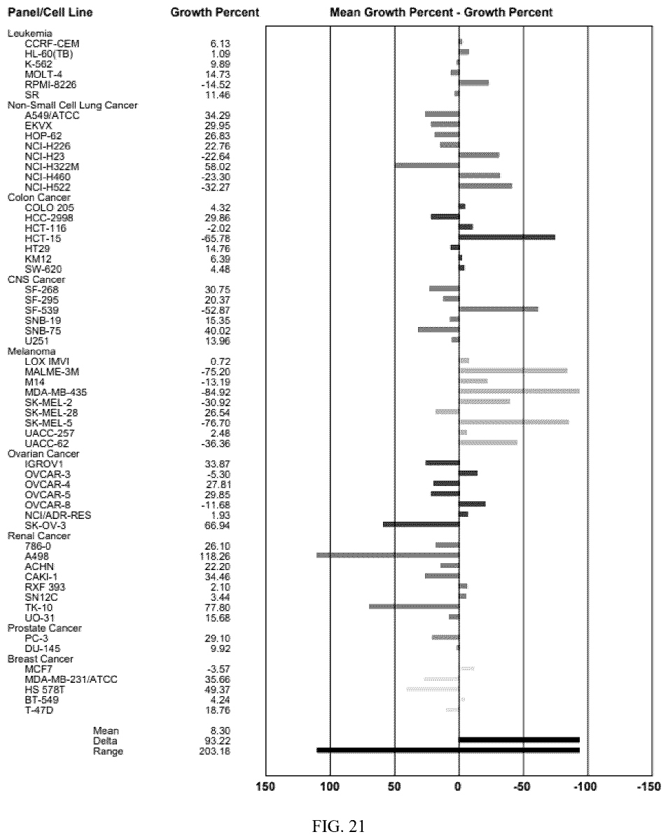

In a previous study evaluating the anti-cancer effects of ABK inhibitors, the National Cancer Institute (NCI) database was explored for existing cytotoxicity results for any compounds bearing α,β-unsaturated ketone moieties against the comprehensive NCI-60 cancer cell panel. Reported EC₅₀ results were found for chalcone and percent inhibition results for several analogs tested at 10 μM. These compounds bearing α,β-unsaturated ketone moieties were most potent and selective against the colorectal and leukemia cancer cells, but showed virtually no activity against other cancer cell types, including prostate, renal, melanoma, ovarian, breast, central nervous system (CNS), and non-small cell lung cancer (NSCLC) cells. For the present study, EC₅₀ results were reported for Nifuroxazide in the NCI database, indicating potency across nearly the entire panel of cancer cell lines (FIG. 9). The HQ-NAH and NF-NAH inhibitor library was explored based on the similarity to Nifuroxazide's scaffold. Ten of the most potent and selective lead candidates for profiling against the NCI-60 panel were submitted, which included 3 PBZ analogs, 1 ABK analog, 1 SCA analog, and 5 matched NF-NAH and HQ-NAH analogs (Table 9), one of which is the corresponding HQ-NAH analog to Nifuroxazide already reported in the NCI database. Initial screening was performed at a single concentration of 10 μM, and cell growth results are presented in aggregate based on cancer cell origin (FIGS. 10A-10J). Detailed results for each individual cell line are presented in FIGS. 12-21.

TABLE 9
Lead candidates for profiling against the NCI-60 panel.

| No. | Series | Structure |
|---|---|---|
| 4 | ABK | 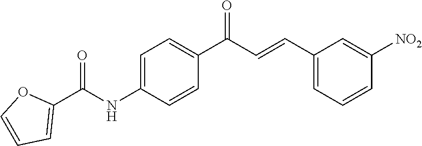 [SMILES: O=C(/C=C/c1cccc([N+](=O)[O-])c1)c1ccc(NC(=O)c2ccco2)cc1] |
| 128 | NF-NAH | 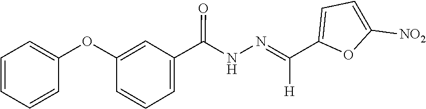 [SMILES: O=C(N/N=C/c1ccc([N+](=O)[O-])o1)c1cccc(Oc2ccccc2)c1] |
| 132 | NF-NAH | 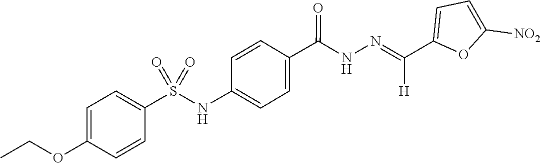 [SMILES: CCOc1ccc(S(=O)(=O)Nc2ccc(C(=O)N/N=C/c3ccc([N+](=O)[O-])o3)cc2)cc1] |
| 139 | HQ-NAH | 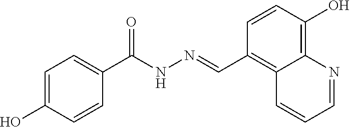 [SMILES: O=C(N/N=C/c1ccc(O)c2ncccc12)c1ccc(O)cc1] |
| 143 | HQ-NAH | 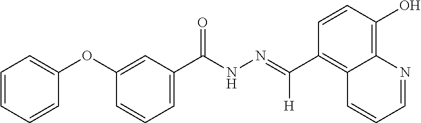 [SMILES: O=C(N/N=C/c1ccc(O)c2ncccc12)c1cccc(Oc2ccccc2)c1] |
| 147 | HQ-NAH | 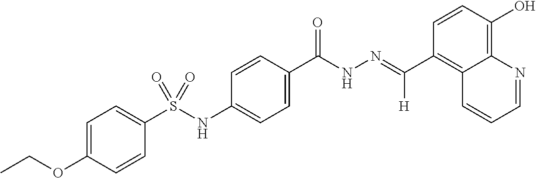 [SMILES: CCOc1ccc(S(=O)(=O)Nc2ccc(C(=O)N/N=C/c3ccc(O)c4ncccc34)cc2)cc1] |
| 162 | PBZ | 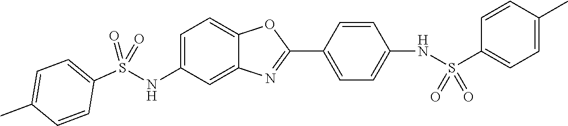 |
| 163 | PBZ | 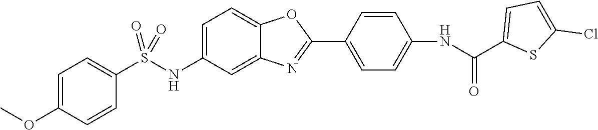 [SMILES: COc1ccc(S(=O)(=O)Nc2ccc3oc(-c4ccc(NC(=O)c5ccc(Cl)s5)cc4)nc3c2)cc1] |
| 164 | PBZ | 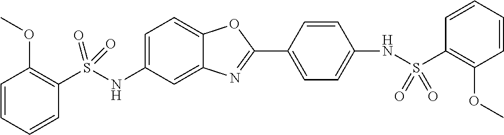 [SMILES: COc1ccccc1S(=O)(=O)Nc1ccc(-c2nc3cc(NS(=O)(=O)c4ccccc4OC)ccc3o2)cc1] |
| 165 | SCA | 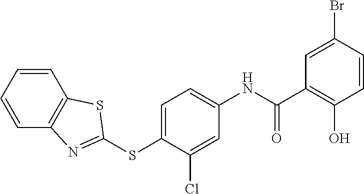 [SMILES: O=C(Nc1ccc(Sc2nc3ccccc3s2)c(Cl)c1)c1cc(Br)ccc1O] |

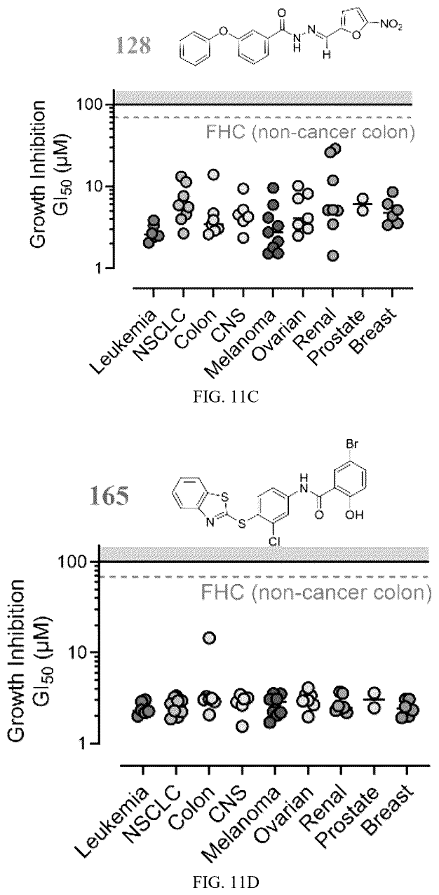

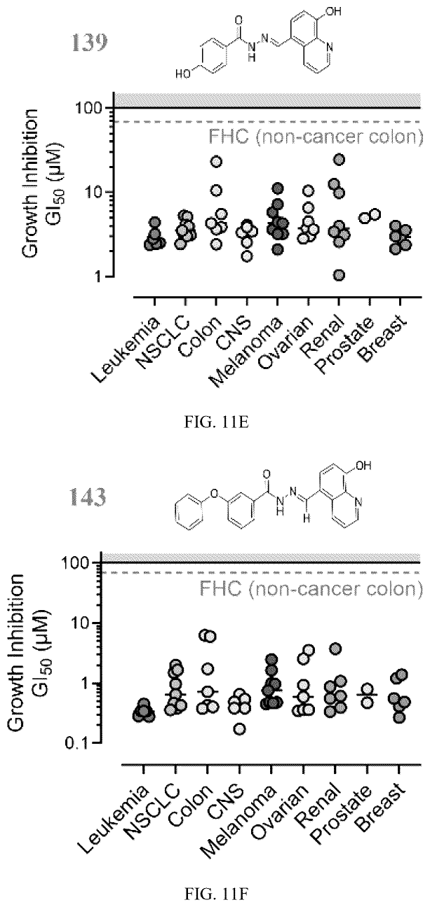

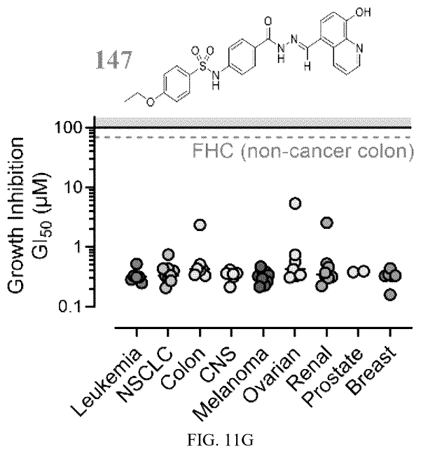

Intriguingly, ABK 4 was largely inactive across the entire panel of cancer cell lines, suggesting that the ABK series overall may not be effective for broad-spectrum cancer inhibition. Conversely, it was observed that lead candidates from the SCA, PBZ, NF, and HQ series displayed notable potency across a majority of cell lines in the NCI-60 panel (58 cell lines analyzed at this time), some of which appeared to display cytocidal effects, while others appeared cytostatic in a single dose proliferation assay (FIGS. 10A-10J). Compounds 162 (PBZ), 165 (SCA), 143 (HQ-NAH), and 147 (HQ-NAH) resulted in negative % growth (FIGS. 10A-10J), indicating a loss of cells due to cell death. Therefore, they appeared to act in a cytocidal manner. In follow-up dose response testing (FIGS. 11A-11G), these compounds were noticeably potent with GI₅₀ values ranging between 1-5 μM for 162 and 165, to sub-micromolar for HQ-NAH compounds 143 and 147 between 0.2-0.4 μM. While 164 (PBZ) and 139 (HQ-NAH) displayed similarly potent GI₅₀ ranges between 1-5 μM (FIGS. 11A-11G), they appeared to exhibit cytostatic effects, with growth % values clustering around 0% (FIGS. 10A-10J), indicating cells were surviving but unable to proliferate. Interestingly, NF-NAH compound 128 appeared to function in a cell line dependent manner, displaying a range of positive, negative, and zero growth, with GI₅₀ values ranging between 1-31 μM suggesting its broad spectrum potential may be limited compared to other molecules considered. HQ-NAH compounds 143 and 147 appeared to exhibit the best broad spectrum potential.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

What is claimed is:

1. A method of treating cancer in a subject in need thereof with a therapeutically effective amount of an antibacterial or an antiparasitic compound.

2. The method of claim 1, wherein the compound is of formula (I), 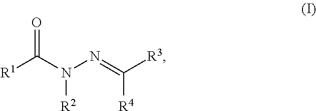
or a pharmaceutically acceptable salt thereof, wherein:
(i) R¹ is aryl or heteroaryl, wherein aryl or heteroaryl is optionally substituted with halogen, —OR⁵, —NR⁵R⁶, —S(O)₂NR⁵R⁶, or —N(R⁵)SO₂R⁶; and
R² is hydrogen; or
(ii) R¹ and R² combine with the atoms to which they are attached to form a 3- to 7-membered heterocyclyl;
R³ is aryl or heteroaryl, wherein aryl or heteroaryl is optionally substituted by —OR^{5′} or nitro;
R⁴ is hydrogen; and
R⁵, R^{5′}, and R⁶ are each independently hydrogen, alkyl, aryl, or heteroaryl, wherein each alkyl, aryl, and heteroaryl is optionally substituted with halogen or —O-alkyl.

3. The method of claim 2, wherein R³ is heteroaryl optionally substituted by nitro or hydroxy.

4. The method of claim 3, wherein R³ is quinolyl substituted by hydroxy.

5. The method of claim 3, wherein R³ is furanyl substituted by nitro.

6. The method of claim 2, wherein the compound is of formula (II) or (III), 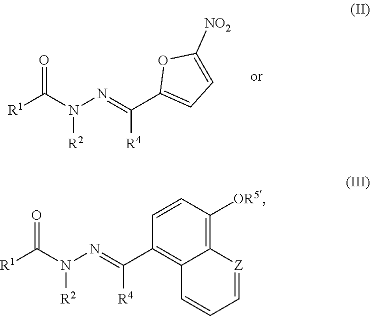
or a pharmaceutically acceptable salt thereof, wherein:
Z is CH or N.

7. The method of claim 6, wherein R¹ is aryl or heteroaryl, each of which is optionally substituted with halogen, —OR⁵, —NR⁵R⁶, —S(O)₂NR⁵R⁶, or —N(R⁵)SO₂R⁶.

8. The method of claim 7, wherein R¹ is C₆-C₁₀ aryl optionally substituted with halo, —OR⁵, —NR⁵R⁶, —S(O)₂NR⁵R⁶, or —N(R⁵)SO₂R⁶.

9. The method of claim 8, wherein R¹ is phenyl optionally substituted by —OH, —OC₁-C₆ alkyl, O—C₆-C₁₀ aryl, —N(C₁-C₆ alkyl)₂, —S(O)₂N(C₁-C₆ alkyl)₂, —N(H)S(O)₂—C₆-C₁₀ aryl, —N(H)S(O)₂-heteroaryl, or —N(H)S(O)₂-heteroaryl, wherein each C₆-C₁₀ aryl or heteroaryl is optionally substituted with halogen or —OC₁-C₆ alkyl.

10. The method of claim 6, wherein R¹ is selected from the group consisting of

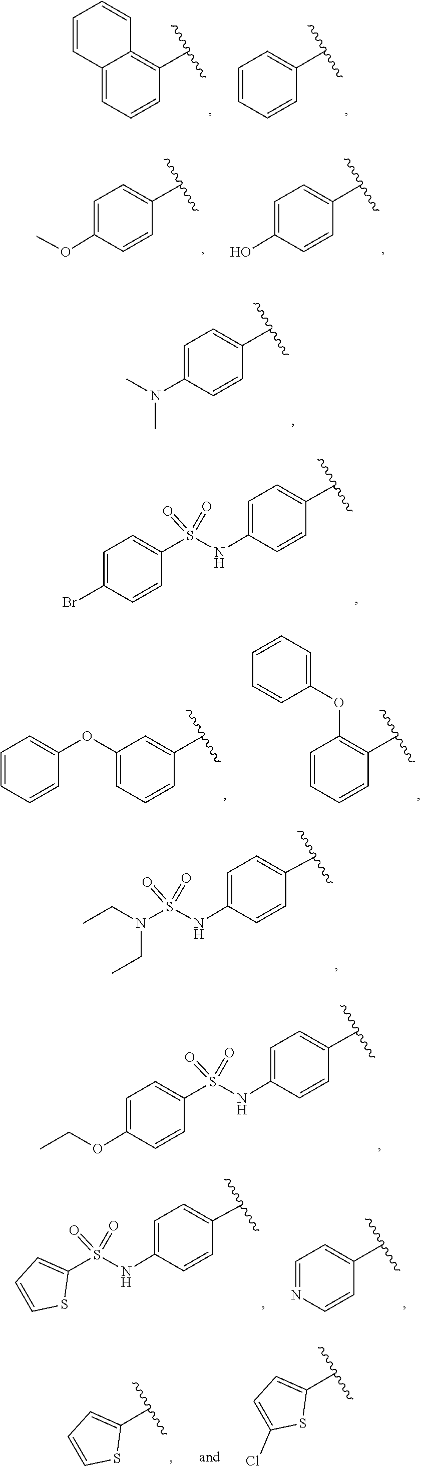

11. The method of claim 1, wherein the compound is of formula (IV), 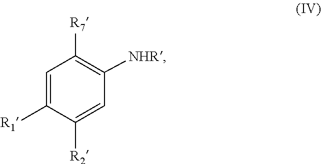
or a pharmaceutically acceptable salt thereof, wherein
R′ is C₁-C₆ alkyl or 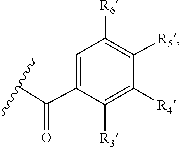
R₁′ is H, —OH, —OC₁-C₆ alkyl, —NHC(O)C₁-C₆ alkyl, —C(O)OC₁-C₆ alkyl, —C(O)OH, C₁-C₆ alkyl, —S-heteroaryl, or 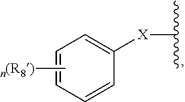
wherein C₁-C₆ alkyl is optionally substituted by —CN,
R₂′ is H or halo,
each of R₃′, R₄′, R₅′, and R₆′ is independently H, —OH, halo, —O—C₁-C₆ alkyl, —NO₂, or —NH₂,
R₇′ is H or C₁-C₆ alkyl,
X is —O—, —S—, —C(R₉′)(R₁₀′)ₘ—, or —C(R₉′)(R₁₀′)ₘO—, optionally X is —O—, —S—, or —C(R₉′)(CN);
R₈′ is halo,
R₉′ is H or —CN,
R₁₀′ is H or —CN,
m is 1 or 2, and
n is 0, 1, or 2.

12. The method of claim 11, wherein the compound is of formula (V), 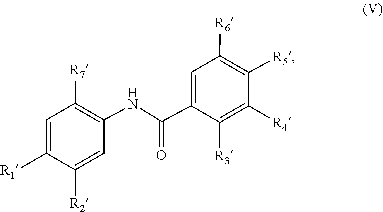
or a pharmaceutically acceptable salt thereof.

13. The method of claim 11, wherein the compound is of formula (X), 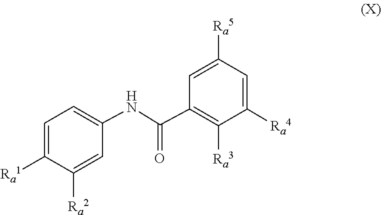
or a pharmaceutically acceptable salt thereof, wherein
Rₐ¹ is H or 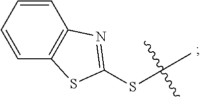 [SMILES: *Sc1nc2ccccc2s1]
Rₐ² is H or Cl;
Rₐ³ is H, OH, or OCH₃;
Rₐ⁴ is H or Br; and
Rₐ⁵ is H or Br;
provided that at least one of Rₐ¹, Rₐ², Rₐ³, Rₐ⁴, and Rₐ⁵ is not H.

14. The method of claim 1, wherein the compound is of formula (XIII) or (XIV), 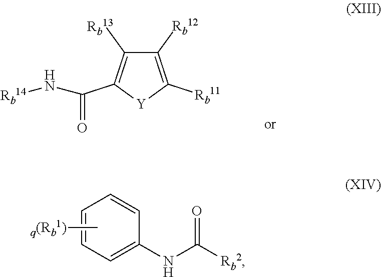
or pharmaceutically acceptable salt thereof, wherein
Y is O or S;
R_{b}¹ is halo, C₁-C₈ alkyl, C₆-C₁₀ aryl, 5- to 12-membered heteroaryl, phenoxy, benzyl, —C(O)R^{A}, OR^{A}, —SR^{A} or —NHR^{A}, or one or more R_{b}¹ in combination with the atoms to which each is attached combine to form a naphthyl or C₉-C₁₂ bicyclic heteroaryl, wherein C₆-C₁₀ aryl, 5- to 12-membered heteroaryl, phenoxy, benzyl, C₁-C₈ alkyl, —C(O)C₂-C₆ alkenylene-phenyl, C₉-C₁₂ aryl and C₉-C₁₂ bicyclic heteroaryl is optionally substituted by halo, cyano, or —NO₂;
R_{b}² is C₆-C₁₀ aryl or 5- to 12-membered heteroaryl, wherein C₆-C₁₀ aryl and 5- to 12-membered heteroaryl is optionally substituted by halo, hydroxy, or —NO₂;
R_{b}¹ is H, nitro, or N(H)COR_{b}³;
R_{b}¹² is H or C(O)OC₁-C₆ alkyl;
R_{b}¹³ is H or C₁-C₆ alkyl;
R_{b}¹⁴ is H, C₆-C₁₀ aryl, or 5 to 12-membered heteroaryl, wherein C₆-C₁₀ aryl and 5 to 12-membered heteroaryl is optionally substituted by halo, 5 to 12-membered heteroaryl, —NHC(O)C₁-C₆ alkyl, C₁-C₆ alkoxy, —C(O)C₁-C₆ alkoxy, —R^{A}, OR^{A}, —SR^{A}—C(O)R^{A} or —NHR^{A};
each R^{A} is independently C₁-C₆ alkyl, C₆-C₁₀ aryl, C₂-C₆ alkenyl-C₆-C₁₀ aryl, —C(O)C₁-C₆ alkyl, C₁-C₆ alkoxy, or 5 to 12-membered heteroaryl, wherein C₁-C₆ alkyl, C₆-C₁₀ aryl, C₂-C₆ alkenyl-C₆-C₁₀ aryl, —C(O)C₁-C₆ alkyl, C₁-C₆ alkoxy, or 5 to 12-membered heteroaryl is optionally substituted by nitro or halo; or two R^{A} combine to form C₄-C₆ cycloalkyl optionally substituted by phenyl;
R_{b}³ is C₆-C₁₀ aryl, 5 to 12-membered heteroaryl, or C₉-C₁₂ bicyclic aryl, wherein C₆-C₁₀ aryl, 5 to 12-membered heteroaryl, and C₉-C₁₂ bicyclic aryl is optionally substituted by halo, C₁-C₆ alkyl, or nitro; and
q is 0, 1, 2, or 3.

15. The method of claim 14, wherein the compound is of formula (XVII), 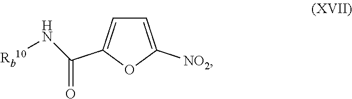
or a pharmaceutically acceptable salt thereof, wherein
R_{b}¹⁰ is aryl or heteroaryl, optionally substituted with halo, phenoxy, —OR_{b}⁵, —NR_{b}⁵R_{b}⁶, —S(O)₂NR_{b}⁵R_{b}⁶, or —NHSO₂R⁷;
R_{b}⁵ and R_{b}⁶ are each independently hydrogen, C₁-C₆ alkyl, aryl, or heteroaryl, wherein C₁-C₆ alkyl, aryl, and heteroaryl is optionally substituted with halogen or —OC₁-C₆ alkyl; and
R_{b}⁷ is aryl or heteroaryl, each optionally substituted with halo, —OR_{b}⁵ or nitro.

16. The method of claim 1, wherein the compound is of formula (XVIII), 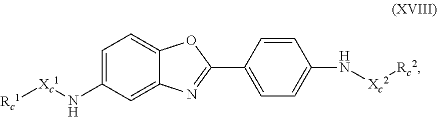
or a pharmaceutically acceptable salt thereof, wherein
each of X_{c}¹ and X_{c}² is independently —SO₂— or —C(O)—; and
each of R_{c}¹ and R_{c}² is independently alkyl, aryl, or heteroaryl, wherein alkyl, aryl, and heteroaryl are optionally substituted with halogen, hydroxy, nitro, amino, cyano, alkyl, aryl, haloalkyl, alkoxy, C(O)OH, or C(O)O-alkyl.

17. The method of claim 16, wherein the compound is of formula (XIX), 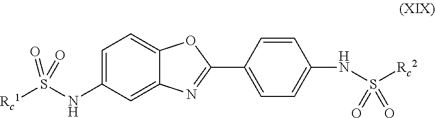
or a pharmaceutically acceptable salt thereof, wherein
each of R_{c}¹ and R_{c}² is independently alkyl, haloalkyl, aryl, or heteroaryl, wherein alkyl, aryl, and heteroaryl are optionally substituted with halogen, hydroxy, nitro, amino, cyano, alkyl, aryl, haloalkyl, alkoxy, C(O)OH, or C(O)O-alkyl.

18. The method of claim 1, wherein the compound is selected from the group consisting of 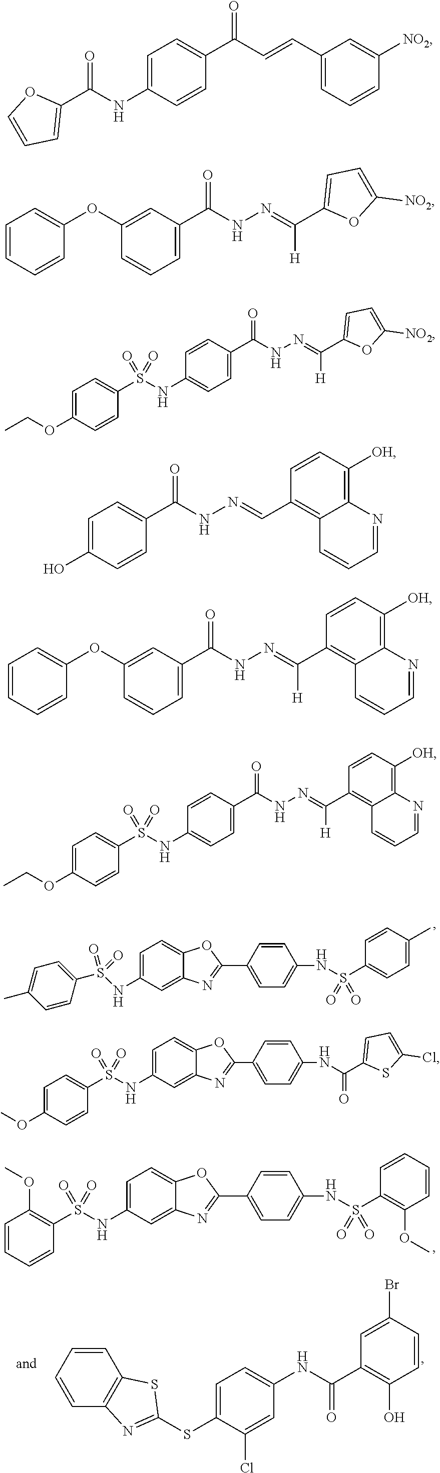
or a pharmaceutically acceptable salt thereof.

19. The method of claim 1, wherein the compound is provided in a composition comprising a pharmaceutically acceptable excipient.

20. The method of claim 1, wherein the cancer is selected from the group consisting of colorectal cancer, lung cancer, prostate cancer, renal cancer, blood cancer, skin cancer, ovarian cancer, breast cancer, CNS cancer, cervical cancer, and gastric cancer.
